(19) United States
(12) Patent Application Publication
(10) Pub. No.: US 2005/0147611 A1
(43) Pub. Date: Jul. 7, 2005

(54) Combination therapy for conditions leading to bone loss

(75) Inventors: William J. Boyle, Moorpark, CA (US); David Lee Lacey, Newbury Park, CA (US); Frank J. Calzone, Westlake Village, CA (US); Ming-Shi Chang, Tainan (TW); Giorgio Senaldi, Thousand Oaks, CA (US)

Correspondence Address: AMGEN INC., MAIL STOP 27-4-A, ONE AMGEN CENTER DRIVE, THOUSAND OAKS, CA, 91320-1799, US

(73) Assignee: Amgen Inc.

(21) Appl. No.: 11/058,073

(22) Filed: Feb. 14, 2005

Related U.S. Application Data
(62) Division of application No. 09/613,591, filed on Jul. 10, 2000.
Application No. 09/613,591 is a continuation-in-part of application No. 09/350,670, filed on Jul. 9, 1999, now abandoned.
Application No. 09/350,670 is a continuation-in-part of application No. 08/706,945, filed on Sep. 3, 1996, now Pat. No. 6,369,027.
Application No. 08/706,945 is a continuation-in-part of application No. 08/577,788, filed on Dec. 22, 1995, now Pat. No. 6,613,544.

(51) Int. Cl. A61K 39/395; A61K 38/17

(52) U.S. Cl. 424/145.1; 514/12

(57) ABSTRACT

The present invention discloses a novel secreted polypeptide, termed osteoprotegerin, which is a member of the tumor necrosis factor receptor superfamily and is involved in the regulation of bone metabolism. Also disclosed are nucleic acids encoding osteoprotegerin, polypeptides, recombinant vectors and host cells for expression, antibodies which bind OPG, and pharmaceutical compositions. The polypeptides are used to treat bone diseases characterized by increased resorption such as osteoporosis. Methods of treatment are described using the polypeptides in conjunction with various agents, including IL-1 inhibitors, TNF-α inhibitors, and serine protease inhibitors.

### CROSS-REFERENCE TO RELATED APPLICATIONS

This application is a divisional of U.S. Ser. No. 09/613,591 filed Jul. 10, 2000, which is a continuation-in-part of U.S. Ser. No. 09/350,670 filed Jul. 9, 1999, which is a continuation-in-part (CIP) of U.S. Ser. No. 08/706,945, filed on Sep. 3, 1996, which in turn is a CIP of U.S. Ser. No. 08/577,788, filed Dec. 22, 1995. Each of the foregoing applications is hereby incorporated by reference.

### FIELD OF THE INVENTION

The invention relates generally to polypeptides involved in the regulation of bone metabolism. More particularly, the invention relates to a novel polypeptide, termed osteoprotegerin, which is a member of the tumor necrosis factor receptor superfamily. The polypeptide is used to treat bone diseases characterized by increased bone loss such as osteoporosis and arthritis.

### BACKGROUND OF THE INVENTION

Polypeptide growth factors and cytokines are secreted factors which signal a wide variety of changes in cell growth, differentiation, and metabolism, by specifically binding to discrete, surface bound receptors. As a class of proteins, receptors vary in their structure and mode of signal transduction. They are characterized by having an extracellular domain that is involved in ligand binding, and cytoplasmic domain which transmits an appropriate intracellular signal. Receptor expression patterns ultimately determine which cells will respond to a given ligand, while the structure of a given receptor dictates the cellular response induced by ligand binding. Receptors have been shown to transmit intracellular signals via their cytoplasmic domains by activating protein tyrosine, or protein serine/threonine phosphorylation (e.g., platelet derived growth factor receptor (PDGFR) or transforming growth factory-β receptor-I (TGFβR-I), by stimulating G-protein activation (e.g., β-adrenergic receptor), and by modulating associations with cytoplasmic signal transducing proteins (e.g., TNFR-I and Fas/APO) (Heldin, Cell 80, 213-223 (1995)).

The tumor necrosis factor receptor (TNFR) superfamily is a group of type I transmembrane proteins which share a conserved cysteine-rich motif which is repeated three to six times in the extracellular domain (Smith, et al. Cell 76, 953-962 (1994)).

Collectively, these repeat units form the ligand binding domains of these receptors (Chen et al., Chemistry 270, 2874-2878 (1995)). The ligands for these receptors are a structurally related group of proteins homologous to TNFα. (Goeddel et al. Cold Spring Harbor Symp. Quart. Biol. 51, 597-609 (1986); Nagata et al. Science 267, 1449-1456 (1995)). TNFα binds to distinct, but closely related receptors, TNFR-I and TNFR-II. TNFα produces a variety of biological responses in receptor bearing cells, including, proliferation, differentiation, and cytotoxicity and apoptosis (Beutler et al. Ann. Rev. Biochem. 57, 505-518 (1988)).

TNFα is believed to mediate acute and chronic inflammatory responses (Beutler et al. Ann. Rev. Biochem. 57, 505-508 (1988)). Systemic delivery of TNFα induces toxic shock and widespread tissue necrosis. Because of this, TNFα may be responsible for the severe morbidity and mortality associated with a variety of infectious diseases, including sepsis. Mutations in FasL, the ligand for the TNFR-related receptor Fas/APO (Suda et al. Cell 75, 1169-1178 (1993)), is associated with autoimmunity (Fisher et al. Cell 81, 935-946 (1995)), while overproduction of FasL may be implicated in drug-induced hepatitis. Thus, ligands to the various TNFR-related proteins often mediate the serious effects of many disease states, which suggests that agents that neutralize the activity of these ligands would have therapeutic value. Soluble TNFR-I receptors, and antibodies that bind TNFα, have been tested for their ability to neutralize systemic TNFα (Loetscher et al. Cancer Cells 3(6), 221-226 (1991)). A naturally occurring form of a secreted TNFR-I mRNA was cloned, and its product tested for its ability to neutralize TNFα activity in vitro and in vivo (Kohno et al. PNAS USA 87, 8331-8335 (1990)). The ability of this protein to neutralize TNFα suggests that soluble TNF receptors function to bind and clear TNF thereby blocking the cytotoxic effects on TNFR-bearing cells.

An object of the invention is to identify new members of the TNFR superfamily. It is anticipated that new family members may be transmembrane proteins or soluble forms thereof comprising extracellular domains and lacking transmembrane and cytoplasmic domains. We have identified a new member of the TNFR superfamily which encodes a secreted protein that is closely related to TNFR-II. By analogy to soluble TNFR-II, the TNFR-II related protein may negatively regulate the activity of its ligand, and thus may be useful in the treatment of certain human diseases.

A further object of this invention is new methods of treatment of inflammatory diseases and medical conditions.

### SUMMARY OF THE INVENTION

A novel member of the tumor necrosis factor receptor (TNFR) superfamily has been identified from a fetal rat intestinal cDNA library. A full-length cDNA clone was obtained and sequenced. Expression of the rat cDNA in a transgenic mouse revealed a marked increase in bone density, particularly in long bones, pelvic bone and vertebrae. The polypeptide encoded by the cDNA is termed Osteoprotegerin (OPG) and plays a role in promoting bone accumulation.

The invention provides for nucleic acids encoding a polypeptide having at least one of the biological activities of OPG. Nucleic acids which hybridize to nucleic acids encoding mouse, rat or human OPG as shown in FIGS. 2B-2C (SEQ ID NO: 120), 9A-9B (SEQ ID NO: 122), and 9C-9D (SEQ ID NO: 124) are also provided. Preferably, OPG is mammalian OPG and more preferably is human OPG. Recombinant vectors and host cells expressing OPG are also encompassed as are methods of producing recombinant OPG. Antibodies or fragments thereof which specifically bind the polypeptide are also disclosed.

Methods of treating bone diseases are also provided by the invention. The polypeptides are useful for preventing bone resorption and may be used to treat any condition resulting in bone loss such as osteoporosis, hypercalcemia, Paget's disease of bone, and bone loss due to rheumatoid arthritis or osteomyelitis, and the like. Bone diseases may also be treated with anti-sense or gene therapy using nucleic acids of the invention. Pharmaceutical compositions comprising OPG nucleic acids and polypeptides are also encompassed.

The invention relates further to treatment of diseases using combination therapy. In particular, the novel polypeptides described herein may be used in conjunction with bone morphogenic proteins BMP-1 through BMP-12; TGF-β and TGF-β family members; IL-1 inhibitors; TNF-α inhibitors; parathyroid hormone and analogs thereof; parathyroid related protein and analogs thereof; E series prostaglandins; bisphosphonates; bone-enhancing minerals; NSAIDs; immunosuppressants; serine protease inhibitors; IL-6 inhibitors; IL-8 inhibitors (e.g., antibodies to IL-8); IL-18 inhibitors; ICE modulators; FGF-1 to FGF-10; FGF modulators; PAF antagonists; KGF, KGF-related molecules, or KGF modulators; MMP modulators; NOS modulators; modulators of glucocorticoid receptor; modulators of glutamate receptor; modulators of LPS levels; and noradrenaline and modulators and mimetics thereof.

### DESCRIPTION OF THE FIGURES

FIG. 1. A. FASTA analysis of novel EST LORF. Shown is the deduced FRI-1 amino acid sequence aligned to the human TNFR-II sequence. B. Profile analysis of the novel EST LORF shown is the deduced FRI-1 amino acid sequence aligned to the TNFR-profile. C. Structural view of TNFR superfamily indicating region which is homologous to the novel FRI-1.

FIG. 2. Structure and sequence of full length rat OPG gene, a novel member of the TNFR superfamily. A. Map of pMOB-B 1.1 insert. Box indicates position of LORF within the cDNA sequence (bold line). Black box indicates signal peptide, and gray ellipses indicate position of cysteine-rich repeat sequences. B, C. Nucleic acid and protein sequence of the Rat OPG cDNA. The predicted signal peptide is underlined, and potential sites of N-linked glycosylation are indicated in bold, underlined letters. D, E. Pileup sequence comparison (Wisconsin GCG Package, Version 8.1) of OPG with other members of the TNFR superfamily, fas (SEQ ID NO: 128); tnfr1 (SEQ ID NO: 129); sfu-t2 (SEQ ID NO: 130); tnfr2 (SEQ ID NO: 131); cd40 (SEQ ID NO: 132); osteo (SEQ ID NO: 133); ngfr (SEQ ID NO: 134); ox40 (SEQ ID NO: 135); 41bb (SEQ ID NO: 136).

FIG. 3. PepPlot analysis (Wisconsin GCG Package, Version 8.1) of the predicted rat OPG sequence. A. Schematic representation of rat OPG showing hydrophobic (up) and hydrophilic (down) amino acids. Also shown are basic (up) and acidic (down) amino acids. B. Display of amino acid residues that are beta-sheet forming (up) and beta-sheet breaking down) as defined by Chou and Fasman (Adv. Enz. 47, 45-147 (1948)). C. Display of propensity measures for alpha-helix and beta-sheet (Chou and Fasman, ibid). Curves above 1.00 show propensity for alpha-helix or beta-sheet structure. Structure may terminate in regions of protein where curves drop below 1.00. D. Display of residues that are alpha-forming (up) or alpha-breaking (down). E. Display of portions of the protein sequence that resemble sequences typically found at the amino end of alpha and beta structures (Chou and Fasman, ibid). F. Display of portions of the protein sequence that resemble sequences typically found at the carboxyl end of alpha and beta structures (Chou and Fasman, ibid). G. Display of portions of the proteins sequence typically found in turns (Chou and Fasman, ibid) H. Display of the helical hydrophobic moment (Eisenberg et al. Proc. Natl. Acad. Sci. USA 81, 140-144 (1984)) at each position in the sequence. I. Display of average hydrophathy based upon Kyte and Doolittle (J. Mol. Biol. 157, 105-132 (1982)) and Goldman et al. (reviewed in Ann. Rev. Biophys. Biophys. Chem. 15, 321-353 (1986)).

FIG. 4. mRNA expression patterns for the OPG cDNA in human tissues. Northern blots were probed with a 32P-labeled rat cDNA insert (A, left two panels), or with the human cDNA insert (B, right panel).

FIG. 5. Creation of transgenic mice expressing the OPG cDNA in hepatocytes. Northern blot expression of HE-OPG transgene in mouse liver.

FIG. 6. Increase in bone density in OPG transgenic mice. Panel A-F. Control Mice. G-J, OPG expressing mice. At necropsy, all animals were radiographed and photographs prepared. In A-F, the radiographs of the control animals and the one transgenic non-expressor (#28) are shown. Note that the bones have a clearly defined cortex and a lucent central marrow cavity. In contrast, the OPG (G-J) animals have a poorly defined cortex and increased density in the marrow zone.

FIG. 7. Increase in trabecular bone in OPG transgenic mice. A-D. Representative photomicrographs of bones from control animals. In A and B, low (4×, 10×) power images of the femurs are shown (Masson Trichrome stain). Stains for tartrate resistant acid phosphatase (TRAP) demonstrate osteoclasts (see arrows) both resorbing cartilage (C) and trabecular bone (D). Note the flattened appearance of osteoclasts on trabecular bone. E-H. Representative photomicrographs of bones from OPG-expressing animals. In E and F, low (4×, 10×) power images of the femurs are shown (Masson Trichrome stain). The clear region is the growth plate cartilage, blue stained area is bone, and the red area is marrow. Note that in contrast to the controls, the trabecular bone has not been resorbed resulting in the absence of the usual marrow cavity. Also, the resulting trabeculae have a variegated appearance with blue and clear areas. The clear areas are remnants of growth plate cartilage that have never been remodelled. Based on TRAP stains, these animals do have osteoclasts (see arrows) at the growth plate (G), which may be reduced in number. However, the surfaces of the trabeculae away from the growth plate are virtually devoid of osteoclasts (H), a finding that stands in direct contrast with the control animals (see D).

FIG. 8. HE-OPG expressors do not have a defect in monocyte-macrophage development. One cause for osteopetrosis in mice is defective M-CSF production due to a point mutation in the M-CSF gene. This results in a marked deficit of circulating and tissue based macrophages. The peripheral blood of OPG expressors contained monocytes as assessed by H1E analysis. To affirm the presence of tissue macrophages, immnohistochemistry was performed using F480 antibodies, which recognize a cell surface antigen on murine macrophages. A and C show low power (4×) photomicrographs of the spleens from normal and CR1 overexpressors. Note that both animals have numerous F480 positive cells. Monocyte-macrophages were also present in the marrow of normal (B) and HE-OPG overexpressors (D) (40×).

FIG. 9. Structure and sequence of mouse and human OPG cDNA clones. A, B. Mouse cDNA and protein sequence. C, D. Human cDNA and protein sequence. The predicted signal peptides are underlined, and potential sites of N-linked glycosylation are indicated in bold. E, F. Sequence alignment and comparison of rat, mouse and human OPG amino acid sequences.

FIG. 10. Comparison of conserved sequences in extracellular domain of TNFR-I and human OPG. PrettyPlot (Wisconsin GCG Package, Version 8.1) of the TNFR1 and OPG alignment described in example 6. Top line, human TNFR1 sequences encoding domains 1-4. Bottom line, human OPG sequences encoding domains 1-4. Conserved residues are highlighted by rectangular boxes.

FIG. 11. Three-dimensional representation of human OPG. Side-view of the Molescript display of the predicted 3-dimensional structure of human OPG residues 25 through 163, (wide line), co-crystallized with human TNF□ (thin line). As a reference for orientation, the bold arrows along the OPG polypeptide backbone are pointing in the N-terminal to C-terminal direction. The location of individual cysteine residue side chains are inserted along the polypeptide backbone to help demonstrate the separate cysteine-rich domains. The TNF□ molecule is aligned as described by Banner et al. (1993).

FIG. 12. Structure of OPG cysteine-rich domains. Alignment of the human (top line SEQ ID NO: 136) and mouse (bottom line) OPG amino acid sequences highlighting the predicted domain structure of OPG. The polypeptide is divided into two halves; the N-terminus (A), and C-terminus (B). The N-terminal half is predicted to contain four cysteine rich domains (labeled 1-4). The predicted intrachain disulfide bonds are indicated by bold lines, labeled “SS1”, “SS2”, or “SS3”. Tyrosine 28 and histidine 75 (underlined) are predicted to form an ionic interaction. Those amino acids predicted to interact with an OPG ligand are indicated by bold dots above the appropriate residue. The cysteine residues located in the C-terminal half of OPG are indicated by rectangular boxes.

FIG. 13. Expression and secretion of full length and truncated mouse OPG-Fc fusion proteins. A. Map indicating points of fusion to the human IgG1 Fc domain are indicated by arrowheads. B. Silver stain of a SDS-polyacrylamide gel of conditioned media obtained from cells expressing either Fl.Fc (Full length OPG fused to Fc at Leucine 401) or CT.Fc (Carboxy-terminal truncated OPG fused to Fc at threonine 180) fusion protein expression vectors. Lane 1, parent pCEP4 expression vector cell line; Lane 2, Fl.Fc vector cell line; Lane 3, CT.Fc vector cell line. C. Western blot of conditioned media obtained from Fl.Fc and CT.Fc fusion protein expression vectors probed with anti-human IgG1 Fc domain (Pierce). Lane 1, parent pCEP4 expression vector cell line; Lane 2, Fl.Fc vector cell line; Lane 3, CT.Fc vector cell line.

FIG. 14. Expression of human OPG in E. coli. A. Construction of a bacterial expression vector. The LORF of the human OPG gene was amplified by PCR, then joined to a oligonucleotide linker fragment (top strand is SEQ ID NO: 137; bottom strand is SEQ ID NO: 127), and ligated into pAMG21 vector DNA. The resulting vector is capable of expressing OPG residues 32-401 linked to a N-terminal methionine residue. B SDS-PAGE analysis of uninduced and induced bacterial harboring the pAMG21-human OPG-32-401 plasmid. Lane 1, MW standards; lane 2, uninduced bacteria; lane 3, 30° C. induction; lane 4, 37° C. induction; lane 5, whole cell lysate from 37° C. induction; lane 6, soluble fraction of whole cell lysate; lane 7, insoluble fraction of whole cell lysate; lane 8, purified inclusion bodies obtained from whole cell lysate.

FIG. 15. Analysis of recombinant murine OPG produced in CHO cells by SDS-PAGE and western blotting. An equal amount of CHO conditioned media was applied to each lane shown, and was prepared by treatment with either reducing sample buffer (left lane), or non-reducing sample buffer (right lane). After electrophoresis, the resolved proteins were transferred to a nylon membrane, then probed with anti-OPG antibodies. The relative positions of the 55 kd monomeric and 100 kd dimeric forms of OPG are indicated by arrowheads.

FIG. 16. Pulse-chase analysis of recombinant murine OPG produced in CHO cells. CHO cells were pulse-labeled with ³⁵S-methionine/cysteine, then chased for the indicated time. Metabolically labeled cultures were separated into both conditioned media and cells, and detergent extracts were prepared from each, clarified, then immunoprecipitated with anti-OPG antibodies. The immunoprecipitates were the resolved by SDS-PAGE, and exposed to film. Top left and right panels; samples analyzed under non-reducing conditions. Lower left and right panels; samples analyzed under reducing conditions. Top and bottom left panels; Cell extracts. Top and bottom right panels; Conditioned media extracts. The relative mobility of the 55 kd monomeric and 100 kd dimeric forms of OPG are indicated by arrowheads.

FIG. 17. Expression of OPG in the CTLL-2 cell line. Serum-free conditioned media from CTLL-2 cells and CHO-mu OPG [1-401] transfected cells was prepared, concentrated, then analyzed by non-reducing SDS-PAGE and western blotting. Left lane; CTLL-2 conditioned media. Right lane; CHO-muOPG conditioned media. The relative mobility of the 55 kd monomeric and 100 kd dimeric forms of OPG are indicated by arrowheads.

FIG. 18. Detection of OPG expression in serum samples and liver extracts obtained from control and OPG transgenic mice. Transgenic mice were constructed as described in Example 4. OPG expression was visualized after SDS-PAGE followed by Western blotting using anti-OPG antibodies.

FIG. 19. Effects of huOPG [22-401]-Fc fusion protein on osteoclast formation in vitro. The osteoclast forming assay was performed as described in Example 11A in the absence (control) or presence of the indicated amounts of huOPG [22-401]-Fc fusion. Osteoclast formation was visualized by histochemical staining for tartrate acid phosphatase (TRAP). ). A. OPG added to 100 ng/ml. D. OPG added to 0.1 ng/ml. E. OPG added to 0.01 ng/ml. F. OPG added to 0.001 ng/ml. G. Control. No OPG added.

FIG. 20. Decrease in osteoclast culture TRAP activity with increasing amounts of OPG. Indicated concentrations of huOPG [22-401]-Fc fusion protein were added to osteoclast forming assay and TRAP activity quantitated as described in Example 11A.

FIG. 21. Effect of OPG on a terminal stage of osteoclast differentiation. huOPG [22-401]-Fc fusion was added to the osteoclast forming assay during the intermediate stage of osteoclast maturation (days 5-6; OPG-CTL) or during the terminal stage of osteoclast maturation (days 7-15; CTL-OPG). TRAP activity was quantitated and compared with the activity observed in the absence of OPG (CTL-CTL) in the presence of OPG throughout (OPG-OPG).

FIG. 22. Effects of IL-1β, IL-1α and OPG on blood ionized calcium in mice. Levels of blood ionized calcium were monitored after injection of IL-1β alone, IL-1α alone, IL-1β plus muOPG [2240 1]-Fc, IL-1α plus MuOPG [22-401]-Fc, and muOPG [22-401]-Fc alone. Control mice received injections of phosphate buffered saline (PBS) only. IL-1β experiment shown in A; IL-1α experiment shown in B.

FIG. 23. Effects of OPG on calvarial osteoclasts in control and IL-1-treated mice. Histological methods for analyzing mice calvarial bone samples are described in Example 11B. Arrows indicate osteoclasts present in day 2-treated mice. Calvarial samples of mice receiving four PBS injections daily (A), one injection of IL-1 and three injections of PBS daily (B), one injection of PBS and three injections of OPG daily (C), one injection of IL-1 and three injections of OPG daily.

FIG. 24. Radiographic analysis of bone accumulation in marrow cavity of normal mice. Mice were injected subcutaneously with saline (A) or muOPG [22-401]-Fc fusion (5mg/kg/d) for 14 days (B) and bone density determined as described in Example 11C.

FIG. 25. Histomorphometric analysis of bone accumulation in marrow cavity of normal mice. Injection experiments and bone histology performed as described in Example 11C.

FIG. 26. Histology analysis of bone accumulation in marrow cavity of normal mice. Injection experiments and bone histology performed as described in Example 11C. A. Saline injection B. Injection of muOPG [22-401]-Fc fusion.

FIG. 27. Activity of OPG administered to ovariectomized rats. In this two week experiment the trend to reduced bone density appears to be blocked by OPG or other anti-resorptive therapies. DEXA measurements were taken at time of ovariectomy and at week 1 and week 2 of treatment. The results are expressed as % change from the initial bone density (Mean±SEM).

FIG. 28. Bone density in the femoral metaphysis, measured by histomorphometric methods, tends to be lower in ovariectomized rats (OVX) than sham operated animals (SHAM) 17 days following ovariectomy. This effect was blocked by OPG-Fc, with OPG-Fc treated ovariectomized rats (OVX+OPG) having significantly higher bone density than vehicle treated ovariectomized rats (OVX). (Mean±SEM).

FIG. 29A through 29G. Sequence of OPG-Fc. DNA and encoded protein sequences are shown. Restriction sites for various nucleases are noted above the DNA sequence.

FIGS. 30A through 30D. Effects of OPG-Fc during the course of adjuvant arthritis I male Lewis rats. Paws from rats with adjuvant arthritis induced by 0.5 mg mycobacteria in oil were analyzed by DEXA for bone mineral density (BMD). Evaluation of BMD, a 29 mm×25 mm box was centered at the calcaneus (expt AdA-14 2/99, Amgen nb#22957 p4749). * compared to normal, # compared to vehicle P<0.05 Mann-Whitney U test.

FIGS. 31A and 31B. Combination treatment with OPG-Fc and sTNF-RI on Adjuvant Arthritis in Male Lewis Rats. Area under the curve (AUC) for measurement of paw swelling and BMD were measured as described above for FIG. 33 and in the examples hereinafter.

### DETAILED DESCRIPTION OF THE INVENTION

OPG Proteins

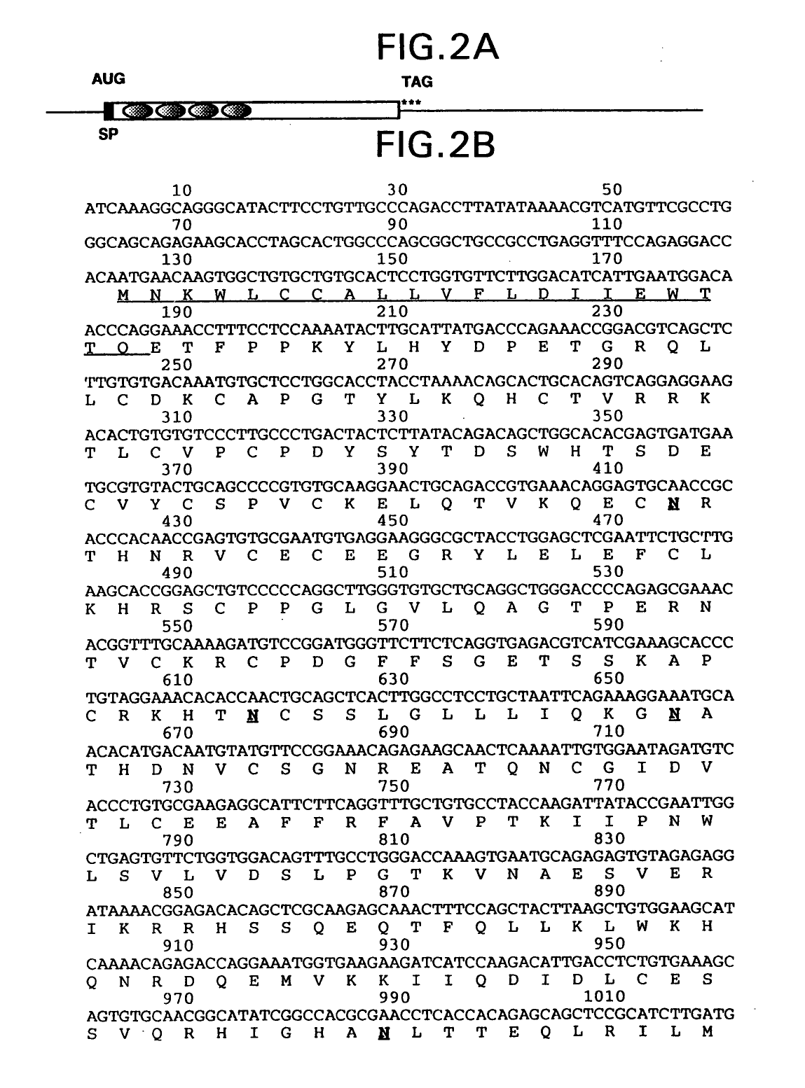

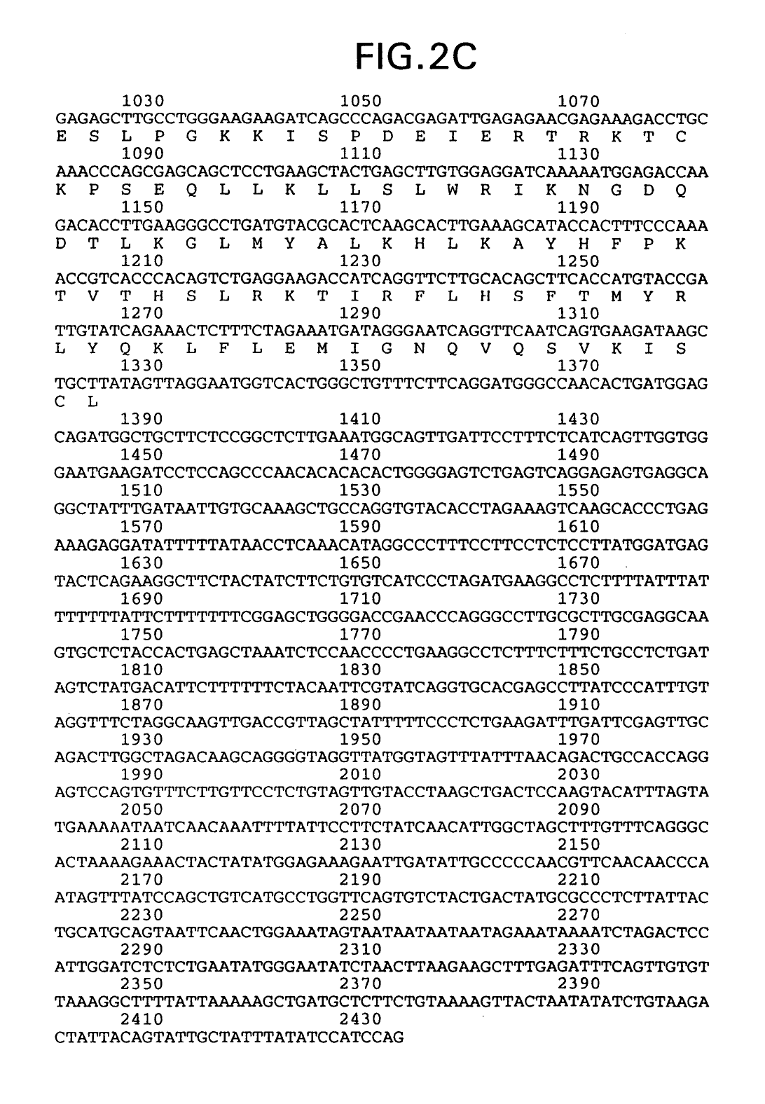

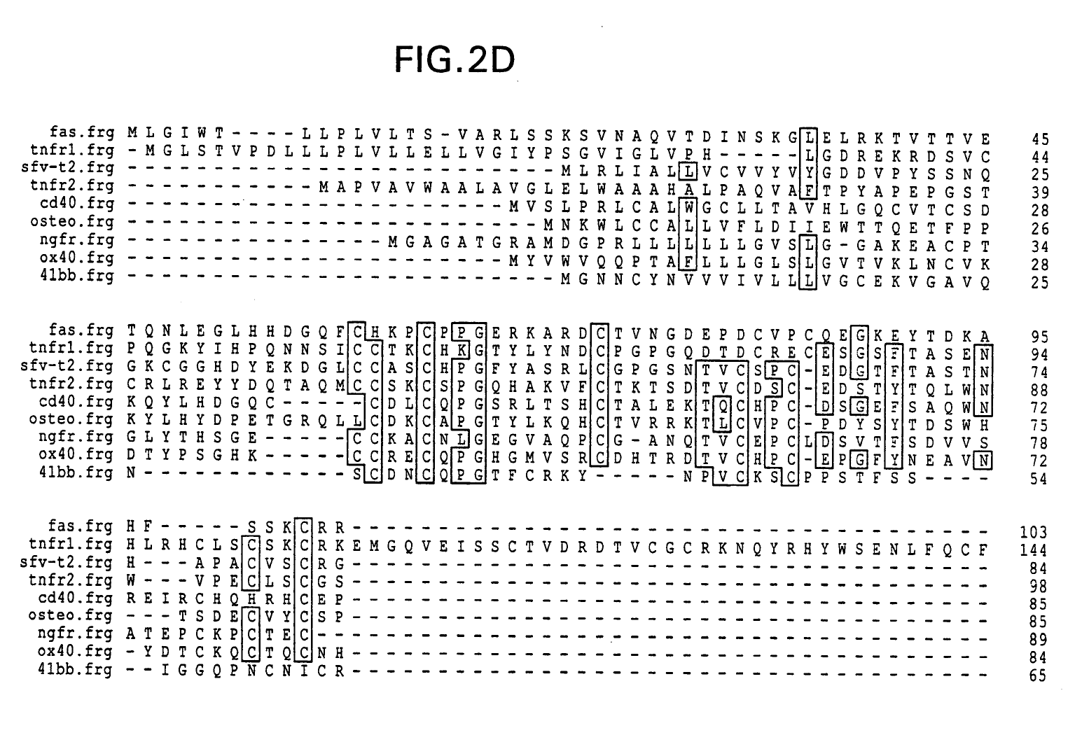

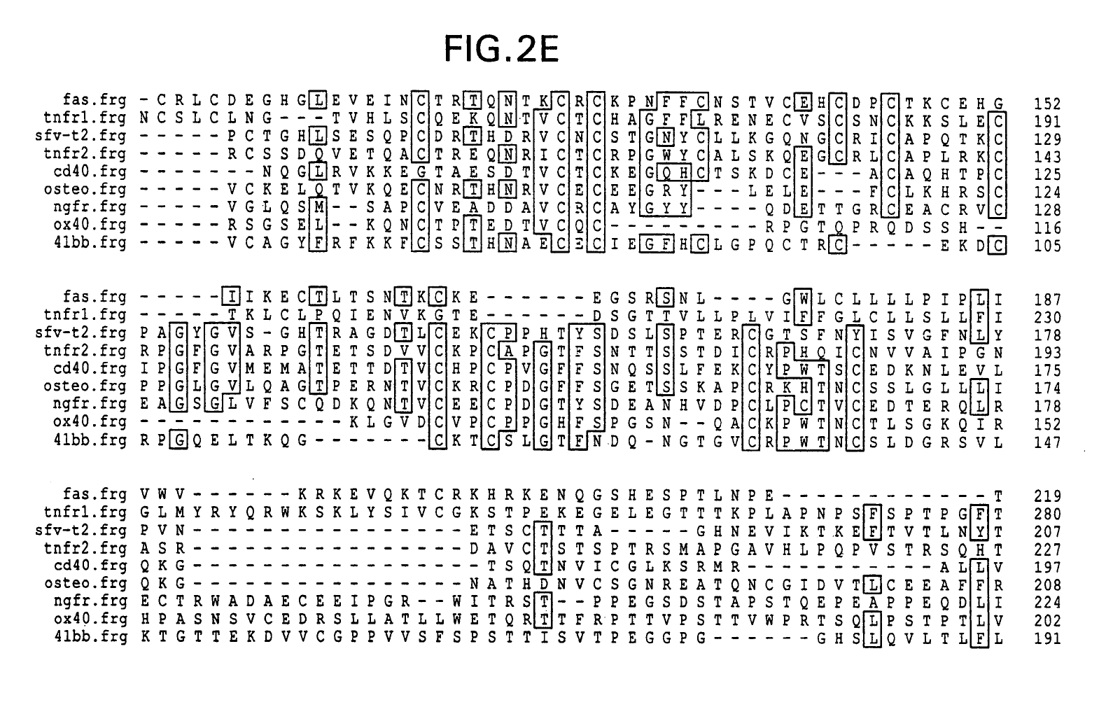

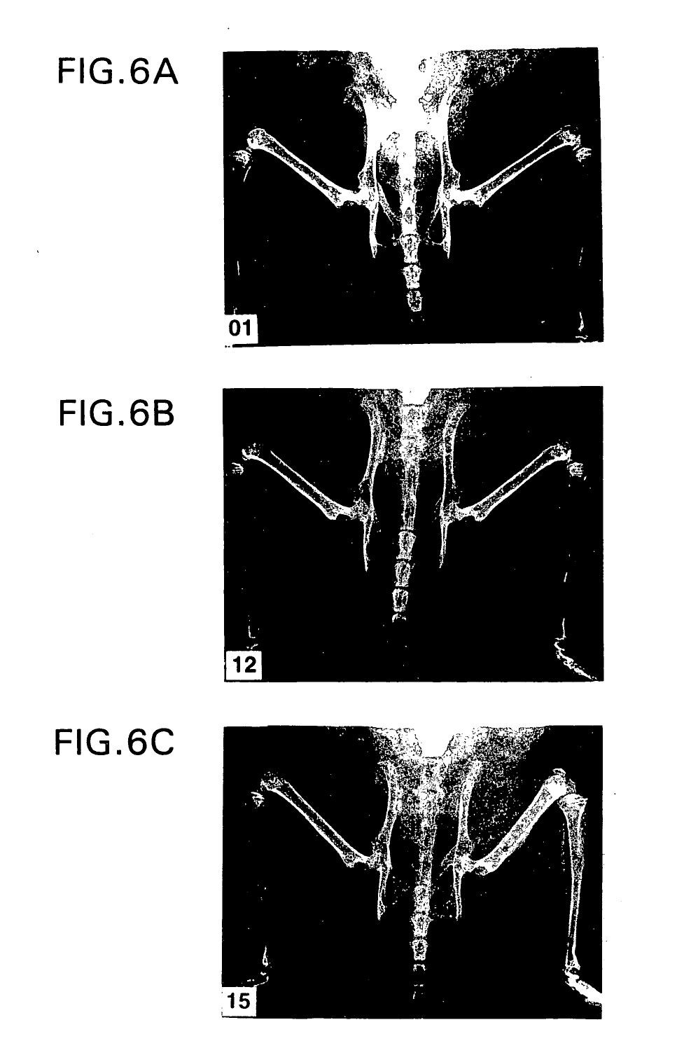

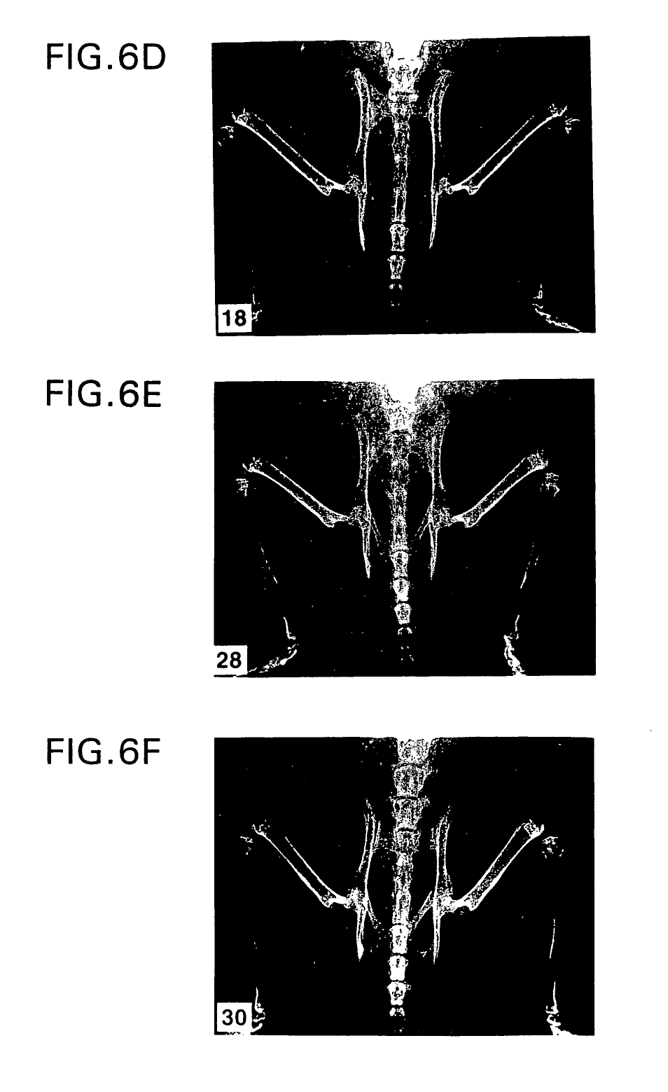

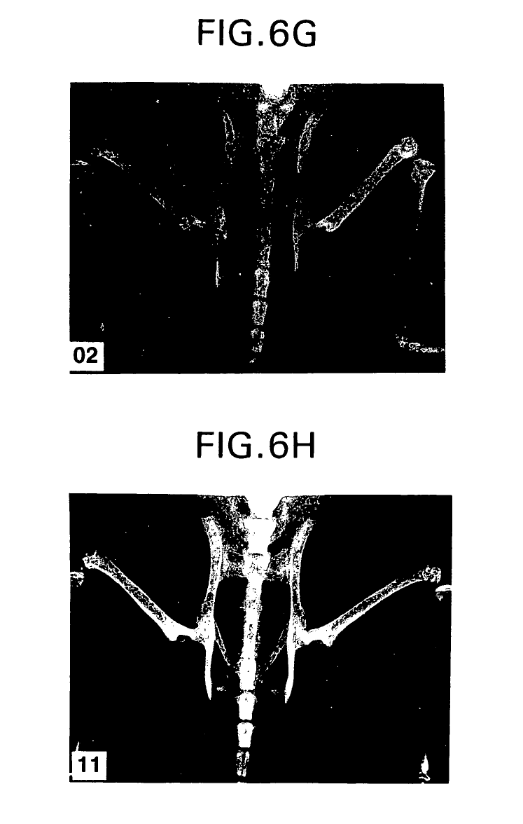

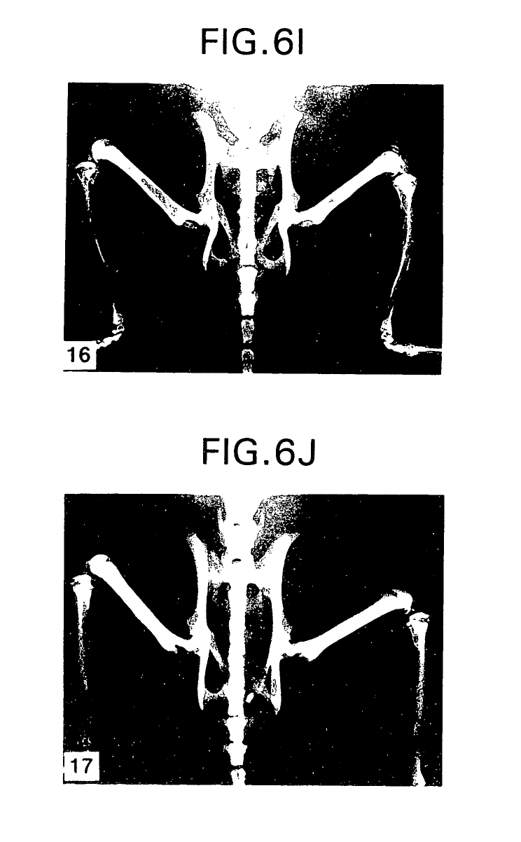

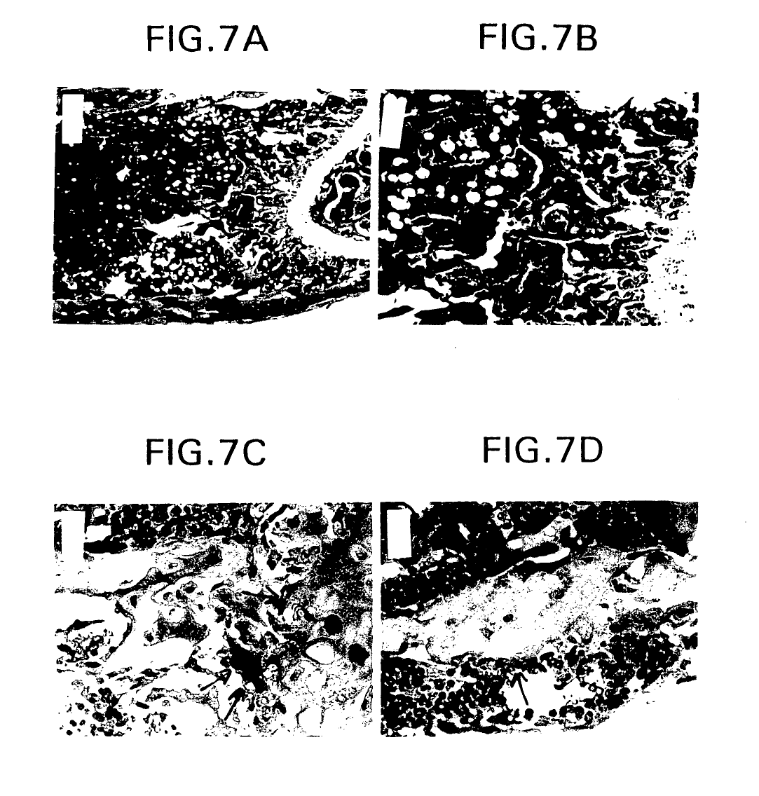

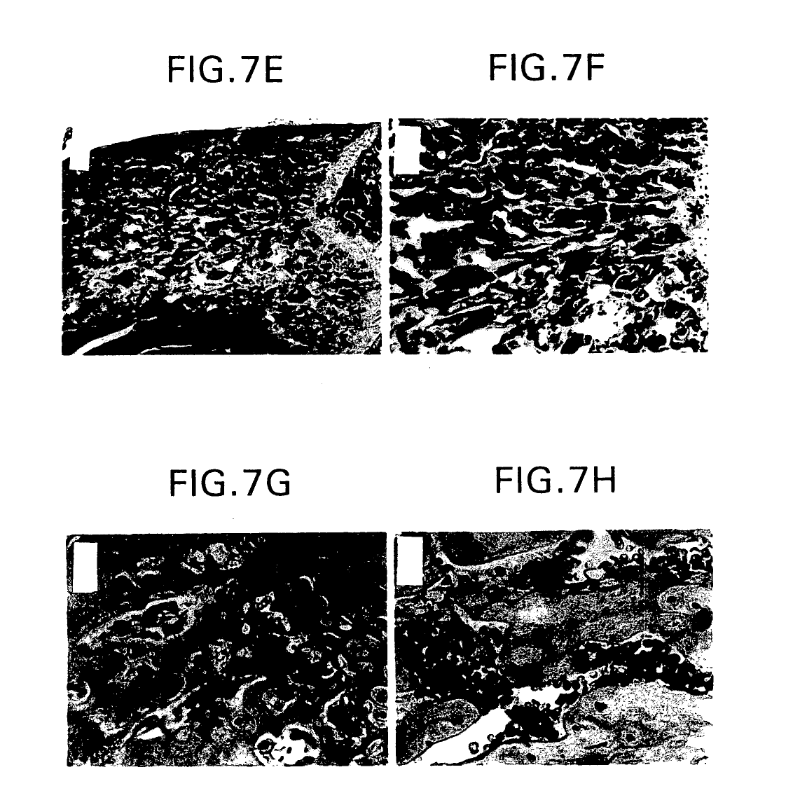

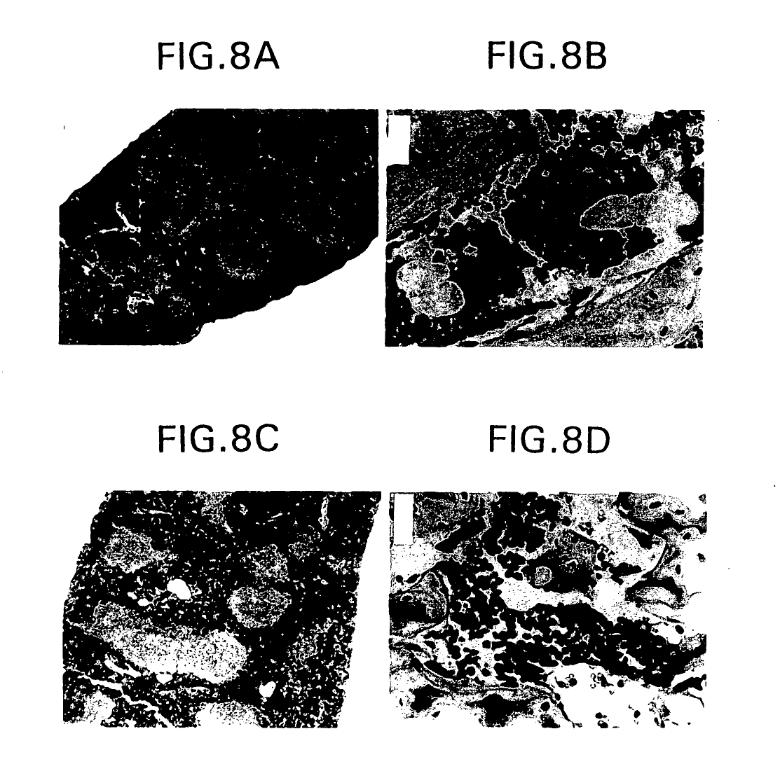

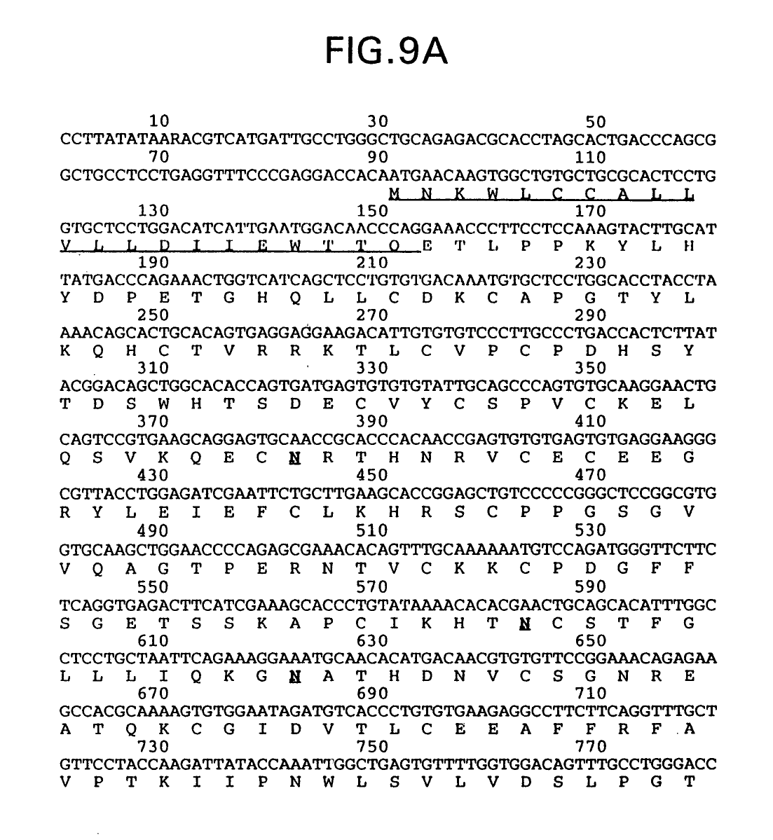

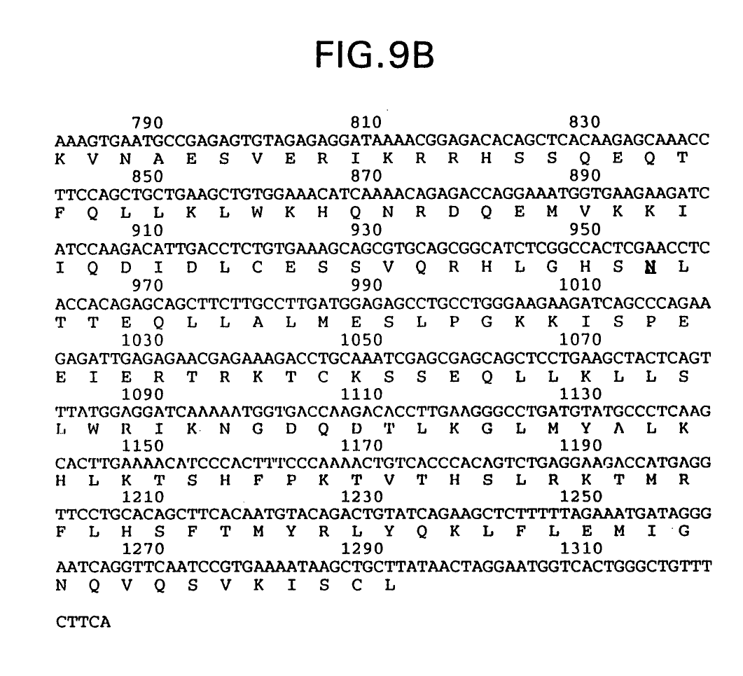

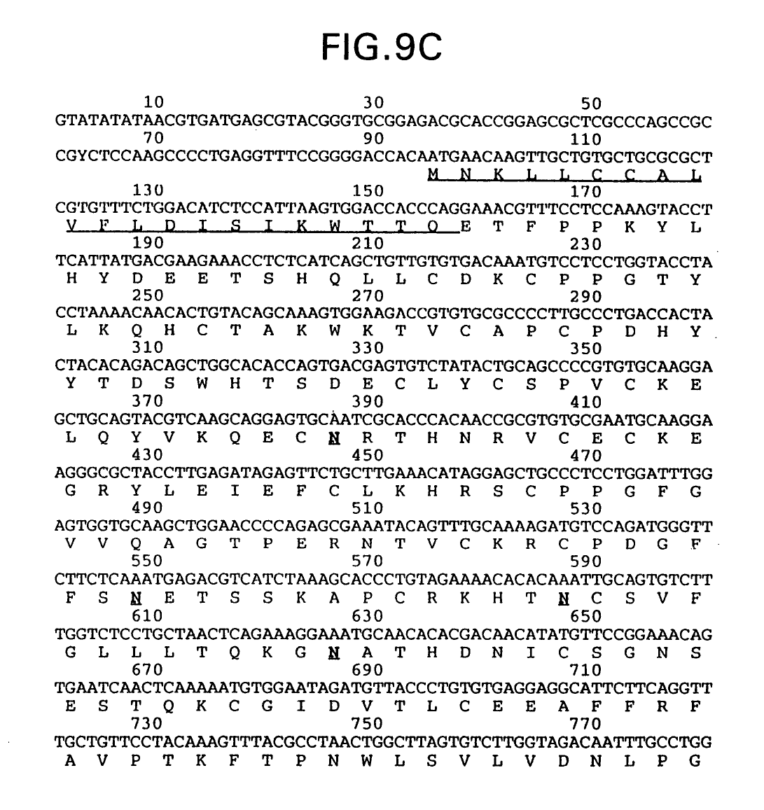

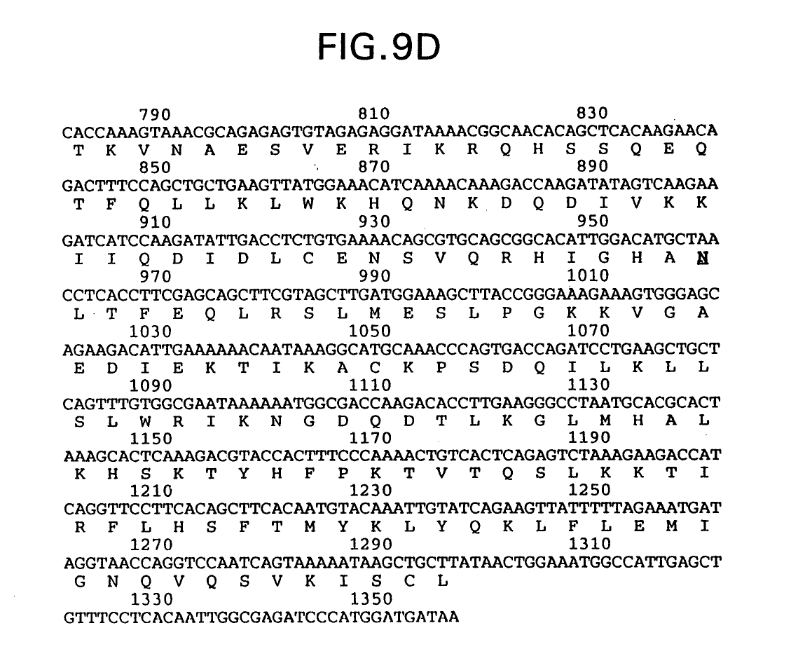

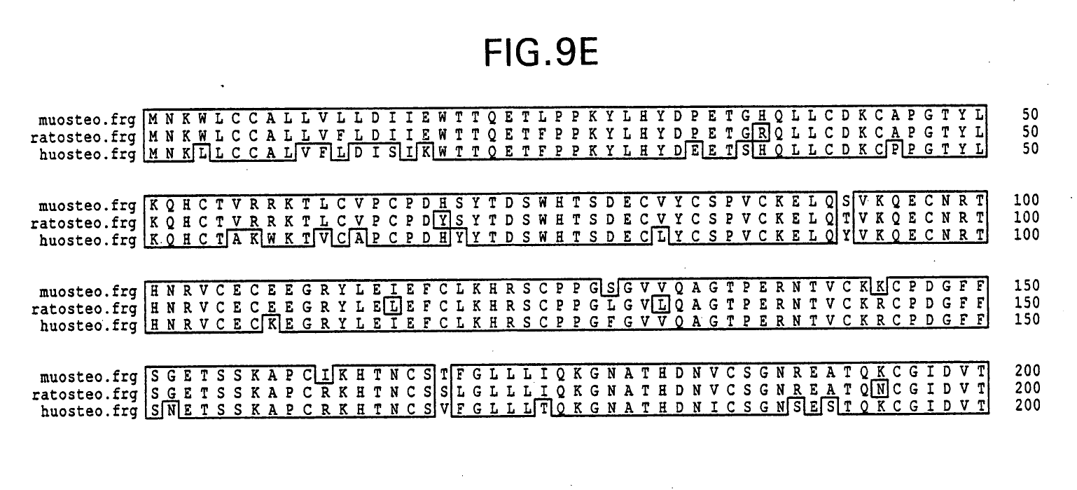

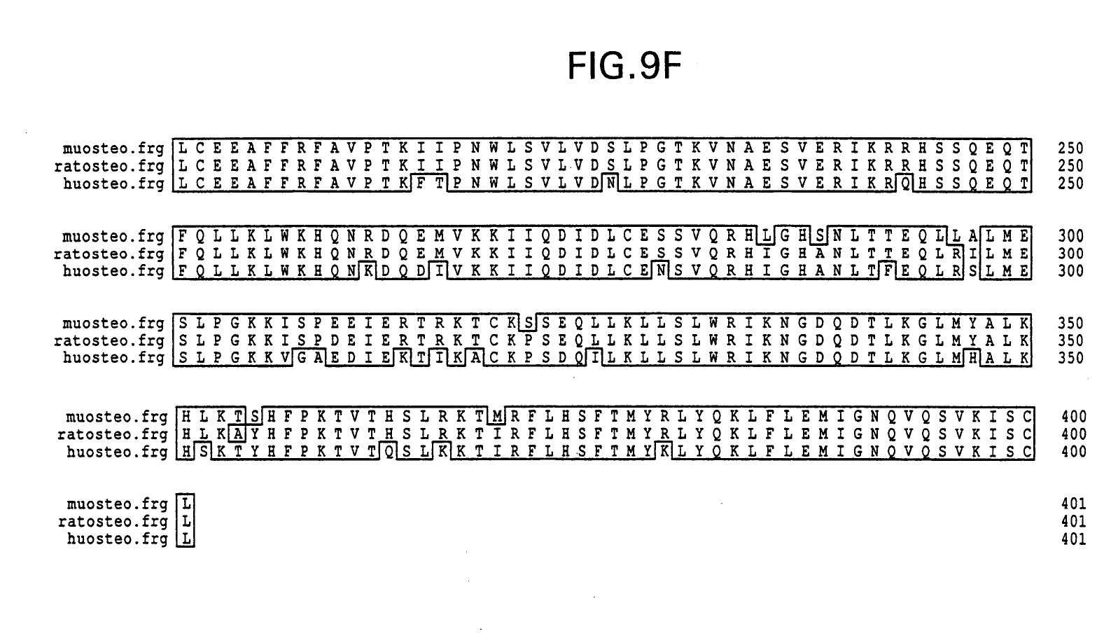

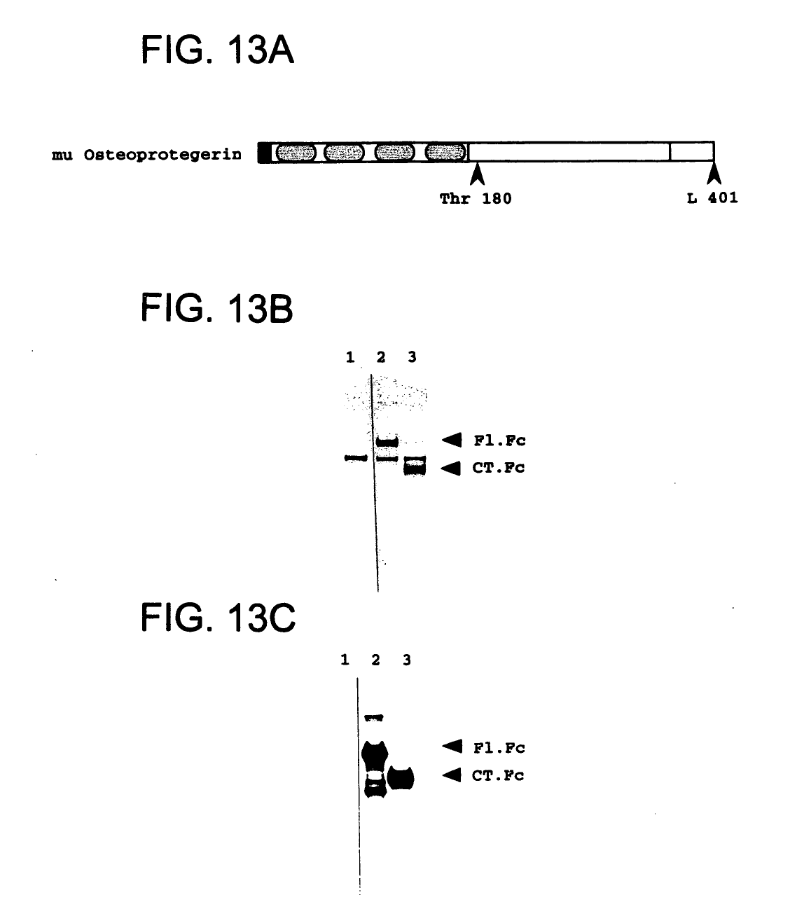

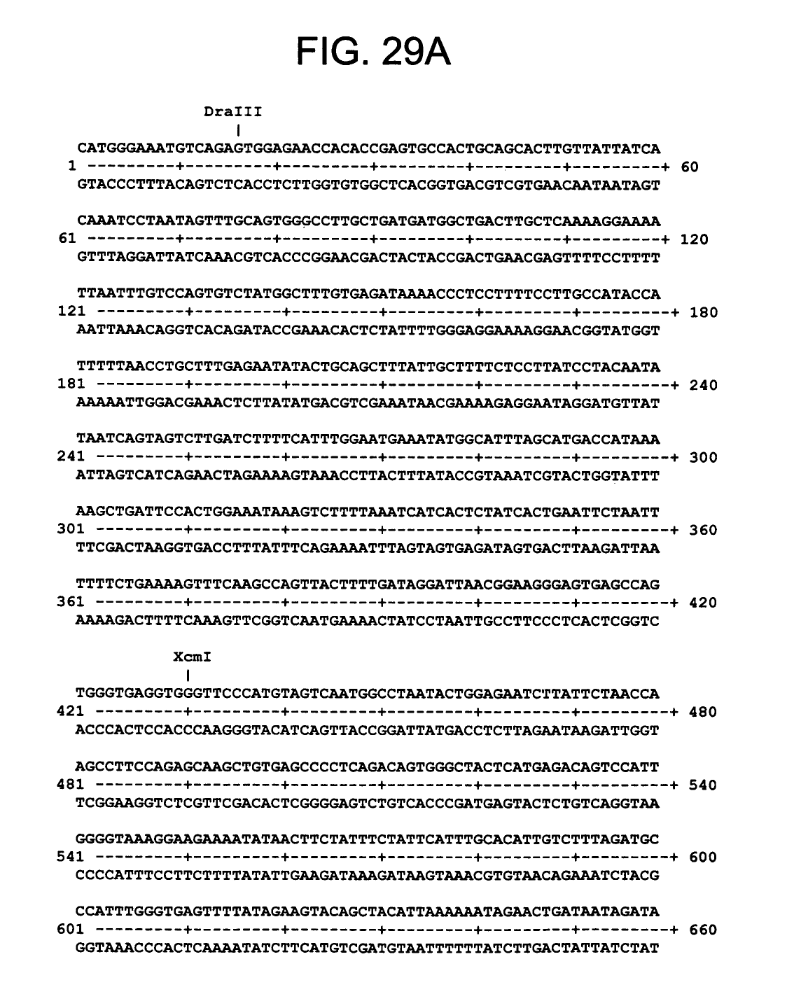

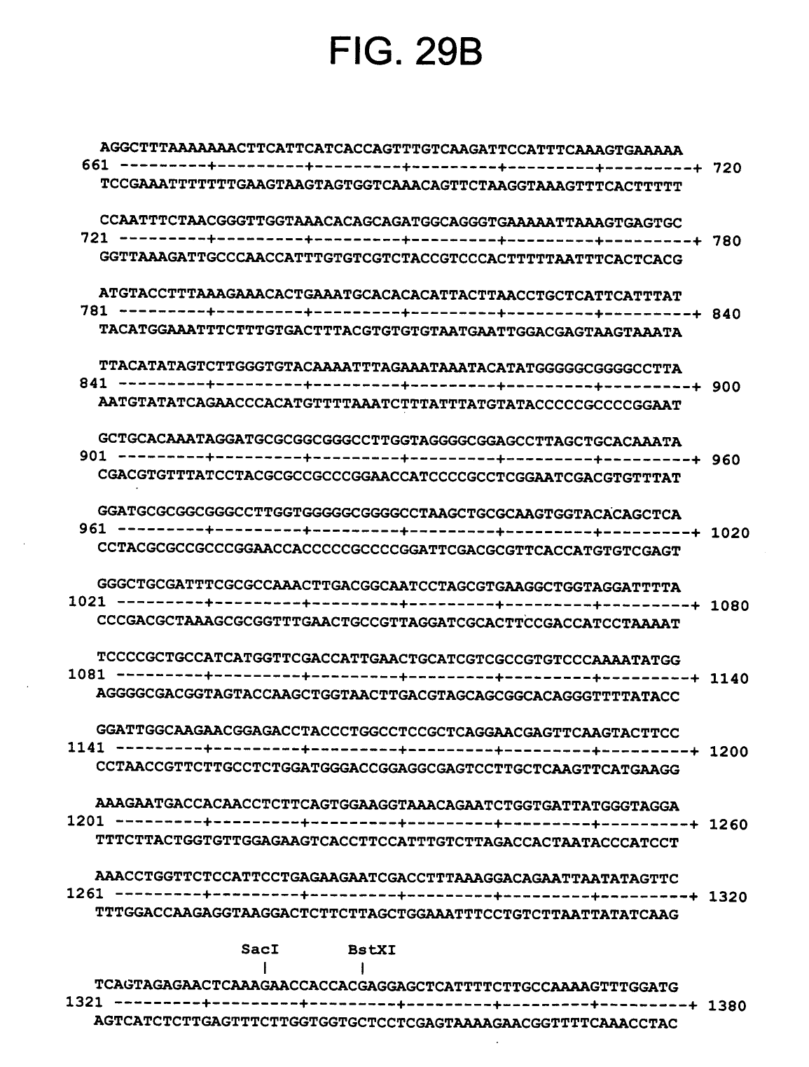

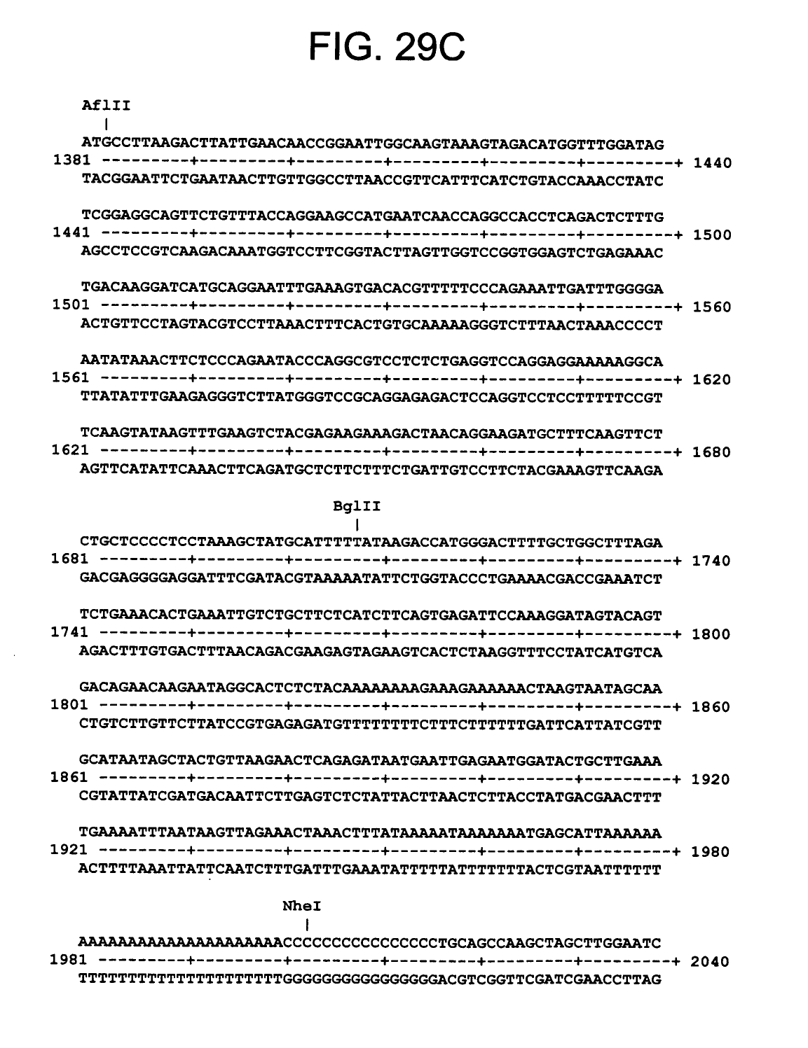

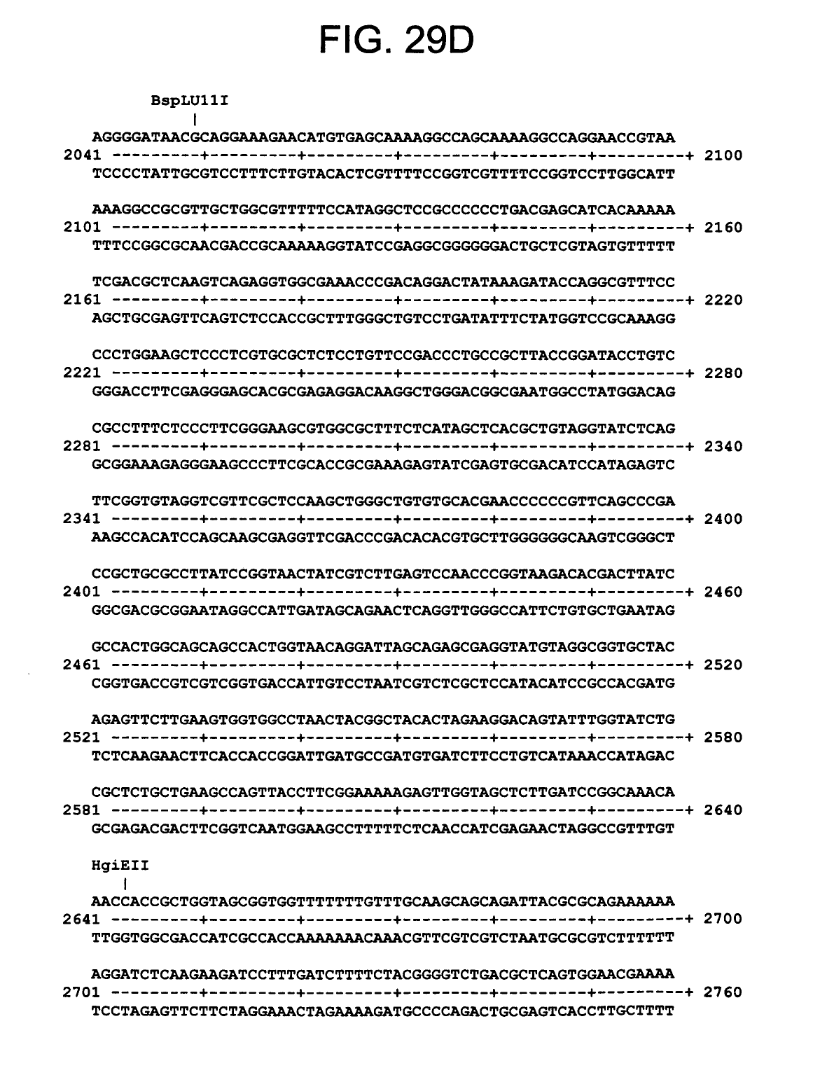

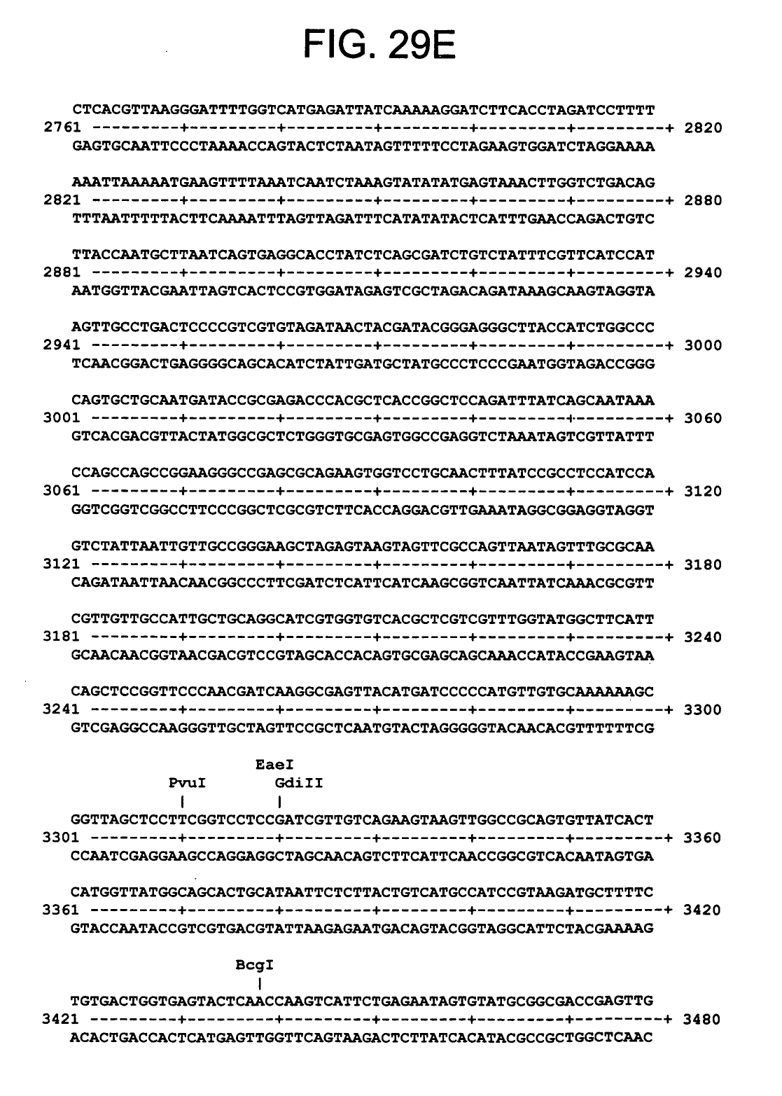

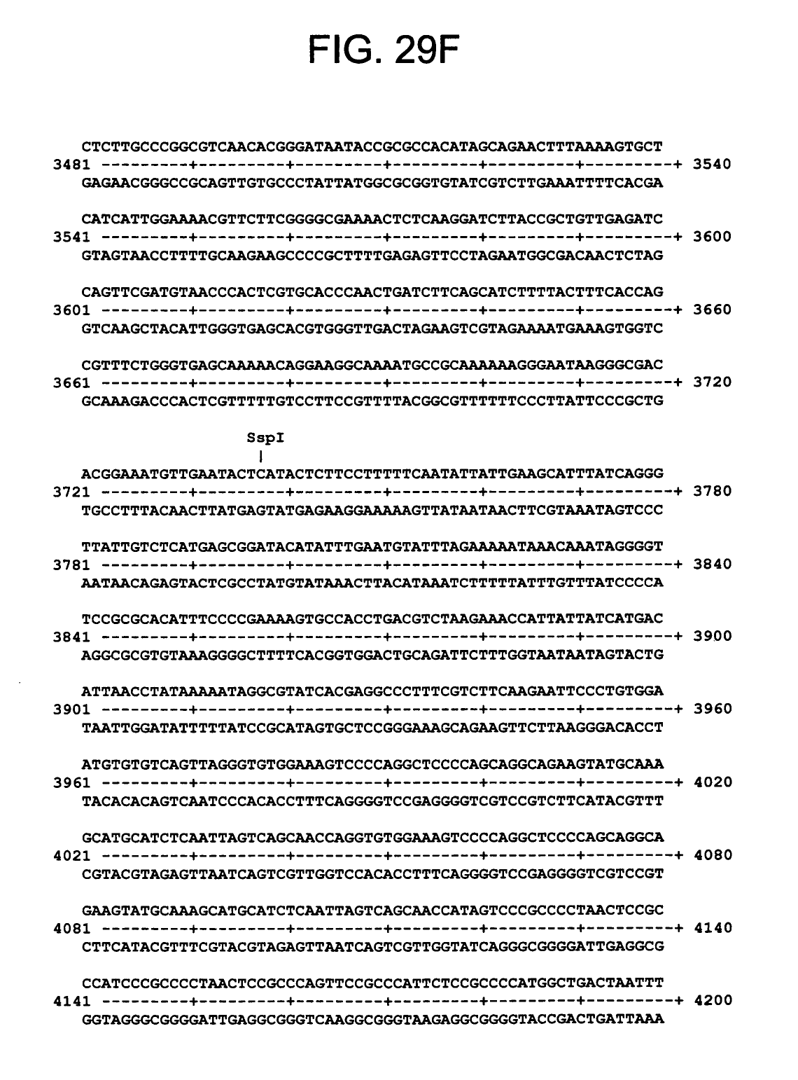

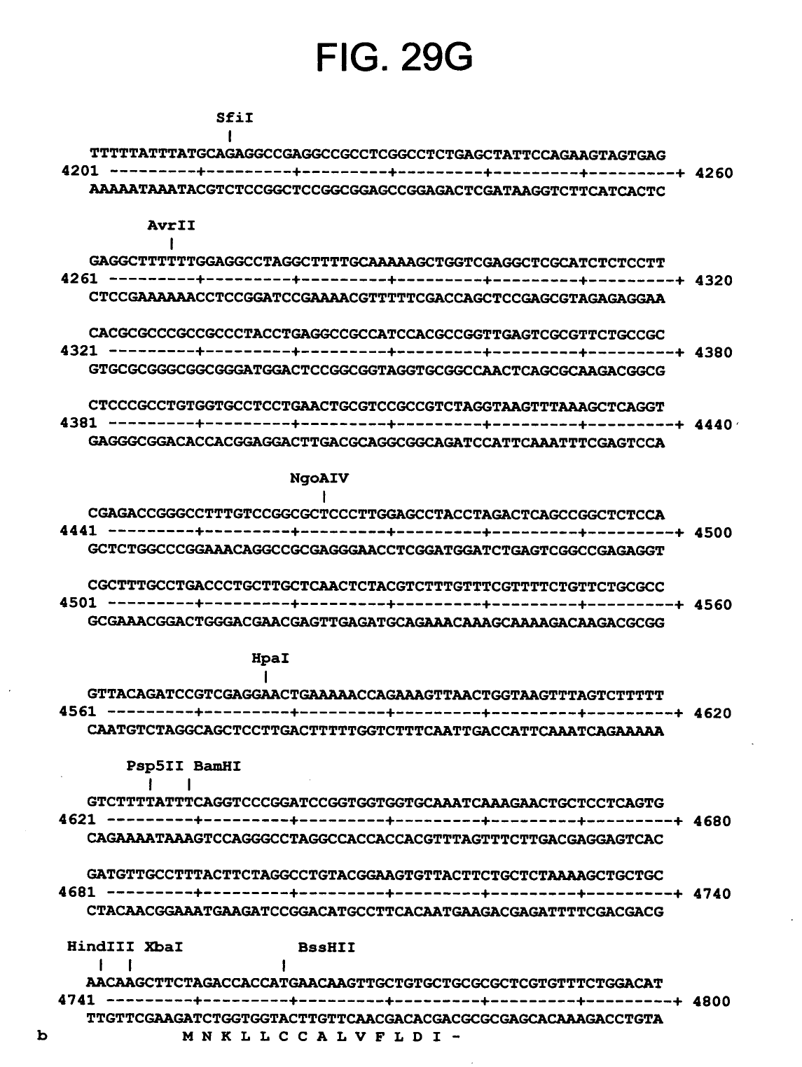

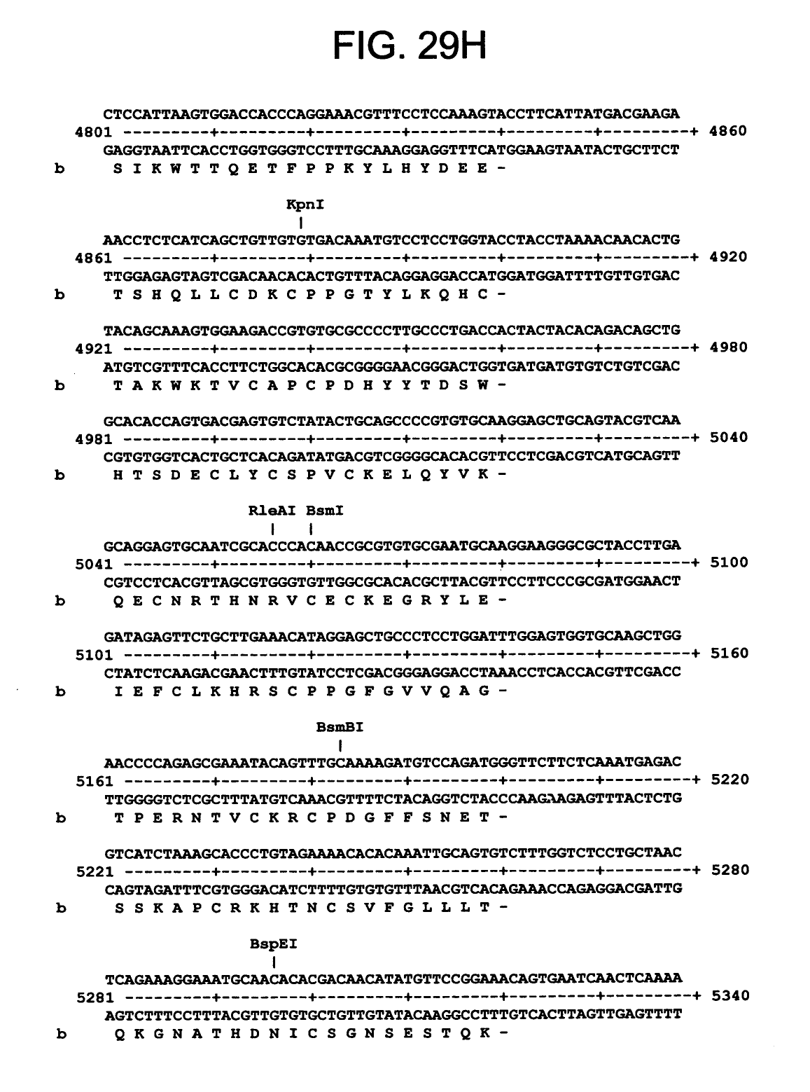

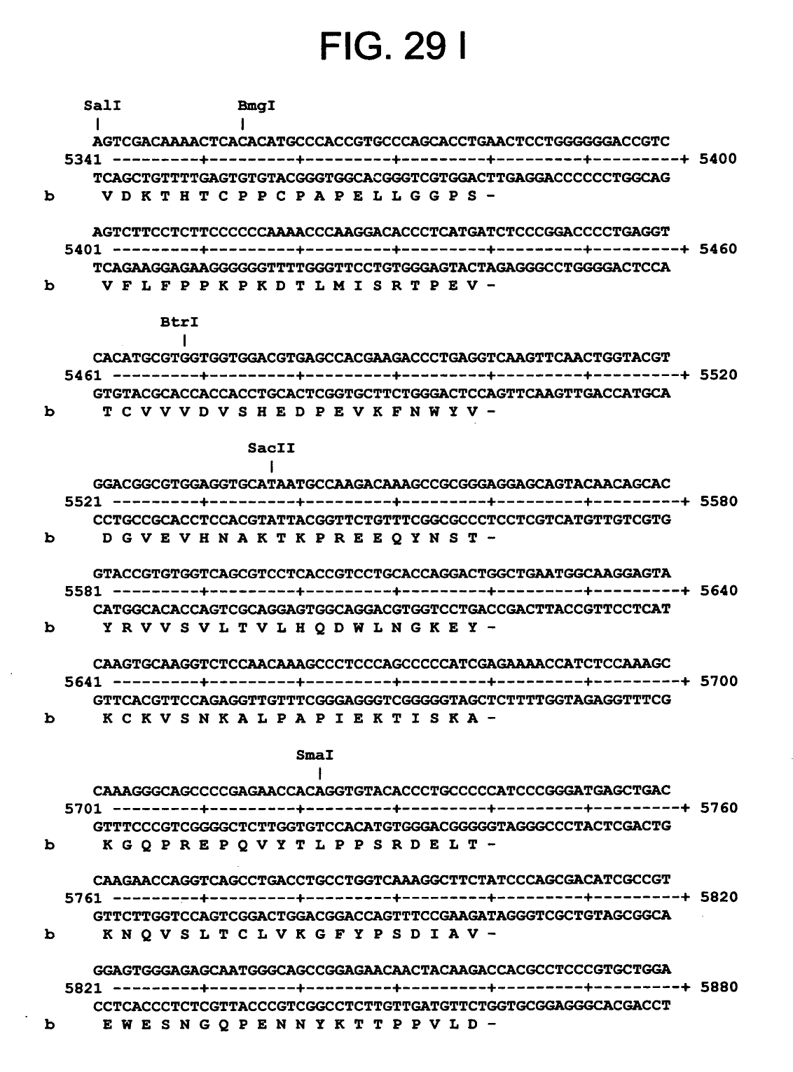

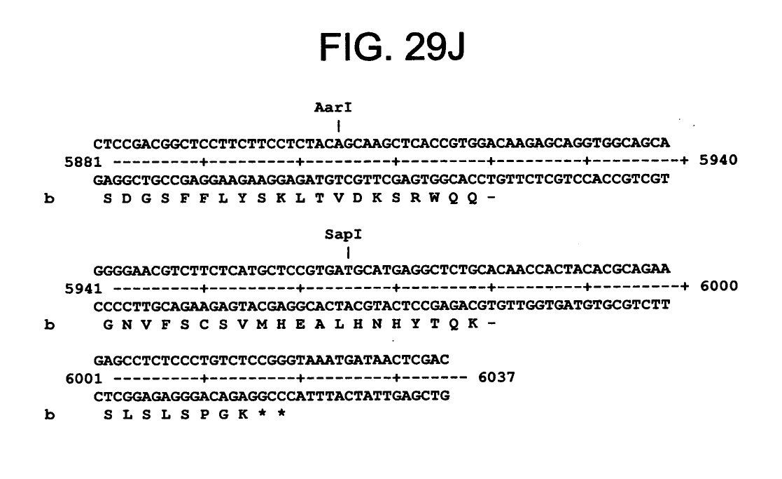

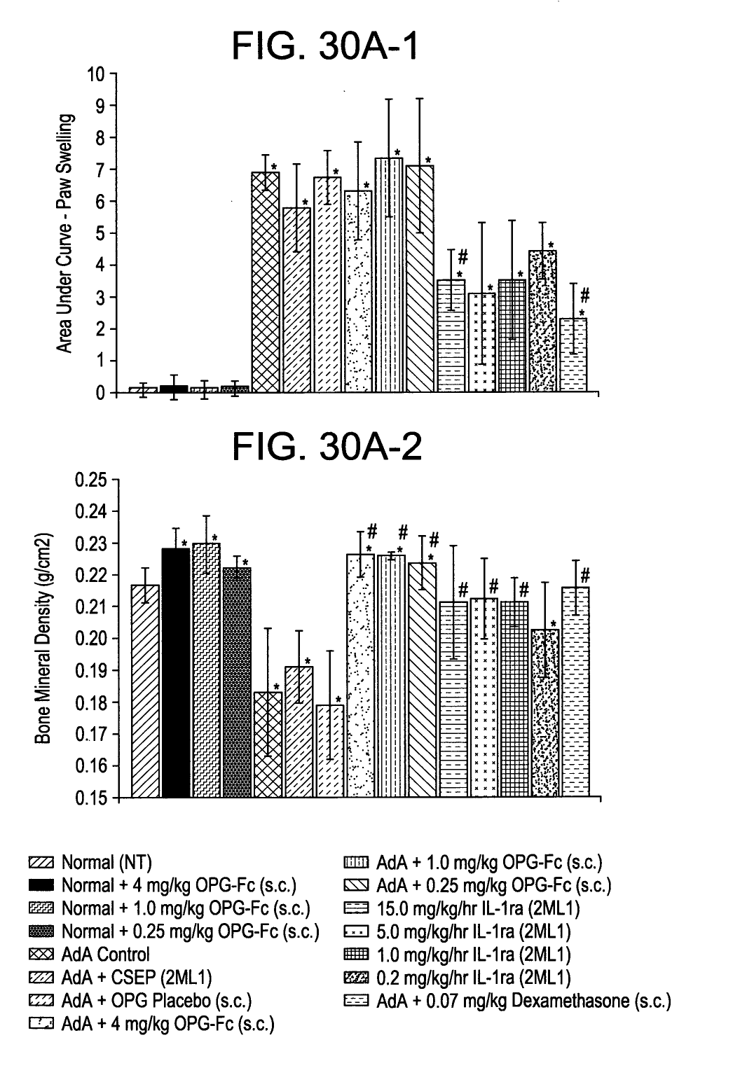

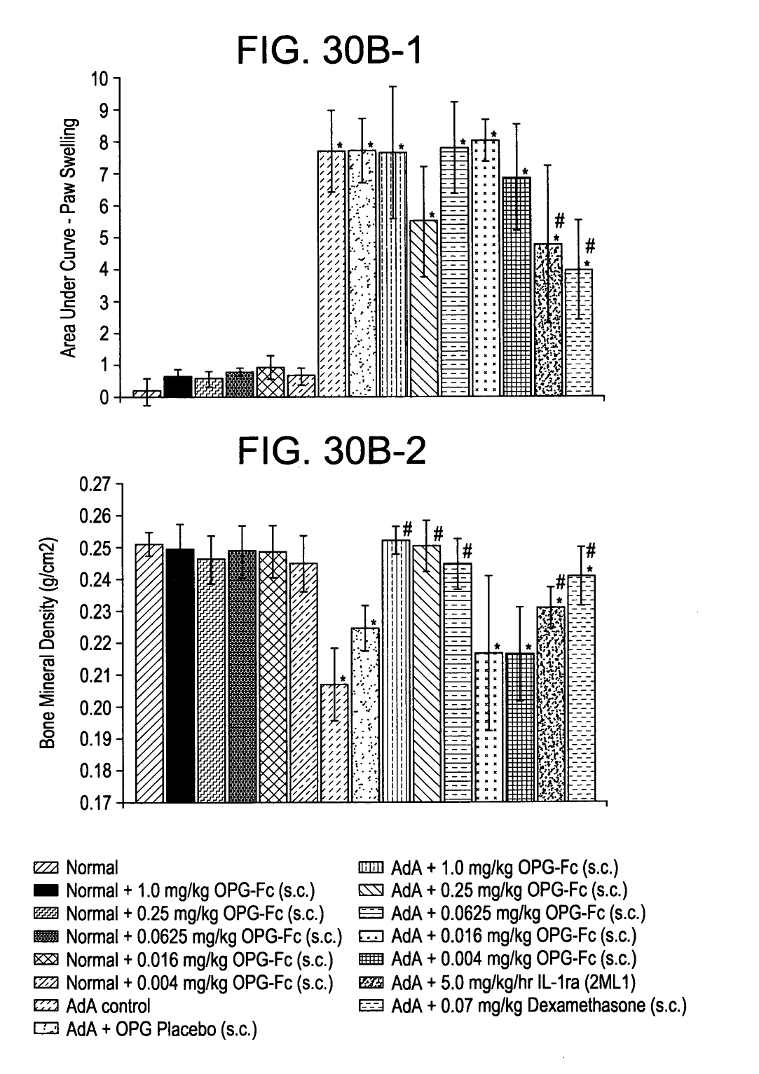

The term “OPG protein” refers collectively to the novel member of the tumor necrosis factor receptor family described hereinafter, variants and truncations thereof that maintain OPG's activity in increasing bone density, and antibodies to OPG ligand that maintain OPG's activity in increasing bone density. An exemplary assay for measuring such activity is shown in FIG. 6 and the accompanying text. Exemplary OPG proteins are polypeptides comprising the consensus of the rat, mouse and human sequences (FIG. 9C), OPG-Fc fusions (FIGS. 13, 29), or the rat, mouse or human OPG sequences (FIGS. 2, 9).

OPG was identified as follows. A novel member of the tumor necrosis factor receptor (TNFR) superfamily was identified as an expressed sequence tag (EST) isolated from a fetal rat intestinal cDNA library. The structures of the full-length rat cDNA clones and the corresponding mouse and human cDNA clones were determined as described in Examples 1 and 6. The rat, mouse and human genes are shown in FIGS. 2B-2C (SEQ ID NO: 120), 9A-9B (SEQ ID NO: 122), and 9C-9D (SEQ ID NO: 124), respectively. All three sequences showed strong similarity to the extracellular domains of TNFR family members. None of the full-length cDNA clones isolated encoded transmembrane and cytoplasmic domains that would be expected for membrane-bound receptors, suggesting that these cDNAs encode soluble, secreted proteins rather than cell surface receptors. A portion of the human gene spanning nucleotides 1200-1353 shown in FIG. 9D was deposited in the Genebank database on Nov. 22, 1995 under accession no. 17188769.

The tissue distribution of the rat and human mRNA was determined as described in Example 2. In rat, mRNA expression was detected in kidney, liver, placenta and heart with the highest expression in the kidney. Expression in skeletal muscle and pancreas was also detected. In humans, expression was detected in the same tissues along with lymph node, thymus, spleen and appendix.

The rat cDNA was expressed in transgenic mice (Example 3) using the liver-specific ApoE promoter expression system. Analysis of expressors showed a marked increase in bone density, particularly in long bones (femurs), vertebrae and flat bones (pelvis). Histological analysis of stained sections of bone showed severe osteopetrosis (see Example 4) indicating a marked imbalance between bone formation and resorption which has led to a marked accumulation of bone and cartilage. A decrease in the number of trabecular osteoclasts in the bones of OPG expressor animals indicate that a significant portion of the activity of the TNFR-related protein may be to prevent bone resorption, a process mediated by osteoclasts. In view of the activity in transgenic expressors, the TNFR-related proteins described herein are termed OPGs.

Using the rat cDNA sequence, mouse and human cDNA clones were isolated (Example 5). Expression of mouse OPG in 293 cells and human OPG in E. coli is described in Examples 7 and 8. Mouse OPG was produced as an Fc fusion which was purified by Protein A affinity chromatography. Also described in Example 7 is the expression of full-length and truncated human and mouse OPG polypeptides in CHO and 293 cells either as fusion polypeptides to the Fc region of human IgG1 or as unfused polypeptides. The expression of full-length and truncated human and mouse OPGs in E. coli either as Fc fusion polypeptides or as unfused polypeptides is described in Example 8. Purification of recombinantly produced mammalian and bacterial OPG is described in Example 10.

The biological activity of OPG was determined using an in vitro osteoclast maturation assay, an in vivo model of interleukin-1 (IL-1) induced hypercalcemia, and injection studies of bone density in normal mice (see Example 11). The following OPG recombinant proteins produced in CHO or 293 cells demonstrated activity in the in E. coli osteoclast maturation assay: muOPG [22-185]-Fc, muOPG [22-194]-Fc, muOPG [22-401]Fc, muOPG [22-401], huOPG [22-201]-Fc, huOPG [22-401]-Fc. muOPG [22-180]-Fc produced in CHO cells and huOPG met[32-401] produced in E. coli did not demonstrate activity in the in vitro assay.

OPG from several sources was produced as a dimer and to some extent as a higher multimer. Rat OPG [22-401] produced in transgenic mice, muOPG [22-401] and huOPG [22-401] produced as a recombinant polypeptide in CHO cells, and OPG expressed as a naturally occurring product from a cytotoxic T cell line were predominantly dimers and trimers when analyzed on nonreducing SDS gels (see Example 9). Truncated OPG polypeptides having deletions in the region of amino acids 186-401 (e.g., OPG [1-185] and OPG [1-194]) were predominantly monomeric suggesting that the region 186-401 may be involved in self-association of OPG polypeptides. However, huOPG met[32-401] produced in E. coli was largely monomeric.

OPG may be important in regulating bone resorption. The protein appears to act as a soluble receptor of the TNF family and may prevent a receptor-ligand interaction involved in the osteolytic pathway. One aspect of the regulation appears to be a reduction in the number of osteoclasts.

OPG proteins encompassed by the invention include rat [1-401], rat [22-180], rat [22-401], rat [22-401]-Fc fusion, rat [1-180]-Fc fusion, mouse [1-401], mouse [1-180], mouse [22-401], human [1-401], mouse [22-180], human [22-401], human [22-180], human [1-180], human [22-180]-Fc fusion and human met-32-401. Amino acid numbering is as shown in SEQ ID NO: 121 (rat), SEQ ID NO: 123 (mouse) and SEQ ID NO: 125 (human). Also encompassed are polypeptide derivatives having deletions or carboxy-terminal truncations of part or all of amino acids residues 180-401 of OPG; one or more amino acid changes in residues 180-401; deletion of part or all of a cysteine-rich domain of OPG, in particular deletion of the distal (carboxy-terminal) cysteine-rich domain; and one or more amino acid changes in a cysteine-rich domain, in particular in the distal (carboxy-terminal) cysteine-rich domain. In one embodiment, OPG has from 1 to about 216 amino acids deleted from the carboxy terminus. In another embodiment, OPG has from 1 to about 10 amino acids deleted from the mature amino terminus (wherein the mature amino terminus is at residue 22) and, optionally, has from 1 to about 216 amino acids deleted from the carboxy terminus.

Additional OPG proteins encompassed by the invention include the following: human [22-180]-Fc fusion, human [22-201]-Fc fusion, human [22-401]-Fc fusion, mouse [22-185]-Fc fusion, mouse [22-194]-Fc fusion. These polypeptides are produced in mammalian host cells, such as CHO or 293 cells, Additional OPG polypeptides encompassed by the invention which are expressed in procaryotic host cells include the following: human met[22-401], Fc-human met[22-401] fusion (Fc region is fused at the amino terminus of the full-length OPG coding sequence as described in Example 8), human met[22-401]-Fc fusion (Fc region fused to the full-length OPG sequence), Fc-mouse met[22-401] fusion, mouse met[22-401]-Fc fusion, human met[27-401], human met[22-185], human met[22-189], human met[22-194], human met[22-194] (P25A), human met [22-194] (P26A), human met[27-185], human met[27-189], human met[27-194], human met-arg-gly-ser-(his)₆[22-401], human met-lys [22-401], human met-(lys)3-[22-401], human met[22-401]-Fc (P25A), human met[22-401](P25A), human met[22-401](P26A), human met[22-401] (P26D), mouse met[22-401], mouse met[27-401], mouse met[32-401], mouse met[27-180], mouse met[22-189], mouse met[22-194, mouse met[27-189], mouse met[27-194], mouse met-lys[22-401], mouse HEK[22-401](A45T), mouse met-lys-(his)₇[22-401], mouse met-lys[22-401]-(his)₇ and mouse met[27-401] (P33E, G36S, A45P). It is understood that the above OPG polypeptides produced in procaryotic host cells have an amino-terminal methionine residue, if such a residue is not indicated. In specific examples, OPG-Fc fusion were produced using a 227 amino acid region of human IgG1-γ1 was used having the sequence as shown in Ellison et al. (1982) Nuc. Acids Res. 10: 4071-9. However, variants of the Fc region of human IgG may also be used.

Analysis of the biological activity of carboxy-terminal OPG truncations fused to the human IgG1 Fc region indicates a portion of OPG of about 164 amino acids which is required for activity. This region encompasses amino acids 22-185, preferably those in FIG. 9C-9D (SEQ ID NO: 125), and comprises four cysteine-rich domains characteristic of the cysteine-rich domains of TNFR extraceullular domains. Proteins comprising this 164 amino acid sequence are within the meaning of “OPG protein” in this invention.

OPG proteins of the invention also may be isolated and purified from other polypeptides present in tissues, cell lines and transformed host cells expressing OPG, or purified from components in cell cultures containing the secreted protein. In one embodiment, the polypeptide is free from association with other human proteins, such as the expression product of a bacterial host cell.

A method for the purification of OPG from natural sources and from transfected host cells is also included. The purification process may employ one or more standard protein purification steps in an appropriate order to obtain purified protein. The chromatography steps can include ion exchange, gel filtration, hydrophobic interaction, reverse phase, chromatofocusing, affinity chromatography employing an anti-OPG antibody or biotin-streptavidin affinity complex and the like.

IL- 1 Inhibitors

One of the most potent inflammatory cytokines yet discovered is interleukin-1 (IL-1). IL-1 is thought to be a key mediator in many diseases and medical conditions. It is manufactured (though not exclusively) by cells of the macrophage/monocyte lineage and may be produced in two forms: IL-1 alpha (IL-1α) and IL-1 beta (IL-1β).

A disease or medical condition is considered to be an “interleukin-1 mediated disease” if the spontaneous or experimental disease or medical condition is associated with elevated levels of IL-1 in bodily fluids or tissue or if cells or tissues taken from the body produce elevated levels of IL-1 in culture. In many cases, such interleukin-1 mediated diseases are also recognized by the following additional two conditions: (1) pathological findings associated with the disease or medical condition can be mimicked experimentally in animals by administration of IL-1 or upregulation of expression of IL-1; and (2) a pathology induced in experimental animal models of the disease or medical condition can be inhibited or abolished by treatment with agents that inhibit the action of IL-1. In most interleukin-1 mediated diseases at least two of the three conditions are met, and in many interleukin-1 mediated diseases all three conditions are met.

A non-exclusive list of acute and chronic interleukin-1 (IL-1)-mediated diseases includes but is not limited to the following:
acute pancreatitis;
ALS;
Alzheimer's disease;
cachexia/anorexia, including AIDS-induced cachexia;
asthma and other pulmonary diseases; atherosclerosis;
autoimmune vasculitis;
chronic fatigue syndrome;
Clostridium associated illnesses, including Clostridium-associated diarrhea;
coronary conditions and indications, including congestive heart failure, coronary restenosis, myocardial infarction, myocardial dysfunction (e.g., related to sepsis), and coronary artery bypass graft;
cancer, such as multiple myeloma and myelogenous (e.g., AML and CML) and other leukemias, as well as tumor metastasis;
diabetes (e.g., insulin diabetes);
endometriosis;
fever;
fibromyalgia;
glomerulonephritis;
graft versus host disease/transplant rejection;
hemohorragic shock;
hyperalgesia;
inflammatory bowel disease;
inflammatory conditions of a joint, including osteoarthritis, psoriatic arthritis and rheumatoid arthritis;
inflammatory eye disease, as may be associated with, for example, corneal transplant;
ischemia, including cerebral ischemia (e.g., brain injury as a result of trauma, epilepsy, hemorrhage or stroke, each of which may lead to neurodegeneration);
Kawasaki's disease;
learning impairment;
lung diseases (e.g., ARDS);
multiple sclerosis;
myopathies (e.g., muscle protein metabolism, esp. in sepsis);
neurotoxicity (e.g., as induced by HIV);
osteoporosis;
pain, including cancer-related pain;
Parkinson's disease;
periodontal disease;
pre-term labor;
psoriasis;
reperfusion injury;
septic shock;
side effects from radiation therapy;
temporal mandibular joint disease;
sleep disturbance;
uveitis;
or an inflammatory condition resulting from strain, sprain, cartilage damage, trauma, orthopedic surgery, infection or other disease processes.

Interleukin-1 inhibitors may be from any protein capable of specifically preventing activation of cellular receptors to IL-1, which may result from any number of mechanisms. Such mechanisms include downregulating IL-1 production, binding free IL-1, interfering with IL-1 binding to its receptor, interfering with formation of the IL-1 receptor complex (i.e., association of IL-1 receptor with IL-1 receptor accessory protein), or interfering with modulation of IL-1 signaling after binding to its receptor. Classes of interleukin-1 inhibitors include:
interleukin-1 receptor antagonists such as IL-1ra, as described below;
anti-IL-1 receptor monoclonal antibodies (e.g., EP 623674), the disclosure of which is hereby incorporated by reference;
L-1 binding proteins such as soluble L-1 receptors (e.g., U. S. Pat. No. 5,492,888, U. S. Pat. No. 5,488,032, and U. S. Pat. No. 5,464,937, U. S. Pat. No. 5,319,071, and U.S. Pat. No. 5,180,812, the disclosures of which are hereby incorporated by reference);
anti-IL-1 monoclonal antibodies (e.g., WO 9501997, WO 9402627, WO 9006371, U.S. Pat. No. 4,935,343, EP 364778, EP 267611 and EP 220063, the disclosures of which are hereby incorporated by reference);
IL-1 receptor accessory proteins and antibodies thereto (e.g., WO 96/23067 and WO 99/37773, the disclosure of which is hereby incorporated by reference);
inhibitors of interleukin-1 beta converting enzyme (ICE) or caspase I (e.g., WO 99/46248, WO 99/47545, and WO 99/47154, the disclosures of which are hereby incorporated by reference), which can be used to inhibit IL-1 beta production and secretion;
interleukin-1beta protease inhibitors;
and other compounds and proteins which block in vivo synthesis or extracellular release of IL-1.

Exemplary IL-1 inhibitors are disclosed in the following references:
U.S. Pat. Nos. 5,747,444; 5,359,032; 5,608,035; 5,843,905; 5,359,032; 5,866,576; 5,869,660; 5,869,315; 5,872,095; 5,955,480; 5,965,564;
International (WO) patent applications 98/21957, 96/09323, 91/17184, 96/40907, 98/32733, 98/42325, 98/44940, 98/47892, 98/56377, 99/03837, 99/06426, 99/06042, 91/17249, 98/32733, 98/17661, 97/08174, 95/34326, 99/36426, 99/36415.
European (EP) patent applications 534978 and 894795.
French patent application FR 2762514.
   The disclosures of all of the aforementioned references are hereby incorporated by reference.

For purposes of the present invention, IL-1ra and variants and derivatives thereof as discussed hereinafter are collectively termed “IL-1ra protein(s)”. The molecules described in the above references and the variants and derivatives thereof discussed hereinafter are collectively termed “IL-1 inhibitors.”

Interleukin-1 receptor antagonist (IL-1ra) is a human protein that acts as a natural inhibitor of interleukin-1 and which is a member of the IL-1 family member which includes IL-1α and IL-1β. Preferred receptor antagonists (including IL-1ra and variants and derivatives thereof), as well as methods of making and using thereof, are described in U.S. Pat. No. 5,075,222; WO 91/08285; WO 91/17184; AU 9173636; WO 92/16221; WO93/21946; WO 94/06457; WO 94/21275; FR 2706772; WO 94/21235; DE 4219626, WO 94/20517; WO 96/22793;WO 97/28828; and WO 99/36541, the disclosures of which are incorporated herein by reference. The proteins include glycosylated as well as non-glycosylated IL-1 receptor antagonists.

Specifically, three useful forms of IL-1ra and variants thereof are disclosed and described in the U.S. Pat. No. 5,075,222 patent. The first of these, called “IL-1i” in the '222 patent, is characterized as a 22-23 kD molecule on SDS-PAGE with an approximate isoelectric point of 4.8, eluting from a Mono Q FPLC column at around 52 mM NaCl in Tris buffer, pH 7.6. The second, IL-1raβ, is characterized as a 22-23 kD protein, eluting from a Mono Q column at 48 mM NaCl. Both IL-1raα and IL-1raβ are glycosylated. The third, IL-1rax, is characterized as a 20 kD protein, eluting from a Mono Q column at 48 mM NaCl, and is non-glycosylated. U.S. Pat. No. 5,075,222 patent also discloses methods for isolating the genes responsible for coding the inhibitors, cloning the gene in suitable vectors and cell types, and expressing the gene to produce the inhibitors.

Those skilled in the art understand that many combinations of deletions, insertions and substitutions (individually or collectively “variant(s)”) can be made within the amino acid sequences of IL-1ra, provided that the resulting molecule is biologically active (e.g., possesses the ability to inhibit IL-1). See “Variants of Proteins” hereinafter.

TNF-α Inhibitors

Many diseases and medical conditions are mediated by TNF and are usually categorized as inflammatory conditions. A “TNF-mediated disease” is a spontaneous or experimental disease or medical condition is associated with elevated levels of TNF in bodily fluids or tissue or if cells or tissues taken from the body produce elevated levels of TNF in culture. In many cases, such TNF-mediated diseases may also be recognized by (1) pathological findings associated with the disease or medical condition can be mimicked experimentally in animals by the administration or upregulation of expression of TNF or (2) a pathology induced in experimental animal models of the disease or medical condition can be inhibited or abolished by treatment with agents that inhibit the action of TNF. A non-exclusive list of acute and chronic TNF-mediated diseases includes but is not limited to the following:
cachexia/anorexia;
cancer (e.g., leukemias);
chronic fatigue syndrome;
coronary conditions and indications, including congestive heart failure, coronary restenosis, myocardial infarction, myocardial dysfunction (e.g., related to sepsis), and coronary artery bypass graft;
depression;
diabetes, including juvenile onset Type 1, diabetes mellitus, and insulin resistance (e.g., as associated with obesity);
endometriosis, endometritis, and related conditions;
fibromyalgia or analgesia;
graft versus host rejection;
hyperalgesia;
inflammatory bowel diseases, including Crohn's disease and Clostridium difficile-associated diarrhea;
ischemia, including cerebral ischemia (brain injury as a result of trauma, epilepsy, hemorrhage or stroke, each of which may lead to neurodegeneration);
lung diseases (e.g., adult respiratory distress syndrome, asthma, and pulmonary fibrosis);
multiple sclerosis;
neuroinflammatory diseases;
ocular diseases and conditions, including corneal transplant, ocular degeneration and uveitis;
pain, including cancer-related pain;
pancreatitis;
periodontal diseases;
Pityriasis rubra pilaris (PRP);
prostatitis (bacterial or non-bacterial) and related conditions;
psoriasis and related conditions;
pulmonary fibrosis;
reperfusion injury;
rheumatic diseases, including rheumatoid arthritis, osteoarthritis, juvenile (rheumatoid) arthritis, seronegative polyarthritis, ankylosing spondylitis, Reiter's syndrome and reactive arthritis, Still's disease, psoriatic arthritis, enteropathic arthritis, polymyositis, dermatomyositis, scleroderma, systemic sclerosis, vasculitis (e.g., Kawasaki's disease), cerebral vasculitis, Lyme disease, staphylococcal-induced (“septic”) arthritis, Sjögren's syndrome, rheumatic fever, polychondritis and polymyalgia rheumatica and giant cell arteritis);
septic shock;
side effects from radiation therapy;
systemic lupus erythematosus (SLE);
temporal mandibular joint disease;
thyroiditis;
tissue transplantation or an inflammatory condition resulting from strain, sprain, cartilage damage, trauma, orthopedic surgery, infection (e.g., HIV, Clostridium difficile and related species) or other disease process.

TNF-α inhibitors may act by downregulating or inhibiting TNF production, binding free TNF, interfering with TNF binding to its receptor, or interfering with modulation of TNF signaling after binding to its receptor. The term “TNF-α inhibitor” thus includes solubilized TNF receptors, antibodies to TNF, antibodies to TNF receptor, inhibitors of TNF-α converting enzyme (TACE), and other molecules that affect TNF activity.

TNF-α inhibitors of various kinds are disclosed in the art, including the following references:
European patent applications 308 378; 422 339; 393 438; 398 327; 412 486; 418 014, 417 563, 433 900; 464 533;512 528; 526 905;568 928; EP 607 776 (use of leflunomide for inhibition of TNF-□); 663 210; 542 795; 818 439; 664 128; 542 795; 741 707; 874 819 ; 882 714; 880 970; 648 783; 731 791; 895 988; 550 376; 882 714; 853 083; 550 376; 943 616; 939 121; 614 984 ; 853 083
U.S. Pat. Nos. 5,136,021; 5,929,117; 5,948,638; 5,807,862; 5,695,953; 5,834,435; 5,817,822; 5830742; 5,834,435; 5,851,556; 5,853,977; 5,359,037; 5,512,544; 5,695,953; 5,811,261; 5,633,145; 5,863,926; 5,866,616; 5,641,673; 5,869,677; 5,869,511; 5,872,146; 5,854,003; 5,856,161; 5,877,222; 5,877,200; 5,877,151; 5,886,010; 5,869,660; 5,859,207; 5,891,883; 5,877,180; 5,955,480; 5,955,476; 5,955,435; 5,994,351; 5,990,119; 5,952,320; 5,962,481;
International(WO)patent applications 90/13575, 91/03553, 92/01002, 92/13095, 92/16221, 93/07863, 93/21946, 93/19777, 95/34326, 96/28546, 98/27298, 98/30541, 96/38150, 96/38150, 97/18207, 97/15561, 97/12902, 96/25861, 96/12735, 96/11209, 98/39326, 98/39316, 98/38859, 98/39315, 98/42659, 98/39329, 98/43959, 98/45268, 98/47863, 96/33172, 96/20926, 97/37974, 97/37973, 97/47599, 96/35711, 98/51665, 98/43946, 95/04045, 98/56377, 97/12244, 99/00364, 99/00363, 98/57936, 99/01449, 99/01139, 98/56788, 98/56756, 98/53842, 98/52948, 98/52937, 99/02510, 97/43250, 99/06410, 99/06042, 99/09022, 99/08688, 99/07679, 99/09965, 99/07704, 99/06041, 99/37818, 99/37625, 97/11668, 99/50238, 99/47672, 99/48491;
Japanese (JP) patent applications 10147531, 10231285, 10259140, and 10130149, 10316570, 11001481, and 127,800/1991;
German (DE) application 19731521;
British (GB) applications 2 218 101, 2 326 881, 2 246 569.

The disclosures of all of the aforementioned references are hereby incorporated by reference.

For purposes of this invention, the molecules disclosed in these references and the sTNFRs and variants and derivatives of the sTNFRs and the molecules disclosed in the references (see below) are collectively termed “TNF-α inhibitors.”

For example, EP 393 438 and EP 422 339 teach the amino acid and nucleic acid sequences of a soluble TNF receptor type I (also known as sTNFR-I or 30 kDa TNF inhibitor) and a soluble TNF receptor type II (also known as sTNFR-II or 40 kDa TNF inhibitor), collectively termed “sTNFRs”, as well as modified forms thereof (e.g., fragments, functional derivatives and variants). EP 393 438 and EP 422 339 also disclose methods for isolating the genes responsible for coding the inhibitors, cloning the gene in suitable vectors and cell types, and expressing the gene to produce the inhibitors.

sTNFR-I and sTNFR-II are members of the nerve growth factor/TNF receptor superfamily of receptors which includes the nerve growth factor receptor (NGF), the B cell antigen CD40, 4-1BB, the rat T-cell antigen MRC OX40, the fas antigen, and the CD27 and CD30 antigens (Smith et al. (1990), Science, 248:1019-1023). The most conserved feature amongst this group of cell surface receptors is the cysteine-rich extracellular ligand binding domain, which can be divided into four repeating motifs of about forty amino acids and which contains 4-6 cysteine residues at positions which are well conserved (Smith et al. (1990), supra).

EP 393 438 teaches a 40 kDa TNF inhibitor Δ51 and a 40 kDa TNF inhibitor Δ53, which are truncated versions of the full-length recombinant 40 kDa TNF inhibitor protein wherein 51 or 53 amino acid residues, respectively, at the carboxyl terminus of the mature protein are removed.

PCT Application No. PCT/US97/12244 teaches truncated forms of sTNFR-I and sTNFR-II which do not contain the fourth domain (amino acid residues Thr¹²⁷-Asn¹⁶¹ of sTNFR-I and amino acid residues Pro¹⁴¹-Thr¹⁷⁹ of sTNFR-II); a portion of the third domain (amino acid residues Asn¹¹¹-Cys¹²⁶ of sTNFR-I and amino acid residues Pro¹²³-Lys¹⁴⁰ of sTNFR-II); and, optionally, which do not contain a portion of the first domain (amino acid residues Asp¹-Cys¹⁹ of sTNFR-I and amino acid residues Leu¹-Cys³² of sTNFR-II). The truncated sTNFRs of the present invention include the proteins represented by the formula R₁-[Cys¹⁹⁹-Cys¹⁰³]-R₂ and R₄-[Cys³²-Cys¹¹⁵]-R₅. These proteins are truncated forms of sTNFR-I and sTNFR-II, respectively.

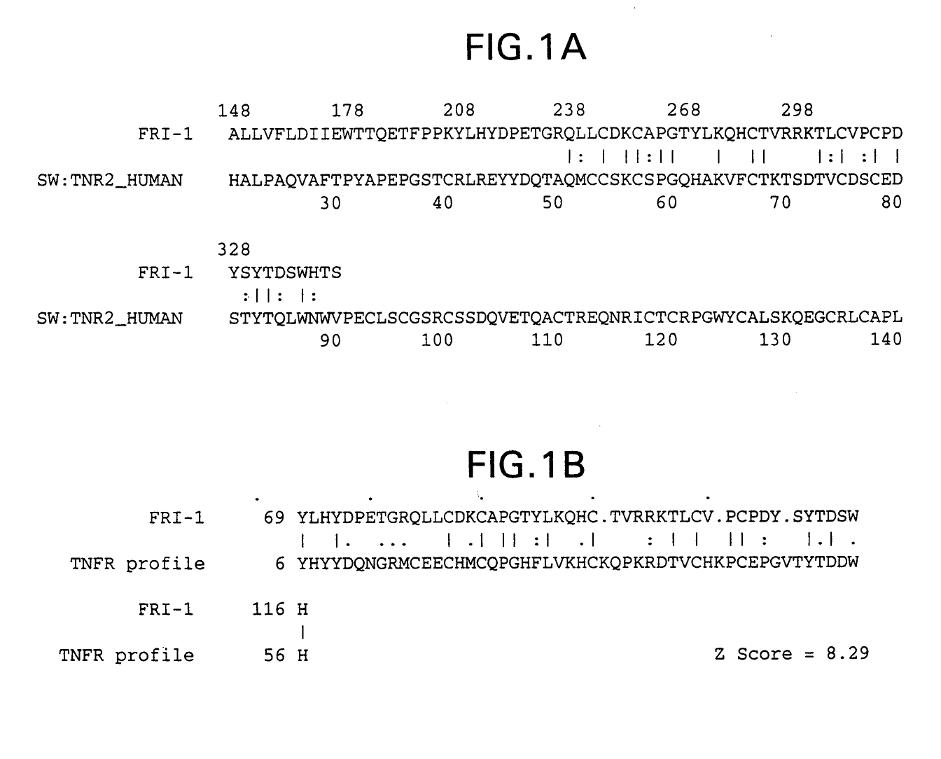

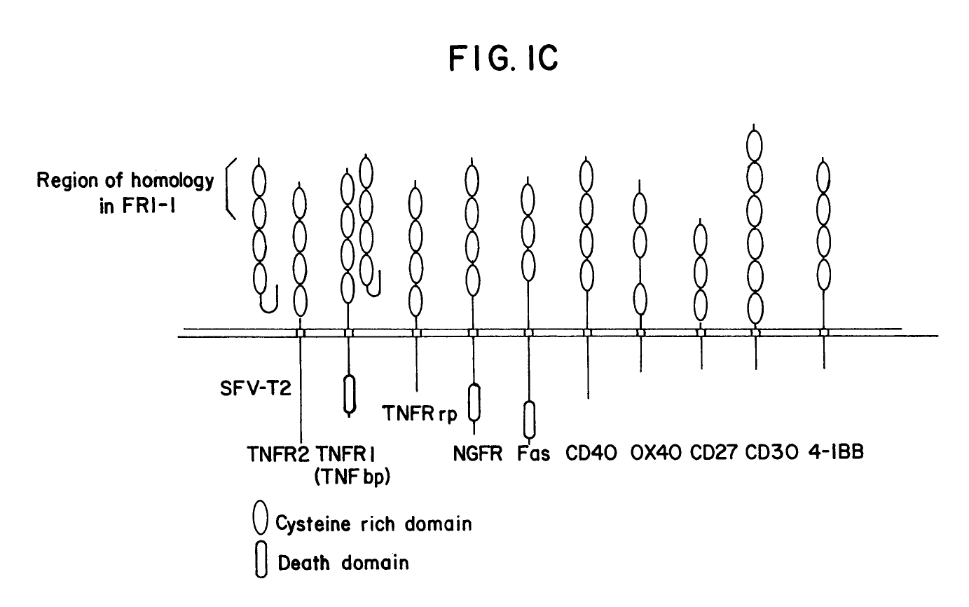

By “R₁-[Cys¹⁹-Cys¹⁰³]-R₂ “is meant one or more proteins wherein [Cys¹⁹-Cys¹⁰³] represents residues 19 through 103 of sTNFR-I, the amino acid residue numbering scheme of which is provided in FIG. 1 to facilitate the comparison; wherein R₁ represents a methionylated or nonmethionylated amine group of Cys¹⁹ or of amino-terminus amino acid residue(s) selected from any one of Cys¹⁸ to Asp¹ and wherein R₂ represents a carboxy group of Cys¹⁰³ or of carboxy-terminal amino acid residues selected from any one of Phe¹⁰⁴ to Leu¹¹⁰.

Exemplary truncated sTNFR-I of the present invention include the following molecules (collectively termed 2.6D sTNFR-I): NH₂-[Asp¹-Cys¹⁰⁵]-COOH (also referred to as sTNFR-I 2.6D/C105); NH₂-[Asp¹-Leu¹⁰⁸]-COOH (also referred to as sTNFR-I 2.6D/C106); NH₂-[Asp¹-Asn¹⁰⁵]-COOH (also referred to as sTNFR-I 2.6D/N105); NH₂-[Tyr⁹-Leu¹⁰⁸]-COOH (also referred to as sTNFR-I 2.3D/d8); NH₂-[Cys¹⁹-Leu¹⁰⁸]-COOH (also referred to as sTNFR-I 2.3D/d18); and NH₂-[Ser¹⁶-Leu¹⁰⁸]-COOH (also referred to as sTNFR-I 2.3D/d15), either methionylated or nonmethionylated, and variants and derivatives thereof.

By “R₃-[Cys³²-Cys¹¹⁵]-R₄” is meant one or more proteins wherein [Cys³²-Cys¹¹⁵] represents residues Cys³² through Cys¹¹⁵ of sTNFR-II, the amino acid residue numbering scheme of which is provided in FIG. 2 to facilitate the comparison; wherein R₃ represents a methionylated or nonmethionylated amine group of Cys³² or of amino-terminus amino acid residue(s) selected from any one of Cys³¹ to Leu¹ and wherein R₄ represents a carboxy group of Cys¹¹⁵ or of carboxy-terminal amino acid residue(s) selected from any one of Ala¹¹⁶ to Arg¹²².

Serine Protease Inhibitors

Endogenous proteolytic enzymes degrade invading organisms, antigen-antibody complexes, and certain tissue proteins that are no longer necessary or useful. Infective agents may introduce additional proteolytic enzymes into the organism. Protease inhibitors regulate both endogenous and invading proteolytic enzymes.

A large number of naturally occurring protease inhibitors serve to control the endogenous proteases by limiting their reactions locally and temporally. In addition, the protease inhibitors may inhibit proteases introduced into the body by infective agents. Tissues that are particularly prone to proteolytic attack and infection, e.g. those of the respiratory tract, are rich in protease inhibitors.

Protease inhibitors comprise approximately 10% of the human plasma proteins. At least eight inhibitors have been isolated from this source and characterized in the literature. These include alpha 2-macroglobulin (alpha 2M), alpha 1-protease inhibitor (alpha 1PI), alpha 1-antichymotrypsin (alpha 1Achy), alpha 1-anticollagenase (alpha 1AC), and inter-alpha-trypsin inhibitor (I-alpha-I).

A disturbance of the protease/protease inhibitor balance can lead to protease-mediated tissue destruction, including emphysema, arthritis, glomerulonephritis, periodontitis, muscular dystrophy, tumor invasion and various other pathological conditions. In certain situations, e.g. severe pathological processes such as sepsis or acute leukemia, the amount of free proteolytic enzymes present increases due to the release of enzyme from the secretory cells. In addition, or separately in other situations, a diminished regulating inhibitor capacity of the organism may also cause alterations in the protease/protease inhibitor balance. An example of such a diminished regulating inhibitor capacity is an alpha 1-protease inhibitor deficiency, which is highly correlated with the development of pulmonary emphysema.

In organisms where such aberrant conditions are present, serious damage to the organism can occur unless measures can be taken to control the proteolytic enzymes. Therefore, protease inhibitors have been sought which are capable of being administered to an organism to control the proteolytic enzymes.

One protease that is of particular pharmacological interest is leukocyte elastase. Leukocyte elastase, when released extracellularly, degrades connective tissue and other valuable proteins. While it is necessary for a normally functioning organism to degrade a certain amount of connective tissue and other proteins, the presence of an excessive amount of leukocyte elastase has been associated with various pathological states, such as emphysema and rheumatoid arthritis. To counteract the effects of leukocyte elastase when it is present in amounts greater than normal, a protease inhibitor has been sought which is specific for leukocyte elastase. Such a protease inhibitor would be especially useful if it were capable of being isolated or prepared in a purified form and in sufficient quantities to be pharmaceutically useful

In the past, at least two leukocyte elastase inhibitors have been identified in the literature. One protein, described in Schiessler et al., “Acid-Stable Inhibitors of Granulocyte Neutral Proteases in Human Mucous Secretions: Biochemistry and Possible Biological Function”, in Neutral Proteases of Human Polymorphoneuclear Leucocytes, Havemann et al. (eds), Urban and Schwarzenberg, Inc. (1978), was isolated from human seminal plasma and sputum and was characterized as being approximately 11 Kda in size with tyrosine as the N-terminal amino acid. The literature reports of this protein have only furnished a partial amino acid sequence, but even this partial sequence indicates that this protein varies substantially from the proteins of the present invention. The reports of the sequence of this protein, in combination with the complete amino acid sequence data for proteins of the present invention, indicate to the present inventors that the product sequenced by Schiessler et al. may have been a degraded protein which was not a single-polypeptide chain.

A second protein, isolated in one instance from human plasma, has been named alpha 1-protease inhibitor. Work on this protein has been summarized in a review by Travis and Salvesen, Ann. Rev. Biochem. 52: 655-709 (1983). The reports of the amino acid sequence of this protein indicate that it too differs substantially from the proteins of the present invention.

Trypsin is another protease of particular interest from a pharmacological standpoint. Trypsin is known to initiate degradation of certain soft organ tissue, such as pancreatic tissue, during a variety of acute conditions, such as pancreatitis. A variety of efforts have been directed toward the treatment of these conditions, without marked success, through the use of proteins which it was hoped would inhibit the action of trypsin. Illustrative of such efforts are attempts to use exogenous bovine trypsin inhibitors in treatment of human pancreatitis. While such techniques have been attempted in Europe, they have not been approved as effective by the U.S. Food and Drug Administration.

Thus, there is a need for a protease inhibitor effective in neutralizing excess trypsin in a variety of acute and chronic conditions. As was the case with the leukocyte elastase inhibitor discussed above, a trypsin inhibitor would be particularly useful if it could be isolated and prepared in a purified form and in sufficient quantities to be pharmaceutically useful.

Cathepsin G is another protease present in large quantities in leukocytes. Cathepsin G is known to be capable of degrading in vitro a variety of valuable proteins, including those of the complement pathway Pancreatic elastase is another protease which may have a role in pancreatitis. Thus, inhibitors for these proteases are also of pharmaceutical value.

Leukocyte elastase, trypsin, cathepsin G and pancreatic elastase are examples of a class of proteases known as serine proteases, which have elements of common structure and mechanism. Their activity against different substrates and their sensitivity to different inhibitors are believed to result from changes in only a few amino acid residues. By analogy, it is possible to conceive of a class of serine protease inhibitors, also having common elements of structure and mechanism, in which changes in a relatively few amino acids will result in inhibition of different proteases, and that at least one member of this class will inhibit every serine protease of the former class.

A particularly preferred serine protease inhibitor is secretory leukocyte protease inhibitor (SLPI) and fragments and analogues thereof. Also preferred are anti-leukoprotease (ALP), mucous protease inhibitor (MPI), human seminal plasma inhibitor-I (HUSI-I), bronchial mucus inhibitor (BMI), cervical mucus inhibitor (CUSI). These molecules are especially well-suited for use in conditions leading to bone loss because they are preferentially directed to the cartilage. Exemplary serine protease inhibitors are described in the following, each of which is hereby incorporated by reference: U.S. Pat. No. 4,760,130, issued Jul. 26, 1988; U.S. Pat. No. 5,900,400, issued May 4, 1999, which discloses preferred SLPI analogues; and U.S. Pat. No. 5,633,227, issued May 27, 1997, which discloses preferred SLPI fragments. The molecules disclosed in the foregoing references as well as any variants or analogues thereof as described hereinafter are collectively termed “serine protease inhibitors.”

IL-18 Inhibitors

IL-18 is a pro-inflammatory cytokine of somewhat recent discovery. IL-18 was found to induce interferon-□ and was originally named interferon gamma inducing factor (IGIF). IL-1 upregulates IL-18 production, and IL-18 induces production of a number of proinflammatory cytokines, including IL-6 and MMP-1. Dinarello et al. (1998), J. Leukocyte Biol. 63: 658-64. Caspase I is also critical for IL-18 production. The art also suggested that TNF-α regulates IL-18 production, and it was found that simultaneous inhibition of TNF-α and IL-18 protected against liver toxicity. Faggioni et al. (2000), PNAS 97: 2367-72.

IL-18 acts in vivo through a receptor system reminiscent of the IL-1 system. IL-18 interacts with a cell surface receptor (IL-18R), which interacts with an accessory protein (IL-18RAcP). IL-18-mediated signaling proceeds upon formation of the complex of IL-18, IL-18R, and IL-18RAcP. A natural inhibitor for IL-18 is IL-18 bp. Although it bears insignificant sequence homology with IL-18R, IL-18 bp's act as a “decoy receptors” by binding to IL-18 molecules and preventing interaction with IL-18 and subsequent IL-18-mediated signaling.

The present invention concerns methods of treatment using IL-18 inhibitors in combination with the other classes of molecules described herein. Such combination therapy is useful for treating inflammation and autoimmune diseases generally, as well as IL-1 mediated diseases and TNF-mediated diseases as defined hereinabove. In particular, combination therapy using IL-18 inhibitors is useful for treating arthritis (particularly rheumatoid arthritis), systemic lupus erythematosus (SLE), graft versus host disease (GvHD), hepatitis and sepsis.

A number of classes of IL-18 inhibitors are known in the art, and all are useful in the present invention. Suitable IL-18 inhibitors include antibodies binding to IL-18; antibodies binding to IL-18R; antibodies binding to IL-18RAcP; IL-18bp; IL-18R fragments (e.g., a solubilized extracellular domain of the IL-18 receptor), peptides binding to IL-18 and preventing its interaction with IL-18R; peptides binding to IL-18R and preventing its interaction with IL-18 or with IL-18RAcP; peptides binding to IL-18RAcP and preventing its interaction with IL-18R; and small molecules preventing IL-18 production or interaction between any of IL-18, IL-18R, and IL-18RAcP. Any of the foregoing, with the exception of small molecules, may be linked to half-life extending vehicles known in the art. Such vehicles include the Fc domain, polyethylene glycol, and dextran. These vehicles are reviewed in a patent application entitled, “Modified Peptides as Therapeutic Agents,” U.S. Ser. No. 09/428,082, PCT appl. no. WO 99/25044, which is hereby incorporated by reference in its entirety.

Useful IL-18 inhibitors are described in the following references, which are hereby incorporated by reference: U.S. Pat. No. 5,912,324, issued Jul. 14, 1994; EP 0 962 531, published Dec. 8, 1999; EP 712 931, published Nov. 15, 1994; U.S. Pat. No. 5,914,253, issued Jul. 14, 1994; WO 97/24441, published Jul. 10, 1997; U.S. Pat. No. 6,060,283, issued May 9, 2000; EP 850 952, published Dec. 26, 1996; EP 864 585, published Sep. 16, 1998; WO 98/41232, published Sep. 24, 1998; U.S. Pat. No. 6,054,487, issued Apr. 25, 2000; WO 99/09063, published Aug. 14, 1997; WO 99/22760, published Nov. 3, 1997; WO 99/37772, published Jan. 23, 1998; WO 99/37773, published Mar. 20, 1998; EP 0 974 600, published Jan. 26, 2000; WO 00/12555, published Mar. 9, 2000; Japanese patent application JP 111,399/94, published Oct. 31, 1997; Israel patent application IL 121554 A0, published Feb. 8, 1998.

Variants of Proteins

Those skilled in the art will understand that one may make many molecules derived in sequence from the aforementioned molecules in which amino acids have been deleted (“deletion variants”), inserted (“addition variants”), or substituted (“substitution variants”). Molecules having such substitutions, additions, deletions, or any combination thereof are termed individually or collectively “variant(s)”). Such variants should, however, maintain at some level (including a reduced level) the relevant activity of the unmodified or “parent” molecule (e.g., an sTNFR variant possesses the ability to bind TNF). Hereinafter, “parent molecule” refers to an unmodified molecule or a variant molecule lacking the particular variation under discussion; for example, when discussing substitution below, the parent molecule may be a deletion variant.

Variants may be rapidly screened to assess their physical properties. It will be appreciated that such variant(s) will demonstrate similar properties to the unmodified molecule, but not necessarily all of the same properties and not necessarily to the same degree as the corresponding parent molecule.

There are two principal variables in the construction of amino acid sequence variant(s): the location of the mutation site and the nature of the mutation. In designing variant(s), the location of each mutation site and the nature of each mutation will depend on the biochemical characteristic(s) to be modified. Each mutation site can be modified individually or in series, e.g., by (1) deleting the target amino acid residue, (2) inserting one or more amino acid residues adjacent to the located site or (3) substituting first with conservative amino acid choices and, depending upon the results achieved, then with more radical selections.

Amino acid sequence deletions generally range from about 1 to 30 amino acid residues, preferably from about 1 to 20 amino acid residues, more preferably from about 1 to 10 amino acid residues and most preferably from about 1 to 5 contiguous residues. Amino-terminal, carboxy-terminal and internal intrasequence deletions are contemplated. Deletions within the amino acid sequences of OPG or the sTNFRs may be made, for example, in regions of low homology with the sequences of other members of the NGF/TNF receptor family. In the case of IL-1ra, deletions may be made in regions of low homology in the IL-1 family (which comprises IL-1 □, IL-1 □, and IL-1ra). Deletions in areas of substantial homology with other members of the family will be more likely to significantly modify the biological activity. Specifically, the sequence similarity among NGF/TNF receptor family members is particularly high in the region corresponding to the first two disulfide loops of domain 1, the whole of domain 2, and the first disulfide loop of domain 3 (Banner et al. (1993), Cell, 73:431445). The number of total deletions and/or consecutive deletions preferably will be selected so as to preserve the tertiary structure in the affected domain, e.g., cysteine crosslinking.

An amino acid sequence addition may include insertions of an amino- and/or carboxyl-terminal fusion ranging in length from one residue to one hundred or more residues, as well as internal intrasequence insertions of single or multiple amino acid residues. Internal additions may range generally from about 1 to 20 amino acid residues, preferably from about 1 to 10 amino acid residues, more preferably from about 1 to 5 amino acid residues, and most preferably from about 1 to 3 amino acid residues. Additions within the amino acid sequences of OPG or the sTNFRs may be made in regions of low homology with the sequences of other members of the NGF/TNF receptor family. Additions within the amino acid sequence of OPG or the sTNFRs in areas of substantial homology with the sequences of other members of the NGF/TNF receptor family will be more likely to significantly modify the biological activity. Additions preferably include amino acid sequences derived from the sequences of the NGF/TNF receptor family members.

An amino-terminus addition is contemplated to include the addition of a methionine (for example, as an artifact of the direct expression in bacterial recombinant cell culture). A further example of an amino-terminal addition includes the fusion of a signal sequence to the amino-terminus of a mature molecule in order to facilitate its secretion from recombinant host cells. Such signal sequences generally will be obtained from and thus be homologous to the intended host cell species. For prokaryotic host cells that do not recognize and process the native signal sequence of the mature molecule, the signal sequence may be substituted by a prokaryotic signal sequence selected, for example, from the group of the alkaline phosphatase, penicillinase or heat-stable enterotoxin II leader sequences. For expression in yeast cells the signal sequence may be selected, for example, from the group of the yeast invertase, alpha factor or acid phosphatase leader sequences. For mammalian cell expression, the native signal sequences (see, e.g., EP 393 438 and EP 422 339 for sTNFRs) are satisfactory, although other mammalian signal sequences may be suitable; for example, sequences derived from other NGF/TNF receptor family members.

An example of an amino- or a carboxy-terminus addition includes chimeric proteins comprising the amino-terminal or carboxy-terminal fusion of the parent molecules with all or part of the constant domain of the heavy or light chain of human immunoglobulin (individually or collectively, (“Fc variant(s)”). Such chimeric polypeptides are preferred wherein the immunoglobulin portion of each comprises all of the domains except the first domain of the constant region of the heavy chain of human immunoglobulin such as IgG (e.g., IgG1 or IgG3), IgA, IgM or IgE. A skilled artisan will appreciate that any amino acid of the immunoglobulin portion can be deleted or substituted with one or more amino acids, or one or more amino acids can be added as long as the parent molecule still maintains some level of its relevant activity and the immunoglobulin portion shows one or more of its characteristic properties.

Another group of variant(s) is amino acid substitution variant(s). These are variant(s) wherein at least one amino acid residue in a parent molecule is removed and a different residue inserted in its place. Substitution variant(s) include allelic variant(s) which are characterized by naturally-occurring nucleotide sequence changes in the species population that may or may not result in an amino acid change. One skilled in the art can use any information known about the binding or active site of the polypeptide in the selection of possible mutation sites.

One method for identifying amino acid residues or regions for mutagenesis of a protein is called “alanine scanning mutagenesis”, as described by Cunningham and Wells (1989), Science, 244:1081-1085, the disclosure of which is hereby incorporated by reference. In this method, an amino acid residue or group of target residues is identified (e.g., charged residues such as Arg, Asp, His, Lys and Glu) and replaced by a neutral or negatively-charged amino acid (most preferably alanine or polyalanine) to affect the interaction of the amino acids with the surrounding aqueous environment in or outside the cell. Those domains/residues demonstrating functional sensitivity to the substitutions are then refined by introducing additional or alternate residues at the sites of substitution. Thus, the site for introducing an amino acid sequence modification is predetermined. To optimize the performance of a mutation at a given site, alanine scanning or random mutagenesis may be conducted and the variant(s) may be screened for the optimal combination of desired activity and degree of activity.

The sites of greatest interest for substitutional mutagenesis include sites in which particular amino acid residues within a parent molecule are substantially different from other species or other family members in terms of side-chain bulk, charge and/or hydrophobicity. Other sites of interest include those in which particular residues of a parent molecule are identical among other species or other family members, as such positions are generally important for the biological activity of a protein.

Other sites of interest include those in which particular residues are similar or identical with proteins with similar structure or activity to the parent molecule. For sTNFR-I, for example, information has been elucidated relevant to sTNFR-I-like molecules (Banner et al. (1993), supra, and Fu et al. (1995), Protein Engineering, 8(12):1233-1241). Residues Tyr⁹, Thr³⁹, His⁵⁵ in Domain 1, residues Phe⁴⁹, Ser⁶³, Asp⁸² in Domain 2 and residues Tyr⁹² and Ser¹⁰⁷ in Domain 3 have been identified as being potentially important for the stabilization of the structure of Domains 1, 2 and 3, respectively. Residues Pro¹² and His⁵⁵ have been identified as potentially interacting with Ser⁸⁶-Tyr⁸⁷ on subunit C of TNF-α. Residues Glu⁴⁵-Phe⁴⁹ have been identified as being in a loop which potentially interacts with residues Leu²⁹-Arg³² of TNF-α subunit A. Residues Gly⁴⁸ has been identified as potentially interacting with Asn¹⁹-Pro²⁰ on subunit A of TNF-A. Residue His⁵⁸-Leu⁶⁰ have been identified as being in an extended strand conformation and side chain interactions with residues Arg³¹-Ala³³ on subunit A of TNF-α have been potentially identified with residue His⁵⁸ of sTNFR-I specifically interacting with residue Arg³¹. Residues Lys⁶⁴-Arg⁶⁶ have been identified as being in an extended strand conformation and have been identified as having side chain and main chain interactions with residues Ala¹⁴⁵-Glu¹⁴⁶ and residue Glu⁴⁶ on subunit A of TNF-α. Residue Met⁶⁹ has been identified as potentially interacting with residue Tyr¹¹⁵ on subunit A of TNF-α. Residues His⁹⁴-Phe¹⁰¹ have been identified as forming a loop which interacts with residues Thr⁷²-Leu⁷⁵ and Asn¹³⁷ of subunit C of TNF-α, with residue Trp⁹⁶ of sTNFR-I specifically interacting with residues Ser⁷¹-Thr⁷² on subunit C of TNF-α, Leu¹⁰⁰ of sTNFR-I being in close proximity with residue Asn¹³⁷ on subunit C of TNF-α and residue Gln¹⁰² of sTNFR-I specifically interacting with residue Pro¹¹³ on subunit A of TNF-α.

In addition to the cysteines forming the 3 pairs of disulfide bonds within each of the four domains of the molecule, there are several other conserved residues that contribute to the stabilization of the tertiary fold of each domain.

There are two main classes into which these stabilizing residues fall. The first type contributes to the shielding of the disulfide bond sulfur atoms from solvent. An example of this residues in domain 3 is Tyr⁹². In domain 4 Phe¹³³ helps to shield the Cys¹²⁸-Cys¹³⁹ disulfide bond. All four domains have either a Tyr or Phe at these same structurally conserved locations. The second class of stabilizing residues form hydrogen bonds within their respective domains. Within domain 3 Asn¹²³ and Ser¹⁰⁷ form a hydrogen bond and Ser¹⁰⁷ forms an additional hydrogen bond with Thr¹²⁴. For domain 4 these residues include Asn¹⁴⁴ and Ser¹⁴¹.

In addition there are hydrogen bonds between domain 3 and 4 that are not seen between other domains. These hydrogens bonds are (1) Asn¹⁰⁵ main-chain oxygen and Asn¹³⁷ side-chain nitrogen and (2) Ser¹⁰⁷ side-chain oxygen and Asn¹³⁷ main-chain nitrogen.

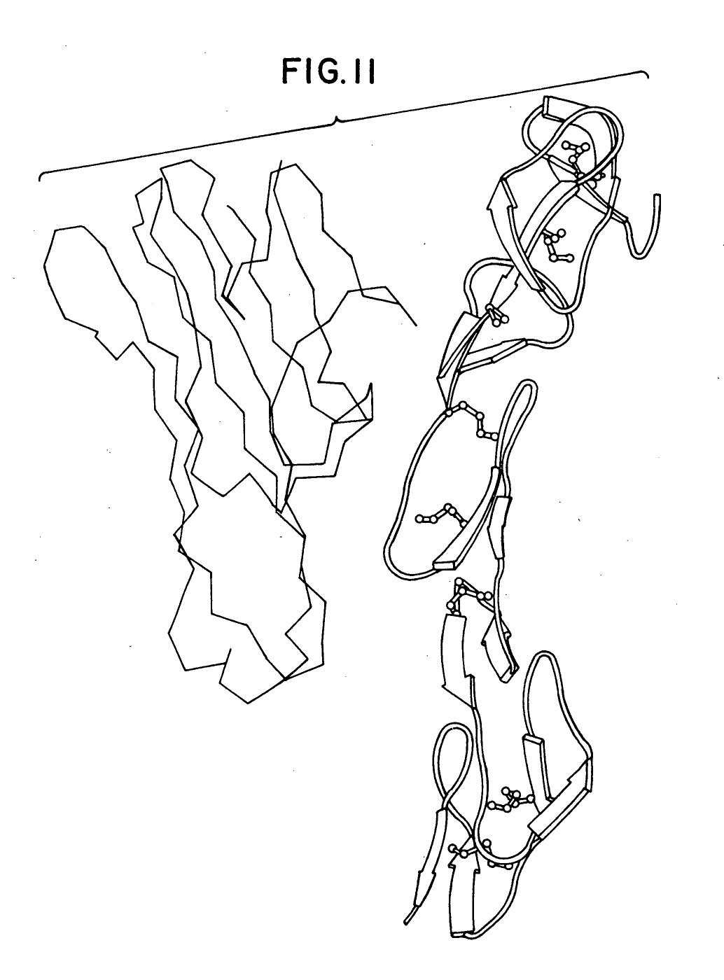

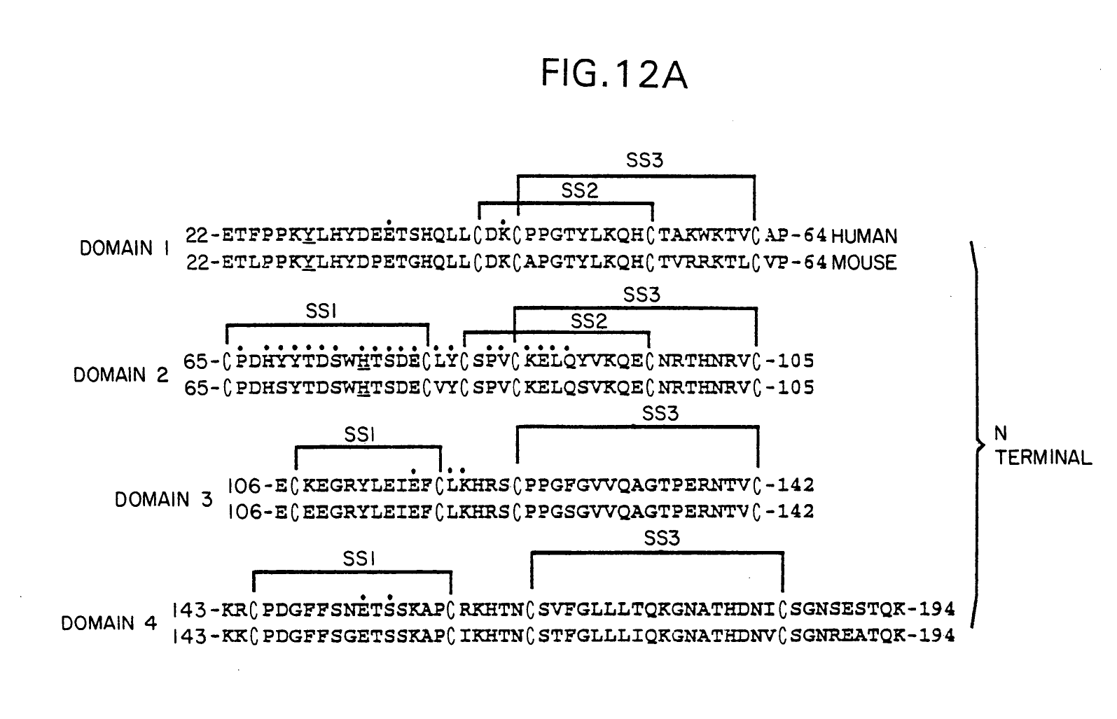

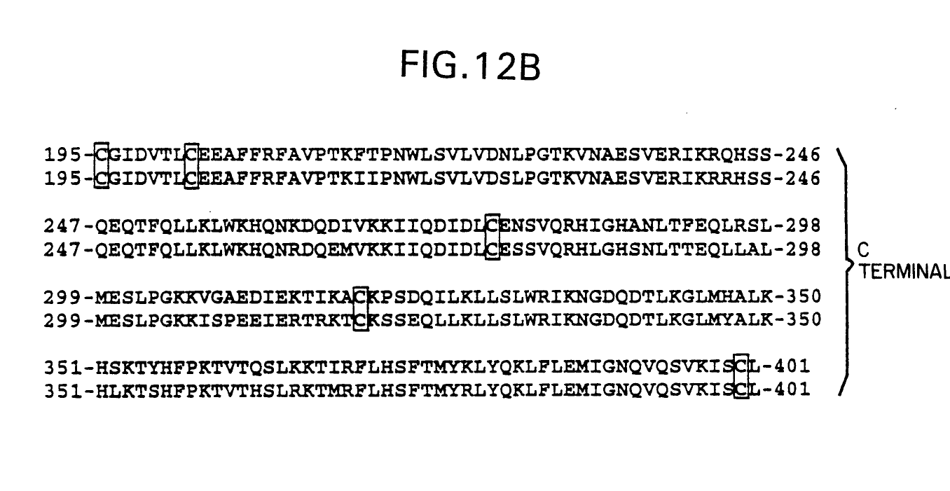

Another useful tool in identifying sites suitable for substitution is molecular modeling. One example of this technique is OPG. Using the homology between OPG and the extracellular ligand binding domains of TNF receptor family members, a three-dimensional model of OPG was generated based upon the known crystal structure of the extracellular domain of TNFR-I (see Example 6). This model was used to identify those residues within OPG which may be important for biological activity. Cysteine residues that are involved in maintaining the structure of the four cysteine-rich domains were identified. The following disulfide bonds were identified in the model: Domain 1: cys41 to cys54, cys44 to cys62, tyr23 and his 66 may act to stabilize the structure of this domain; Domain 2: cys65 to cys80, cys83 to cys98, cys87 to cys105; Domain 3: cys107 to cys118, cys124 to cys142; Domain 4: cys145 to cys160, cys166 to cys185. Residues were also identified which were in close proximity to TNF□ as shown in FIGS. 11 and 12A-12B. In this model, it is assumed that OPG binds to a corresponding ligand; TNF□ was used as a model ligand to simulate the interaction of OPG with its ligand. Based upon this modeling, the following residues in OPG may be important for ligand binding: glu34, lys43, pro66 to gln91 (in particular, pro66, his68, tyr69, tyr70, thr71, asp72, ser73, his76, ser77, asp78, glu79, leu81, tyr82, pro85, va186, lys88, glu90 and gln91), glu153 and ser155.

Alterations in these amino acid residues, either singly or in combination, may alter the biological activity of OPG. For example, changes in specific cysteine residues may alter the structure of individual cysteine-rich domains, whereas changes in residues important for ligand binding may affect physical interactions of OPG with ligand. Structural models can aid in identifying analogs which have more desirable properties, such as enhanced biological activity, greater stability, or greater ease of formulation.

A skilled artisan will appreciate that initially sites should be modified by substitution in a relatively conservative manner. Such conservative substitutions are shown in Table 1 under the heading of “Preferred Substitutions”. If such substitutions result in a change in biological activity, then more substantial changes (Exemplary Substitutions) may be introduced and/or other additions/deletions may be made and the resulting products screened.

TABLE 1
Amino Acid Substitutions

| Original | Preferred | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Lys; Arg |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Arg | Asn; Gln; Lys; Arg |
| Ile (I) | Leu | Leu; Val; Met; Ala; |
| | | Phe; norleucine |
| Leu (L) | Ile | norleucine; Ile; Val; |
| | | Met; Ala; Phe |
| Lys (K) | Arg | Arg; Gln; Asn |
| Met (M) | Leu | Leu; Phe; Ile |
| Phe (F) | Leu | Leu; Val; Ile; Ala |
| Pro (P) | Gly | Gly |
| Ser (S) | Thr | Thr |
| Thr (T) | Ser | Ser |
| Trp (W) | Tyr | Tyr |
| Tyr (Y) | Phe | Trp; Phe; Thr; Ser |
| Val (V) | Leu | Ile; Leu; Met; Phe; Ala; |
| | | norleucine |

In making such changes, the hydropathic index of amino acids may be considered. The importance of the hydropathic amino acid index in conferring interactive biological function on a protein is generally understood in the art (Kyte and Doolittle (1982), J. Mol. Biol., 157:105-131, the disclosure of which is incorporated herein by reference). It is known that certain amino acids may be substituted for other amino acids having a similar hydropathic index or score and still retain a similar biological activity.

It is also understood in the art that the substitution of like amino acids can be made effectively on the basis of hydrophilicity, particularly where the functionally equivalent protein or peptide thereby created is intended for use in immunological embodiments, as in the present case. U.S. Pat. No. 4,554,101, the disclosure of which is incorporated herein by reference, states that the greatest local average hydrophilicity of a protein, as governed by the hydrophilicity of its adjacent amino acids, correlates with its immunogenicity and antigenicity, i.e., with a biological property of the protein.

U.S. Pat. No. 4,554,101 also teaches the identification and preparation of epitopes from primary amino acid sequences on the basis of hydrophilicity. Through the methods disclosed in U.S. Pat. No. 4,554,101 a skilled artisan would be able to identify epitopes, for example, within the amino acid sequence of an sTNFR. These regions are also referred to as “epitopic core regions”. Numerous scientific publications have been devoted to the prediction of secondary structure, and to the identification of epitopes, from analyses of amino acid sequences (Chou and Fasman (1974), Biochemistry, 13(2):222-245; Chou and Fasman (1974), Biochemistry, 13(2):211-222; Chou and Fasman (1978), Adv. Enzymol. Relat. Areas Mol. Biol., 47:45-148; Chou and Fasman (1978), Ann. Rev. Biochem., 47:251-276 and Chou and Fasman (1979), Biophys. J., 26:367-384, the disclosures of which are incorporated herein by reference). Moreover, computer programs are currently available to assist with predicting antigenic portions and epitopic core regions of proteins. Examples include those programs based upon the Jameson-Wolf analysis (Jameson and Wolf (1988), Comput. Appl. Biosci., 4(1):181-186 and Wolf et al. (1988), Comput. Appl. Biosci., 4(1):187-191, the disclosures of which are incorporated herein by reference); the program PepPlot® (Brutlag et al. (1990), CABS, 6:237-245 and Weinberger et al. (1985), Science, 228:740-742, the disclosures of which are incorporated herein by reference); and other programs for protein tertiary structure prediction (Fetrow and Bryant (1993), BIOTECHNOLOGY, 11:479-483, the disclosure of which is incorporated herein by reference).

In contrast, substantial modifications in the functional and/or chemical characteristics of a parent molecule may be accomplished by selecting substitutions that differ significantly in their effect on maintaining (a) the structure of the polypeptide backbone in the area of the substitution, for example, as a sheet or helical conformation, (b) the relative charge or hydrophobicity of the protein at the target site or (c) the bulk of the side chain. Naturally-occurring residues are divided into groups based on common side chain properties:
1) hydrophobic: norleucine, Met, Ala, Val, Leu, Ile;
2) neutral hydrophilic: Cys, Ser, Thr;
3) acidic: Asp, Glu;
4) basic: Asn, Gln, His, Lys, Arg;
5) aromatic: Trp, Tyr, Phe; and
6) residues that influence chain orientation: Gly, Pro.

Non-conservative substitutions may involve the exchange of a member of one of these groups for another. For example, substituted residues may be introduced into regions of OPG or the sTNFRs that are homologous with other NGF/TNF receptor family members or into non-homologous regions of the protein.

A variety of amino acid substitutions or deletions may be made to modify or add N-linked or O-linked glycosylation sites, resulting in a protein with altered glycosylation. The sequence may be modified to add glycosylation sites to or to delete N-linked or O-linked glycosylation sites from the parent molecule. An asparagine-linked glycosylation recognition site comprises a tripeptide sequence which is specifically recognized by appropriate cellular glycosylation enzymes. These tripeptide sequences are either Asn-Xaa-Thr or Asn-Xaa-Ser, where Xaa can be any amino acid other than Pro. In the 30 kDa TNF inhibitor, for example, proven or predicted asparagine residues exist at positions 14, 105 and 111.

Specific mutations of the sequences of the parent molecules may involve substitution of a non-native amino acid at the amino-terminus, carboxy-terminus or at any site of the protein that is modified by the addition of an N-linked or O-linked carbohydrate. Such modifications may be of particular utility in the addition of an amino acid (e.g., cysteine), which is advantageous for the linking of a water-soluble polymer to form a derivative. For example, WO 92/16221 describes the preparation of sTNFR-I muteins, e.g., wherein an asparagine residue at position 105 of the native human protein is changed to cysteine (c105 sTNFR-I).

In a specific embodiment, a variant polypeptide will preferably be substantially homologous to the amino acid of the parent molecule from which it is derived. The term “substantially homologous” as used herein means a degree of homology that is in excess of 80%, preferably in excess of 90%, more preferably in excess of 95% or most preferably even 99%. The percentage of homology as described herein is calculated as the percentage of amino acid residues found in the smaller of the two sequences which align with identical amino acid residues in the sequence being compared when four gaps in a length of 100 amino acids may be introduced to assist in that alignment, as set forth by Dayhoff (1972), Atlas of Protein Sequence and Structure, 5:124, National Biochemical Research Foundation, Washington, D.C., the disclosure of which is hereby incorporated by reference. Also included within the term “substantially homologous” are variant(s) of parent molecules that may be isolated by cross-reactivity with antibodies to the parent molecule amino acid sequences or whose genes may be isolated through hybridization with the DNA of parent molecules or segments thereof.

Polypeptide Derivatives

This invention also comprises chemically modified derivatives of the parent molecule(s) in which the protein is linked to a nonproteinaceous moiety (e.g., a polymer) in order to modify properties. These chemically modified parent molecules are referred to herein as “derivatives”. Such derivatives may be prepared by one skilled in the art given the disclosures herein. Conjugates may be prepared using glycosylated, non-glycosylated or de-glycosylated parent molecule(s) and suitable chemical moieties. Typically non-glycosylated parent molecules and water-soluble polymers will be used. Other derivatives encompassed by the invention include post-translational modifications (e.g., N-linked or O-linked carbohydrate chains, processing of N-terminal or C-terminal ends), attachment of chemical moieties to the amino acid backbone, and chemical modifications of N-linked or O-linked carbohydrate chains. The polypeptides may also be modified with a detectable label, such as an enzymatic, fluorescent, isotopic or affinity label to allow for detection and isolation of the protein.

Water-soluble polymers are desirable because the protein to which each is attached will not precipitate in an aqueous environment, such as a physiological environment. Preferably, the polymer will be pharmaceutically acceptable for the preparation of a therapeutic product or composition. One skilled in the art will be able to select the desired polymer based on such considerations as whether the polymer/protein conjugate will be used therapeutically and, if so, the therapeutic profile of the protein (e.g., duration of sustained release; resistance to proteolysis; effects, if any, on dosage; biological activity; ease of handling; degree or lack of antigenicity and other known effects of a water-soluble polymer on a therapeutic proteins).

Suitable, clinically acceptable, water-soluble polymers include but are not limited to polyethylene glycol (PEG), polyethylene glycol propionaldehyde, copolymers of ethylene glycol/propylene glycol, monomethoxy-polyethylene glycol, carboxymethylcellulose, dextran, polyvinyl alcohol (PVA), polyvinyl pyrrolidone, poly-1,3-dioxolane, poly-1,3,6-trioxane, ethylene/maleic anhydride copolymer, poly(β-amino acids) (either homopolymers or random copolymers), poly(n-vinyl pyrrolidone)polyethylene glycol, polypropylene glycol homopolymers (PPG) and other polyalkylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and mixtures thereof. As used herein, polyethylene glycol is meant to encompass any of the forms that have been used to derivatize other proteins, such as mono-(C1-C10) alkoxy- or aryloxy-polyethylene glycol. Polyethylene glycol propionaldehyde may have advantages in manufacturing due to its stability in water.

The water-soluble polymers each may be of any molecular weight and may be branched or unbranched. Generally, the higher the molecular weight or the more branches, the higher the polymer:protein ratio. The water-soluble polymers each typically have an average molecular weight of between about 2 kDa to about 100 kDa (the term “about” indicating that in preparations of a water-soluble polymer, some molecules will weigh more, some less, than the stated molecular weight). The average molecular weight of each water-soluble polymer preferably is between about 5 kDa and about 40 kDa, more preferably between about 10 kDa and about 35 kDa and most preferably between about l5 kDa and about 30 kDa.

There are a number of attachment methods available to those skilled in the art, including acylation reactions or alkylation reactions (preferably to generate an amino-terminal chemically modified protein) with a reactive water-soluble molecule. See, for example, EP 0 401 384; Malik et al. (1992), Exp. Hematol., 20:1028-1035; Francis (1992), Focus on Growth Factors, 3(2):4-10, published by Mediscript, Mountain Court, Friern Barnet Lane, London N20 OLD, UK; EP 0 154 316; EP 0 401 384; WO 92/16221; WO 95/34326; WO 95/13312; WO 96/11953; WO 96/19459 and WO 96/19459 and the other publications cited herein that relate to pegylation, the disclosures of which are hereby incorporated by reference.

Pegylation also may be specifically carried out using water-soluble polymers having at least one reactive hydroxy group (e.g. polyethylene glycol). The water-soluble polymer can be reacted with an activating group, thereby forming an “activated linker” useful in modifying various proteins. The activated linkers can be monofunctional, bifunctional, or multifunctional.

Activating groups which can be used to link the water-soluble polymer to two or more proteins include the following: sulfone, maleimide, sulfhydryl, thiol, triflate, tresylate, azidirine, oxirane and 5-pyridyl. Useful reagents having a reactive sulfone group that can be used in the methods include, without limitation, chlorosulfone, vinylsulfone and divinylsulfone. These PEG derivatives are stable against hydrolysis for extended periods in aqueous environments at pHs of about 11 or less, and can form linkages with molecules to form conjugates which are also hydrolytically stable. Useful homobifunctional derivatives are PEG-bis-chlorosulfone and PEG-bis-vinylsulfone (see WO 95/13312).

WO 97/04003, the disclosure of which is hereby incorporated by reference, teaches methods of making sulfone-activated linkers by obtaining a compound having a reactive hydroxyl group and converting the hydroxyl group to a reactive Michael acceptor to form an activated linker, with tetrahydrofuran as the solvent for the conversion. The application also teaches a process for purifying the activated linkers which utilizes hydrophobic interaction chromatography to separate the linkers based on size and end-group functionality.

As an example, chemically modified derivatives of OPG may provide such advantages as increased stability, increased time in circulation, or decreased immunogenicity (see U.S. Pat. No. 4,179,337). The chemical moieties for derivitization may be selected from water-soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

One may specifically desire N-terminally chemically modified protein. Using polyethylene glycol as an illustration of the present compositions, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective N-terminal chemically modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Polyvalent Forms

Polyvalent forms, i.e., molecules comprising more than one active moiety, may be constructed. In one embodiment, an sTNFR variant may possess multiple tumor necrosis factor binding sites for the TNF ligand. Additionally, the molecule may possess at least one tumor necrosis factor binding site and, depending upon the desired characteristic of polyvalent form, at least one site of another molecule (e.g., a TN\overline{F}□ inhibitor(s), and an OPG).

Active moieties may be linked using conventional coupling techniques (see WO 92/16221, WO 95/13312 and WO 95/34326, the disclosures of which are hereby incorporated by reference). For example, WO 92/16221 and WO 95/34326 describe the preparation of various dimerized sTNFR-I molecules, e.g., dimerized c105 sTNFR-I. Techniques for formation of polyvalent forms include photochemical crosslinking (e.g., exposure to ultraviolet light), chemical crosslinking (e.g., with bifunctional linker molecules such as polyethylene glycol), and mutagenesis (e.g., introduction of additional cysteine residues).

Polyvalent forms may be constructed by chemically coupling at least one parent molecule and another moiety with any clinically accepted linker (e.g., a water-soluble polymer). In principle, the linker must not impart new immunogenicity. The linker also must not, by virtue of the new amino acid residues, alter the hydrophobicity and charge balance of the structure, which affects its biodistribution and clearance. A variety of chemical crosslinkers may be used depending upon which properties of the protein dimer are desired. For example, crosslinkers may be short and relatively rigid or longer and more flexible, may be biologically reversible, and may provide reduced immunogenicity or longer pharmacokinetic half-life.

In one example, OPG molecules are linked through the amino terminus by a two step synthesis (see Example 12). In the first step, OPG is chemically modified at the amino terminus to introduce a protected thiol, which after purification is deprotected and used as a point of attachment for site-specific conjugation through a variety of crosslinkers with a second OPG molecule. Amino-terminal crosslinks include, but are not limited to, a disulfide bond, thioether linkages using short-chain, bis-functional aliphatic crosslinkers, and thioether linkages to variable length, bifunctional polyethylene glycol crosslinkers (PEG “dumbbells”). Also encompassed by PEG dumbbell synthesis of OPG dimers is a byproduct of such synthesis, termed a “monobell”. An OPG monobell consists of a monomer coupled to a linear bifunctional PEG with a free polymer terminus. Alternatively, OPG may be crosslinked directly through a variety of amine specific homobifunctional crosslinking techniques which include reagents such as: diethylenetriaminepentaacetic dianhydride (DTPA), p-benzoquinone (pBQ) or bis(sulfosuccinimidyl) suberate (BS³) as well as others known in the art. It is also possible to thiolate OPG directly with reagents such as iminothiolane in the presence of a variety of bifunctional, thiol specific crosslinkers, such as PEG bismaleimide, and achieve dimerization and/or dumbbells in a one step process.

The water-soluble polymers for this polyvalent form can be, based on the monomers listed herein, homopolymers, random or block copolymers, terpolymers straight chain or branched, substituted or unsubstituted. The polymer can be of any length or molecular weight, but these characteristics can affect the biological properties.

Polymer average molecular weights particularly useful for decreasing clearance rates in pharmaceutical applications are in the range of 2,000 to 35,000 daltons. In addition, the length of the polymer can be varied to optimize or confer the desired biological activity.

Alternatively, a bivalent molecule may consist of two tandem repeats of parent molecules separated by a polypeptide linker region. The design of the polypeptide linkers is similar in design to the insertion of short loop sequences between domains in the de novo design of proteins (Mutter (1988), TIBS, 13:260-265 and Regan and DeGrado (1988), Science, 241:976-978, the disclosures of which are hereby incorporated by reference). Several different linker constructs have been assembled and shown to be useful for forming single chain antibodies; the most functional linkers vary in size from 12 to 25 amino acids (amino acids having unreactive side groups, e.g., alanine, serine and glycine) which together constitute a hydrophilic sequence, have a few oppositely charged residues to enhance solubility and are flexible (Whitlow and Filpula (1991), Methods: A Companion to Methods in Enzymology, 2:97-105; and Brigido et al. (1993), J. Immunol., 25 150:469479, the disclosures of which are hereby incorporated by reference). It has been shown that a linker suitable for single chain antibodies is effective to produce a dimeric form of the human sTNFR-II (Neve et al. (1996), Cytokine, 8(5):365-370, the disclosure of which is hereby incorporated by reference).

Self-associating variants are another example of polyvalent forms. Such self-associating variants may be bound covalently (typically by disulfide bonds) or noncovalently. Analysis of carboxy-terminal deletions of OPG, for example, suggest that at least a portion of the region 186-401 is involved in association of OPG polypeptides. Substitution of part or all of the region of OPG amino acids 186-401 with an amino acid sequence capable of self-association is also encompassed by the invention.

Polyvalent forms may also be formed using substitution variants. Parent molecules may be modified to form dimers or multimers by site-directed mutagenesis to create unpaired cysteine residues for interchain disulfide bond formation.

Additionally, a parent molecule may be chemically coupled to biotin, and the resulting conjugate may then be allowed to bind to avidin, resulting in tetravalent avidin/biotin/parent molecules. A parent molecule may also be covalently coupled to dinitrophenol (DNP) or trinitrophenol (TNP) and the resulting conjugates precipitated with anti-DNP or anti-TNP-IgM to form decameric conjugates.

In yet another embodiment, recombinant fusion proteins may also be produced wherein each recombinant chimeric molecule has a parent molecule(s) sequence amino-terminally or carboxy-terminally fused to all or part of the constant domains, but at least one constant domain, of the heavy or light chain of human immunoglobulin. For example, a chimeric TN\overline{F}α inhibitor(s)/IgG1 (or IgG1/TN\overline{F}α inhibitor(s)) fusion protein may be produced from a light chain-containing chimeric gene: a TN\overline{F}α inhibitor(s)/human kappa light chain chimera (TN\overline{F}α inhibitor(s)/Ck) or a human kappa light chain/TN\overline{F}α inhibitor(s) chimera (Ck/TNFα inhibitor(s)); or a heavy chain-containing chimeric gene: a TN\overline{F}α inhibitor(s)/human gamma-1 heavy chain chimera (TN\overline{F}α inhibitor(s)/Cg-1) or a human gamma-1 heavy chain/T\overline{F}α inhibitor(s) chimera (Cg-1/TN\overline{F}α inhibitor(s)). Alternatively, an OPG-Fc chimera may be formed as described in WO 97/23614, which is hereby incorporated by reference. Following transcription and translation of a heavy-chain chimeric gene, or of a light chain-containing gene and a heavy-chain chimeric gene, the gene products may be assembled into a single chimeric molecule having a parent molecule(s) displayed bivalently. Additional details relating to the construction of such chimeric molecules are disclosed in U.S. Pat. No. 5,116,964, WO 89/09622, WO 91/16437, WO 97/23614 and EP 315062, the disclosures of which are hereby incorporated by reference.

In yet a further embodiment, recombinant fusion proteins may also be produced wherein each recombinant chimeric molecule has at least one TN\overline{F}α inhibitor(s), as described herein, and at least a portion of the region 186-401 of osteoprotogerin or a variant thereof, as described in European Patent Application No. 96309363.8, the disclosures of which are hereby incorporated by reference. Either the TN\overline{F}α inhibitor(s) or the portion of osteoprotogerin may be at the amino-terminus or the carboxy-terminus of the chimeric molecule.

Nucleic Acids

The invention provides for an isolated nucleic acid encoding a polypeptide having at least one of the biological activities of OPG. As described herein, the biological activities of OPG include, but are not limited to, any activity involving bone metabolism and in particular, include increasing bone density. The nucleic acids of the invention are selected from the following:
a) the nucleic acid sequences as shown in FIGS. 2B-2C (SEQ ID NO: 120), 9A-9B (SEQ ID NO: 122), and 9C-9D (SEQ ID NO: 124) or complementary strands thereof;
b) the nucleic acids which hybridize under stringent conditions with the polypeptide-encoding region in FIGS. 2B-2C (SEQ ID NO: 120), 9A-9B (SEQ ID NO: 122), and 9C-9D (SEQ ID NO: 124); and
c) nucleic acids which hybridize under stringent conditions with nucleotides 148 through 337 inclusive as shown in FIG. 1A.
d) the nucleic acid sequences which are degenerate to the sequences in (a) and (b).

The invention provides for nucleic acids which encode rat, mouse and human OPG as well as nucleic acid sequences hybridizing thereto which encode a polypeptide having at least one of the biological activities of OPG. Also provided for are nucleic acids which hybridize to a rat OPG EST encompassing nucleotides 148-337 as shown in FIG. 1A. The conditions for hybridization are generally of high stringency such as 5×SSC, 50% formamide and 42° C. described in Example 1 of the specification. Equivalent stringency to these conditions may be readily obtained by adjusting salt and organic solvent concentrations and temperature. The nucleic acids in (b) encompass sequences encoding OPG-related polypeptides which do not undergo detectable hybridization with other known members of the TNF receptor superfamily. In a preferred embodiment, the nucleic acids are as shown in FIGS. 2B-2C (SEQ ID NO: 120), 9A-9B (SEQ ID NO: 122), and 9C-9D (SEQ ID NO: 124).

The length of hybridizing nucleic acids of the invention may be variable since hybridization may occur in part or all of the polypeptide-encoding regions as shown in FIGS. 2B-2C (SEQ ID NO: 120), 9A-9B (SEQ ID NO: 122), and 9C-9D (SEQ ID NO: 124), and may also occur in adjacent noncoding regions. Therefore, hybridizing nucleic acids may be truncations or extensions of the sequences shown in FIGS. 2B-2C (SEQ ID NO: 120), 9A-9B (SEQ ID NO: 122), and 9C-9D (SEQ ID NO: 124). Truncated or extended nucleic acids are encompassed by the invention provided they retain one or more of the biological properties of OPG. The hybridizing nucleic acids may also include adjacent noncoding regions which are 5′ and/or 3′ to the OPG coding region. The noncoding regions include regulatory regions involved in OPG expression, such as promoters, enhance, translational initiation sites, transcription termination sites and the like.

Hybridization conditions for nucleic acids are described in Sambrook et al., Molecular Cloning: A Laboratory Manual, 2^{nd} ed. Cold Spring Harbor Laboratory Press, Cold Spring Harbor, N.Y. (1989)

DNA encoding rat OPG was provided in plasmid pMO-B1.1 deposited with the American Type Culture Collection, Rockville, Md. on Dec. 27, 1995 under ATCC accession no. 69970. DNA encoding mouse OPG was provided in plasmid pRcCMV-murine OPG deposited with the American Type Culture Collection, Rockville, Md. on Dec. 27, 1995 under accession no. 69971. DNA encoding human OPG was provided in plasmid pRcCMV—human OPG deposited with the American Type Culture Collection, Rockville, Md. on Dec. 27, 1995 under accession no. 69969. The nucleic acids of the invention will hybridize under stringent conditions to the DNA inserts of ATCC accession nos. 69969, 69970, and 69971 and have at least one of the biological activities of OPG.

Also provided by the invention are derivatives of the nucleic acid sequences as shown in FIGS. 2B, 9A and 9B. As used herein, derivatives include nucleic acid sequences having addition, substitution, insertion or deletion of one or more residues such that the resulting sequences encode polypeptides having one or more amino acid residues which have been added, deleted, inserted or substituted and the resulting polypeptide has the activity of OPG. The nucleic acid derivatives may be naturally occurring, such as by splice variation or polymorphism, or may be constructed using site-directed mutagenesis techniques available to the skilled worker. One example of a naturally occurring variant of OPG is a nucleic acid encoding a lys to asn change at residue 3 within the leader sequence (see Example 5). It is anticipated that nucleic acid derivatives will encode amino acid changes in regions of the molecule which are least likely to disrupt biological activity. Other derivatives include a nucleic acid encoding a membrane-bound form of OPG having an extracellular domain as shown in FIGS. 2B-2C (SEQ ID NO: 120), 9A-9B (SEQ ID NO: 122), and 9C-9D (SEQ ID NO: 124) along with transmembrane and cytoplasmic domains.

In one embodiment, derivatives of OPG include nucleic acids encoding truncated forms of OPG having one or more amino acids deleted from the carboxy terminus. Nucleic acids encoding OPG may have from 1 to 216 amino acids deleted from the carboxy terminus. Optionally, an antibody Fc region may extend from the new carboxy terminus to yield a biologically active OPG-Fc fusion polypeptide. (see Example 11). In preferred embodiments, nucleic acids encode OPG having the amino acid sequence from residues 22-185, 22-189, 22-194 or 22-201 (using numbering in FIG. 9E-F) and optionally, encoding an Fc region of human IgG.

Also included are nucleic acids encoding truncated forms of OPG having one or more amino acids deleted from the amino terminus. Truncated forms include those lacking part or all the 21 amino acids comprising the leader sequence. Additionally, the invention provides for nucleic acids encoding OPG having from 1 to 10 amino acids deleted from the mature amino terminus (at residue 22) and optionally, having from 1 to 216 amino acids deleted from the carboxy terminus (at residue 401). Optionally, the nucleic acids may encode a methionine residue at the amino terminus. Examples of such OPG truncated polypeptides are described in Example 8.

Examples of the nucleic acids of the invention include cDNA, genomic DNA, synthetic DNA and RNA. cDNA is obtained from libraries prepared from mRNA isolated from various tissues expressing OPG. In humans, tissue sources for OPG include kidney, liver, placenta and heart. Genomic DNA encoding OPG is obtained from genomic libraries which are commercially available from a variety of species. Synthetic DNA is obtained by chemical synthesis of overlapping oligonucleotide fragments followed by assembly of the fragments to reconstitute part or all of the coding region and flanking sequences (see U.S. Pat. No. 4,695,623 describing the chemical synthesis of interferon genes). RNA is obtained most easily by procaryotic expression vectors which direct high-level synthesis of mRNA, such as vectors using T7 promoters and RNA polymerase.

Nucleic acid sequences of the invention are used for the detection of OPG sequences in biological samples in order to determine which cells and tissues are expressing OPG mRNA. The sequences may also be used to screen cDNA and genomic libraries for sequences related to OPG. Such screening is well within the capabilities of one skilled in the art using appropriate hybridization conditions to detect homologus sequences. The nucleic acids are also useful for modulating the expression of OPG levels by anti-sense therapy or gene therapy. The nucleic acids are also used for the development of transgenic animals which may be used for the production of the polypeptide and for the study of biological activity (see Example 3).

Vectors and Host Cells

Expression vectors containing nucleic acid sequences encoding OPG, host cells transformed with said vectors and methods for the production of OPG are also provided by the invention. An overview of expression of recombinant proteins is found in Methods of Enzymology v. 185, Goeddel, D. V. ed. Academic Press (1990).

Host cells for the production of OPG include procaryotic host cells, such as E. coli, yeast, plant, insect and mammalian host cells. E. coli strains such as HB101 or JM101 are suitable for expression. Preferred mammalian host cells include COS, CHOd-, 293, CV-1, 3T3, baby hamster kidney (BHK) cells and others. Mammalian host cells are preferred when post-translational modifications, such as glycosylation and polypeptide processing, are important for OPG activity. Mammalian expression allows for the production of secreted polypeptides which may be recovered from the growth medium.

Vectors for the expression of OPG contain at a minimum sequences required for vector propogation and for expression of the cloned insert. These sequences include a replication origin, selection marker, promoter, ribosome binding site, enhancer sequences, RNA splice sites and transcription termination site. Vectors suitable for expression in the aforementioned host cells are readily available and the nucleic acids of the invention are inserted into the vectors using standard recombinant DNA techniques. Vectors for tissue-specific expression of OPG are also included. Such vectors include promoters which function specifically in liver, kidney or other organs for production in mice, and viral vectors for the expression of OPG in targeted human cells.

Using an appropriate host-vector system, OPG is produced recombinantly by culturing a host cell transformed with an expression vector containing nucleic acid sequences encoding OPG under conditions such that OPG is produced, and isolating the product of expression. OPG is produced in the supernatant of transfected mammalian cells or in inclusion bodies of transformed bacterial host cells. OPG so produced may be purified by procedures known to one skilled in the art as described below. The expression of OPG in mammalian and bacterial host systems is described in Examples 7 and 8. Expression vectors for mammalian hosts are exemplified by plasmids such as pDSR□ described in PCT Application No. 90/14363. Expression vectors for bacterial host cells are exemplified by plasmids pAMG21 and pAMG22-His described in Example 8. Plasmid pAMG21 was deposited with the American Type Culture Collection, Rockville, Md. on Jul. 24, 1996 under accession no. 98113. Plasmid pAMG22-His was deposited with the American Type Culture Collection, Rockville, Md. on Jul. 24, 1996 under accession no. 98112. It is anticipated that the specific plasmids and host cells described are for illustrative purposes and that other available plasmids and host cells could also be used to express the polypeptides.

The invention also provides for expression of OPG from endogenous nucleic acids by in vivo or ex vivo recombination events to allow modulation of OPG from the host chromosome. Expression of OPG by the introduction of exogenous regulatory sequences (e.g. promoters or enhancers) capable of directing the production of OPG from endogenous OPG coding regions is also encompassed. Stimulation of endogenous regulatory sequences capable of directing OPG production (e.g. by exposure to transcriptional enhancing factors) is also provided by the invention.

Antibodies

Also encompassed by the invention are antibodies specifically binding to OPG.

Antigens for the generation of antibodies may be full-length polypeptides or peptides spanning a portion of the OPG sequence. Immunological procedures for the generation of polyclonal or monoclonal antibodies reactive with OPG are known to one skilled in the art (see, for example, Harlow and Lane, Antibodies: A Laboratory Manual Cold Spring Harbor Laboratory Press, Cold Spring Harbor N.Y. (1988)). Antibodies so produced are characterized for binding specificity and epitope recognition using standard enzyme-linked immunosorbent assays. Antibodies also include chimeric antibodies having variable and constant domain regions derived from different species. In one embodiment, the chimeric antibodies are humanized antibodies having murine variable domains and human constant domains. Also encompassed are complementary determining regions grafted to a human framework (so-called CDR-grafted antibodies). Chimeric and CDR-grafted antibodies are made by recombinant methods known to one skilled in the art. Also encompassed are human antibodies made in mice.

Anti-OPG antibodies of the invention may be used as an affinity reagent to purify OPG from biological samples (see Example 10). In one method, the antibody is immobilized on CnBr-activated Sepharose and a column of antibody-Sepharose conjugate is used to remove OPG from liquid samples. Antibodies are also used as diagnostic reagents to detect and quantitate OPG in biological samples by methods described below.

Pharmaceutical Compositions

The invention also provides for pharmaceutical compositions comprising a therapeutically effective amount of a polypeptide comprising OPG or the other therapeutic molecules used (e.g., IL-1ra, sTNF-RI, or SLPI) together with a pharmaceutically acceptable diluent, carrier, solubilizer, emulsifier, preservative and/or adjuvant. Two or more of the therapeutic molecules (e.g., OPG, IL-1ra, sTNF-RI, or SLPI) can be formulated together or packaged together in a kit. The term “therapeutically effective amount” means an amount which provides a therapeutic effect for a specified condition and route of administration. The composition may be in a liquid or lyophilized form and comprises a diluent (Tris, acetate or phosphate buffers) having various pH values and ionic strengths, solubilizer such as Tween or Polysorbate, carriers such as human serum albumin or gelatin, preservatives such as thimerosal or benzyl alcohol, and antioxidants such as ascrobic acid or sodium metabisulfite. Also encompassed are compositions comprising any of the therapeutic molecules modified with water-soluble polymers to increase solubility or stability. Compositions may also comprise incorporation of any of the therapeutic molecules into liposomes, microemulsions, micelles or vesicles for controlled delivery over an extended period of time.

Specifically, compositions herein may comprise incorporation into polymer matrices such as hydrogels, silicones, polyethylenes, ethylene-vinyl acetate copolymers, or biodegradable polymers. Examples of hydrogels include polyhydroxyalkylmethacrylates (p-HEMA), polyacrylamide, polymethacrylamide, polyvinylpyrrolidone, polyvinyl alcohol and various polyelectrolyte complexes. Examples of biodegradable polymers include polylactic acid (PLA), polyglycolic acid (PGA), copolymers of PLA and PGA, polyamides and copolymers of polyamides and polyesters. Other controlled release formulations include microcapsules, microspheres, macromolecular complexes and polymeric beads which may be administered by injection.

Selection of a particular composition will depend upon a number of factors, including the condition being treated, the route of administration and the pharmacokinetic parameters desired. A more extensive survey of component suitable for pharmaceutical compositions is found in Remington's Pharmaceutical Sciences, 18^{th} ed. A. R. Gennaro, ed. Mack, Easton, Pa. (1980).

Compositions of the invention may be administered by injection, either subcutaneous, intravenous or intramuscular, or by oral, nasal, pulmonary or rectal administration. The route of administration eventually chosen will depend upon a number of factors and may be ascertained by one skilled in the art.

The invention also provides for pharmaceutical compositions comprising a therapeutically effective amount of the nucleic acids of the invention together with a pharmaceutically acceptable adjuvant. Nucleic acid compositions will be suitable for the delivery of part or all of the therapeutic molecule coding region to cells and tissues as part of an anti-sense or gene therapy regimen.

Methods of Treatment

Bone tissue provides support for the body and consists of mineral (largely calcium and phosphorous), a matrix of collagenous and noncollagenous proteins, and cells. Three types of cells found in bone, osteocytes, osteoblasts and osteoclasts, are involved in the dynamic process by which bone is continually formed and resorbed. Osteoblasts promote formation of bone tissue whereas osteoclasts are associated with resorption. Resorption, or the dissolution of bone matrix and mineral, is a fast and efficient process compared to bone formation and can release large amounts of mineral from bone. Osteoclasts are involved in the regulation of the normal remodeling of skeletal tissue and in resorption induced by hormones. For instance, resorption is stimulated by the secretion of parathyroid hormone in response to decreasing concentrations of calcium ion in extracellular fluids. In contrast, inhibition of resorption is the principal function of calcitonin. In addition, metabolites of vitamin D alter the responsiveness of bone to parathyroid hormone and calcitonin.

After skeletal maturity, the amount of bone in the skeleton reflects the balance (or imbalance) of bone formation and bone resorption. Peak bone mass occurs after skeletal maturity prior to the fourth decade. Between the fourth and fifth decades, the equilibrium shifts and bone resorption dominates. The inevitable decrease in bone mass with advancing years starts earlier in females than males and is distinctly accelerated after menopause in some females (principally those of Caucasian and Asian descent).

Osteopenia is a condition relating generally to any decrease in bone mass to below normal levels. Such a condition may arise from a decrease in the rate of bone synthesis or an increase in the rate of bone destruction or both. The most common form of osteopenia is primary osteoporosis, also referred to as postmenopausal and senile osteoporosis. This form of osteoporosis is a consequence of the universal loss of bone with age and is usually a result of increase in bone resorption with a normal rate of bone formation. About 25 to 30 percent of all white females in the United States develop symptomatic osteoporosis. A direct relationship exists between osteoporosis and the incidence of hip, femoral, neck and inter-trochanteric fracture in women 45 years and older. Elderly males develop symptomatic osteoporosis between the ages of 50 and 70, but the disease primarily affects females.

The cause of postmenopausal and senile osteoporosis is unknown. Several factors have been identified which may contribute to the condition. They include alteration in hormone levels accompanying aging and inadequate calcium consumption attributed to decreased intestinal absorption of calcium and other minerals. Treatments have usually included hormone therapy or dietary supplements in an attempt to retard the process. To date, however, an effective treatment for bone loss does not exist.

The invention provides for a method of treating a bone disorder using a therapeutically effective amount of OPG. The bone disorder may be any disorder characterized by a net bone loss (osteopenia or osteolysis). In general, treatment with OPG is anticipated when it is necessary to suppress the rate of bone resorption. Thus treatment may be done to reduce the rate of bone resorption where the resorption rate is above normal or to reduce bone resorption to below normal levels in order to compensate for below normal levels of bone formation.

Conditions which are treatable with OPG include the following:
Osteoporosis, such as primary osteoporosis, endocrine osteoporosis (hyperthyroidism, hyperparathyroidism, Cushing's syndrome, and acromegaly), hereditary and congenital forms of osteoporosis (osteogenesis imperfecta, homocystinuria, Menkes' syndrome, and Riley-Day syndrome) and osteoporosis due to immobilization of extremities.
Paget's disease of bone (osteitis deformans) in adults and juveniles
Osteomyelitis, or an infectious lesion in bone, leading to bone loss.
Hypercalcemia resulting from solid tumors (breast, lung and kidney) and hematologic malignacies (multiple myeloma, lymphoma and leukemia), idiopathic hypercalcemia, and hypercalcemia associated with hyperthyroidism and renal function disorders.
Osteopenia following surgery, induced by steroid administration, and associated with disorders of the small and large intestine and with chronic hepatic and renal diseases.
Osteonecrosis, or bone cell death, associated with traumatic injury or nontraumatic necrosis associated with Gaucher's disease, sickle cell anemia, systemic lupus erythematosus, rheumatoid arthritis, periodontal disease, osteolytic metastasis, and other conditions

It is understood that OPG may be used alone or in conjunction with other factors for the treatment of bone disorders. In one embodiment, osteoprotegerin is used in conjunction with a therapeutically effective amount of a factor which stimulates bone formation. Such factors include but are not limited to the bone morphogenic factors designated BMP-1 through BMP-12; transforming growth factor-β (TGF-β) and TGF-β family members; interleukin-1 (IL-1) inhibitors; TNF□ inhibitors; parathyroid hormone and analogs thereof, parathyroid related protein and analogs thereof; E series prostaglandins; bisphosphonates (such as alendronate and others); bone-enhancing minerals such as fluoride and calcium; non-steroidal anti-inflammatory drugs (NSAIDs), including COX-2 inhibitors, such as Celebrex™ and Vioxx™; immunosuppressants, such as methotrexate or leflunomide; serine protease inhibitors such as secretory leukocyte protease inhibitor (SLPI); IL-6 inhibitors (e.g., antibodies to IL-6), IL-8 inhibitors (e.g., antibodies to IL-8); IL-18 inhibitors (e.g., IL-18 binding protein or IL-18 antibodies); Interleukin-1 converting enzyme (ICE) modulators; fibroblast growth factors FGF-1 to FGF-10 and FGF modulators; PAF antagonists; keratinocyte growth factor (KGF), KGF-related molecules, or KGF modulators; matrix metalloproteinase (MMP) modulators; Nitric oxide synthase (NOS) modulators, including modulators of inducible NOS; modulators of glucocorticoid receptor; modulators of glutamate receptor; modulators of lipopolysaccharide (LPS) levels; and noradrenaline and modulators and mimetics thereof.

The invention also relates to treatment of IL-1 mediated disease by treatment with an IL-1 inhibitor in conjunction with a serine protease inhibitor. In particular, this method is useful for treatment of asthma and rheumatoid arthritis.

The invention relates further to treatment of TNF-mediated disease by treatment with a TNF inhibitor in conjunction with a serine protease inhibitor. In particular, this method is useful for treatment of rheumatoid arthritis.

In preferred embodiments, a polypeptide comprising OPG is used in conjunction with particular therapeutic molecules to treat various inflammatory conditions, autoimmune conditions, and other conditions leading to bone loss. Depending on the condition and the desired level of treatment, two, three, or more agents may be administered. These agents may be provided together by inclusion in the same formulation or inclusion in a treatment kit, or they may be provided separately. When administered by gene therapy, the genes encoding the protein agents may be included in the same vector, optionally under the control of the same promoter region, or in separate vectors. Particularly preferred molecules in the aforementioned classes are as follows.
IL-1 inhibitors: IL-1ra proteins and soluble IL-1 receptors. The most preferred IL-1 inhibitor is anakinra.
TNF-α inhibitors: soluble tumor necrosis factor receptor type I (sTNF-RI; -RI is also called the p55 receptor); soluble tumor necrosis factor receptor type II (also called the p75 receptor); and monoclonal antibodies that bind the TNF receptor. Most preferred is sTNF-RI as described in WO 98/24463, etanercept (Enbrel®), and Avakine®. Exemplary TNF-® inhibitors are described in EP 422 339, EP 308 35 378, EP 393 438, EP 398 327, and EP 418 014.
serine protease inhibitors: SLPI, ALP, MPI, HUSI-I, BMI, and CUSI. These inhibitors also may be viewed as exemplary LPS modulators, as SLPI has been shown to inhibit LPS responses. Jin et al. (1997), Cell 88(3): 417-26 (incorporated by reference).

Particularly preferred methods of treatment concern use of TNF-α inhibitors and IL-1 inhibitors in conjunction with polypeptides comprising OPG. Such polypeptides may be used with either or both TNF-β inhibitors and IL-1 inhibitors for treatment of conditions such as rheumatoid arthritis and multiple sclerosis.

The following examples are offered to more fully illustrate the invention, but are not construed as limiting the scope thereof.

### EXAMPLE 1

### Identification and Isolation of the Rat OPG cDNA

Materials and methods for cDNA cloning and analysis are described in Maniatis et al, ibid. Polymerase chain reactions (PCR) were performed using a Perkin-Elmer 9600 thermocycler using PCR reaction mixture (Boehringer-Mannheim) and primer concentrations specified by the manufacturer. In general, 25-50 □1 reactions were denatured at 94°C., followed by 20-40 cycles of 94° C. for 5 seconds, 50-60° C. for 5 seconds, and 72° C. for 3-5 minutes. Reactions were the treated for 72° C. for 3-5 minutes. Reactions were then analyzed by gel electrophoresis as described in Maniatis et al., ibid.

A cDNA library was constructed using mRNA isolated from embryonic d20 intestine for EST analysis (Adams et al. Science 252, 1651-1656 (1991)). Rat embryos were dissected, and the entire developing small and large intestine removed and washed in PBS. Total cell RNA was purified by acid guanidinium thiocyanate-phenol-chloroform extraction (Chomczynski and Sacchi Anal. Biochem. 162. 156-159, (1987)). The poly (A+) mRNA fraction was obtained from the total RNA preparation by adsorption to, and elution from, Dynabeads Oligo (dT)25 (Dynal Corp) using the manufacturer's recommended procedures. A random primed cDNA library was prepared using the Superscript Plasmid System (Gibco BRL, Gaithersburg, Md.). The random cDNA primer containing an internal Not I restriction site was used to initiate first strand synthesis and had the following sequence:

| | |
|---|---|
| 5′-AAAGGAAGGAAAAAAGCGGCCGCTACANNNNNNNNT-3′ | (SEQ ID NO:1) |
| Not I | |

For the first strand synthesis three separate reactions were assembled that contained 2.5 μg of poly(A) RNA and 120 ng, 360 ng or 1,080 ng of random primer. After second strand synthesis, the reaction products were separately extracted with a mixture of phenol:choroform:isoamyl alcohol (25:24:1 ratio), and then ethanol precipitated. The double strand (ds) cDNA products of the three reactions were combined and ligated to the following ds oligonucleotide adapter:

| | |
|---|---|
| 5′-TCGACCCACGCGTCCG-3′ | (SEQ ID NO:2) |
| 3′-GGGTGCGCAGGCp-5′ | (SEQ ID NO:3) |

After ligation the cDNA was digested to completion with Not I, extracted with phenol:chloroform:isoamyl (25:24:1) alcohol and ethanol precipitated. The resuspended cDNA was then size fractionated by gel filtration using premade columns provided with the Superscript Plasmid System (Gibco BRL, Gaithersburg, Md.) as recommended by the manufacturer. The two fractions containing the largest cDNA products were pooled, ethanol precipitated and then directionally ligated into Not I and Sal I digested pMOB vector DNA (Strathmann et al, 1991). The ligated cDNA was introduced into competent ElectroMAX DH10B E. coli (Gibco BRL, Gaithersburg, Md.) by electroporation. For automated sequence analysis approximately 10,000 transformants were plated on 20 cm×20 cm agar plates containing ampicillin supplemented LB nutrient media. The colonies that arose were picked and arrayed onto 96 well microtiter plates containing 200 ml of L-broth, 7.5% glycerol, and 50 μg/ml ampicillin. The cultures were grown overnight at 37° C., a duplicate set of microtiter plates were made using a sterile 96 pin replicating tool, then both sets were stored at −80° C. for further analysis. For full-length cDNA cloning approximately one million transformants were plated on 96 bacterial ampicillin plates containing about 10,000 clones each. The plasmid DNA from each pool was separately isolated using the Qiagen Plasmid Maxi Kit (Qiagen Corp.,Germany) and arrayed into 96 microtiter plates for PCR analyses.

To sequence random fetal rat intestine cDNA clones, glycerol stocks were thawed, and small aliquots diluted 1:25 in distilled. Approximately 3.0 ul of diluted bacterial cultures were added to PCR reaction mixture (Boehringer-Mannheim) containing the following oligonucleotides:

| | |
|---|---|
| 5′-TGTAAAACGACGGCCAGT-3′ | (SEQ ID NO:4) |
| 5′-CAGGAAACAGCTATGACC-3′ | (SEQ ID NO:5) |

The reactions were incubated in a thermocycler (Perkin-Elmer 9600) with the following cycle conditions: 94 C for 2 minutes; 30 cycles of 94° C. for 5 seconds, 50° C. for seconds, and 72° C. for 3 minutes.; 72° C. for 4 minutes. After incubation in the thermocycler, the reactions were diluted with 2.0 nL of water. The amplified DNA fragments were further purified using Centricon columns (Princeton Separations) using the manufacturer's recommended procedures. The PCR reaction products were sequenced on an Applied Biosystems 373A automated DNA sequencer using T3 primer (oligonucleotide 353-23; 5′-CAATTAACCCTCACTAAAGG-3′) (SEQ ID NO:6) Taq dye-terminator reactions (Applied Biosystems) following the manufacturer's recommended procedures.

The resulting 5′ nucleotide sequence obtained from randomly picked cDNA clones translated and then compared to the existing database of known protein sequences using a modified version of the FASTA program (Pearson et al. Meth. Enzymol. 183, (1990)). Translated sequences were also analysed for the presence of a specific cysteine-rich protein motif found in all known members of the tumor necrosis factor receptor (TNFR) superfamily (Smith et al. (1994) Cell 76: 959-62), using the sequence profile method of Gribskov et al. (1987), Proc. Natl. Acad. Sci. USA 83: 4355-9), as modified by Luethy et al. (1994), Protein Science 3: 139-46.

Using the FASTA and Profile search data, an EST, FRI-1 (Fetal Rat Intestine-1), was identified as a possible new member of the TNFR superfamily. FRI-1 contained an approximately 600 bp insert with a LORF of about 150 amino acids. The closest match in the database was the human type II TNFR (TNFR-II). The region compared showed an about 43% homology between TNFR-II and FRI-I over this 150 aa LORF. Profile analysis using the first and second cysteine-rich repeats of the TNFR superfamily yielded a Z score of about 8, indicating that the FRI-i gene possibly encodes a new family member.

To deduce the structure of the FRI-1 product, the fetal rat intestine cDNA library was screened for full length clones. The following oligonucleotides were derived from the original FRI-1 sequence:

| | |
|---|---|
| 5′-GCATTATGACCCAGAAACCGGAC-3′ | (SEQ ID NO:7) |
| 5′-AGGTAGCGCCCTTCCTCACATTC-3′ | (SEQ ID NO:8) |

These primers were used in PCR reactions to screen 96 pools of plasmid DNA, each pool containing plasmid DNA from 10,000 independent cDNA clones. Approximately 1 ug of plasmid pool DNA was amplified in a PCR reaction mixture (Boehringer-Mannheim) using a Perkin-Elmer 96 well thermal cycler with the following cycle conditions: 2 min at 94° C., 1 cycle; 15 sec at 94° C., then 45 sec at 65° C., 30 cycles; 7 min at 65° C., 1 cycle. PCR reaction products were analysed by gel electrophoresis. 13 out of 96 plasmid DNA pools gave rise to amplified DNA products with the expected relative molecular mass.

DNA from one positive pool was used to transform competent ElectroMAX DH10B E. coli (Gibco BRL, Gaithersburg, Md.) as described above. Approximately 40,000 transformants were plated onto sterile nitrocellulose filters (BA-85, Schleicher and Schuell), and then screened by colony hybridization using a ³²P-dCTP labeled version of the PCR product obtained above. Filters were prehybridized in 5×SSC, 50% deionized formamide, 5× Denhardt's solution, 0.5% SDS, and 100 ug/ml denatured salmon sperm DNA for 24 hours at 42° C. Filters were then hybridized in 5×SSC, 50% deionized formamide, 2× Denhardt's solution, 0.1% SDS, 100 □g/ml denatured salmon sperm DNA, and about 5 ng/ml of labelled probe for about 18 hours at 42° C. The filters were then washed in 2×SSC for 10 min at RT, 1×SSC for 10 minutes at 55° C., and finally in 0.5×SSC for 10-15 min at 55° C. Hybridizing clones were detected following autoradiography, and then replated onto nitrocellulose filters for secondary screening. Upon secondary screening, a plasmid clone (pB1.1) was isolated, then amplified in L-broth media containing 100 ug/ml ampicillin and the plasmid DNA obtained. Both strands of the 2.4 kb pB1.1 insert were sequenced.

The pB1.1 insert sequence was used for a FASTA search of the public database to detect any existing sequence matches and/or similarities. No matches to any known genes or EST's were found, although there was an approximate 45% similarity to the human and mouse TNFR-II genes. A methionine start codon is found at bp 124 of the nucleotide sequence, followed by a LORF encoding 401 aa residues that terminates at bp 1327. The 401 aa residue product is predicted to have a hydrophobic signal peptide of approximately 31 residues at its N-terminus, and 4 potential sites of N-linked glycosylation. No hydrophobic transmembrane spanning sequence was identified using the PepPlot program (Wisconsin GCG package, version 8.1). The deduced 401 aa sequence was then used to search the protein database. Again, there were no existing matches, although there appeared to be a strong similarity to many members of the TNFR superfamily, most notably the human and mouse TNFR-II. A sequence alignment of this novel protein with known members of the TNFR-superfamily was prepared using the Pileup program, and then modified by PrettyPlot (Wisconsin GCG package, version 8.1). This alignment shows a clear homology between the full length FRI-i gene product and all other TNFR family members. The homologus region maps to the extracellular domain of TNFR family members, and corresponds to the three or four cysteine-rich repeats found in the ligand binding domain of these proteins. This suggested that the FRI-1 gene encoded a novel TNFR family member. Since no transmembrane spanning region was detected we predicted that this may be a secreted receptor, similar to TNFR-I derived soluble receptors (Kohno et al. (1990), Proc. Natl. Acad. Sci. USA 87: 8331-5). Due to the apparent biological activity of the FRI-1 gene (vide infra), the product was named Osteoprotegerin (OPG).

### EXAMPLE 2

### OPG mRNA Expression Patterns in Tissues

Multiple human tissue northern blots (Clonetech) were probed with a ³²P-dCTP labelled FRI-1 PCR product to detect the size of the human transcript and to determine patterns of expression. Northern blots were prehybridized in 5×SSPE, 50% formamide, 5× Denhardt's solution, 0.5% SDS, and 100 μg/ml denatured salmon sperm DNA for 2-4 hr at 42° C. The blots were then hybridized in 5×SSPE, 50% formamide, 2× Denhardt's solution, 0.1% SDS, 100 μg/ml denatured salmon sperm DNA, and 5 ng/ml labelled probe for 18-24 hr at 42° C. The blots were then washed in 2×SSC for 10 min at room temperature, 1×SSC for 10 min at 50° C., then in 0.5×SSC for 10-15 min.

Using a probe derived from the rat gene, a predominant mRNA species with a relative molecular mass of about 2.4 kb is detected in several tissues, including kidney, liver, placenta, and heart. Highest levels are detected in the kidney. A large mRNA species of Mr 4.5 and 7.5 kb was detected in skeletal muscle and pancreas. In human fetal tissue, kidney was found to express relatively high levels of the 2.4 kb mRNA. Using a human probe (vide infra), only the 2.4 kb transcript is detected in these same tissues. In addition, relatively high levels of the 2.4 kb transcript was detected in the lymph node, thymus, spleen and appendix. The size of the transcript detected by both the rat and human Osteosprotegerin gene is almost identical to the length of the rat pB1.1 FRI-1 insert, suggesting it was a full length cDNA clone.

### EXAMPLE 3

### Systemic Delivery of OPG in Transgenic Mice

The rat OPG clone pB1.1 was used as template to PCR amplify the coding region for subcloning into an ApoE-liver specific expression vector (Simonet et al. J. Clin. Invest. 94, 1310-1319 (1994), and PCT Application No. US94/11675 and co-owned U.S. Ser. No. 08/221,767. The following 5′ and 3′ oligonucleotide primers were used for PCR amplification, respectively:

| | |
|---|---|
| 5′-GACTAGTCCCACAATGAACAAGTGGCTGTG-3′ | (SEQ ID NO:9) |
| 5′-ATAAGAATGCGGCCGCTAAACTATGAAACAGCC | (SEQ ID NO:10) |
| CAGTGACCATTC-3′ | |

The PCR reaction mixture (Boehringer-Mannheim) was treated as follows: 94° C. for 1 minute, 1 cycle; 94° C. for 20 sec, 62° C. for 30 sec, and 74 C for 1 minute, 25 cycles. Following amplification, the samples were purified over Qiagen PCR columns and digested overnight with SpeI and NotI restriction enzymes. The digested products were extracted and precipitated and subcloned into the ApoE promoter expression vector. Prior to microinjecting the resulting clone, HE-OPG, it was sequenced to ensure it was mutation-free.

The HE-OPG plasmid was purified through two rounds of CsCl density gradient centrifugation. The purified plasmid DNA was digested with XhoI and Ase I, and the 3.6 kb transgene insert was purified by gel electrophoresis. The purified fragment was diluted to a stock injection solution of 1 μg/ml in 5 mM Tris, pH 7.4, 0.2 MM EDTA. Single-cell embryos from BDF1×BDF1-bred mice were injected essentially as described (Brinster et al. (1985), Proc. Natl. Acad. Sci. USA 82: 4338), except that injection needles were beveled and siliconized before use. Embryos were cultured overnight in a CO₂ incubator and 15 to 20 2-cell embryos were transferred to the oviducts of pseudopregnant CD1 female nice.

Following term pregnancy, 49 offspring were obtained from implantation of microinjected embryos. The offspring were screened by PCR amplification of the integrated transgene in genomic DNA samples. The target region for amplification was a 369 bp region of the human Apo E intron which was included in the expression vector. The oligos used for PCR amplification were:

| | |
|---|---|
| 5′-GCC TCT AGA AAG AGC TGG GAC-3′ | (SEQ ID NO:11) |
| 5′-CGC CGT GTT CCA TTT ATG AGC-3′ | (SEQ ID NO:12) |

The conditions for PCR were: 94° C. for 2 minute, 1 cycle; 94° C. for 1 min, 63° C. for 20 sec, and 72° C. for 30 sec, 30 cycles. Of the 49 original offspring, 9 were identified as PCR positive transgenic founders.

At 8-10 weeks of age, five transgenic founders (2, 11, 16, 17, and 28) and five controls ( 1, 12, 15, 18, and 30) were sacrificed for necropsy and pathological analysis.

Liver was isolated from the remaining 4 founders by partial hepatectomy. For partial hepatectomy, the mice were anesthetized and a lobe of liver was surgically removed.

Total cellular RNA was isolated from livers of all transgenic founders, and 5 negative control littermates as described (McDonald et al. Meth. Enzymol. 152, 219 (1987)). Northern blot analysis was performed on these samples to assess the level of transgene expression. Approximately lOug of total RNA from each animal liver was resolved by electrophoresis denaturing gels (Ogden et al. Meth. Enzymol 152, 61 (1987)), then transferred to HYBOND-N nylon membrane (Amersham), and probed with ³²P dCTP-labelled pB1.1 insert DNA. Hybridization was performed overnight at 42° C. in 50% Formamide, 5×SSPE, 0.5% SDS, 5× Denhardt's solution, 100 μg/ml denatured salmon sperm DNA and 2-4×10⁶ cpm of labeled probe/ml of hybridization buffer. Following hybridization, blots were washed twice in 2×SSC, 0.1% SDS at room temperature for 5 min each, and then twice in 0.1×SSC, 0.1% SDS at 55° C. for 5-10 min each. Expression of the transgene in founder and control littermates was determined following autoradiography.

The northern blot data indicate that 7 of the transgenic founders express detectable levels of the transgene mRNA (animal #'s 2,11,16,17,22,33,and 45). The negative control mice and one of the founders (#28) expressed no transgene-related mRNA. Since OPG is predicted to be a secreted protein, overexpression of transgene mRNA should be a proxy for the level of systemically delivered gene product. Of the PCR and northern blot positive mice, animal 2, 17 and 22 expressed the highest levels of transgene mRNA, and may show more extensive biological effects on host cells and tissues.

### EXAMPLE 4

### Biological Activity of OPG

Five of the transgenic mice (animals 2,11,16,17 and 28) and 5 control littermates (animals 1,12,15,18, and 30) were sacrificed for necropsy and pathological analysis using the following procedures:

Prior to euthanasia, all animals had their identification numbers verified, then were weighed, anesthetized and blood drawn. The blood was saved as both serum and whole blood for a complete serum chemistry and hematology panel. Radiography was performed just after terminal anesthesia by lethal CO2 inhalation, and prior to the gross dissection. Following this, tissues were removed and fixed in 10% buffered Zn-Formalin for histological examination. The tissues collected included the liver, spleen, pancreas, stomach, duodenum, ileum, colon, kidney, reproductive organs, skin and mammary glands, bone, brain, heart, lung, thymus, trachea, eosphagus, thyroid, jejunem, cecum, rectum, adrenals, urinary bladder, and skeletal muscle. Prior to fixation the whole organ weights were determined for the liver, stomach, kidney, adrenals, spleen, and thymus. After fixation the tissues were processed into paraffin blocks, and 3 um sections were obtained. Bone tissue was decalcified using a formic acid solution, and all sections were stained with hematoxylin and eosin. In addition, staining with Gomori's reticulin and Masson's trichrome were performed on certain tissues. Enzyme histochemistry was performed to determine the expression of tartrate resistant acid phosphatase (TRAP), an enyzme highly expressed by osteoclasts, multinucleated bone-resorbing cells of monocyte-macrophage lineage. Immunohistochemistry for BrdU and F480 monocyte-macrophage surface antigen was also performed to detect replicating cells and cells of the monocyte-macrophage lineage, respectively. To detect F480 surface antigen expression, formalin fixed, paraffin embedded 4 μm sections were deparaffinized and hydrated to deionized water. The sections were quenched with 3% hydrogen peroxide, blocked with Protein Block (Lipshaw, Pittsburgh, Pa.), and incubated in rat monoclonal anti-mouse F480 (Harlan, Indianapolis, Ind.). This antibody was detected by biotinylated rabbit anti-rat immunoglobulins, peroxidase conjugated strepavidin (BioGenex San Ramon, Calif.) with DAB as chromagen (BioTek, Santa Barbara, Calif.). Sections were counterstained with hematoxylin.

Upon gross dissection and observation of visceral tissues, no abnormalities were found in the transgene expressors or control littermates. Analysis of organ weight indicate that spleen size increased by approximately 38% in the transgenic mice relative to controls. There was a slight enlargement of platelet size and increased circulating unstained cells in the transgene expressors. There was a marginal decrease in platelet levels in the transgene expressors. In addition, the serum uric acid, urea nitrogen, and alkaline phosphatase levels all trended lower in the transgene expressors. The expressors were found to have increased radiodensity of the skeleton, including long bones (femurs), vertebrae, and flat bones (pelvis). The relative size of femurs in the expressors were not different from the the control mice.

Histological analysis of stained sections of bone from the OPG expressors show severe osteopetrosis with the presence of cartilage remnants from the primary spongiosa seen within bone trabeculae in the diaphysis of the femur. A clearly defined cortex was not identifiable in the sections of femur. In normal animals, the central diaphysis is filled with bone marrow. Sections of vertebra also show osteopetrotic changes implying that the OPG-induced skeletal changes were systemic. The residual bone marrow showed predominantly myeloid elements. Megakaryocytes were present. Reticulin stains showed no evidence for reticulin deposition. Immunohistochemistry for F480, a cell surface antigen expressed by cells of monocyte-macrophage derivation in the mouse, showed the presence of F480 positive cells in the marrow spaces. Focally, flattened F480 positive cells could be seen directly adjacent to trabecular bone surfaces.

The mesenchymal cells lining the bony trabeculae were flattened and appeared inactive. Based on H&E and TRAP stains, osteoclasts were rarely found on the trabecular bone surfaces in the OPG expressors. In contrast, osteoclasts and/or chondroclasts were seen in the region of the growth plate resorbing cartilage, but their numbers may be reduced compared to controls. Also, osteoclasts were present on the cortical surface of the metaphysis where modelling activity is usually robust. The predominant difference between the expressors and controls was the profound decrease in trabecular osteoclasts, both in the vertebrae and femurs. The extent of bone accumulation was directly correlated with the level of OPG transgene mRNA detected by northern blotting of total liver RNA.

The spleens from the OPG expressors had an increased amount of red pulp with the expansion due to increased hematopoiesis. All hematopoietic lineages are represented. F480 positive cells were present in both control and OPG expressors in the red pulp. Two of the expressors (2 and 17) had foci of extramedullary hematopoiesis within the liver and this is likely due to the osteopetrotic marrow.

There were no observable abnormalities in the thymus, lymph nodes, gastrointestinal tract, pancreato-hepatobiliary tract, respiratory tract, reproductive system, genito-urinary system, skin, nervous system, heart and aorta, breast, skeletal muscle and fat.

### EXAMPLE 5

### Isolation of Mouse and Human OPG cDNA

A cDNA clone corresponding to the 5′ end of the mouse OPG mRNA was isolated from a mouse kidney cDNA library (Clontech) by PCR amplification. The oligonucleotides were derived from the rat OPG cDNA sequence and are shown below:

| | |
|---|---|
| 5′-ATCAAAGGCAGGGCATACTTCCTG-3′ | (SEQ ID NO:13) |
| 5′-GTTGCACTCCTGTTTCACGGTCTG-3′ | (SEQ ID NO:14) |
| 5′-CAAGACACCTTGAAGGGCCTGATG-3′ | (SEQ ID NO:15) |
| 5′-TAACTTTTACAGAAGAGCATCAGC-3′ | (SEQ ID NO:16) |
| 5′-AGCGCGGCCGCATGAACAAGTGGCTGTGCT | (SEQ ID NO:17) |
| GCG-3′ | |
| 5′-AGCTCTAGAGAAACAGCCCAGTGACCATTC | (SEQ ID NO:18) |
| C-3′ | |

The partial and full-length cDNA products obtained in this process were sequenced. The full-length product was digested with Not I and XbaI, then directionally cloned into the plasmid vector pRcCMV (Invitrogen). The resulting plasmid was named pRcCMV-Mu-OPG. The nucleotide sequence of the cloned product was compared to the rat OPG cDNA sequence. Over the 1300 bp region spanning the OPG LORF, the rat and mouse DNA sequences are approximately 88% identical. The mouse cDNA sequence contained a 401 aa LORF, which was compared to the rat OPG sequence and found to be about 94% identical without gaps. This indicates that the mouse cDNA sequence isolated encodes the murine OPG, and that the sequence and structure has been highly conserved throughout evolution. The mouse OPG sequence contains an identical putative signal peptide at its N-terminus, and all 4 potential sites of N-linked glycosylation are conserved.

A partial human OPG cDNA was cloned from a human kidney cDNA library using the following rat-specific oligonucleotides:

| | |
|---|---|
| 5′-GTG AAG CTG TGC AAG AAC CTG | (SEQ ID NO:19) |
| ATG-3′ | |
| 5′-ATC AAA GGC AGG GCA TAC TTC | (SEQ ID NO:20) |
| CTG-3′ | |

This PCR product was sequenced and used to design primers for amplifying the 3′ end of the human cDNA using a human OPG genomic clone in lambda as template:

| | |
|---|---|
| 5′-TCCGTAAGAAACAGCCCAGTGACC-3′ | (SEQ ID NO:29) |
| 5′-CAGATCCTGAAGCTGCTCAGTTTG-3′ | (SEQ ID NO:21) |

The amplified PCR product was sequenced, and together with the 5′ end sequence, was used to design 5′ and 3′ human-specific primers useful for amplifying the entire human OPG cDNA coding sequences:

| | |
|---|---|
| 5′-AGCGCGGCCGCGGGGACCACAATGAACAAGTT | (SEQ ID NO:22) |
| G-3′ | |
| 5′-AGCTCTAGAATTGTGAGGAAACAGCTCAATGG | (SEQ ID NO:23) |
| C-3′ | |

The full-length human PCR product was sequenced, then directionally cloned into the plasmid vector pRcCMV (Invitrogen) using Not I and Xba I. The resulting plasmid was named pRcCMV-human OPG. The nucleotide sequence of the cloned product was compared to the rat and mouse OPG cDNA sequences. Over the 1300 bp region spanning the OPG LORF, the rat and mouse DNA sequences are approximately 78-88% identical to the human OPG cDNA. The human OPG cDNA sequence also contained a 401 aa LORF, and it was compared to the rat and mouse protein sequences.

The predicted human OPG is approximatlely 85% identical, and about 90% identical to the rat and mouse proteins, respectively. Sequence alignment of rat, mouse and human proteins show that they have been highly conserved during evolution. The human protein is predicted to have a N-terminal signal peptide, and 5 potential sites of N-linked glycosylation, 4 of which are conserved between the rat and mouse OPG.

The DNA and predicted amino acid sequence of mouse OPG is shown in FIGS. 9A and 9B (SEQ ID NO: 122). The DNA and predicted amino acid sequence of human OPG is shown in FIGS. 9C an 9D (SEQ ID NO: 124). A comparison of the rat, mouse and human OPG amino acid sequences is shown in FIGS. 9E and 9F.

Isolation of additional human OPG cDNA clones revealed the presence of a G to C base change at position 103 of the DNA sequence shown in FIG. 9C. This nucleotide change results in substitution of an asparagine for a lysine at position 3 of the amino acid sequence shown in FIG. 9C. The remainder of the sequence in clones having this change was identical to that in FIGS. 9C and 9D.

### EXAMPLE 6

### OPG Three-Dimensional Structure Modelling

The amino-terminal portion of OPG has homology to the extracellular portion of all known members of the TNFR superfamily (FIG. 1C). The most notable motif in this region of TNFR-related genes is an about 40 amino acid, cysteine-rich repeat sequence which folds into distinct structures (Banner et al. (1993), Cell 73: 43145). This motif is usually displayed in four (range 3-6) tandem repeats (see FIG. 1C), and is known to be involved in ligand binding (Beutler and van Huffel (1994), Science 264: 667-73). Each repeat usually contains six interspaced cysteine residues, which are involved in forming three intradomain disulfide bonds, termed SS1, SS2, and SS3 (Banner et al., ibid). In some receptors, such as TNFR2, CD30 and CD40, some of the repeat domains contain only two intrachain disulfide bonds (SS1 and SS3).

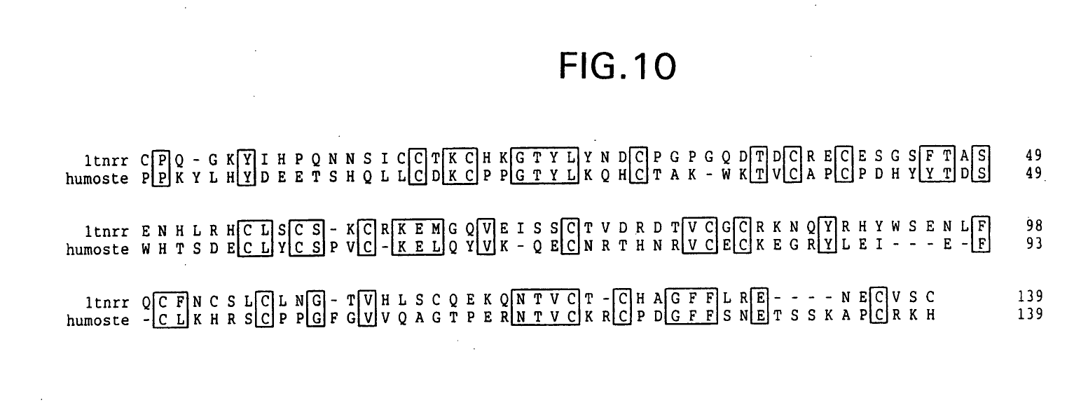

The human OPG sequence was aligned to a TNFR1 extracellular domain profile using methods described by Luethy, et al., ibid, and the results were graphically displayed using the PrettyPlot program from the Wisconsin Package, version 8.1 (Genetics Computer Group, Madison, Wis.) (FIG. 10). The alignment indicates a clear conservation of cysteine residues involved in formation of domains 1-4. This alignment was then used to construct a three-dimensional (3-D) model of the human OPG N-terminal domain using the known 3-D structure of the extracellular domain of p55 TNFR1 (Banner et al., ibid) as the template. To do this the atomic coordinates of the peptide backbone and side chains of identical residues were copied from the crystal structure coordinates of TNFR1. Following this, the remaining coordinates for the insertions and different side chains were generated using the LOOK program (Molecular Applications Group, Palo Alto, Calif.). The 3-D model was then refined by minimizing its conformational energy using LOOK.

By analogy with other TNFR family members, it is assumed that OPG binds to a ligand. For the purpose of modelling the interaction of OPG with its ligand, the crystal structure of TNF-β was used to simulate a 3-D representation of an “OPG ligand”. This data was graphically displayed (see FIG. 11) using Molscript (Kraulis (1991), J. Appl. Cryst. 24: 946-50). A model for the OPG/ligand complex with 3 TNFβ and 3 OPG molecules was constructed where the relative positions of OPG are identical to TNFR1 in the crystal structure. This model was then used to find the residues of OPG that could interact with its ligand using the following approach: The solvent accessible area of all residues in the complex and one single OPG model were calculated. The residues that have different accessibility in the complex than in the monomer are likely to interact with the ligand.

The human and mouse OPG amino acid sequences were realigned using this information to highlight sequences comprising each of the cysteine rich domains 1-4 (FIGS. 12A and 12B). Each domain has individual structural characteristics which can be predicted.

Domain 1: Contains 4 cysteines involved in SS2 (C41 to C54) and SS3 (C44 to C62) disulfide bonds. Although no SS1 bond is evident based on disulfide bridges, the conserved tyrosine at position 28 is homologous to Y20 in TNFR1, which is known to be involved in interacting with H66 to aid in domain formation. OPG has a homologous histidine at position 75, suggesting OPG Y28 and H75 stack together in the native protein, as do the homologous residues in TNFR1. Therefore, both of these residues may indeed be important for biological activity, and N-terminal OPG truncations up to and beyond Y28 may have altered activity. In addition, residues E34 and K43 are predicted to interact with a bound ligand based on our 3-dimensional model.

Domain 2: Contains six cysteines and is predicted to contain SS1 (C65 to C80), SS2 (C83 to C98) and SS3 (C87 to C105) disulfide bonds. This region of OPG also contains an region stretching from P66-Q91 which aligns to the portion of TNFR1 domain 2 which forms close contacts with TNFβ (see above), and may interact with an OPG ligand. In particular residues P66, H68, Y69, Y70, T71, D72, S73, H75, T76, S77, D78, E79, L81, Y82, P85, V86, K88, E89, L90, and Q91 are predicted to interact with a bound ligand based on our structural data.

Domain 3: Contains 4 cysteines involved in SS1 (C107 to C118) and SS3 (C124 to C142) disulfide bonds, but not an SS2 bond. Based on our structural data, residues E115, L118 and K119 are predicted in to interact with an OPG ligand.

Domain 4: Contains 4 cysteines involved in SS1 (C145 to C160) and SS3 (C166 to C185) disulfide bonds, but not an SS2 bond, similar to domain 3. Our structural data predict that E153 and S155 interact with an OPG ligand.

Thus, the predicted structural model for OPG identifies a number of highly conserved residues which are likely to be important for its biological activity.

### EXAMPLE 7

### Production of Recombinant Secreted

### OPG in Mammalian Cells

To determine if OPG is actually a secreted protein, mouse OPG cDNA was fused to the human IgG1 Fc domain as a tag (Capon et al. Nature 337, 525-531 (1989)), and expressed in human 293 fibroblasts. Fc fusions were carried out using the vector pFc-A3. pFc-A3 contains the region encoding the Fc portion of human immunoglobulin IgG-yl heavy chain (Ellison et al. ibid) from the first amino acid of the hinge domain (Glu-99) to the carboxyl terminus and is flanked by a 5′-NotI fusion site and 3′-SalI and XbaI sites. The plasmid was constructed by PCR amplification of the human spleen cDNA library (Clontech). PCR reactions were in a final volume of 100 μl and employed 2 units of Vent DNA polymerase (New England Biolabs) in 20 mM Tris-HCl (pH 8.8), 10 mM KCl, 10 μM (NH₄)2SO₄, 2 mM MgSO₄, 0.1% Triton X-100 with 400 μM each dNTP and 1 ng of the cDNA library to be amplified together with 1 μM of each primer. Reactions were initiated by denaturation at 95° C. for 2 min, followed by 30 cycles of 95° C. for 30 s, 55° C. for 30 s, and 73° C. for 2 min. The 5′ primer

| | |
|---|---|
| 5′ ATAGCGGCCGCTGAGCCCAAATCTTGTGACAAA | (SEQ ID NO:24) |
| ACTCAC 3′ | |

incorporated a NotI site immediately 5′ to the first residue (Glu-99) of the hinge domain of IgG-γ1. The 3′ primer

| | |
|---|---|
| 5′-TCTAGAGTCGACTTATCATTTACCCGGAGACAG | (SEQ ID NO:25) |
| GGAGAGGCTCTT-3′ | |

incorporated SalI and XbaI sites. The 717-bp PCR product was digested with NotI and SalI, isolated by electrophoresis through 1% agarose (FMC Corp.),purified by the Geneclean procedure (BIO 101, Inc.) and cloned into NotI, SalI-digested pBluescript II KS vector (Stratagene). The insert in the resulting plasmid, pFc-A3, was sequenced to confirm the fidelity of the PCR reaction.

The cloned mouse cDNA in plasmid pRcCMV-MuOPG was amplified using the following two sets of primer pairs:

| | |
|---|---|
| Pair 1: | |
| 5′-CCTCTGAGCTCAAGCTTCCGAGGACCACAA | (SEQ ID NO:26) |
| TGAACAAG-3′ | |
| 5′-CCTCTGCGGCCGCTAAGCAGCTTATTTTCA | (SEQ ID NO:27) |
| CGGATTGAACCTG-3′ | |
| Pair 2: | |
| 5′-CCTCTGAGCTCAAGCTTCCGAGGACCACAA | (SEQ ID NO:28) |
| TGAACAAG-3′ | |
| 5′-CCTCTGCGGCCGCTGTTGCATTTCCTTTCT | (SEQ ID NO:30) |
| G-3′ | |

The first pair amplifies the entire OPG LORF, and creates a NotI restriction site which is compatible with the in-frame Not I site in Fc fusion vector pFcA3. pFcA3 was prepared by engineering a NotI restriction site 5′ to aspartic acid reside 216 of the human IgG1 Fc cDNA. This construct introduces a linker which encodes two irrelevant amino acids which span the junction between the OPG and IgG Fc region. This product, when linked to the Fc portion, would encode all 401 OPG residues directly followed by all 227 amino acid residues of the human IgG1 Fc region (Fl.Fc). The second primer pair amplifies the DNA sequences encoding the first 180 amino acid residues of OPG, which encompasses its putative ligand binding domain. As above, the 3′ primer creates an artificial Not I restriction site which fuses the C-terminal truncated OPG LORF at position threonine 180 directly to the IgG1 Fc domain (CT.fc).

The amino acid sequence junction linking OPG residue 401 and aseptic acid residue 221 of the human Fc region can be modified as follows: The DNA encoding residues 216-220 of the human Fc region can be deleted as described below, or the cysteine residue corresponding to C220 of the human Fc region can be mutated to either serine or alanine. OPF-Fc fusion protein encoded by these modifed vectors can be transfected into human 293 cells, or CHO cells, and recombinant OPG-Fc fusion protein purified as described below.

Both products were directionally cloned into the plasmid vector pCEP4 (Invitrogen). pCEP4 contains the Epstein-Barr virus origin of replication, and is capable of episomal replication in 293-EBNA-1 cells. The parent pCEP4, and pCEP4-Fl.Fc and pCEP4-CT.Fc vectors were lipofected into 293-EBNA-1 cells using the manufacturer's recommended methods. The transfected cells were then selected in 100 μg/ml hygromycin to select for vector expression, and the resulting drug-resistant mass cultures were grown to confluence. The cells were then cultured in serum-free media for 72 hr, and the conditioned media removed and analysed by SDS-PAGE. A silver staining of the polyacrylamide gel detects the major conditioned media proteins produced by the drug resistant 293 cultures. In the pCEP4-Fl.Fc and the pCEP4-CT.Fc conditioned media, unique bands of the predicted sizes were abundantly secreted (see FIGS. 13B and 13C).

The full-length Fc fusion protein accumulated to a high concentration, indicating that it may be stable. Both Fc fusion proteins were detected by anti-human IgG1 Fc antibodies (Pierce) on western blots, indicating that they are recombinant OPG products. The full length OPG-Fc fusion protein was purified by Protein-A column chromatography (Pierce) using the manufacturers recommended procedures. The protein was then subjected to N-terminal sequence analysis by automated Edman degradation as essentially described by Matsudaira et al. (J. Biol. Chem. 262, 10-35 (1987)). The following amino acid sequence was read after 19 cycles:

| | |
|---|---|
| NH₂-E T L P P K Y L H Y D P E T G H | (SEQ ID NO:31) |
| Q L L-CO₂H | |

This sequence was identical to the predicted mouse OPG amino acid sequence beginning at amino acid residue 22, suggesting that the natural mammalian leader cleavage site is between amino acid residues Q21-E22, not between Y31-D32 as originally predicted. The expression experiments performed in 293-EBNA cells with pCEP4-Fl.Fc and pCEP4-CT.Fc demonstrate that OPG is a secreted protein, and may act systemically to bind its ligand.

Procedures similar to those used to construct and express the muOPG[22-180]-Fc and muOPG[22-401]-Fc fusions were employed for additional mouse and human OPG-Fc fusion proteins.

Murine OPG cDNA encoding amino acids 1-185 fused to the Fc region of human IgG1 [muOPG Ct(185).Fc] was constructed as follows. Murine OPG cDNA from plasmid pRcCMV Mu Osteoprotegerin (described in Example 5) was amplified using the following primer pair in a polymerase chain reaction as described above:

| | |
|---|---|
| 1333-82: | |
| 5′-TCC CTT GCC CTG ACC ACT CTT-3′ | (SEQ ID NO:32) |
| 1333-80: | |
| 5′-CCT CTG CGG CCG CAC ACA CGT TGT | (SEQ ID NO:33) |
| CAT GTG TTG C-3′ | |

This primer pair amplifies the murine OPG cDNA region encoding amino acid residues 63-185 (corresponding to bp 278-645) of the OPG reading frame as shown in FIG. 9A. The 3′ primer contains a Not I restriction site which is compatible with the in-frame Not I site of the Fc fusion vector pFcA3. The product also spans a unique EcoRI restriction site located at bp 436. The amplified PCR product was purified, cleaved with NotI and EcoRI, and the resulting EcoRI-NotI restriction fragment was purified. The vector pCEP4 having the murine 1-401 OPG-Fc fusion insert was cleaved with EcoRI and NotI, purified, and ligated to the PCR product generated above. The resulting pCEP4-based expression vector encodes OPG residues 1-185 directly followed by all 227 amino acid residues of the human IgG1 Fc region. The murine OPG 1-185.Fc fusion vector was transfected into 293 cells, drug selected, and conditioned media was produced as described above. The resulting secreted murine OPG 1-185.Fc fusion product was purified by Protein-A column chromatography (Pierce) using the manufacturers recommended procedures.

Murine OPG DNA encoding amino acid residues 1-194 fused to the Fc region of human IgG1 (muOPG Ct(194).Fc) was constructed as follows. Mouse OPG cDNA from plasmid pRcCMV Mu-Osteoprotegerin was amplified using the following primer pairs:

| | |
|---|---|
| 1333-82: | |
| 5′-TCC CTT GCC CTG ACC ACT CTT-3′ | (SEQ ID NO:34) |
| 1333-81: | |
| 5′-CCT CTG CGG CCG CCT TTT GCG TGG | (SEQ ID NO:35) |
| CTT CTC TGT T-3′ | |

This primer pair amplifies the murine OPG cDNA region encoding amino acid residues 70-194 (corresponding to bp 298-672) of the OPG reading frame. The 3′ primer contains a Not I restriction site which is compatible with the in-frame Not I site of the Fc fusion vector pFcA3. The product also spans a unique EcoRI restriction site located at bp 436. The amplified PCR product was cloned into the murine OPG[1-401] Fc fusion vector as described above. The resulting pCEP4-based expression vector encodes OPG residues 1-194 directly followed by all 227 amino acid residues of the human IgG1 Fc region. The murine OPG 1-194.Fc fusion vector was transfected into 293 cells, drug selected, and conditioned media was produced. The resulting secreted fusion product was purified by Protein-A column chromatography (Pierce) using the manufacturers recommended procedures.

Human OPG DNA encoding amino acids 1-401 fused to the Fc region of human IgG1 was constructed as follows. Human OPG DNA in plasmid pRcCMV-hu osteoprotegerin (described in Example 5) was amplified using the following oligonucleotide primers:

| | |
|---|---|
| 1254-90: | |
| 5′ CCT CTG AGC TCA AGC TTG GTT TCC | (SEQ ID NO:36) |
| GGG GAC CAC AAT G-3′ | |
| 1254-95: | |
| 5′-CCT CTG CGG CCG CTA AGC AGC TTA | (SEQ ID NO:37) |
| TTT TTA CTG AAT GG-3′ | |

The resulting PCR product encodes the full-length human OPG and creates a Not I restriction site which is compatible with the in-frame Not I site Fc fusion vector FcA3. The PCR product was directionally cloned into the plasmid vector pCEP4 as described above. The resulting expression vector encodes human OPG residues 1-401 directly followed by 227 amino acid residues of the human IgG1 Fc region. Conditioned media from transfected and drug selected cells was produced and the huOPG Fl.Fc fusion product was purified by Protein-A column chromatography (Pierce) using the manufacturers recommended procedures.

Human OPG DNA encoding amino acid residues 1-201 fused to the Fc region of human IgG1 [huOPG Ct(201).Fc] was constructed as follows. The cloned human OPG cDNA from plasmid pRrCMV-hu osteoprotegerin was amplified by PCR using the following oligonucleotide primer pair:

| | |
|---|---|
| 1254-90: | |
| 5′-CCT CTG AGC TCA AGC TTG GTT TCC | (SEQ ID NO:38) |
| GGG GAC CAC AAT G-3′ | |
| 1254-92: | |
| 5′-CCT CTG CGG CCG CCA GGG TAA CAT | (SEQ ID NO:39) |
| CTA TTC CAC-3′ | |

This primer pair amplifies the human OPG cDNA region encoding amino acid residues 1-201 of the OPG reading frame, and creates a Not I restriction site at the 3′ end which is compatable with the in-frame Not I site Fc fusion vector FcA3. This product, when linked to the Fc portion, encodes OPG residues 1-201 directly followed by all 221 amino acid residues of the human IgG1 Fc region. The PCR product was directionally cloned into the plasmid vector pCEP4 as described above. Conditioned media from transfected and drug selected cells was produced, and the hu OPG Ct(201).Fc fusion products purified by Protein-A column chromatography (Pierce) using the manufacturer's recommended procedures.

The following procedures were used to construct and express unfused mouse and human OPG.

A plasmid for mammalian expression of full-length murine OPG (residues 1-401) was generated by PCR amplification of the murine OPG cDNA insert from pRcCMV Mu-Osteoprotegerin and subcloned into the expression vector pDSRα (DeClerck et. atl. J. Biol. Chem. 266, 3893 (1991)). The following oligonucleotide primers were used:

| | |
|---|---|
| 1295-26: | |
| 5′-CCG AAG CTT CCA CCA TGA ACA AGT | (SEQ ID NO:40) |
| GGC TGT GCT GC-3′ | |
| 1295-27: | |
| 5′-CCT CTG TCG ACT ATT ATA AGC AGC | (SEQ ID NO:41) |
| TTA TTT TCA CGG ATT G-3′ | |

The murine OPG full length reading frame was amplified by PCR as described above. The PCR product was purified and digested with restriction endonucleases Hind III and XbaI (Boehringer Mannheim, Indianapolis, Ind.) under the manufacturers recommended conditions, then ligated to Hind III and Xba I digested pDSR□. Recombinant clones were detected by restriction endonuclease digestion, then sequenced to ensure no mutations were produced during the PCR amplification steps.

The resulting plasmid, pDSRα-muOPG was introduced into Chinese hamster ovary (CHO) cells by calcium mediated transfection (Wigler et al. (1977), Cell 11: 233). Individual colonies were selected based upon expression of the dihydrofolate reductase (DHFR) gene in the plasmid vector and several clones were isolated. Expression of the murine OPG recombinant protein was monitored by western blot analysis of CHO cell conditioned media. High expressing cells were selected, and OPG expression was further amplified by treatment with methotrexate as described (DeClerck et al., ibid.). Conditioned media from CHO cell lines was produced for further purification of recombinant secreted murine OPG.

A plasmid for mammalian expression of full-length human OPG (amino acids 1-401) was generated by subcloning the cDNA insert in pRcCMV-hu Osteoprotegerin directly into vector pDSR□ (DeClerck et al., ibid). The pRcCMV-OPG plasmid was digested to completion with Not I, blunt ended with Klenow, then digested to completion with XbaI. Vector DNA was digested with HindIII, blunt ended with Klenow, then digested with XbaI, then ligated to the OPG insert. Recombinant plasmids were then sequenced to confirm proper orientation of the human OPG cDNA.

The resulting plasmid pDSRα-huOPG was introduced into Chinese hamster ovary (CHO) cells as described above. Individual colonies were selected based upon expression of the dihydrofolate reductase (DHFR) gene in the plasmid vector and several clones were isolated. Expression of the human OPG recombinant protein was monitored by western blot analysis of CHO cell conditioned media. High expressing clones were selected, and OPG expression was further amplified by treatment with methotrexate. Conditioned media from CHO cell lines expressing human OPG was produced for protein purification.

Expression vectors for murine OPG encoding residues 1-185 were constructed as follows. Murine OPG cDNA from pRcCMV-Mu OPG was amplified using the following oligonucleotide primers:

| | |
|---|---|
| 1333-82: | |
| 5′-TCC CTT GCC CTG ACC ACT CTT-3′ | (SEQ ID NO:42) |
| 1356-12: | |
| 5′-CCT CTG TCG ACT TAA CAC ACG TTG | (SEQ ID NO:43) |
| TCA TGT GTT GC-3′ | |

This primer pair amplifies the murine OPG cDNA region encoding amino acids 63-185 of the OPG reading frame (bp 278-645) and contains an artificial stop codon directly after the cysteine codon (C185), which is followed by an artificial Sal I restriction endonuclease site. The predicted product contains an internal Eco RI restriction site useful for subcloning into a pre-existing vector. After PCR amplification, the resulting purified product was cleaved with Eco RI and Sal I restriction endonucleases, and the large fragment was gel purified. The purified product was then subcloned into the large restriction fragment of an Eco RI and Sal I digest of pBluescript-muOPG Fl.Fc described above. The resulting plasmid was digested with Hind III and Xho I and the small fragment was gel purified. This fragment, which contains a open reading frame encoding residues 1-185 was then subcloned into a Hind III and Xho I digest of the expression vector pCEP4. The resulting vector, pmuOPG [1-185], encodes a truncated OPG polypeptide which terminates at a cysteine residue located at position 185. Conditioned media from transfected and drug selected cells was produced as described above.

| | |
|---|---|
| 1333-82: | |
| 5′-TCC CTT GCC CTG ACC ACT CTT-3′ | (SEQ ID NO:44) |
| 1356-13: | |
| 5′-CCT CTG TCG ACT TAC TTT TGC GTG | (SEQ ID NO:45) |
| GCT TCT CTG TT-3′ | |

This primer pair amplifies the murine OPG cDNA region encoding amino acids 70-194 of the OPG reading frame (bp 298-672) and contains an artificial stop codon directly after the lysine codon (K194), which is followed by an artificial Sal I restriction endonuclease site. The predicted product contains an internal Eco RI restriction site useful for subcloning into a pre-existing vector. After PCR amplification, the resulting purified product was cleaved with Eco RI and Sal I restriction endonucleases, and the large fragment was gel purified. The purified product was then subcloned into the large restriction fragment of an Eco RI and Sal I digest of pBluescript-muOPG Fl.Fc described above. The resulting plasmid was digested with Hind III and Xho I and the small fragment was gel purified. This fragment, which contains a open reading frame encoding residues 1-185 was then subcloned into a Hind III and Xho I digest of the expression vector pCEP4. The resulting vector, pmuOPG [1-185], encodes a truncated OPG polypeptide which terminates at a lysine at position 194. Conditioned media from transfected and drug selected cells was produced as described above.

Several mutations were generated at the 5′ end of the huOPG [22-401]-Fc gene that introduce either amino acid substitutions, or deletions, of OPG between residues 22 through 32. All mutations were generated with the “QuickChange™ Site-Directed Mutagenesis Kit” (Stratagene, San Diego, Calif.) using the manfacturer's recommended conditions. Briefly, reaction mix containing huOPG [22-401]-Fc plasmid DNA template and mutagenic primers were treated with Pfu polymerase in the presence of deoxynucleotides, then amplified in a thermocycler as described above. An aliqout of the reaction is then transfected into competent E. coli XL1-Blue by heatshock, then plated. Plasmid DNA from transformants was then sequenced to verify mutations.

The following primer pairs were used to delete residues 22-26 of the human OPG gene, resulting in the production of a huOPG [27-401]-Fc fusion protein:

| | |
|---|---|
| 1436-11: | |
| 5′-TGG ACC ACC CAG AAG TAC CTT CAT | (SEQ ID NO:140) |
| TAT GAC-3′ | |
| 1436-12: | |
| 5′-GTC ATA ATG AAG GTA CTT CTG GGT | (SEQ ID NO:141) |
| GGT CCA-3′ | |

The following primer pairs were used to delete residues 22-28 of the human OPG gene, resulting in the production of a huOPG [29-401]-Fc fusion protein:

| | |
|---|---|
| 1436-17: | |
| 5′-GGA CCA CCC AGC TTC ATT ATG ACG | (SEQ ID NO:142) |
| AAG AAA C-3′ | |
| 1436-18: | |
| 5′-GTT TCT TCG TCA TAA TGA AGC TGG | (SEQ ID NO:143) |
| GTG GTC C-3′ | |

The following primer pairs were used to delete residues 22-31 of the human OPG gene, resulting in the production of a huOPG [32-401]-Fc fusion protein:

| | |
|---|---|
| 1436-27: | |
| 5′-GTG GAC CAC CCA GGA CGA AGA AAC | (SEQ ID NO:144) |
| CTC TC-3′ | |
| 1436-28: | |
| 5′-GAG AGG TTT CTT CGT CCT GGG TGG | (SEQ ID NO:145) |
| TCC AC-3′ | |

The following primer pairs were used to change the codon for tyrosine residue 28 to phenylalanine of the human OPG gene, resulting in the production of a huOPG [22-401]-Fc Y28F fusion protein:

| | |
|---|---|
| 1436-29: | |
| 5′-CGT TTC CTC CAA AGT TCC TTC ATT | (SEQ ID NO:146) |
| ATG AC-3′ | |
| 1436-30: | |
| 5′-GTC ATA ATG AAG GAA CTT TGG AGG | (SEQ ID NO:147) |
| AAA CG-3′ | |

The following primer pairs were used to change the codon for proline residue 26 to alanine of the human OPG gene, resulting in the production of a huOPG [22-401]-Fc P26A fusion protein:

| | |
|---|---|
| 1429-83: | |
| 5′-GGA AAC GTT TCC TGC AAA GTA CCT | (SEQ ID NO:148) |
| TCA TTA TG-3 | |
| 1429-84: | |
| 5′-CAT AAT GAA GGT ACT TTG CAG GAA | (SEQ ID NO:149) |
| ACG TTT CC-3′ | |

Each resulting muOPG [22-401]-Fc plasmid containing the appropriate mutation was then transfected into human 293 cells, the mutant OPG-Fc fusion protein purified from conditioned media as described above. The biological activity of each protein was assessed the in vitro osteoclast forming assay described in Example 11.

### EXAMPLE 8

### Expression of OPG in E. coli

A. Bacterial Expression Vectors

pAMG21

The expression plasmid pAMG21 can be derived from the Amgen expression vector pCFM1656 (ATCC #69576) which in turn be derived from the Amgen expression vector system described in U.S. Pat. No. 4,710,473. The pCFM1656 plasmid can be derived from the described pCFM836 plasmid (U.S. Pat. No. 4,710,473) by: (a) destroying the two endogenous NdeI restriction sites by end filling with T4 polymerase enzyme followed by blunt end ligation; (b) replacing the DNA sequence between the unique AatII and ClaI restriction sites containing the synthetic P_{L} promoter with a similar fragment obtained from pCFM636 (U.S. Pat. No. 4,710,473) containing the PL promoter

| | |
|---|---|
| AatII | |
| 5′ CTAATTCCGCTCTCACCTACCAAACAATGCCCCCCTGCAAAAAATAAATTCATAT | (SEQ ID NO:53) |
| 3′TGCAGATTAAGGCGAGAGTGGATGGTTTGTTACGGGGGGACGTTTTTTATTTAAGTATA | (SEQ ID NO:54) |
| AAAAAACATACAGATAACCATCTGCGGTGATAAATTATCTCTGGCGGTGTTGACATAAA | |
| TTTTTTGTATGTCTATTGGTAGACGCCACTATTTAATAGAGACCGCCACAACTGTATTT | |
| TACCACTGGCGGTGATACTGAGCACAT 3′ | |
| ATGGTGACCGCCACTATGACTCGTGTAGC5′ | |
| ClaI | |

and then (c) substituting the small DNA sequence between the unique ClaI and KpnI restriction sites with the following oligonucleotide:

| | |
|---|---|
| 5′CGATTTGATTCTAGAAGGAGGAATAACATATGGTTAACGCGTTGGAATTCGGTAC3′ | (SEQ ID NO:48) |
| 3′ TAAACTAAGATCTTCCTCCTTATTGTATACCAATTGCGCAACCTTAAGC 5′ | (SEQ ID NO:49) |
| ClaI KpnI | |

The expression plasmid pAMG21 can then be derived from pCFM1656 by making a series of site directed base changes by PCR overlapping oligo mutagenesis and DNA sequence substitutions. Starting with the BglII site (plasmid bp # 180) immediately 5′ to the plasmid replication promoter PcopB and proceeding toward the plasmid replication genes, the base pair changes are as follows:

TABLE 4

| | | |
|---|---|---|
| pAMG21 bp # | bp in pCFM1656 | bp changed to in pAMG21 |

| | | |
|---|---|---|
| # 204 | T/A | C/G |
| # 428 | A/T | G/C |
| # 509 | G/C | A/T |
| # 617 | — | insert two G/C bp |
| # 679 | G/C | T/A |
| # 980 | T/A | C/G |
| # 994 | G/C | A/T |
| # 1004 | A/T | C/G |
| # 1007 | C/G | T/A |
| # 1028 | A/T | T/A |
| # 1047 | C/G | T/A |
| # 1178 | G/C | T/A |
| # 1466 | G/C | T/A |
| # 2028 | G/C | bp deletion |
| # 2187 | C/G | T/A |
| # 2480 | A/T | T/A |
| # 2499-2502 | AGTG | GTCA |
| | TCAC | CAGT |
| # 2642 | TCCGAGC | 7 bp deletion |
| | AGGCTCG | |
| # 3435 | G/C | A/T |
| # 3446 | G/C | A/T |
| # 3643 | A/T | T/A |

The DNA sequence between the unique AatII (position #4364 in pCFM1656) and SacII (position #4585 in pCFM1656) restriction sites is substituted with the following DNA sequence:

| | |
|---|---|
| [AatII sticky end] 5′GCGTAACGTATGCATGGTCTCC | (SEQ ID NO:50) |
| (position #4358 in pAMG21) 3′ TGCACGCATTGCATACGTACCAGAGG | (SEQ ID NO:46) |
| CCATGCGAGAGTAGGGAACTGCCAGGCATCAAATAAAACGAAAGGCTCAGTCGAAAGACT | |
| GGTACGCTCTCATCCCTTGACGGTCCGTAGTTTATTTTGCTTTCCGAGTCAGCTTTCTGA | |
| GGGCCTTTCGTTTTATCTGTTGTTTGTCGGTGAACGCTCTCCTGAGTAGGACAAATCCGC | |
| CCCGGAAAGCAAAATAGACAACAAACAGCCACTTGCGAGAGGACTCATCCTGTTTAGGCG | |
| CGGGAGCGGATTTGAACGTTGCGAAGCAACGGCCCGGAGGGTGGCGGGCAGGACGCCCGC | |
| GCCCTCGCCTAAACTTGCAACGCTTCGTTGCCGGGCCTCCCACCGCCCGTCCTGCGGGCG | |
| CATAAACTGCCAGGCATCAAATTAAGCAGAAGGCCATCCTGACGGATGGCCTTTTTGCGT | |
| GTATTTGACGGTCCGTAGTTTAATTCGTCTTCCGGTAGGACTGCCTACCGGAAAAACGCA | |
| AatII | |
| TTCTACAAACTCTTTTGTTTATTTTTCTAAATACATTCAAATATGGACGTCGTACTTAAC | |
| AAGATGTTTGAGAAAACAAATAAAAAGATTTATGTAAGTTTATACCTGCAGCATGAATTG | |
| TTTTAAAGTATGGGCAATCAATTGCTCCTGTTAAAATTGCTTTAGAAATACTTTGGCAGC | |
| AAAATTTCATACCCGTTAGTTAACGAGGACAATTTTAACGAAATCTTTATGAAACCGTCG | |
| GGTTTGTTGTATTGAGTTTCATTTGCGCATTGGTTAAATGGAAAGTGACCGTGCGCTTAC | |
| CCAAACAACATAACTCAAAGTAAACGCGTAACCAATTTACCTTTCACTGGCACGCGAATG | |
| TACAGCCTAATATTTTTGAAATATCCCAAGAGCTTTTTCCTTCGCATGCCCACGCTAAAC | |
| ATGTCGGATTATAAAAACTTTATAGGGTTCTCGAAAAAGGAAGCGTACGGGTGCGATTTG | |
| ATTCTTTTTCTCTTTTGGTTAAATCGTTGTTTGATTTATTATTTGCTATATTTATTTTTC | |
| TAAGAAAAAGAGAAAACCAATTTAGCAACAAACTAAATAATAAACGATATAAATAAAAAG | |
| GATAATTATCAACTAGAGAAGGAACAATTAATGGTATGTTCATACACGCATGTAAAAATA | |
| CTATTAATAGTTGATCTCTTCCTTGTTAATTACCATACAAGTATGTGCGTACATTTTTAT | |
| AACTATCTATATAGTTGTCTTTCTCTGAATGTGCAAAACTAAGCATTCCGAAGCCATTAT | |
| TTGATAGATATATCAACAGAAAGAGACTTACACGTTTTGATTCGTAAGGCTTCGGTAATA | |
| TAGCAGTATGAATAGGGAAACTAAACCCAGTGATAAGACCTGATGATTTCGCTTCTTTAA | |
| ATCGTCATACTTATCCCTTTGATTTGGGTCACTATTCTGGACTACTAAAGCGAAGAAATT | |
| TTACATTTGGAGATTTTTTATTTACAGCATTGTTTTCAAATATATTCCAATTAATCGGTG | |
| AATGTAAACCTCTAAAAAATAAATGTCGTAACAAAAGTTTATATAAGGTTAATTAGCCAC | |
| AATGATTGGAGTTAGAATAATCTACTATAGGATCATATTTTATTAAATTAGCGTCATCAT | |
| TTACTAACCTCAATCTTATTAGATGATATCCTAGTATAAAATAATTTAATCGCAGTAGTA | |
| AATATTGCCTCCATTTTTTAGGGTAATTATCCAGAATTGAAATATCAGATTTAACCATAG | |
| TTATAACGGAGGTAAAAAATCCCATTAATAGGTCTTAACTTTATAGTCTAAATTGGTATC | |
| AATGAGGATAAATGATCGCGAGTAAATAATATTCACAATGTACCATTTTAGTCATATCAG | |
| TTACTCCTATTTACTAGCGCTCATTTATTATAAGTGTTACATGGTAAAATCAGTATAGTC | |
| ATAAGCATTGATTAATATCATTATTGCTTCTACAGGCTTTAATTTTATTAATTATTCTGT | |
| TATTCGTAACTAATTATAGTAATAACGAAGATGTCCGAAATTAAAATAATTAATAAGACA | |
| AAGTGTCGTCGGCATTTATGTCTTTCATACCCATCTCTTTATCCTTACCTATTGTTTGTC | |
| TTCACAGCAGCCGTAAATACAGAAAGTATGGGTAGAGAAATAGGAATGGATAACAAACAG | |
| GCAAGTTTTGCGTGTTATATATCATTAAAACGGTAATAGATTGACATTTGATTCTAATAA | |
| CGTTCAAAACGCACAATATATAGTAATTTTGCCATTATCTAACTGTAAACTAAGATTATT | |
| ATTGGATTTTTGTCACACTATTATATCGCTTGAAATACAATTGTTTAACATAAGTACCTG | |
| TAACCTAAAAACAGTGTGATAATATAGCGAACTTTATGTTAACAAATTGTATTCATGGAC | |
| TAGGATCGTACAGGTTTACGCAAGAAAATGGTTTGTTATAGTCGATTAATCGATTTGATT | |
| ATCCTAGCATGTCCAAATGCGTTCTTTTACCAAACAATATCAGCTAATTAGCTAAACTAA | |
| CTAGATTTGTTTTAACTAATTAAAGGAGGAATAACATATGGTTAACGCGTTGGAATTCGA | |
| GATCTAAACAAAATTGATTAATTTCCTCCTTATTGTATACCAATTGCGCAACCTTAAGCT | |
| SacII | |
| GCTCACTAGTGTCGACCTGCAGGGTACCATGGAAGCTTACTCGAGGATCCGCGGAAAGAA | |
| CGAGTGATCACAGCTGGACGTCCCATGGTACCTTCGAATGAGCTCCTAGGCGCCTTTCTT | |
| GAAGAAGAAGAAGAAAGCCCGAAAGGAAGCTGAGTTGGCTGCTGCCACCGCTGAGCAATA | |
| CTTCTTCTTCTTCTTTCGGGCTTTCCTTCGACTCAACCGACGACGGTGGCGACTCGTTAT | |
| ACTAGCATAACCCCTTGGGGCCTCTAAACGGGTCTTGAGGGGTTTTTTGCTGAAAGGAGG | |
| TGATCGTATTGGGGAACCCCGGAGATTTGCCCAGAACTCCCCAAAAAACGACTTTCCTCC | |
| AACCGCTCTTCACGCTCTTCACGC 3′ [SacII sticky end] | |
| TTGGCGAGAAGTGCGAGAAGTG 5′ (position #5904 in pAMG21) | |

During the ligation of the sticky ends of this substitution DNA sequence, the outside AatII and SacII sites are destroyed. There are unique AatII and SacII sites in the substituted DNA.

pAMG22-His

The expression plasmid pAMG22-His can be derived from the Amgen expression vector pAMG22 by substituting the small DNA sequence between the unique NdeI (#4795) and EcoRI (#4818) restriction sites of pAMG22 with the following oligonucleotide duplex:

| | |
|---|---|
| NdeI NheI EcoRI | |
| 5′ TATGAAACATCATCACCATCACCATCATGCTAGCGTTAACGCGTTGG 3′ | (SEQ ID NO:51) |
| 3′ ACTTTGTAGTAGTGGTAGTGGTAGTACGATCGCAATTGCGCAACCTTAA 5′ | (SEQ ID NO:52) |
| MetLysHisHisHisHisHisHisHisAlaSerValAsnAlaLeuGlu | (SEQ ID NO:168) |

pAMG22

The expression plasmid pAMG22 can be derived from the Amgen expression vector pCFM1656 (ATCC #69576) which in turn be derived from the Amgen expression vector system described in U.S. Pat. No. 4,710,473 granted Dec. 1, 1987. The pCFM1656 plasmid can be derived from the described pCFM836 plasmid (U.S. Pat. No. 4,710,473) by: (a) destroying the two endogenous NdeI restriction sites by end filling with T4 polymerase enzyme followed by blunt end ligation; (b) replacing the DNA sequence between the unique AatII and ClaI restriction sites containing the synthetic PL promoter with a similar fragment obtained from pCFM636 (U.S. Pat. No. 4,710,473) containing the PL promoter

| | |
|---|---|
| AatII | |
| 5′ CTAATTCCGCTCTCACCTACCAAACAATGCCCCCCTGCAAAAAATAAATTCATAT | (SEQ ID NO:53) |
| 3′TGCAGATTAAGGCGAGAGTGGATGGTTTGTTACGGGGGGACGTTTTTTATTTAAGTATA | (SEQ ID NO:54) |
| AAAAAACATACAGATAACCATCTGCGGTGATAAATTATCTCTGGCGGTGTTGACATAAA | |
| TTTTTTGTATGTCTATTGGTAGACGCCACTATTTAATAGAGACCGCCACAACTGTATTT | |
| TACCACTGGCGGTGATACTGAGCACAT 3′ | |
| ATGGTGACCGCCACTATGACTCGTGTAGC5′ | |
| ClaI | |

and then (c) substituting the small DNA sequence between the unique ClaI and KpnI restriction sites with the following oligonucleotide:

| | |
|---|---|
| 5′ CGATTTGATTCTAGAAGGAGGAATAACATATGGTTAACGCGTTGGAATTCGGTAC 3′ | (SEQ ID NO:55) |
| 3′ TAAACTAAGATCTTCCTCCTTATTGTATACCAATTGCGCAACCTTAAGC 5′ | (SEQ ID NO:56) |
| ClaI KpnI | |

The expression plasmid pAMG²2 can then be derived from pCFM1656 by making a series of site directed base changes by PCR overlapping oligo mutagenesis and DNA sequence substitutions. Starting with the BglII site (plasmid bp # 180) immediately 5′ to the plasmid replication promoter PcopB and proceeding toward the plasmid replication genes, the base pair changes are as follows:

TABLE 5

| | | |
|---|---|---|
| pAMG22 bp # | bp in pCFM1656 | bp changed to in pAMG22 |

| | | |
|---|---|---|
| # 204 | T/A | C/G |
| # 428 | A/T | G/C |
| # 509 | G/C | A/T |
| # 617 | — | insert two G/C bp |
| # 679 | G/C | T/A |
| # 980 | T/A | C/G |
| # 994 | G/C | A/T |
| # 1004 | A/T | C/G |
| # 1007 | C/G | T/A |
| # 1028 | A/T | T/A |
| # 1047 | C/G | T/A |
| # 1178 | G/C | T/A |
| # 1466 | G/C | T/A |
| # 2028 | G/C | bp deletion |
| # 2187 | C/G | T/A |
| # 2480 | A/T | T/A |
| # 2499-2502 | AGTG | GTCA |
| | TCAC | CAGT |
| # 2642 | TCCGAGC | 7 bp deletion |
| | AGGCTCG | |
| # 3435 | G/C | A/T |
| # 3446 | G/C | A/T |
| # 3643 | A/T | T/A |

The DNA sequence between the unique AatII (position #4364 in pCFM1656) and SacII (position #4585 in pCFM1656) restriction sites is substituted with the following DNA sequence:

| | |
|---|---|
| [AatII sticky end] (position #4358 in pAMG22) | |
| 5′ GCGTAACGTATGCATGGTCTCCCCATGCGAGAGTAGGGAACTGCCAGGCATCAA | (SEQ ID NO:58) |
| 3′ TGCACGCATTGCATACGTACCAGAGGGGTACGCTCTCATCCCTTGACGGTCCGTAGTT | (SEQ ID NO:57) |
| ATAAAACGAAAGGCTCAGTCGAAAGACTGGGCCTTTCGTTTTATCTGTTGTTTGTCGGTG | |
| TATTTTGCTTTCCGAGTCAGCTTTCTGACCCGGAAAGCAAAATAGACAACAAACAGCCAC | |
| AACGCTCTCCTGAGTAGGACAAATCCGCCGGGAGCGGATTTGAACGTTGCGAAGCAACGG | |
| TTGCGAGAGGACTCATCCTGTTTAGGCGGCCCTCGCCTAAACTTGCAACGCTTCGTTGCC | |
| CCCGGAGGGTGGCGGGCAGGACGCCCGCCATAAACTGCCAGGCATCAAATTAAGCAGAAG | |
| GGGCCTCCCACCGCCCGTCCTGCGGGCGGTATTTGACGGTCCGTAGTTTAATTCGTCTTC | |
| GCCATCCTGACGGATGGCCTTTTTGCGTTTCTACAAACTCTTTTGTTTATTTTTCTAAAT | |
| CGGTAGGACTGCCTACCGGAAAAACGCAAAGATGTTTGAGAAAACAAATAAAAAGATTTA | |
| AatII | |
| ACATTCAAATATGGACGTCTCATAATTTTTAAAAAATTCATTTGACAAATGCTAAAATTC | |
| TGTAAGTTTATACCTGCAGAGTATTAAAAATTTTTTAAGTAAACTGTTTACGATTTTAAG | |
| TTGATTAATATTCTCAATTGTGAGCGCTCACAATTTATCGATTTGATTCTAGATTTGTTT | |
| AACTAATTATAAGAGTTAACACTCGCGAGTGTTAAATAGCTAAACTAAGATCTAAACTCA | |
| TAACTAATTAAAGGAGGAATAACATATGGTTAACGCGTTGGAATTCGAGCTCACTAGTGT | |
| ATTGATTAATTTCCTCCTTATTGTATACCAATTGCGCAACCTTAAGCTCGAGTGATCACA | |
| SacII | |
| CGACCTGCAGGGTACCATGGAAGCTTACTCGAGGATCCGCGGAAAGAAGAAGAAGAAGAA | |
| GCTGGACGTCCCATGGTACCTTCGAATGAGCTCCTAGGCGCCTTTCTTCTTCTTCTTCTT | |
| GAAAGCCCGAAAGGAAGCTGAGTTGGCTGCTGCCACCGCTGAGCAATAACTAGCATAACC | |
| CTTTCGGGCTTTCCTTCGACTCAACCGACGACGGTGGCGACTCGTTATTGATCGTATTGG | |
| CCTTGGGGCCTCTAAACGGGTCTTGAGGGGTTTTTTGCTGAAAGGAGGAACCGCTCTTCA | |
| GGAACCCCGGAGATTTGCCCAGAACTCCCCAAAAAACGACTTTCCTCCTTGGCGAGAAGT | |
| CGCTCTTCACGC 3′ | |
| GCGAGAAGTG 5′ | |
| [SacII sticky end] (position #5024 in pAMG22) | |

During the ligation of the sticky ends of this substitution DNA sequence, the outside AatII and SacII sites are destroyed. There are unique AatII and SacII sites in the substituted DNA.

B. Human OPG Met[32-401]

In the example, the expression vector used was pAMG21, a derivative of pCFM1656 (ATCC accession no. 69576) which contains appropriate restriction sites for insertion of genes downstream from the lux PR promoter. (See U.S. Pat. No. 5,169,318 for description of the lux expression system). The host cell used was GM120 (ATCC accession no. 55764). This host has the lacIQ promoter and lacI gene integrated into a second site in the host chromosome of a prototrophic E. coli K12 host. Other commonly used E. coli expression vectors and host cells are also suitable for expression.

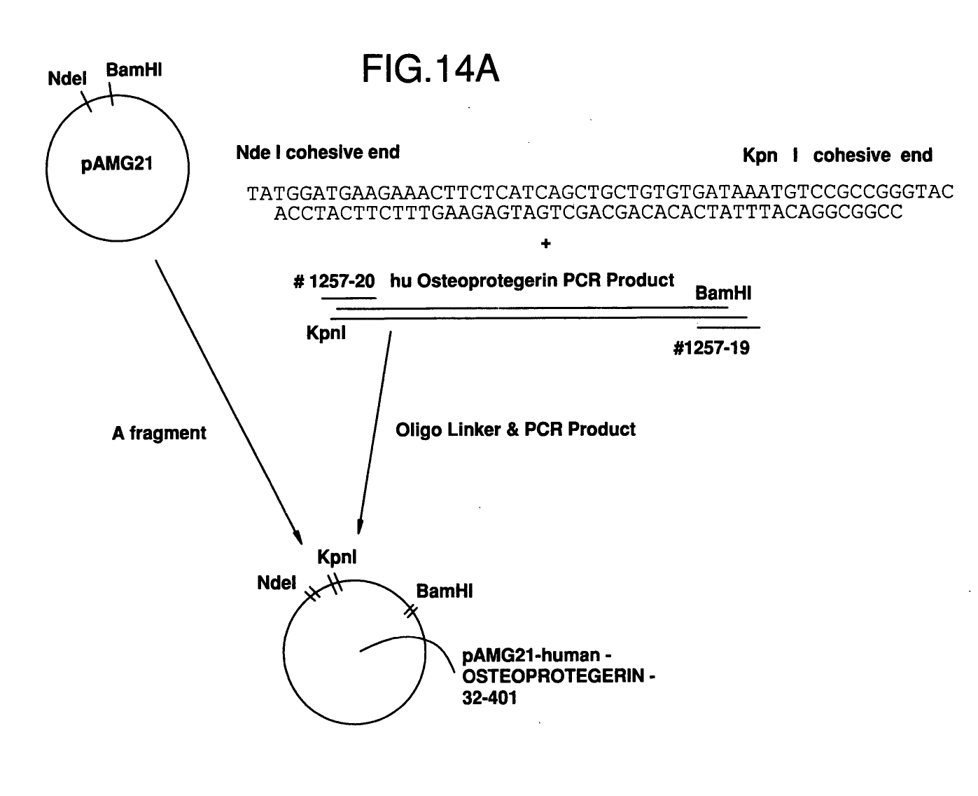

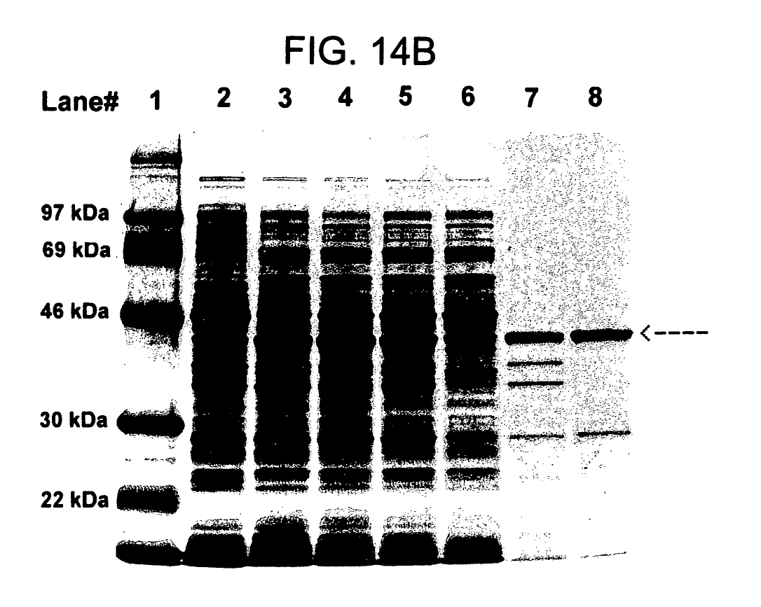

A DNA sequence coding for an N-terminal methionine and amino acids 32-401 of the human OPG polypeptide was placed under control of the luxPR promoter in the plasmid expression vector pAMG21 as follows. To accomplish this, PCR using oligonucleotides #1257-20 and #1257-19 as primers was performed using as a template plasmid pRcCMV-Hu OPG DNA containing the human OPG cDNA and thermocycling for 30 cycles with each cycle being: 94° C. for 20 seconds, followed by 37° C. for 30 seconds, followed by 72° C. for 30 seconds. The resulting PCR sample was resolved on an agarose gel, the PCR product was excised, purified, and restricted with KpnI and BamHI restriction endonucleases and purified. Synthetic oligonucleotides #1257-21 and #1257-22 were phophorylated individually using T4 polynucleotide kinase and ATP, and were then mixed together, heated at 94° C. and allowed to slow cool to room temperature to form an oligonucleotide linker duplex containing NdeI and KpnI sticky ends. The phosphorylated linker duplex formed between oligonucleotides #1257-21 and #1257-22 containing NdeI and KpnI cohesive ends (see FIG. 14A) and the KpnI and BamHI digested and purified PCR product generated using oligo primers #1257-20 and #1257-19 (see above) was directionally inserted between two sites of the plasmid vector pAMG21, namely the NdeI site and BamHI site, using standard recombinant DNA methodology (see FIG. 14A and sequences below). The synthetic linker utilized E. coli codons and provided for a N-terminal methionine.

Two clones were selected and plasmid DNA isolated, and the human OPG insert was subsequently DNA sequence confirmed. The resulting pAMG21 plasmid containing amino acids 32-401 of the human OPG polypeptide immediately preceded in frame by a methionine is referred to as pAMG21-huOPG met[32-401] or pAMG21-huOPG met[32-401].

| | |
|---|---|
| Oligo #1257-19: | |
| 5′-TACGCACTGGATCCTTATAAGCAGCTTATTTTT | (SEQ ID NO:59) |
| ACTGATTGGAC-3′ | |
| Oligo #1257-20: | |
| 5′-GTCCTCCTGGTACCTACCTAAAACAAC-3′ | (SEQ ID NO:60) |
| Oligo #1257-21: | |
| 5′-TATGGATGAAGAAACTTCTCATCAGCTGCTGTG | (SEQ ID NO:61) |
| TGATAAATGTCCGCCGGGTAC-3′ | |
| Oligo #1257-22: | |
| 5′-CCGGCGGACATTTATCACACAGCAGCTGATGAG | (SEQ ID NO:47) |
| AAGTTTCTTCATCCA-3′ | |

Cultures of pAMG21-huOPG met[32-401] in E. coli GM120 in 2XYT media containing 20 μg/ml kanamycin were incubated at 30° C. prior to induction. Induction of huOPG met[32-401] gene product expression from the luxPR promoter was achieved following the addition of the synthetic autoinducer N-(3-oxohexanoyl)-DL-homoserine lactone to the culture media to a final concentration of 30 ng/ml and cultures were incubated at either 30° C. or 37° C. for a further 6 hours. After 6 hours, the bacterial cultures were examined by microscopy for the presence of inclusion bodies and were then pelletted by centrifugation. Refractile inclusion bodies were observed in induced cultures indicating that some of the recombinant huOPG met[32-401] gene product was produced insolubly in E. coli.

Some bacterial pellets were resuspended in 10 mM Tris-HCl/pH8, 1 mM EDTA and lysed directly by addition of 2× Laemlli sample buffer to 1× final, and β-mercaptoethanol to 5% final concentration, and analyzed by SDS-PAGE. A substantially more intense coomassie stained band of approximately 42 kDa was observed on a SDS-PAGE gel containing total cell lysates of 30° C. and 37° C. induced cultures versus lane 2 which is a total cell lysate of a 30° C. uninduced culture (FIG. 14B). The expected gene product would be 370 amino acids in length and have an expected molecular weight of about 42.2 kDa.

Following induction at 37 ° C. for 6 hours, an additional culture was pelleted and either processed for isolation of inclusion bodies (see below) or processed by microfluidizing. The pellet processed for microfluidizing was resuspended in 25 mM Tris-HCl/pH8, 0.5M NaCl buffer and passed 20 times through a Microfluidizer Model 1108 (Microfluidics Corp.) and collected. An aliquot was removed of the collected sample (microfluidized total lysate), and the remainder was pelleted at 20,000× g for 20 minutes. The supernatant following centrifugation was removed (microfluidized soluble fraction) and the pellet resuspended in a 25 mM Tris-HCl/pH8, 0.5M NaCl, 6M urea solution (microfluidized insoluble fraction). To an aliquot of either the total soluble, or insoluble fraction was added to an equal volume of 2× Laemalli sample buffer and β-mercaptoethanol to 5% final concentration. The samples were then analyzed by SDS-PAGE. A significant amount of recombinant huOPG met[32-401] gene product appeared to be found in the insoluble fraction.

To purify the recombinant protein, inclusion bodies were purified as follows:

Bacterial cells were separated from media by density gradient centrifugation in a Beckman J-6B centrifuge equipped with a JS4.2 rotor at 4,900× g for 15 minutes at 4° C. The bacterial pellet was resuspended in 5 ml of water and then diluted to a final volume of 10 ml with water. This suspension was transferred to a stainless steel cup cooled in ice and subjected to sonic disruption using a Branson Sonifier equipped with a standard tip (power setting=5, duty cycle=95%, 80 bursts). The sonicated cell suspension was centrifuged in a Beckman Optima TLX ultracentrifuge equipped with a TLA 100.3 rotor at 195,000×g for 5 to 10 minutes at 23° C. The supernatant was discarded and the pellet rinsed with a stream of water from a squirt bottle. The pellets were collected by scraping with a micro spatula and transferred to a glass homogenizer (15 ml capacity). Five ml of Percoll solution (75% liquid Percoll, 0.15 M sodium chloride) was added to the homogenizer and the contents are homogenized until uniformly suspended. The volume was increased to 19.5 ml by the addition of Percoll solution, mixed, and distributed into 3 Beckman Quick-Seal tubes (13×32 mm). Tubes were sealed according to manufacturers instructions. The tubes were spun in a Beckman TLA 100.3 rotor at 23° C., 20,000 rpm (21,600×g), 30 minutes. The tubes were examined for the appropriate banding pattern. To recover the refractile bodies, gradient fractions were recovered and pooled, then diluted with water. The inclusion bodies were pelleted by centrifugation, and the protein concentration estimated following SDS-PAGE.

An aliquot of inclusion bodies isolated as described below was dissolved into 1× Laemili sample buffer with 5% β-mercaptoethanol and resolved on a SDS-PAGE gel and the isolated inclusion bodies provide a highly purified recombinant huOPG[32-401] gene product. The major ˜42 kDa band observed after resolving inclusion bodies on a SDS-polyacrylamide gel was excised from a separate gel and the N-terminal amino acid sequence determined essentially as described (Matsudaira et al. J. Biol. Chem. 262, 10-35 (1987)). The following sequence was determined after 19 cycles:

| | |
|---|---|
| NH₂-MDEETSHQLLCDKCPPGTY-COOH | (SEQ ID NO:62) |

This sequence was found to be identical to the first 19 amino acids encoded by the pAMG21 Hu-OPG met[32-401] expression vector, produced by a methionine residue provided by the bacterial expression vector.
C. Human OPG met[22-401]

A DNA sequence coding for an N-terminal methionine and amino acids 22 through 401 of human OPG was placed under control of the luxPR promoter in a prokaryotic plasmid expression vector pAMG21 as follows. Isolated plasmid DNA of pAMG21-huOPG met[32-401] (see Section B) was cleaved with KpnI and BamHI restriction endonucleases and the resulting fragments were resolved on an agarose gel. The B fragment (about 1064 bp fragment) was isolated from the gel using standard methodology. Synthetic oligonucleotides (oligos) #1267-06 and #1267-07 were phosphorylated individually and allowed to form an oligo linker duplex, which contained NdeI and KpnI cohesive ends, using methods described in Section B. The synthetic linker duplex utilized E. coli codons and provided for an N-terminal methionine. The phosphorylated oligo linker containing NdeI and KpnI cohesive ends and the isolated about 1064 bp fragment of pAMG21-huOP met[32-401] digested with KpnI and BamHI restriction endonucleases were directionally inserted between the NdeI and BamHI sites of pAMG21 using standard recombinant DNA methodology. The ligation mixture was transformed into E. coli host 393 by electroporation utilizing the manufacturer's protocol.

Clones were selected, plasmid DNA was isolated, and DNA sequencing was performed to verify the DNA sequence of the huOPG-met[22-401] gene.

| | |
|---|---|
| Oligo #1267-06: | |
| 5′-TAT GGA AAC TTT TCC TCC AAA ATA | (SEQ ID NO:63) |
| TCT TCA TTA TGA TGA AGA AAC TTC TCA | |
| TCA GCT GCT GTG TGA TAA ATG TCC GCC | |
| GGG TAC-3′ | |
| Oligo #1267-07: | |
| 5′-CCG GCG GAC ATT TAT CAC ACA GCA | (SEQ ID NO:64) |
| GCT GAT GAG AAG TTT CTT CAT CAT AAT | |
| GAA GAT ATT TTG GAG GAA AAG TTT | |
| CCA-3′ | |

Cultures of pAMG21-huOPG-met[22-401] in E. coli host 393 were placed in 2XYT media containing 20 μg/ml kanamycin and were incubated at 30° C. prior to induction. Induction of recombinant gene product expression from the luxPR promoter of vector pAMG21 was achieved following the addition of the synthetic autoinducer N-(3-oxohexanoyl)-DL-homoserine lactone to the culture media to a final concentration of 30 ng/ml and incubation at either 30° C. or 37° C. for a further 6 hours. After 6 hours, bacterial cultures were pelleted by centrifugation (=30° C. I+6 or 37° C. I+6). Bacterial cultures were also either pelleted just prior to induction (=30° C. PreI) or alternatively no autoinducer was added to a separate culture which was allowed to incubate at 30° C. for a further 6 hours to give an uninduced (UI) culture (=30° C. UI). Bacterial pellets of either 30° C. PreI, 30° C. UI, 30° C. I+6, or 37° C. I+6 cultures were resuspended, lysed, and analyzed by SDS-polyacrylamide gel electrophoresis (PAGE) as described in Section B. Polyacrylamide gels were either stained with coomassie blue and/or Western transferred to nitrocellulose and immunoprobed with rabbit anti-mu OPG-Fc polyclonal antibody as described in Example 10. The level of gene product following induction compared to either an uninduced (30° C. UI) or pre-induction (30° C. PreI) sample.

D. Murine OPG met[22-401]

A DNA sequence coding for an N-terminal methionine and amino acids 22 through 401 of the murine (mu) OPG (OPG) polypeptide was placed under control of the luxPR promoter in a prokaryotic plasmid expression vector pAMG21 as follows. PCR was performed using oligonucleotides #1257-16 and #1257-15 as primers, plasmid pRcCMV-Mu OPG DNA as a template and thermocycling conditions as described in Section B. The PCR product was purified and cleaved with KpnI and BamHI restriction endonucleases as described in Section B. Synthetic oligos #1260-61 and #1260-82 were phosphorylated individually and allowed to form an oligo linker duplex with NdeI and KpnI cohesive ends using methods described in Section B. The synthetic linker duplex utilized E. coli codons and provided for an N-terminal methionine. The phosphorylated linker duplex formed between oligos #1260-61 and #1260-82 containing NdeI and KpnI cohesive ends and the KpnI and BamHI digested and purified PCR product generated using oligo primers #1257-16 and #1257-15 were directionally inserted between the NdeI and BamHI sites of pAMG21 using standard methodology. The ligation mixture was transformed into E. coli host 393 by electroporation utilizing the manufacturer's protocol. Clones were selected, plasmid DNA was isolated, and DNA sequencing was performed to verify the DNA sequence of the MuOPG met[22-401] gene.

Expression of recombinant muOPG met[22-401] polypeptide from cultures of 393 cells harboring plasmid pAMG21-MuOPG met[22-401] following induction was determined using methods described in Section C.

| | |
|---|---|
| Oligo #1257-15: | |
| 5′-TAC GCA CTG GAT CCT TAT AAG CAG | (SEQ ID NO:65) |
| CTT ATT TTC ACG GAT TGA AC-3′ | |
| Oligo #1257-16: | |
| 5′-GTG CTC CTG GTA CCT ACC TAA AAC | (SEQ ID NO:66) |
| AGC ACT GCA CAG TG-3′ | |
| Oligo #1260-61: | |
| 5′-TAT GGA AAC TCT GCC TCC AAA ATA | (SEQ ID NO:67) |
| CCT GCA TTA CGA TCC GGA AAC TGG TCA | |
| TCA GCT GCT GTG TGA TAA ATG TGC TCC | |
| GGG TAC-3′ | |
| Oligo #1260-82: | |
| 5′-CCG GAG CAC ATT TAT CAC ACA GCA | (SEQ ID NO:68) |
| GCT GAT GAC CAG TTT CCG GAT CGT AAT | |
| GCA GGT ATT TTG GAG GCA GAG TTT | |
| CCA-3′ | |

E. Murine OPG met[32-401]

A DNA sequence coding for an N-terminal methionine and amino acids 32 through 401 of murine OPG was placed under control of the luxPR promoter in a prokaryotic plasmid expression vector pAMG21 as follows. To accomplish this, Synthetic oligos #1267-08 and #1267-09 were phosphorylated individually and allowed to form an oligo linker duplex using methods described in Section B. The synthetic linker duplex utilized E. coli codons and provided for an N-terminal methionine. The phosphorylated linker duplex formed between oligos #1267-08 and #1267-09 containing NdeI and KpnI cohesive ends, and the KpnI and BamHI digested and purified PCR product described earlier (see Section D), was directionally inserted between the NdeI and BamHI sites of pAMG21 using standard methodology. The ligation mixture was transformed into E. coli host 393 by electroporation utilizing the manufacturer's protocol. Clones were selected, plasmid DNA was isolated, and DNA sequencing was performed to verify the DNA sequence of the muOPG-met[32-401] gene.

Expression of recombinant muOPG-met [32-401] polypeptide from cultures of 393 cells harboring the pAMG21 recombinant plasmid following induction was determined using methods described in Section C.

| | |
|---|---|
| Oligo #1267-08: | |
| 5′-TAT GGA CCC AGA AAC TGG TCA TCA | (SEQ ID NO:69) |
| GCT GCT GTG TGA TAA ATG TGC TCC GGG | |
| TAC-3′ | |
| Oligo #1267-09: | |
| 5′-CCG GAG CAC ATT TAT CAC ACA GCA | (SEQ ID NO:70) |
| GCT GAT GAC CAG TTT CTG GGT CCA-3′ | |

F. Murine OPG met-lys[22-401]

A DNA sequence coding for an N-terminal methionine followed by a lysine residue and amino acids 22 through 401 of murine OPG was placed under control of the lux PR promoter in prokaryotic expression vector pAMG21 as follows. Synthetic oligos #1282-95 and #1282-96 were phosphorylated individually and allowed to form an oligo linker duplex using methods described in Section B. The synthetic linker duplex utilized E. coli codons and provided for an N-terminal methionine. The phosphorylated linker duplex formed between oligos #1282-95 and #1282-96 containing NdeI and KpnI cohesive ends and the KpnI and BamHI digested and purified PCR product described in Section D was directionally inserted between the NdeI and BamHI sites in pAMG21 using standard methodology. The ligation mixture was transformed into E. coli host 393 by electroporation utilizing the manufacturer's protocol. Clones were selected, plasmid DNA was isolated, and DNA sequencing was performed to verify the DNA sequence of the MuOPG-Met-Lys[22-401] gene.

Expression of recombinant MuOPG Met-Lys[22-401] polypeptide from transformed 393 cells harboring the recombinant pAMG21 plasmid following induction was determined using methods described in Section C.

| | |
|---|---|
| Oligo #1282-95: | |
| 5′-TAT GAA AGA AAC TCT GCC TCC AAA | (SEQ ID NO:71) |
| ATA CCT GCA TTA CGA TCC GGA AAC TGG | |
| TCA TCA GCT GCT GTG TGA TAA ATG TGC | |
| TCC GGG TAC-3′ | |
| Oligo #1282-96: | |
| 5′-CCG GAG CAC ATT TAT CAC ACA GCA | (SEQ ID NO:72) |
| GCT GAT GAC CAG TTT CCG GAT CGT AAT | |
| GCA GGT ATT TTG GAG GCA GAG TTT CTT | |
| TCA-3′ | |

G. Murine OPG met-lys-(his)₇[22-401]

A DNA sequence coding for N-terminal residues Met-Lys-His-His-His-His-His-His-His (=MKH) followed by amino acids 22 through 401 of Murine OPG was placed under control of the lux PR promoter in prokaryotic expression vector pAMG21 as follows. PCR was performed using oligonucleotides #1300-50 and #1257-15 as primers and plasmid pAMG21-muOPG-met[22-401] DNA as template. Thermocycling conditions were as described in Section B. The resulting PCR sample was resolved on an agarose gel, the PCR product was excised, purified, cleaved with NdeI and BamHI restriction endonucleases and purified. The NdeI and BamHI digested and purified PCR product generated using oligo primers #1300-50 and #1257-15 was directionally inserted between the NdeI and BamHI sites of pAMG21 using standard DNA methodology. The ligation mixture was transformed into E. coli host 393 by electroporation utilizing the manufacturer's protocol. Clones were selected, plasmid DNA was isolated, and DNA sequencing performed to verify the DNA sequence of the muOPG-MKH[22-401] gene.

Expression of recombinant MuOPG-MKH[22-401] polypeptide from transformed 393 cultures harboring the recombinant pAMG21 plasmid following induction was determined using methods described in Section C.

| | |
|---|---|
| Oligo #1300-50: | |
| 5′-GTT CTC CTC ATA TGA AAC ATC ATC | (SEQ ID NO:73) |
| ACC ATC ACC ATC ATG AAA CTC TGC CTC | |
| CAA AAT ACC TGC ATT ACG AT-3′ | |

Oligo #1257-15: see Section D

H. Murine OPG met-lys[22-401](his)₇

A DNA sequence coding for a N-terminal met-lys, amino acids 22 through 401 murine OPG, and seven histidine residues following amino acid 401 (=muOPG MK[22-401]-H₇), was placed under control of the lux PR promoter in prokaryotic expression vector pAMG21 as follows. PCR was performed using oligonucleotides #1300-49 and #1300-51 as primers and pAMG21-muOPG met[22-401] DNA as template. Thermocycling conditions were as described in Section B. The resulting PCR sample was resolved on an agarose gel, the PCR product was excised, purified, restricted with NdeI and BamHI restriction endonucleases, and purified. The NdeI and BamHI digested and purified PCR product was directionally inserted between the NdeI and BamHI sites in pAMG21 using standard methodology. The ligation was transformed into E. coli host 393 by electroporation utilizing the manufacturer's protocol. Clones were selected, plasmid DNA was isolated, and DNA sequencing was performed to verify the DNA sequence of the muOPG MK[22-401]-H₇ gene.

Expression of the recombinant muOPG MK-[22-401]-H7 polypeptide from a transformed 393 cells harboring the recombinant pAMG21 plasmid following induction was determined using methods described in Section C.

| | |
|---|---|
| Oligo #1300-49: | |
| 5′-GTT CTC CTC ATA TGA AAG AAA CTC | (SEQ ID NO:74) |
| TGC CTC CAA AAT ACC TGC A-3′ | |
| Oligo #1300-51: | |
| 5′-TAC GCA CTG GAT CCT TAA TGA TGG | (SEQ ID NO:75) |
| TGA TGG TGA TGA TGT AAG CAG CTT ATT | |
| TTC ACG GAT TGA ACC TGA TTC CCT A-3′ | |

I. Murine OPG met[27-401]

A DNA sequence coding for a N-terminal methionine and amino acids 27 through 401 of murine OPG was placed under control of the lux PR promoter of prokaryotic expression vector pAMG21 as follows. PCR was performed with oligonucleotides #1309-74 and #1257-15 as primers and plasmid pAMG21-muOPG-met[22-401] DNA as template. Thermocycling conditions were as described in Section B. The resulting PCR sample was resolved on an agarose gel, the PCR product was excised, purified, cleaved with NdeI and BamHI restriction endonucleases, and purified. The NdeI and BamHI digested and purified PCR product was directionally inserted between the NdeI and BamHI sites of pAMG21 using standard methodology. The ligation mixture was transformed into E. coli host 393 by electroporation utilizing the manufacturer's protocol. Clones were selected, plasmid DNA was isolated, and DNA sequencing was performed to verify the DNA sequence of the muOPG-met[27-401] gene.

Expression of recombinant muOPG-met[27-401] polypeptide from a transfected 393 culture harboring the recombinant pAMG21 plasmid following induction was determined using methods described in Section C.

| | |
|---|---|
| Oligo #1309-74: | |
| 5′-GTT CTC CTC ATA TGA AAT ACC TGC | (SEQ ID NO:76) |
| ATT ACG ATC CGG AAA CTG GTC AT-3′ | |

Oligo#1257-15: See Section D

J. Human OPG met[27-401]

A DNA sequence coding for a N-terminal methionine and amino acids 27 through 401 of human OPG was placed under control of the lux PR promoter of prokaryotic expression vector pAMG21 as follows. PCR was performed using oligonucleotides #1309-75 and #1309-76 as primers and plasmid pAMG21-huOPG-met[22-401] DNA as template. Thermocycling conditions were as described in Section B. The resulting PCR sample was resolved on an agarose gel, the PCR product was excised, purified, restricted with AseI and BamHI restriction endonucleases, and purified. The AseI and BamHI digested and purified PCR product above was directionally inserted between the NdeI and BamHI sites of pAMG21 using standard methodology. The ligation mixture was transformed into E. coli host 393 by electroporation utilizing the manufacturer's protocol. Clones were selected, plasmid DNA was isolated, and DNA sequencing was performed to verify the DNA sequence of the huOPG-met[27-401] gene.

Expression of the recombinant huOPG-met[27-401] polypeptide following induction of from transfected 393 cells harboring the recombinant pAMG21 plasmid was determined using methods described in Section C.

| | |
|---|---|
| Oligo #1309-75: | |
| 5′-GTT CTC CTA TTA ATG AAA TAT CTT | (SEQ ID NO:77) |
| CAT TAT GAT GAA GAA ACT T-3′ | |
| Oligo #1309-76: | |
| 5′-TAC GCA CTG GAT CCT TAT AAG CAG | (SEQ ID NO:78) |
| CTT ATT TTT ACT GAT T-3′ | |

K. Murine OPG met[22-180]

A DNA sequence coding for a N-terminal methionine and amino acids 22 through 180 of murine OPG was placed under control of the lux PR promoter of prokaryotic expression vector pAMG21 as follows. PCR was performed with oligonucleotides #1309-72 and #1309-73 as primers and plasmid pAMG21-muOPG-met[22-401] DNA as template. Thermocycling conditions were as described in Section B. The resulting PCR sample was resolved on an agarose gel, the PCR product was excised, purified, restricted with NdeI and BamHI restriction endonucleases, and purified. The NdeI and BamHI digested and purified PCR product above was directionally inserted between the NdeI and BamHI sites of pAMG21 using standard methodology. The ligation was transformed into E. coli host 393 by electroporation utilizing the manufacturer's protocol. Clones were selected, plasmid DNA was isolated, and DNA sequencing was performed to verify the DNA sequence of the muOPG-met[22-180] gene.

Expression of recombinant muOPG-met[22-180] polypeptide from transformed 393 cultures harboring the recombinant pAMG21 plasmid following induction was determined using methods described in Section C.

| | |
|---|---|
| Oligo #1309-72: | |
| 5′-GTT CTC CTC ATA TGG AAA CTC TGC | (SEQ ID NO:79) |
| CTC CAA AAT ACC TGC A-3′ | |
| Oligo #1309-73: | |
| 5′-TAC GCA CTG GAT CCT TAT GTT GCA | (SEQ ID NO:80) |
| TTT CCT TTC TGA ATT AGC A-3′ | |

L. Murine OPG met[27-180]

A DNA sequence coding for a N-terminal methionine and amino acids 27 through 180 of murine OPG was placed under the control of the lux PR promoter of prokaryotic expression vector pAMG21 as follows. PCR was performed using oligonucleotides #1309-74 (see Section I) and #1309-73 (see Section K) as primers and plasmid pAMG21-muOPG met[22-401] DNA as template. Thermocycling conditions were as described in Section B. The resulting PCR sample was resolved on an agarose gel, the PCR product excised, purified, restricted with NdeI and BamHI restriction endonucleases, and purified. The NdeI and BamHI digested and purified PCR product above was directionally inserted between the NdeI and BamHI sites in pAMG21 using standard methodology. The ligation mixture was transformed into E. coli host 393 by electroporation utilizing the manufacturer's protocol. Clones were selected, plasmid DNA was isolated, and DNA sequencing was performed to verify the DNA sequence of the muOPG met[27-180] gene.

Expression of recombinant muOPG met[27-180] polypeptide from cultures of transformed 393 cells harboring the recombinant pAMG21 plasmid following induction was determined using methods described in Section C.

M. Murine OPG met[22-1891 and met[22-194]

A DNA sequence coding for a N-terminal methionine and either amino acids 22 through 189, or 22 through 194 of murine OPG was placed under control of the lux PR promoter of prokaryotic expression vector pAMG21 as follows. The pair of synthetic oligonucleotides #1337-92 and #1337-93 (=muOPG-189 linker) or #1333-57 and #1333-58 (=muOPG-194 linker) were phosphorylated individually and allowed to form an oligo linker duplex pair using methods described in Section B. Purified plasmid DNA of pAMG21-muOPG-met[22-401] was cleaved with KpnI and BspEI restriction endonucleases and the resulting DNA fragments were resolved on an agarose gel. The ˜413 bp B fragment was isolated using standard recombinant DNA methodology. The phosphorylated oligo linker duplexes formed between either oligos #1337-92 and #1337-93 (muOPG-189 linker) or oligos #1333-57 and #1333-58 (muOPG-194 linker) containing BspEI and BamHI cohesive ends, and the isolated ˜413 bp B fragment of plasmid pAMG21-muOPG-met[22-401] digested with KpnI and BspEI restriction endonucleases above, was directionally inserted between the KpnI and BamHI sites of pAMG21-muOPG met[22-401] using standard methodology. Each ligation mixture was transformed into E. coli host 393 by electroporation utilizing the manufacturer's protocol. Clones were selected, plasmid DNA was isolated, and DNA sequencing was performed to verify the DNA sequence of either the muOPG-met[22-189] or muOPG-met[22-194] genes.

Expression of recombinant muOPG-met[22-189] and muOPG-met[22-194] polypeptides from recombinant pAMG21 plasmids transformed into 393 cells was determined using methods described in Section C.

| | |
|---|---|
| Oligo #1337-92: | |
| 5′-CCG GAA ACA GAT AAT GAG-3′ | (SEQ ID NO:81) |
| Oligo #1337-93: | |
| 5′-GAT CCT CAT TAT CTG TTT-3′ | (SEQ ID NO:82) |
| Oligo #1333-57: | |
| 5′-CCG GAA ACA GAG AAG CCA CGC AAA | (SEQ ID NO:83) |
| AGT AAG-3′ | |
| Oligo #1333-58: | |
| 5′-GAT CCT TAC TTT TGC GTG GCT TCT | (SEQ ID NO:84) |
| CTG TTT-3′ | |

N. Murine OPG met[27-189] and met[27-194]

A DNA sequence coding for a N-terminal methionine and either amino acids 27 through 189, or 27 through 194 of murine OPG was placed under control of the lux PR promoter of prokaryotic expression vector pAMG21 as follows. Phosphorylated oligo linkers either “muOPG-189 linker” or “muOPG-194 linker” (see Section M) containing BspEI and BamHI cohesive ends, and the isolated ˜413 bp B fragment of plasmid pAMG21-muOPG-met[22-401] digested with KpnI and BspEI restriction endonucleases were directionally inserted between the KpnI and BamHI sites of plasmid pAMG21-muOPG-met[27-401] using standard methodology. Each ligation was transformed into E. coli host 393 by electroporation utilizing the manufacturer's protocol. Clones were selected, plasmid DNA was isolated, and DNA sequencing was performed to verify the DNA sequence of either the muOPG met[27-189] or muOPG met[27-194] genes.

Expression of recombinant muOPG met[27-189] and muOPG met[27-194] following induction of 393 cells harboring recombinant pAMG21 plasmids was determined using methods described in Section C.

O. Human OPG met[22-1851. met[22-1891, met[22-194]

A DNA sequence coding for a N-terminal methionine and either amino acids 22 through 185, 22 through 189, or 22 through 194 of the human OPG polypeptide was placed under control of the lux PR promoter of prokaryotic expression vector pAMG21 as follows. The pair of synthetic oligonucleotides #1331-87 and #1331-88 (=huOPG-185 linker), #1331-89 and #1331-90 (=huOPG-189 linker), or #1331-91 & #1331-92 (=huOPG-194 linker) were phosphorylated individually and each allowed to form an oligo linker duplex pair using methods described in Section B. Purified plasmid DNA of pAMG21-huOPG-met[27-401] was restricted with KpnI and NdeI restriction endonucleases and the resulting DNA fragments were resolved on an agarose gel. The ˜407 bp B fragment was isolated using standard recombinant DNA methodology. The phophorylated oligo linker duplexes formed between either oligos #1331-87 and #1331-88 (huOPG-185 linker), oligos #1331-89 and #1331-90 (huOPG-189 linker), or oligos #1331-91 and #1331-92 (huOPG-194 linker)[each linker contains NdeI and BamHI cohesive ends], and the isolated ˜407 bp B fragment of plasmid pAMG21-huOPG-met[27-401] digested with KpnI and NdeI restriction endonucleases above, was directionally inserted between the KpnI and BamHI sites of plasmid pAMG21-huOPG-met[22-401] using standard methodology. Each ligation was transformed into E. coli host 393 by electroporation utilizing the manufacturer's protocol. Clones were selected, plasmid DNA was isolated, and DNA sequencing was performed to verify the DNA sequence of either the huOPG-met[22-185], huOPG-met[22-189], or huOPG-met[22-194] genes.

Expression of recombinant huOPG-met[22-185], huOPG-met[22-189] or huOPG-met[22-194] in transformed 393 cells harboring recombinant pAMG21 plasmids following induction was determined using methods described in Section C.

| | |
|---|---|
| Oligo #1331-87: | |
| 5′-TAT GTT AAT GAG-3′ | (SEQ ID NO:85) |
| Oligo #1331-88: | |
| 5′-GAT CCT CAT TAA CA-3′ | (SEQ ID NO:86) |
| Oligo #1331-89: | |
| 5′-TAT GTT CCG GAA ACA GTT AAG-3′ | (SEQ ID NO:87) |
| Oligo #1331-90: | |
| 5′-GAT CCT TAA CTG TTT CCG GAA CA-3′ | (SEQ ID NO:88) |
| Oligo #1331-91: | |
| 5′-TAT GTT CCG GAA ACA GTG AAT CAA | (SEQ ID NO:89) |
| CTC AAA AAT AAG-3′ | |
| Oligo #1331-92: | |
| 5′-GAT CCT TAT TTT TGA GTT GAT TCA | (SEQ ID NO:90) |
| CTG TTT CCG GAA CA-3′ | |

P. Human OPG met[27-1851. met[27-189], met [27-194]

A DNA sequence coding for a N-terminal methionine and either amino acids 27 through 185, 27 through 189, or 27 through 194 of the human OPG polypeptide was placed under control of the lux PR promoter of prokaryotic expression vector pAMG21 as follows. Phosphorylated oligo linkers “huOPG-185 linker”, “huOPG-189 linker”, or “huOPG-194 linker” (See Section O) each containing NdeI and BamHI cohesive ends, and the isolated ˜407 bp B fragment of plasmid pAMG21-huOPG-met[27-401] digested with KpnI and NdeI restriction endonucleases (See Section O) were directionally inserted between the KpnI and BamHI sites of plasmid pAMG21-huOPG-met[27-401] (See Section J) using standard methodology. Each ligation was transformed into E. coli host 393 by electroporation utilizing the manufacturer's protocol. Clones were selected, plasmid DNA isolated, and DNA sequencing performed to verify the DNA sequence of either the huOPG-met[27-185], huOPG-met[27-189], or huOPG-met[27-194] genes.

Expression of recombinant huOPG-met[27-185], huOPG-met[27-189], and huOPG-met[27-194] from recombinant pAMG21 plasmids transformed into 393 cells was determined using methods described in Section C.

Q. Murine OPG met[27-401] (P33E, G36S A45P)

A DNA sequence coding for an N-terminal methionine and amino acids 27 through 48 of human OPG followed by amino acid residues 49 through 401 of murine OPG was placed under control of the lux PR promoter of prokaryotic expression vector pAMG21 as follows. Purified plasmid DNA of pAMG21-huOPG-met[27-401] (See Section J) was cleaved with AatII and KpnI restriction endonucleases and a ˜1075 bp B fragment isolated from an agarose gel using standard recombinant DNA methodology. Additionally, plasmid pAMG21-muOPG-met[22-401] DNA (See Section D) was digested with KpnI and BamHI restriction endonucleases and the ˜1064 bp B fragment isolated as described above. The isolated ˜1075 bp pAMG21-huOPG-met[27-401] restriction fragment containing AatII & KpnI cohesive ends (see above), the ˜1064 bp pAMG21-muOPG-met[22-401] restriction fragment containing KpnI and BamHI sticky ends and a ˜5043 bp restriction fragment containing AatII and BamHI cohesive ends and corresponding to the nucleic acid sequence of pAMG21 between AatII & BamHI were ligated using standard recombinant DNA methodology. The ligation was transformed into E. coli host 393 by electroporation utilizing the manufacturer's protocol. Clones were selected, and the presence of the recombinant insert in the plasmid verified using standard DNA methodology. muOPG-27-401 (P33E, G36S, A45P) gene. Amino acid changes in muOPG from proline-33 to glutamic acid-33, glycine-36 to serine-36, and alanine45 to proline45, result from replacement of muOPG residues 27 through 48 with huOPG residues 27 through 48.

Expression of recombinant muOPG-met[27-401] (P33E, G36S, A45P) from transformed 393 cells harboring the recombinant pAMG21 plasmid was determined using methods described in Section C.

R. Murine OPG met-lys-(his)₇-ala-ser-(asp)₄-lys[22-401](A45T)

A DNA sequence coding for an N-terminal His tag and enterokinase recognition sequence which is (NH₂ to COOH terminus): Met-Lys-His-His-His-His-His-His-His-Ala-Ser-Asp-Asp-Asp-Asp-Lys (=HEK), followed by amino acids 22 through 401 of the murine OPG polypeptide was placed under control of the lac repressor regulated Ps4 promoter as follows. pAMG22-His (See Section A) was digested with NheI and BamHI restriction endonucleases, and the large fragment (the A fragment) isolated from an agarose gel using standard recombinant DNA methodology. Oligonucleotides #1282-91 and #1282-92 were phosphorylated individually and allowed to form an oligo linker duplex using methods previously described (See Section B). The phosphorylated linker duplex formed between oligos #1282-91 and #1282-92 containing NheI and KpnI cohesive ends, the KpnI and BamHI digested and purified PCR product described (see Section D), and the A fragment of vector pAMG22-His digested with NheI and BamHI were ligated using standard recombinant DNA methodology. The ligation was transformed into E. coli host GM 120 by electroporation utilizing the manufacturer's protocol. Clones were selected, plasmid DNA isolated and DNA sequencing performed to verify the DNA sequence of the muOPG-HEK[22-401] gene. DNA sequencing revealed a spurious mutation in the natural muOPG sequence that resulted in a single amino acid change of Alanine-45 of muOPG polypeptide to a Threonine.

Expression of recombinant muOPG-HEK[22-401] (A45T) from GM120 cells harboring the recombinant pAMG21 plasmid was determined using methods similar to those described in Section C, except instead of addition of the synthetic autoinducer, IPTG was added to 0.4 mM final to achieve induction.

| | |
|---|---|
| Oligo #1282-91: | |
| 5′-CTA GCG ACG ACG ACG ACA AAG AAA | (SEQ ID NO:91) |
| CTC TGC CTC CAA AAT ACC TGC ATT ACG | |
| ATC CGG AAA CTG GTC ATC AGC TGC TGT | |
| GTG ATA AAT GTG CTC CGG GTA C-3′ | |
| Oligo #1282-92: | |
| 5′-CCG GAG CAC ATT TAT CAC ACA GCA | (SEQ ID NO:92) |
| GCT GAT GAC CAG TTT CCG GAT CGT AAT | |
| GCA GGT ATT TTG GAG GCA GAG TTT CTT | |
| TGT CGT CGT CGT CG-3′ | |

S. Human OPG met-arg-gly-ser-(his)₆[22-401]

Eight oligonucleotides (1338-09 to 1338-16 shown below) were designed to produce a 175 base fragment as overlapping, double stranded DNA. The oligos were annealed, ligated, and the 5′ and 3′ oligos were used as PCR primers to produce large quantities of the 175 base fragment. The final PCR gene products were digested with restriction endonucleases ClaI and KpnI to yield a fragment which replaces the N-terminal 28 codons of human OPG. The ClaI and KpnI digested PCR product was inserted into pAMG21-huOPG [27-401] which had also been cleaved with ClaI and KpnI. Ligated DNA was transformed into competent host cells of E. coli strain 393. Clones were screened for the ability to produce the recombinant protein product and to possess the gene fusion having the correct nucleotide sequence. Protein expression levels were determined from 50 ml shaker flask studies. Whole cell lysate and sonic pellet were analyzed for expression of the construct by Coomassie stained PAGE gels and Western analysis with murine anti-OPG antibody. Expression of huOPG Met-Arg-Gly-Ser-(His)₆ [22-401] resulting in the formation of large inclusion bodies and the protein was localized to the insoluble (pellet) fraction.

| | |
|---|---|
| 1338-09: | |
| ACA AAC ACA ATC GAT TTG ATA CTA GA | (SEQ ID NO:93) |
| 1338-10: | |
| TTT GTT TTA ACT AAT TAA AGG AGG | (SEQ ID NO:94) |
| AAT AAA ATA TGA GAG GAT CGC ATC | |
| AC | |
| 1338-11: | |
| CAT CAC CAT CAC GAA ACC TTC CCG | (SEQ ID NO:95) |
| CCG AAA TAC CTG CAC TAC GAC GAA | |
| GA | |
| 1338-12: | |
| AAC CTC CCA CCA GCT GCT GTG CGA | (SEQ ID NO:96) |
| CAA ATG CCC GCC GGG TAC CCA AAC A | |
| 1338-13: | |
| TGT TTG GGT ACC CGG CGG GCA TTT GT | (SEQ ID NO:97) |
| 1338-14: | |
| CGC ACA GCA GCT GGT GGG AGG TTT | (SEQ ID NO:98) |
| CTT CGT CGT AGT GCA GGT ATT TCG GC | |
| 1338-15: | |
| GGG AAG GTT TCG TGA TGG TGA TGG | (SEQ ID NO:99) |
| TGA TGC GAT CCT CTC ATA TTT TAT T | |
| 1338-16: | |
| CCT CCT TTA ATT AGT TAA AAC AAA | (SEQ ID NO:100) |
| TCT AGT ATC AAA TCG ATT GTG TTT GT | |

T. Human OPG met-lys[22-401] and met(lys)₃[22-401]

To construct the met-lys and met-(lys)₃ versions of human OPG[22-401], overlapping oligonucleotides were designed to add the appropriate number of lysine residues. The two oligos for each construct were designed to overlap, allowing two rounds of PCR to produce the final product. The template for the first PCR reaction was a plasmid DNA preparation containing the human OPG 22-401 gene. The first PCR added the lysine residue(s). The second PCR used the product of the first round and added sequence back to the first restriction site, ClaI.

The final PCR gene products were digested with restriction endonucleases ClaI and KpnI, which replace the N-terminal 28 codons of hu OPG, and then ligated into plasmid pAMG21-hu OPG [27-401] which had been also digested with the two restriction endonucleases. Ligated DNA was transformed into competent host cells of E. coli strain 393. Clones were screened for the ability to produce the recombinant protein product and to possess the gene fusion having the correct nucleotide sequence. Protein expression levels were determined from 50 ml shaker flask studies. Whole cell lysate and sonic pellet were analyzed for expression of the construct by Coomassie stained PAGE gels and Western analysis with murine anti-OPG antibody. Neither construct had a detectable level of protein expression and inclusion bodies were not visible. The DNA sequences were confirmed by DNA sequencing.

| | |
|---|---|
| Oligonucleotide | |
| primers to prepare | |
| Met-Lys huOPG[22-401]: | |
| 1338-17: | |
| ACA AAC ACA ATC GAT TTG ATA CTA | (SEQ ID NO:101) |
| GAT TTG TTT TAA CTA ATT AAA GGA | |
| GGA ATA AAA TG | |
| 1338-18: | |
| CTA ATT AAA GGA GGA ATA AAA TGA | (SEQ ID NO:102) |
| AAG AAA CTT TTC CTC CAA AAT ATC | |
| 1338-20: | |
| TGT TTG GGT ACC CGG CGG ACA TTT | (SEQ ID NO:103) |
| ATC ACA C | |
| Oligonucleotide | |
| primers to prepare | |
| Met-(Lys)₃-huOPG[22-401]: | |
| 1338-17: | |
| ACA AAC ACA ATC GAT TTG ATA CTA | (SEQ ID NO:104) |
| GAT TTG TTT TAA CTA ATT AAA GGA | |
| GGA ATA AAA TG | |
| 1338-19: | |
| CTA ATT AAA GGA GGA ATA AAA TGA | (SEQ ID NO:105) |
| AAA AAA AAG AAA CTT TTC CTC CAA | |
| AAT ATC | |
| 1338-20: | |
| TGT TTG GGT ACC CGG CGG ACA TTT | (SEQ ID NO:106) |
| ATC ACA C | |

U. Human and Murine OPG [22-401]/Fc Fusions

Four OPG-Fc fusions were constructed where the Fc region of human IgG1 was fused at the N-terminus of either human or murine Osteoprotegerin amino acids 22 to 401 (referred to as Fc/OPG [22-401]) or at the C-terminus (referred to as OPG[22-401]/Fc). Fc fusions were constructed using the fusion vector pFc-A3 described in Example 7.

All fusion genes were constructed using standard PCR technology. Template for PCR reactions were plasmid preparations containing the target genes. Overlapping oligos were designed to combine the C-terminal portion of one gene with the N terminal portion of the other gene. This process allows fusing the two genes together in the correct reading frame after the appropriate PCR reactions have been performed. Initially one “fusion” oligo for each gene was put into a PCR reaction with a universal primer for the vector carrying the target gene. The complimentary “fusion” oligo was used with a universal primer to PCR the other gene. At the end of this first PCR reaction, two separate products were obtained, with each individual gene having the fusion site present, creating enough overlap to drive the second round of PCR and create the desired fusion. In the second round of PCR, the first two PCR products were combined along with universal primers and via the overlapping regions, the full length fusion DNA sequence was produced.

The final PCR gene products were digested with restriction endonucleases XbaI and BamHI, and then ligated into the vector pAMG21 having been also digested with the two restriction endonucleases. Ligated DNA was transformed into competent host cells of E. coli strain 393. Clones were screened for the ability to produce the recombinant protein product and to possess the gene fusion having the correct nucleotide sequence. Protein expression levels were determined from 50 ml shaker flask studies. Whole cell lysate, sonic pellet, and supernatant were analyzed for expression of the fusion by Coomassie stained PAGE gels and Western analysis with murine anti-OPG antibody.

Fc/huOPG [22-401]

Expression of the Fc/hu OPG [22-401] fusion peptide was detected on a Coomassie stained PAGE gel and on a Western blot. The cells have very large inclusion bodies, and the majority of the product is in the insoluble (pellet) fraction. The following primers were used to construct this OPG-Fc fusion:

| | |
|---|---|
| 1318-48: | |
| CAG CCC GGG TAA AAT GGA AAC GTT | (SEQ ID NO:107) |
| TCC TCC AAA ATA TCT TCA TT | |
| 1318-49: | |
| CGT TTC CAT TTT ACC CGG GCT GAG | (SEQ ID NO:108) |
| CGA GAG GCT CTT CTG CGT GT | |

Fc/muOPG [22-401]

Expression of the fusion peptide was detected on a Coomassie stained gel and on a Western blot. The cells have very large inclusion bodies, and the majority of the product is in the insoluble (pellet) fraction. The following primers were used to construct this OPG-Fc fusion:

| | |
|---|---|
| 1318-50: | |
| CGC TCA GCC CGG GTA AAA TGG AAA | (SEQ ID NO:109) |
| CGT TGC CTC CAA AAT ACC TGC | |
| 1318-51: | |
| CCA TTT TAC CCG GGC TGA GCG AGA | (SEQ ID NO:110) |
| GGC TCT TCT GCG TGT | |

muOPG [22-401]/Fc

Expression of the fusion peptide was detected on a Coomassie stained gel and on a Western blot. The amount of recombinant product was less than the OPG fusion proteins having the Fc region in the N terminal position. Obvious inclusion bodies were not detected. Most of the product appeared to be in the insoluble (pellet) fraction. The following primers were used to construct this OPG-Fc fusion:

| | |
|---|---|
| 1318-54: | |
| GAA AAT AAG CTG CTT AGC TGC AGC | (SEQ ID NO:111) |
| TGA ACC AAA ATC | |
| 1318-55: | |
| CAG CTG CAG CTA AGC AGC TTA TTT | (SEQ ID NO:112) |
| TCA CGG ATT G | |

huOPG [22-401]/Fc

Expression of the fusion peptide was not detected on a Coomassie stained gel, although a faint Western positive signal was present. Obvious inclusion bodies were not detected. The following primers were used to prepare this OPG-Fc fusion:

| | |
|---|---|
| 1318-52: | |
| AAA AAT AAG CTG CTT AGC TGC AGC | (SEQ ID NO:113) |
| TGA ACC AAA ATC | |
| 1318-53: | |
| CAG CTG CAG CTA AGC AGC TTA TTT | (SEQ ID NO:114) |
| TTA CTG ATT GG | |

V. Human OPG met[22-401]-Fc Fusion (P25A)

This construct combines a proline to alanine amino acid change at position 25 (P25A) with the huOPG met[22-401]-Fc fusion. The plasmid was digested with restriction endonucleases ClaI and KpnI, which removes the N-terminal 28 codons of the gene, and the resulting small (less than 200 base pair) fragment was gel purified. This fragment containing the proline to alanine change was then ligated into plasmid pAMG21-huOPG [22-401]-Fc fusion which had been digested with the two restriction endonucleases. The ligated DNA was transformed into competent host cells of E. coli strain 393. Clones were screened for the ability to produce the recombinant protein product and to possess the gene fusion having the correct nucleotide sequence. Protein expression levels were determined from 50 ml shaker flask studies. Whole cell lysate and sonic pellet were analyzed for expression of the construct by Coomassie stained PAGE gels and Western analysis with murine anti-OPG antibody. The expression level of the fusion peptide was detected on a Coomassie stained PAGE gel and on a Western blot. The protein was in the insoluble (pellet) fraction. The cells had large inclusion bodies.

W. Human OPG met[22-401] (P25A)

A DNA sequence coding for an N-terminal methionine and amino acids 22 through 401 of human OPG with the proline at position 25 being substituted by alanine under control of the lux PR promoter in prokaryotic expression vector pAMG21 was constructed as follows: Synthetic oligos # 1289-84 and 1289-85 were annealed to form an oligo linker duplex with XbaI and KpnI cohesive ends. The synthetic linker duplex utilized optimal E. coli codons and encoded an N-terminal methionine. The linker also included an SpeI restriction site which was not present in the original sequence. The linker duplex was directionally inserted between the XbaI and KpnI sites in pAMG21-huOPG-22401 using standard methods. The ligation mixture was introduced into E. coli host GM221 by transformation. Clones were initially screened for production of the recombinant protein. Plasmid DNA was isolated from positive clones and DNA sequencing was performed to verify the DNA sequence of the huOPG-Met[22-401](P25A) gene. The following oligonucleotides were used to generate the XbaI-KpnI linker:

| | |
|---|---|
| Oligo #1289-84: | |
| 5′-CTA GAA GGA GGA ATA ACA TAT GGA | (SEQ ID NO:115) |
| AAC TTT TGC TCC AAA ATA TCT TCA | |
| TTA TGA TGA AGA AAC TAG TCA TCA | |
| GCT GCT GTG TGA TAA ATG TCC GCC | |
| GGG TAC-3′ | |
| Oligo #1289-85: | |
| 5′-CCG GCG GAC ATT TAT CAC ACA GCA | (SEQ ID NO:116) |
| GCT GAT GAC TAG TTT CTT CAT CAT | |
| AAT GAA GAT ATT TTG GAG CAA AAG | |
| TTT CCA TAT GTT ATT CCT CCT T-3′ | |

X. Human OPG met[22-401] (P26A) and (P26D)

A DNA sequence coding for an N-terminal methionine and amino acids 22 through 401 of human OPG with the proline at position 26 being substituted by alanine under control of the lux P_{R} promoter in prokaryotic expression vector pAMG21 was constructed as follows: Synthetic oligos # 1289-86 and 1289-87 were annealed to form an oligo linker duplex with XbaI and SpeI cohesive ends. The synthetic linker duplex utilized optimal E. coli codons and encoded an N-terminal methionine. The linker duplex was directionally inserted between the XbaI and SpeI sites in pAMG21-huOPG[22-401](P25A) using standard methods. The ligation mixture was introduced into E. coli host GM221 by transformation. Clones were initially screened for production of the recombinant protein. Plasmid DNA was isolated from positive clones and DNA sequencing was performed to verify the DNA sequence of the huOPG-met[22-401](P26A) gene. One of the clones sequenced was found to have the proline at position 26 substituted by aspartic acid rather than alanine, and this clone was designated huOPG-met[22-401](P26D). The following oligonucleotides were used to generate the XbaI-SpeI linker:

| | |
|---|---|
| Oligo #1289-86: | |
| 5′-CTA GAA GGA GGA ATA ACA TAT GGA | (SEQ ID NO:117) |
| AAC TTT TCC TGC TAA ATA TCT TCA | |
| TTA TGA TGA AGA AA-3′ | |
| Oligo #1289-87: | |
| 5′-CTA GTT TCT TCA TCA TAA TGA AGA | (SEQ ID NO:118) |
| TAT TTA GCA GGA AAA GTT TCC ATA | |
| TGT TAT TCC TCC TT-3′ | |

Y. Human OPG met[22-194] (P25A)

A DNA sequence coding for an N-terminal methionine and amino acids 22 through 194 of human OPG with the proline at position 25 being substituted by alanine under control of the lux P_{R} promoter in prokaryotic expression vector pAMG21 was constructed as follows: The plasmids pAMG21-huOPG[27-194] and pAMG21-huOPG[22-401] (P25A) were each digested with KpnI and BamHI endonucleases. The 450 bp fragment was isolated from pAMG21-huOPG[27-194] and the 6.1 kbp fragment was isolated from pAMG21-huOPG[22-401] (P25A). These fragments were ligated together and introduced into E. coli host GM221 by transformation. Clones were initially screened for production of the recombinant protein. Plasmid DNA was isolated from positive clones and DNA sequencing was performed to verify the DNA sequence of the huOPG-Met[22-194](P25A) gene.

### EXAMPLE 9

### Association of OPG Monomers

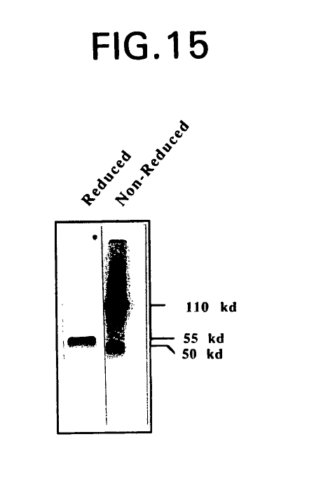

CHO cells engineered to overexpress muOPG [22-401] were used to generate conditioned media for the analysis of secreted recombinant OPG using rabbit polyclonal anti-OPG antibodies. An aliquot of conditioned media was concentrated 20-fold, then analysed by reducing and non-reducing SDS-PAGE (FIG. 15). Under reducing conditions, the protein migrated as a Mr 50-55 kd polypeptide, as would be predicted if the mature product was glycosylated at one or more of its consensus N-linked glycosylation sites. Suprisingly, when the same samples were analysed by non-reducing SDS-PAGE, the majority of the protein migrated as an approximately 100 kd polypeptide, twice the size of the reduced protein. In addition, there was a smaller amount of the Mr 50-55 kd polypeptide. This pattern of migration on SDS-PAGE was consistent with the notion that the OPG product was forming dimers through oxidation of a free sulfhydryl group(s).

The predicted mature OPG polypeptide contains 23 cysteine residues, 18 of which are predicted to be involved in forming intrachain disulfide bridges which comprise the four cysteine-rich domains (FIG. 12A). The five remaining C-terminal cysteine residues are not involved in secondary structure which can be predicted based upon homology with other TNFR family members. Overall there is a net uneven number of cysteine residues, and it is formally possible that at least one residue is free to form an intermolecular disulfide bond between two OPG monomers.

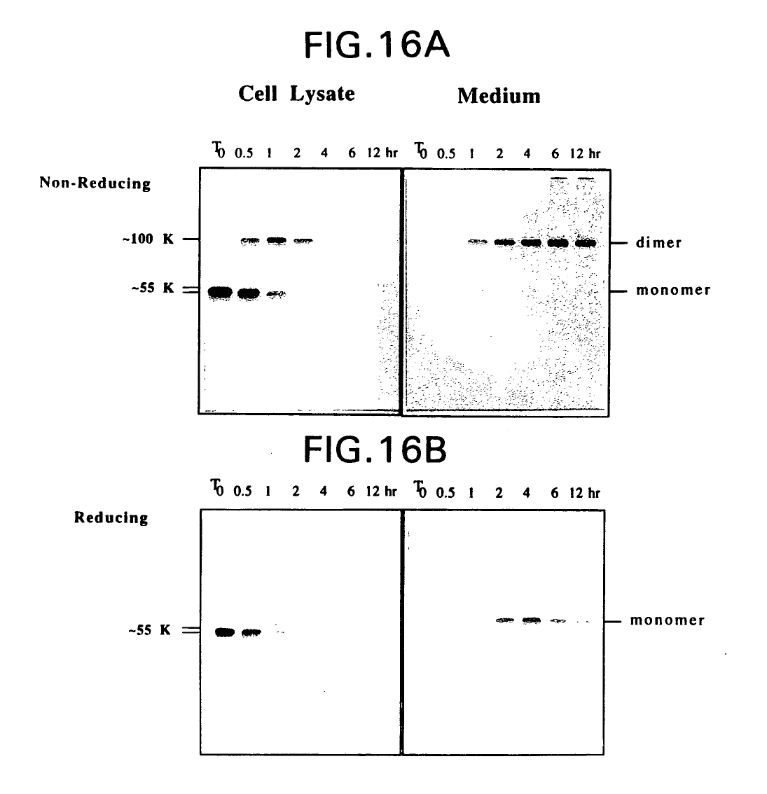

To help elucidate patterns of OPG kinesis and monomer association, a pulse-chase labelling study was performed. CHO cells expressing muOPG [22-401] were metabolically labelled as described above in serum-free medium containing ³⁵S methionine and cysteine for 30 min. After this period, the media was removed, and replaced with complete medium containing unlabelled methionine and cysteine at levels approximately 2,000-fold excess to the original concentration of radioactive amino acids. At 30 min, 1 hr, 2 hr, 4 hr, 6 hr and 12 hr post addition, cultures were harvested by the removal of the conditioned media, and lysates of the conditioned media and adherent monolayers were prepared. The culture media and cell lysates were clarified as described above, and then immunoprecipitated using anti-OPG antibodies as described above. After the immunoprecipitates were washed, they were released by boiling in non-reducing SDS-PAGE buffer then split into two equal halves. To one half, the reducing agent □-mercaptothanol was added to 5% (v/v) final concentration, while the other half was maintained in non-reducing conditions. Both sets of immunoprecipitates were analysed by SDS-PAGE as described above, then processed for autoradiography and exposed to film. The results are shown in FIG. 16. The samples analysed by reducing SDS-PAGE are depicted in the bottom two panels. After synthesis, the OPG polypeptide is rapidly processed to a slightly larger polypeptide, which probably represents modification by N-linked glycoslyation. After approximately 1-2 hours, the level of OPG in the cell decreases dramatically, and concomitantly appears in the culture supernatant. This appears to be the result of the vectoral transport of OPG from the cell into the media over time, consistent with the notion that OPG is a naturally secreted protein. Analysis of the same immunoprecipitates under nonreducing conditions reveals the relationship between the formation of OPG dimers and secretion into the conditioned media (FIG. 16, upper panels). In the first 30-60 minutes, OPG monomers are processed in the cell by apparent glycoslylation, followed by dimer formation. Over time, the bulk of OPG monomers are driven into dimers, which subsequently disappear from the cell. Beginning about 60 minutes after synthesis, OPG dimers appear in the conditioned media, and accumulate over the duration of the experiment. Following this period, OPG dimers are formed, which are then secreted into the culture media. OPG monomers persist at a low level inside the cell over time, and small amounts also appear in the media. This does not appear to be the result of breakdown of covalent OPG dimers, but rather the production of sub-stoichiometric amounts of monomers in the cell and subsequent secretion.

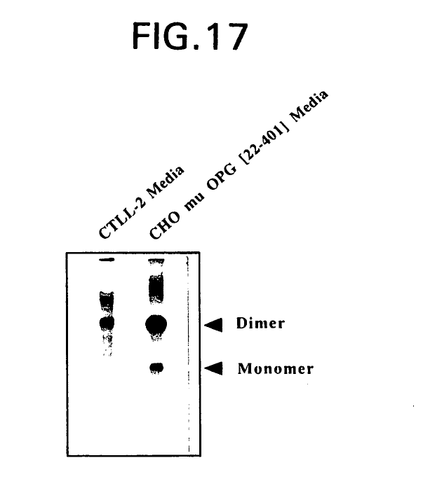

Recombinantly produced OPG from transfected CHO cells appears to be predominantly a dimer. To determine if dimerization is a natural process in OPG synthesis, we analysed the conditioned media of a cell line found to naturally express OPG. The CTLL-2 cell line, a murine cytotoxic T lymphocytic cell line (ATCC accession no. TIB-214), was found to express OPG mRNA in a screen of tissue and cell line RNA. The OPG transcript was found to be the same as the cloned and sequenced 2.5-3.0 kb RNA identified from kidney and found to encode a secreted molecule. Western blot analysis of conditioned media obtained from CTLL-2 cells shows that most, if not all, of the OPG secreted is a dimer (FIG. 17). This suggests that OPG dimerization and secretion is not an artifact of overexpression in a cell line, but is likely to be the main form of the product as it is produced by expressing cells.

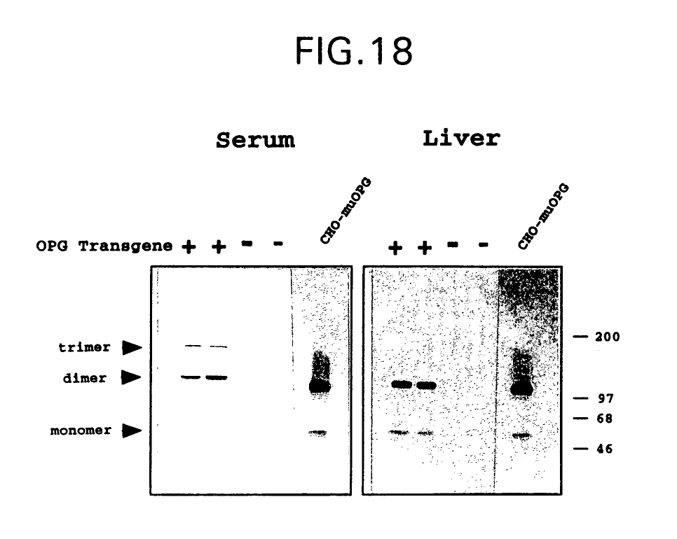

Normal and transgenic mouse tissues and serum were analysed to determine the nature of the OPG molecule expressed in OPG transgenic mice. Since the rat OPG cDNA was expressed under the control of a hepatocyte control element, extracts made from the parenchyma of control and transgenic mice under non-reducing conditions were analysed (FIG. 18). In extract from transgenic, but not control mice, OPG dimers are readily detected, along with substoichiometric amounts of monomers. The OPG dimers and monomers appear identical to the recombinant murine protein expressed in the genetically engineered CHO cells. This strongly suggests that OPG dimers are indeed a natural form of the gene product, and are likely to be key active components. Serum samples obtained from control and transgenic mice were similarly analysed by western blot analysis. In control mice, the majority of OPG migrates as a dimer, while small amounts of monomer are also detected. In addition, significant amounts of a larger OPG related protein is detected, which migrates with a relative molecular mass consistent with the predicted size of a covalently-linked trimer. Thus, recombinant OPG is expressed predominantly as a dimeric protein in OPG transgenic mice, and the dimer form may be the basis for the osteopetrotic phenotype in OPG mice. OPG recombinant protein may also exist in higher molecular weight “trimeric” forms.

To determine if the five C-terminal cysteine residues of OPG play a role in homodimerization, the murine OPG codons for cytsteine residues 195 (C195), C202, C277, C319, and C400 were changed to serine using the QuickChange™ Site-Directed Mutagenesis Kit (Stratagene, San Diego, Calif.) as described above. The muOPG gene was subcloned between the Not I and Xba I sites of the pcDNA 3.1 (+) vector (Invitrogen, San Diego, Calif.). The resulting plasmid, pcDNA3.1-muOPG, and mutagenic primers were treated with Pfu polymerase in the presence of deoxynucleotides, then amplified in a thermocycler as described above. An aliqout of the reaction is then transfected into competent E. coli XL1-Blue by heatshock, then plated. Plasmid DNA from transformants was then sequenced to verify mutations.

The following primer pairs were used to change the codon for cysteine residue 195 to serine of the murine OPG gene, resulting in the production of a muOPG [22-401] C195S protein:

| | |
|---|---|
| 1389-19: | |
| 5′-CAC GCA AAA GTC GGG AAT AGA TGT | (SEQ ID NO:150) |
| CAC-3′ | |
| 1406-38: | |
| 5′-GTG ACA TCT ATT CCC GAC TTT TGC | (SEQ ID NO:151) |
| GTG-3′ | |

The following primer pairs were used to change the codon for cysteine residue 202 to serine of the murine OPG gene, resulting in the production of a muOPG [22-401] C202S protein:

| | |
|---|---|
| 1389-21: | |
| 5′-CAC CCT GTC GGA AGA GGC CTT CTT | (SEQ ID NO:152) |
| C-3′ | |
| 1389-22: | |
| 5′-GAA GAA GGC CTC TTC CGA CAG GGT | (SEQ ID NO:153) |
| G-3′ | |

The following primer pairs were used to change the codon for cysteine residue 277 to serine of the murine OPG gene, resulting in the production of a muOPG [22-401] C277S protein:

| | |
|---|---|
| 1389-23: | |
| 5′-TGA CCT CTC GGA AAG CAG CGT | (SEQ ID NO:154) |
| GCA-3′ | |
| 1389-24: | |
| 5′-TGC ACG CTG CTT TCC GAG AGG | (SEQ ID NO:155) |
| TCA-3′ | |

The following primer pairs were used to change the codon for cysteine residue 319 to serine of the murine OPG gene, resulting in the production of a muOPG [22-401] C319S protein:

| | |
|---|---|
| 1389-17: | |
| 5′-CCT CGA AAT CGA GCG AGC AGC | (SEQ ID NO:156) |
| TCC-3′ | |
| 1389-18: | |
| 5′-CGA TTT CGA GGT CTT TCT CGT TCT | (SEQ ID NO:157) |
| C-3′ | |

The following primer pairs were used to change the codon for cysteine residue 400 to serine of the murine OPG gene, resulting in the production of a muOPG [22-401] C400S protein:

| | |
|---|---|
| 1406-72: | |
| 5′-CCG TGA AAA TAA GCT CGT TAT AAC | (SEQ ID NO:158) |
| TAG GAA TGG-3′ | |
| 1406-75: | |
| 5′-CCA TTC CTA GTT ATA ACG AGC TTA | (SEQ ID NO:159) |
| TTT TCA CGG-3′ | |

Each resulting muOPG [22-401] plasmid containing the appropriate mutation was then transfected into human 293 cells, the mutant OPG-Fc fusion protein purified from conditioned media as described above. The biological activity of each protein was assessed the in vitro osteoclast forming assay described in example 11. Conditioned media from each transfectant was analysed by non-reducing SDS-PAGE and western blotting with anti-OPG antibodies.

Mutation of any of the five C-terminal cysteine residues results in the production of predominantly (>90%) monomeric 55 kd OPG molecules. This strongly suggests that the C-terminal cysteine residues together play a role in OPG homodimerization.

C-terminal OPG deletion mutants were constructed to map the region(s) of the OPG C-terminal domain which are important for OPG homodimerization. These OPG mutants were constructed by PCR amplification using primers which introduce premature stop translation signals in the C-terminal region of murine OPG. The 5′ oligo was designed to the MuOPG start codon (containing a HindIII restriction site) and the 3′ oligonucleotides (containing a stop codon and XhoI site) were designed to truncate the C-terminal region of muOPG ending at either threonine residue 200 (CT 200), proline 212 (CT212), glutamic acid 293 (CT-293), or serine 355 (CT-355).

The following primers were used to construct muOPG [22-200]:

| | |
|---|---|
| 1091-39: | |
| 5′-CCT CTG AGC TCA AGC TTC CGA GGA | (SEQ ID NO:160) |
| CCA CAA TGA ACA AG-3′ | |
| 1391-91: | |
| 5′-CCT CTC TCG AGT CAG GTG ACA TCT | (SEQ ID NO:161) |
| ATT CCA CAC TTT TGC GTG GC-3′ | |

The following primers were used to construct muOPG [22-212]:

| | |
|---|---|
| 1091-39: | |
| 5′-CCT CTG AGC TCA AGC TTC CGA GGA | (SEQ ID NO:162) |
| CCA CAA TGA ACA AG-3′ | |
| 1391-90: | |
| 5′-CCT CTC TCG AGT CAA GGA ACA GCA | (SEQ ID NO:163) |
| AAC CTG AAG AAG GC-3′ | |

The following primers were used to construct muOPG [22-293]:

| | |
|---|---|
| 1091-39: | |
| 5′-CCT CTG AGC TCA AGC TTC CGA GGA | (SEQ ID NO:164) |
| CCA CAA TGA ACA AG-3′ | |
| 1391-89: | |
| 5′-CCT CTC TCG AGT CAC TCT GTG GTG | (SEQ ID NO:165) |
| AGG TTC GAG TGG CC-3′ | |

The following primers were used to construct muOPG [22-355]:

| | |
|---|---|
| 1091-39: | |
| 5′-CCT CTG AGC TCA AGC TTC CGA GGA | (SEQ ID NO:166) |
| CCA CAA TGA ACA AG-3′ | |
| 1391-88: | |
| 5′ CCT CTC TCG AGT CAG GAT GTT TTC | (SEQ ID NO:167) |
| AAG TGC TTG AGG GC-3′ | |

Each resulting muOPG-CT plasmid containing the appropriate truncation was then transfected into human 293 cells, the mutant OPG-Fc fusion protein purified from conditioned media as described above. The biological activity of each protein was assessed the in vitro osteoclast forming assay described in example 11. The conditioned medias were also analysed by non-reducing SDS-PAGE and western blotting using anti-OPG antibodies.

Truncation of the C-terminal region of OPG effects the ability of OPG to form homodimers. CT 355 is predominantly monomeric, although some dimer is formed. CT 293 forms what appears to be equal molar amounts of monomer and dimer, and also high molecular weight aggregates. However, CT 212 and CT 200 are monomeric.

### EXAMPLE 10

### Purification of OPG

A. Purification of Mammalian OPG-Fc Fusion Proteins

5 L of conditioned media from 293 cells expressing an OPG-Fc fusion protein were prepared as follows. A frozen sample of cells was thawed into 10 ml of 293S media (DMEM-high glucose, 1×L-glutamine, 10% heat inactivated fetal bovine serum (FBS) and 100 ug/ml hygromycin) and fed with fresh media after one day. After three days, cells were split into two T175 flasks at 1:10 and 1:20 dilutions. Two additional 1:10 splits were done to scale up to 200 T175 flasks. Cells were at 5 days post-thawing at this point. Cells were grown to near confluency (about three days) at which time serum-containing media was aspirated, cells were washed one time with 25 ml PBS per flask and 25 ml of SF media (DMEM-high glucose, 1×L-glutamine) was added to each flask. Cells were maintained at 5% C02 for three days at which point the media was harvested, centrifuged, and filtered through 0.45m cellulose nitrate filters (Corning).

OPG-Fc fusion proteins were purified using a Protein G Sepharose column (Pharmacia) equilibrated in PBS. The column size varied depending on volume of starting media. Conditioned media prepared as described above was loaded onto the column, the column washed with PBS, and pure protein eluted using 100 mM glycine pH 2.7. Fractions were collected into tubes containing 1M Tris pH 9.2 in order to neutralize as quickly as possible. Protein containing fractions were pooled, concentrated in either an Amicon Centricon 10 or Centriprep 10 and diafiltered into PBS. The pure protein is stored at −80° C.

Murine [22-401]-Fc, Murine [22-180]-Fc, Murine [22-194]-Fc, human [22-401]-Fc and human [22-201]Fc were purified by this procedure. Murine [22-185]-Fc is purified by this procedure.

B. Preparation of anti-OPG Antibodies

Three New Zealand White rabbits (5-8 lbs initial wt) were injected subcutaneously with muOPG[22-401]-Fc fusion protein. Each rabbit was immunized on day 1 with 50 □g of antigen emulsified in an equal volume of Freunds complete adjuvant. Further boosts (Days 14 and 28) were performed by the same procedure with the substitution of Freunds incomplete adjuvant. Antibody titers were monitored by EIA. After the second boost, the antisera revealed high antibody titers and 25 ml production bleeds were obtained from each animal. The sera was first passed over an affinity column to which murine OPG-Fc had be immobilized. The anti-OPG antibodies were eluted with Pierce Gentle Elution Buffer containing 1% glacial acetic acid. The eluted protein was then dialyzed into PBS and passed over a Fc column to remove any antibodies specific for the Fc portion of the OPG fusion protein. The run through fractions containing anti-OPG specific antibodies were dialyzed into PBS.

C. Purification of Murine OPG[22-401]

Antibody Affinity Chromatography

Affinity purified anti-OPG antibodies were diafiltered into coupling buffer (0.1M sodium carbonate pH 8.3, 0.5M NaCl), and mixed with CNBr-activated sepharose beads (Pharmacia) for two hours at room temperature. The resin was then washed with coupling buffer extensively before blocking unoccupied sited with 1M ethanolamine (pH 8.0) for two hours at room temperature. The resin was then washed with low pH (0.1M sodium acetate pH 4.0, 0.5M NaCl) followed by a high pH wash (0.1M Tris-HCl pH 8.0, 0.5M NaCl). The last washes were repeated three times. The resin was finally equilibrated with PBS before packing into a column. Once packed, the resin was washed with PBS. A blank elution was performed with 0.1M glycine-HCl, pH 2.5), followed by re-equilibration with PBS.

Concentrated conditioned media from CHO cells expressing muOPG[22-410] was applied to the column at a low flow rate. The column was washed with PBS until UV absorbable measured at 280 nm returned to baseline. The protein was eluted from the column first with 0.1M glycine-HCl (pH 2.5), re-equilibrated with PBS, and eluted with a second buffer (0.1M CAPS, pH 10.5), 1M NaCl). The two elution pools were diafiltered separately into PBS and sterile filtered before freezing at −20° C.

Conventional Chromatography

CHO cell conditioned media was concentrated 23× in an Amicon spiral wound cartridge (S10Y10) and diafiltered into 20 mM tris pH 8.0. The diafiltered media was then applied to a Q-sepharose HP (Pharmacia) column which had been equilibrated with 20 mM tris pH 8.0. The column was then washed until absorbence at 280 nm reached baseline. Protein was eluted with a 20 column volume gradient of 0-300 mM NaCl in tris pH 8.0. OPG was detected using a western blot of column fractions.

Fractions containing OPG were pooled and brought to a final concentration of 300 mM NaCl, 0.2 mM DTT. A NiNTA superose (Qiagen) column was equilibrated with 20 mM tris pH 8.0, 300 mM NaCl, 0.2 mM DTT after which the pooled fractions were applied. The column was washed with equilibration buffer until baseline absorbence was reached. Proteins were eluted from the column with a 0-30 mM Imidazole gradient in equilibration buffer. Remaining proteins were washed off the column with 1M Imidazole. Again a western blot was used to detect OPG containing fractions.

Pooled fractions from the NiNTA column were dialyzed into 10 mm potassium phosphate pH 7.0, 0.2 mM DTT. The dialyzed pool was then applied to a ceramic hydroxyapatite column (Bio-Rad) which had been equilibrated in 10 mM phosphate buffer. After column washing, the protein was eluted with a 10-100 mM potassium phosphate gradient over 20 column volumes. This was then followed by a 20 column volume gradient of 100-400 mM phosphate.

OPG was detected by coomassie blue staining of SDS-polyacrylamide gels and by western blotting. Fractions were pooled and diafiltered onto PBS and frozen at −80° C. The purified protein runs as a monomer and will remain so after diafiltration into PBS. The monomer is stable when stored frozen or at pH 5 at 4° C. However if stored at 4° C. in PBS, dimers and what appears to be trimers and tetramers will form after one week.

D. Purification of Human OPG met[22-401] from E. coli

The bacterial cell paste was suspended into 10 mM EDTA to a concentration of 15% (w/v) using a low shear homogenizer at 5° C. The cells were then disrupted by two homogenizations at 15,000 psi each at 5° C. The resulting homogenate was centrifuged at 5,000×g for one hour at 5° C. The centrifugal pellet was washed by low shear homogenization into water at the original homogenization volume followed by centrifugation as before. The washed pellet was then solubilized to 15% (w/v) by a solution of (final concentration) 6 M guanidine HCl, 10 mM dithiothreitol, 10 mM TrisHCl, pH 8.5 at ambient temperature for 30 minutes. This solution was diluted 30-fold into 2M urea containing 50 mM CAPS, pH 10.5, 1 mM reduced glutathione and then stirred for 72 hours at 5° C. The OPG was purified from this solution at 25° C. by first adjustment to pH 4.5 with acetic acid and then chromatography over a column of SP-HP Sepharose resin equilibrated with 25 mM sodium acetate, pH 4.5. The column elution was carried out with a linear sodium chloride gradient from 50 mM to 550 mM in the same buffer using 20 column volumes at a flow rate of 0.1 column volumes/minute. The peak fractions containing only the desired OPG form were pooled and stored at 5° C. or buffer exchanged into phosphate buffered saline, concentrated by ultrafiltration, and then stored at 5° C. This material was analyzed by reverse phase HPLC, SDS-PAGE, limulus amebocyte lysate assay for the presence of endotoxin, and N-terminal sequencing. In addition, techniques such as mass spectrometry, pH/temperature stability, fluoresence, circular dichroism, differential scanning calorimetry, and protease profiling assays may also be used to examine the folded nature of the protein.

### EXAMPLE 11

### Biological Activity of Recombinant OPG

Based on histology and histomorphometry, it appeared that hepatic overexpression of OPG in transgenic nice markedly decreased the numbers of osteoclasts leading to a marked increase in bone tissue (see Example 4). To gain further insight into potential mechanism(s) underlying this in vivo effect, various forms of recombinant OPG have been tested in an in vitro culture model of osteoclast formation (osteoclast forming assay). This culture system was originally devised by Udagawa (Udagawa et al. Endocrinology 125, 1805-1813 (1989), Proc. Natl. Acad. Sci. USA 87, 7260-7264 (1990)) and employs a combination of bone marrow cells and cells from bone marrow stromal cell lines. A description of the modification of this culture system used for these studies has been previously published (Lacey et al. Endocrinology 136, 2367-2376 (1995)). In this method, bone marrow cells, flushed from the femurs and tibiae of mice, are cultured overnight in culture media (alpha MEM with 10% heat inactivated fetal bovine serum) supplemented with 500 U/ml CSF-1 (colony stimulating factor 1, also called M-CSF), a hematopoietic growth factor specific for cells of the monocyte/macrophage family lineage. Following this incubation, the non-adherent cells are collected, subjected to gradient purification, and then cocultured with cells from the bone marrow cell line ST2 (1×10⁶ non-adherent cells: 1×10⁵ ST2 cells/ml media). The media is supplemented with dexamethasone (100 nM) and the biologically-active metabolite of vitamin D3 known as 1,25 dihydroxyvitamin D3 (1.25 (OH)2 D3, 10 nM). To enhance osteoclast appearance, prostaglandin E2 (250 nM) is added to some cultures. The coculture period usually ranges from 8-10 days and the media, with all of the supplements freshly added, is renewed every 3-4 days. At various intervals, the cultures are assessed for the presence of tartrate acid phosphatase (TRAP) using either a histochemical stain (Sigma Kit # 387A, Sigma, St. Louis, Mo.) or TRAP solution assay.

The TRAP histochemical method allows for the identification of osteoclasts phenotypically which are multinucleated (≧3 nuclei) cells that are also TRAP+. The solution assay involves lysing the osteoclast-containing cultures in a citrate buffer (100 mM, pH 5.0) containing 0.1% Triton X-100. Tartrate resistant acid phosphatase activity is then measured based on the conversion of p-nitrophenylphosphate (20 nM) to p-nitrophenol in the presence of 80 mM sodium tartrate which occurs during a 3-5 minute incubation at RT. The reaction is terminated by the addition of NaOH to a final concentration of 0.5 M. The optical density at 405 nm is measured and the results are plotted.

Previous studies (Udagawa et al. ibid) using the osteoclast forming assay have demonstrated that these cells express receptors for ¹²⁵I-calcitonin (autoradiography) and can make pits on bone surfaces, which when combined with TRAP positivity confirm that the multinucleated cells have an osteoclast phenotype. Additional evidence in support of the osteoclast phenotype of the multinucleated cells that arise in vitro in the osteoclast forming assay are that the cells express αv and β3 integrins by immunocytochemistry and calcitonin receptor and TRAP mRNA by in situ hybridization (ISH).

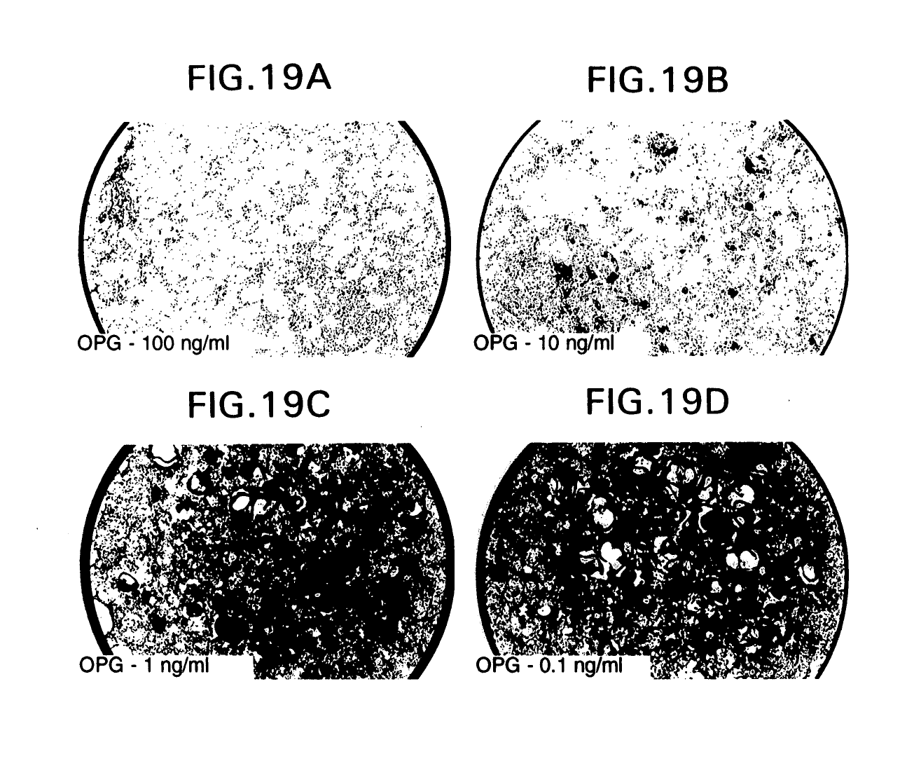

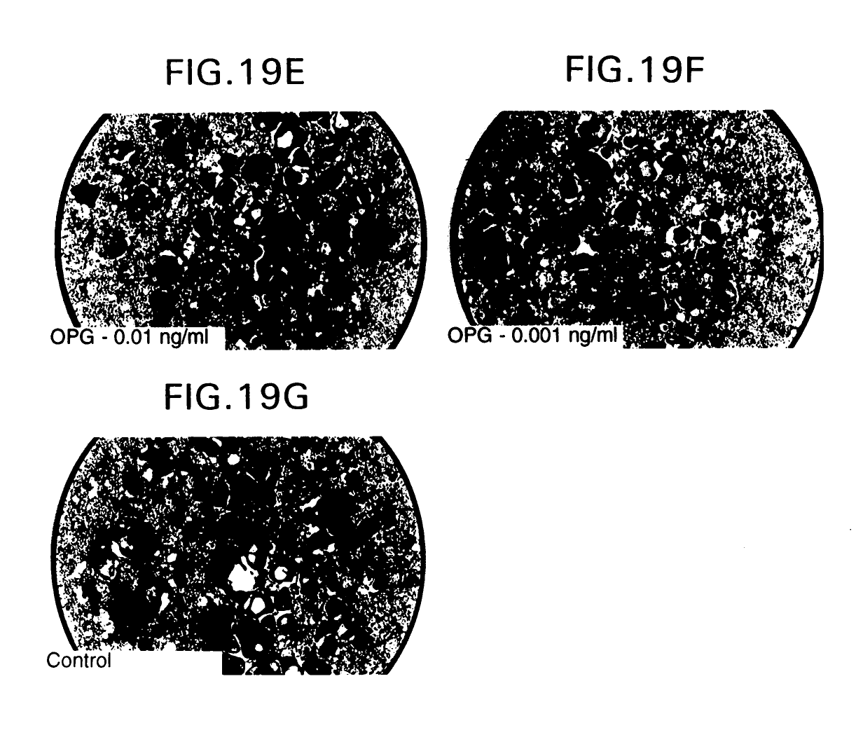

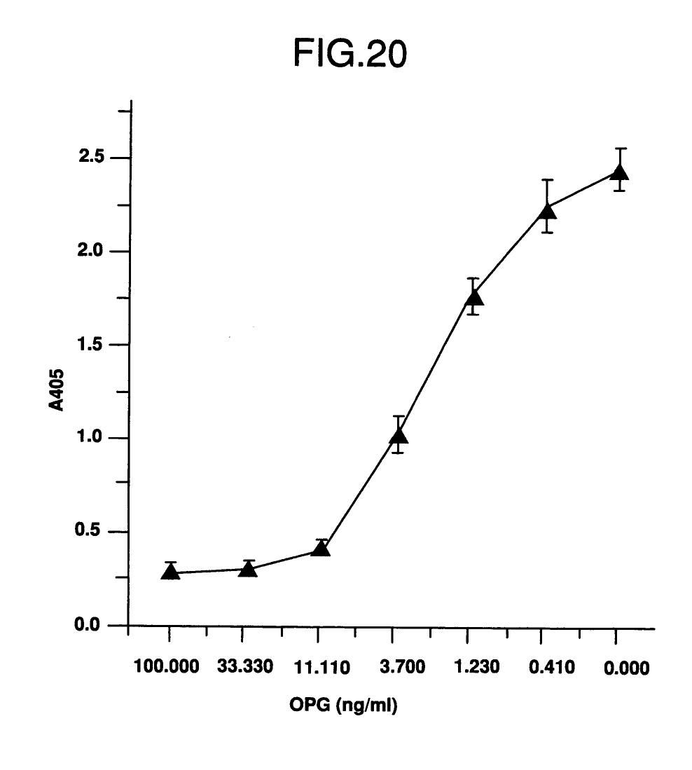

The huOPG [22-401]-Fc fusion was purified from CHO cell conditioned media and subsequently utilized in the osteoclast forming assay. At 100 ng/ml of huOPG [22-401]-Fc, osteoclast formation was virtually 100% inhibited (FIG. 19A). The levels of TRAP measured in lysed cultures in microtitre plate wells were also inhibited in the presence of OPG with an ID₅₀ of approximately 3 ng/ml (FIG. 20). The level of TRAP activity in lysates appeared to correlate with the relative number of osteoclasts seen by TRAP cytochemistry (compare FIGS. 19A-19G and 20). Purified human IgG1 and TN\overline{F}□ inhibitor were also tested in this model and were found to have no inhibitory or stimulatory effects suggesting that the inhibitory effects of the huOPG [22-401]-Fc were due to the OPG portion of the fusion protein. Additional forms of the human and murine molecules have been tested and the cumulative data are summarized in Table 3.

TABLE 3
Effects of various OPG forms on in vitro osteoclast formation

| OPG Construct | Relative Bioactivity in vitro |
|---|---|
| muOPG [22-401]-Fc | +++ |
| muOPG [22-194]-Fc | +++ |
| muOPG [22-185]-Fc | ++ |
| muOPG [22-180]-Fc | − |
| muOPG [22-401] | +++ |
| muOPG [22-401] C195 | +++ |
| muOPG [22-401] C202 | + |
| muOPG [22-401] C277 | − |
| muOPG [22-401] C319 | + |
| muOPG [22-401] C400 | + |
| muOPG [22-185] | − |
| muOPG [22-194] | ++ |
| muOPG [22-200] | ++ |
| muOPG [22-212] | − |
| muOPG [22-293] | +++ |
| muOPG [22-355] | +++ |
| huOPG [22-401]-Fc | +++ |
| huOPG [22-201]-Fc | +++ |
| huOPG [22-401]-Fc P26A | +++ |
| huOPG [22-401]-Fc Y28F | +++ |
| huOPG [22-401] | +++ |
| huOPG [27-401]-Fc | ++ |
| huOPG [29-401]-Fc | ++ |
| huOPG [32-401]-Fc | +/− |

+++, ED₅₀ = 0.4-2 ng/ml
++, ED₅₀ = 2-10 ng/ml
+, ED₅₀ = 10-100 ng/ml
−, ED₅₀ > 100 ng/ml

The cumulative data suggest that murine and human OPG amino acid sequences 22-401 are fully active in vitro, when either fused to the Fc domain, or unfused. They inhibit in a dose-dependent manner and possess half-maximal activities in the 2-10 ng/ml range. Truncation of the murine C-terminus at threonine residue 180 inactivates the molecule, whereas truncations at cysteine 185 and beyond have full activity. The cysteine residue located at position 185 is predicted to form an SS3 bond in the domain 4 region of OPG. Removal of this residue in other TNFR-related proteins has previously been shown to abrogate biological activity (Yan et al. (1994), J. Biol. Chem. 266: 12099-104). Our finding that muOPG[22-180]-Fc is inactive while muOPG[22-185]-Fc is active is consistent with these findings. This suggests that amino acid residues 22-185 define a region for OPG activity.

These findings indicate that like transgenically-expressed OPG, recombinant OPG also suppressed osteoclast formation as tested in the osteoclast forming assay. Time course experiments examining the appearance of TRAP+ cells, β3+ cells, F480+ cells in cultures continuously exposed to OPG demonstrate that OPG blocks the appearance TRAP+ and β3+ cells, but not F480+ cells. In contrast, TRAP+ and β3+ cells begin to appear as early as day 4 following culture establishment in control cultures. Only F480+ cells can be found in OPG-treated cultures and they appear to be present at qualitatively the same numbers as the control cultures. Thus, the mechanism of OPG effects in vitro appears to involve a blockade in osteoclast differentiation at a step beyond the appearance of monocyte-macrophages but before the appearance of cells expressing either TRAP or β3 integrins. Collectively these findings indicate that OPG does not interfere with the general growth and differentiation of monocyte-macrophage precursors from bone marrow, but rather suggests that OPG specifically blocks the selective differentiation of osteoclasts from monocyte-macrophage precursors.

To determine more specifically when in the osteoclast differentiation pathway that OPG was inhibitory, a variation of the in vitro culture method was employed. This variation, described in (Lacey et al. supra), employs bone marrow macrophages as osteoclast precursors. The osteoclast precursors are derived by taking the nonadherent bone marrow cells after an overnight incubation in CSF-1/M-CSF, and culturing the cells for an additional 4 days with 1,000-2,000 U/ml CSF-1. Following 4 days of culture, termed the growth phase, the non-adherent cells are removed. The adherent cells, which are bone marrow macrophages, can then be exposed for up to 2 days to various treatments in the presence of 1,000-2,000 U/ml CSF-1. This 2 day period is called the intermediate differentiation period. Thereafter, the cell layers are again rinsed and then ST-2 cells (1×10⁵ cell/ml), dexamethasone (100 nM) and 1,25 (OH)2 D3 (10 nM) are added for the last 8 days for what is termed the terminal differentiation period. Test agents can be added during this terminal period as well. Acquisition of phenotypic markers of osteoclast differentiation are acquired during this terminal period (Lacey et al. ibid).

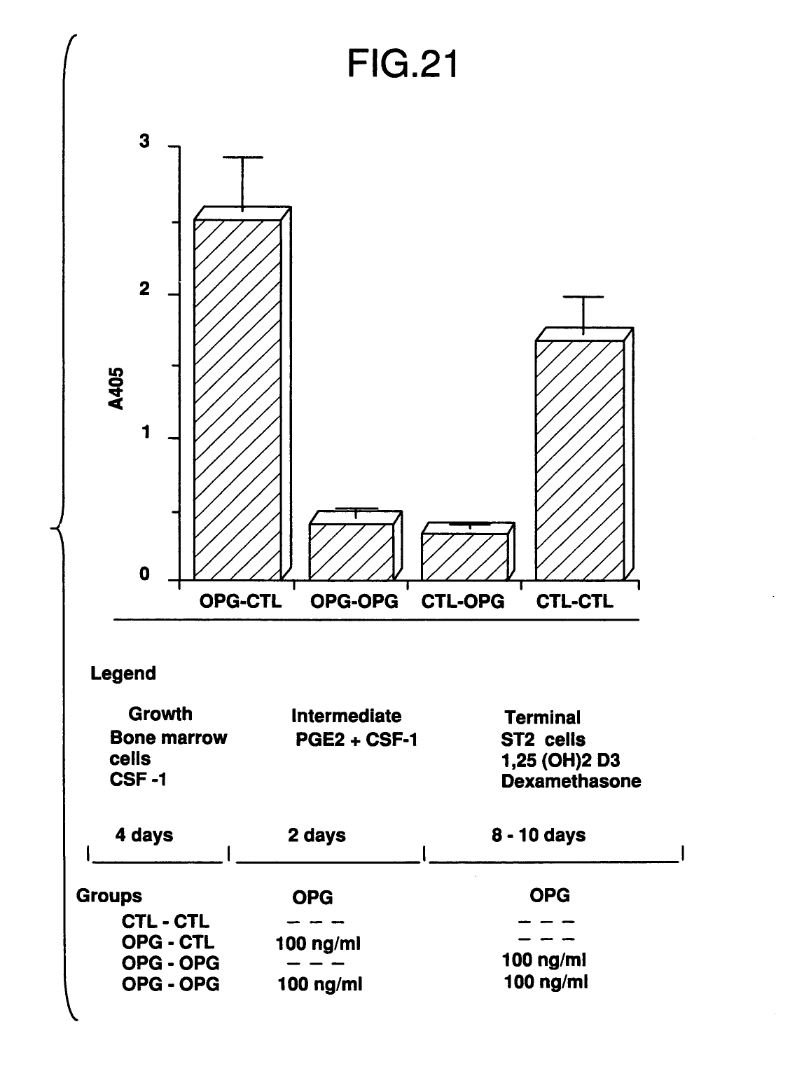

huOPG [22-401]-Fc (100 ng/ml) was tested for its effects on osteoclast formation in this model by adding it during either the intermediate, terminal or, alternatively, both differentiation periods. Both TRAP cytochemistry and solution assays were performed. The results of the solution assay are shown in FIG. 21. HuOPG [22-401]-Fc inhibited the appearance of TRAP activity when added to both the intermediate and terminal or only the terminal differentiation phases. When added to the intermediate phase and then removed from the cultures by rinsing, huOPG [22-401]-Fc did not block the appearance of TRAP activity in culture lysates. The cytochemistry results parallel the solution assay data. Collectively, these observations indicate that huOPG [22-401]-Fc only needs to be present during the terminal differentiation period for it to exert its all of its suppressive effects on osteoclast formation.

B. In vivo IL-1-α and IL-1-β Challenge Experiments

IL-1 increases bone resorption both systemically and locally when injected subcutaneously over the calvaria of mice (Boyce et al. (1989), Endocrinology 125: 1142-50). The systemic effects can be assessed by the degree of hypercalcemia and the local effects histologically by assessing the relative magnitude of the osteoclast-mediated response. The aim of these experiments was to determine if recombinant muOPG [22-401]-Fc could modify the local and/or systemic actions of IL-1 when injected subcutaneously over the same region of the calvaria as IL-1.

IL-1β Experiment

Male mice (ICR Swiss white) aged 4 weeks were divided into the following treatment groups (5 mice per group): Control group: IL-1 treated animals (mice received 1 injection/day of 2.5 ug of IL-1-β); Low dose muOPG [22-401]-Fc treated animals (mice received 3 injections/day of 1 μg of muOPG [22-401]-Fc); Low dose muopg [22-401]-Fc and IL-1-β; High dose muOPG [22-401]-Fc treated animals (mice receive 3 injections/day of 10 βg muOPG [22-401]-Fc); High dose muOPG [22-401]-Fc and IL-1-β. All mice received the same total number of injections of either active factor or vehicle (0.1% bovine serum albumin in phosphate buffered saline). All groups are sacrificed on the day after the last injection. The weights and blood ionized calcium levels are measured before the first injections, four hours after the second injection and 24 hours after the third IL-1 injection, just before the animals were sacrificed. After sacrifice the calvaria were removed and processed for paraffin sectioning.

IL-1 α Experiment

Male mice (ICR Swiss white) aged 4 weeks were divided into the following treatment groups (5 mice per group): Control group; IL-1-α treated animals (mice received 1 injection/day of 5 ug of IL-1-α); Low dose muOPG [22-401]-Fc treated animals (mice received 1 injection/day of 10 μg of muOPG [22-401]-Fc; Low dose muopg [22-401]-Fc and IL-1-α, (dosing as above); High dose muopg [22-401]-Fc treated animals (mice received 3 injections/day of 10 μg muOPG [22-401]-Fc; High dose muOPG [22-401]-Fc and IL-1-α. All mice received the same number of injections/day of either active factor or vehicle. All groups were sacrificed on the day after the last injection. The blood ionized calcium levels were measured before the first injection, four hours after the second injection and 24 hours after the third IL-1 injection, just before the animals were sacrificed. The animal weights were measured before the first injection, four hours after the second injection and 24 hours after the third IL-1 injection, just before the animals were sacrificed. After sacrifice the calvaria were removed and processed for paraffin sectioning.

Histological Methods

Calvarial bone samples were fixed in zinc formalin, decalcified in formic acid, dehydrated through ethanol and mounted in paraffin. Sections (5μm thick) were cut through the calvaria adjacent to the lambdoid suture and stained with either hematoxylin and eosin or reacted for tartrate resistant acid phosphatase activity (Sigma Kit# 387A) and counterstained with hematoxylin. Bone resorption was assessed in the IL-1 α treated mice by histomorphometric methods using the Osteomeasure (Osteometrics, Atlanta, Ga.) by tracing histologic features onto a digitizor platen using a microscope-mounted camera lucida attachment. Osteoclast numbers, osteoclast lined surfaces, and eroded surfaces were determined in the marrow spaces of the calvarial bone. The injected and non-injected sides of the calvaria were measured separately.

Results

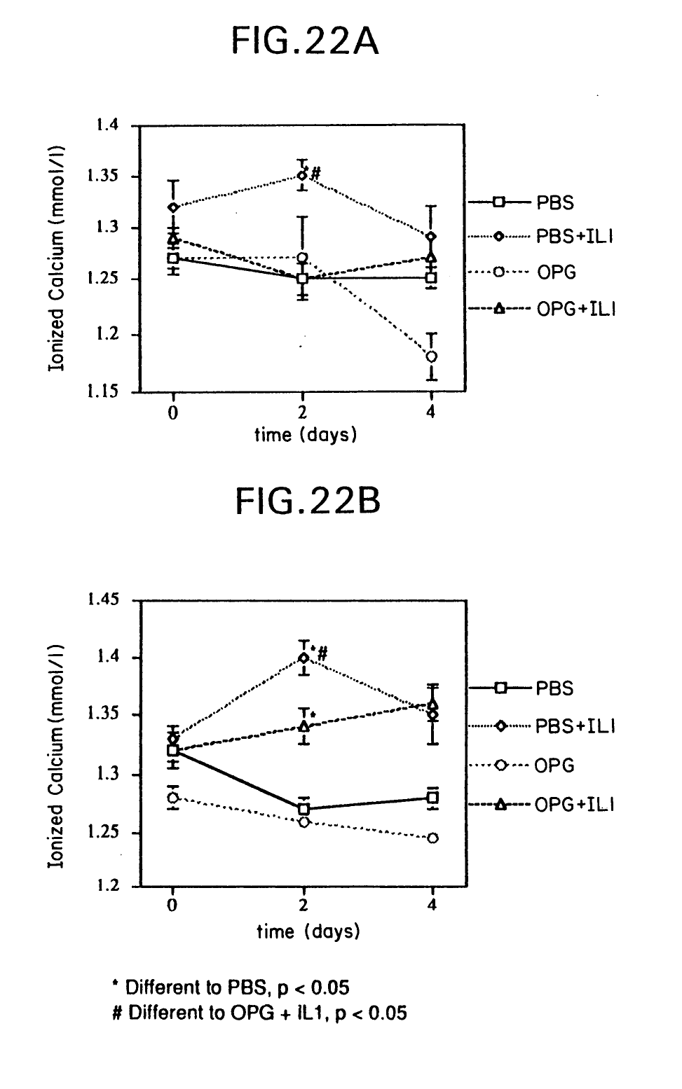

IL-1 α and IL-1 β produced hypercalcemia at the doses used, particularly on the second day, presumably by the induction of increased bone resorption systemically. The hypercalcemic response was blocked by muOPG [22-401]-Fc in the IL-1 beta treated mice and significantly diminished in mice treated with IL-1α, an effect most apparent on day 2 (FIG. 22A-22B).

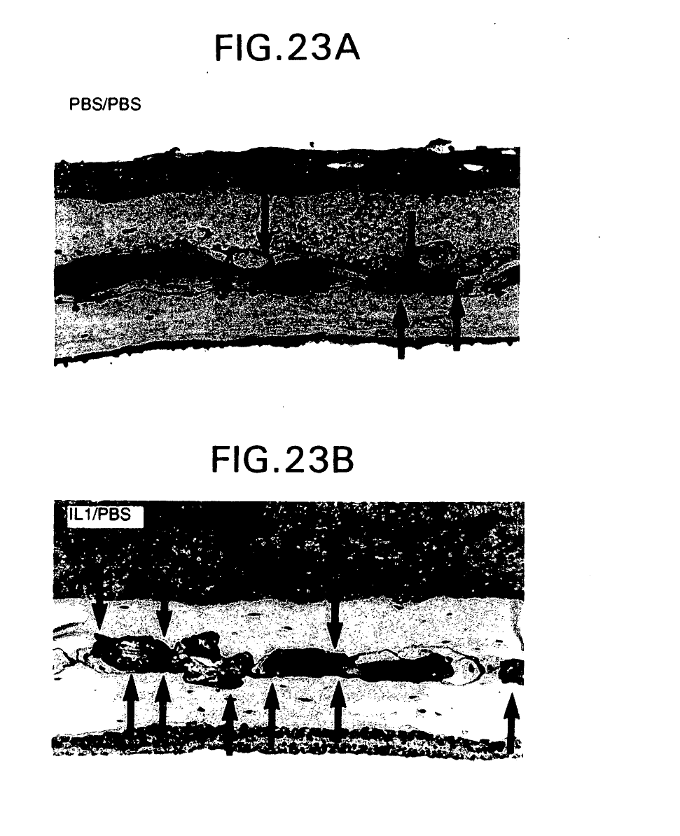

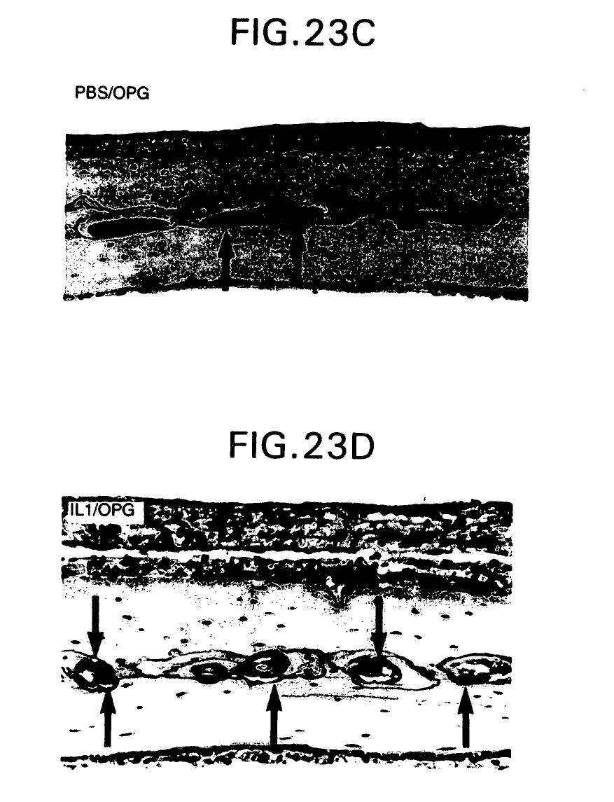

Histologic analysis of the calvariae of mice treated with IL-1α and beta shows that IL-1 treatments alone produce a marked increase in the indices of bone resorption including: osteoclast number, osteoclast lined surface, and eroded surface (surfaces showing deep scalloping due to osteoclastic action (FIG. 23B). In response to IL-1 α or IL-1 β, the increases in bone resorption were similar on the injected and non-injected sides of the calvaria. Muopg [22-401]-Fc injections reduced bone resorption in both IL-\tilde{1}α and beta treated mice and in mice receiving vehicle alone but this reduction was seen only on the muopg [22-401]-Fc injected sides of the calvariae.

The most likely explanation for these observations is that muOPG [22-401]-Fc inhibited bone resorption, a conclusion supported by the reduction of both the total osteoclast number and the percentage of available bone surface undergoing bone resorption, in the region of the calvaria adjacent to the muOPG [22-401]-Fc injection sites. The actions of muOPG [22-401]-Fc appeared to be most marked locally by histology, but the fact that muOPG [22-401]-Fc also blunted IL-1 induced hypercalcemia suggests that muOPG [22-401]-Fc has more subtle effects on bone resorption systemically.

C. Systemic Effects of muOPG [22-401]-Fc in Growing Mice

Male BDF1 mice aged 34 weeks, weight range 9.2- 15.7 g were divided into groups of ten mice per group. These mice were injected subcutaneously with saline or muOPG [22-401]-Fc 2.5 mg/kg bid for 14 days (5 mg/kg/day). The mice were radiographed before treatment, at day 7 and on day 14. The mice were sacrificed 24 hours after the final injection. The right femur was removed, fixed in zinc formalin, decalcified in formic acid and embedded in paraffin. Sections were cut through the mid region of the distal femoral metaphysis and the femoral shaft. Bone density, by histomorphometry, was determined in six adjacent regions extending from the metaphyseal limit of the growth plate, through the primary and secondary spongiosa and into the femoral diaphysis (shaft). Each region was 0.5×0.5 mm².

Radiographic Changes

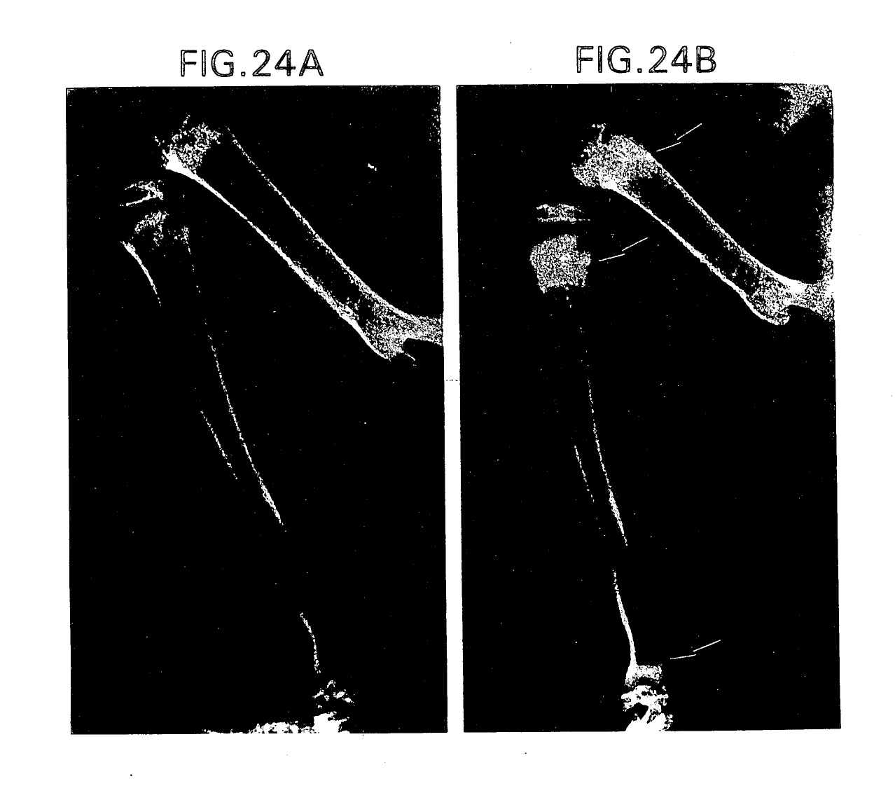

After seven days of treatment there was evidence of a zone of increased bone density in the spongiosa associated with the growth plates in the OPG treated mice relative to that seen in the controls. The effects were particularly striking in the distal femoral and the proximal tibial metaphases (FIG. 24A-24B). However bands of increased density were also apparent in the vertebral bodies, the iliac crest and the distal tibia. At 14 days, the regions of opacity had extended further into the femoral and tibial shafts though the intensity of the radio-opacity was diminished. Additionally, there were no differences in the length of the femurs at the completion of the experiment or in the change in length over the duration of the experiment implying that OPG does not alter bone growth.

Histological Changes

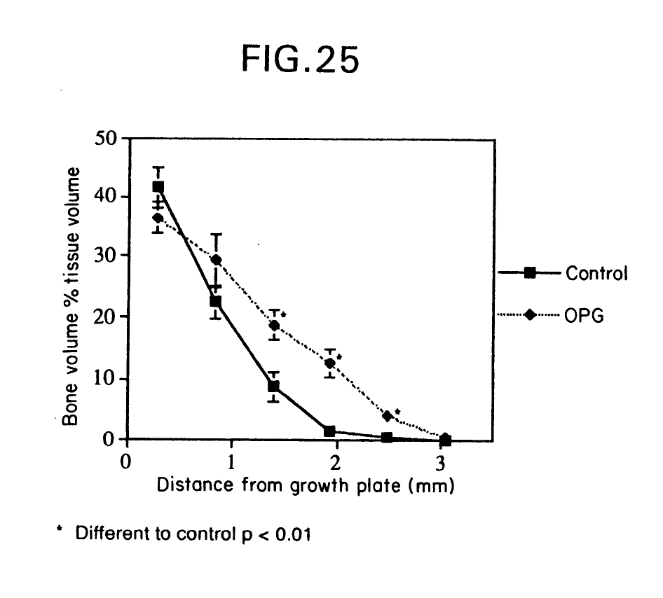

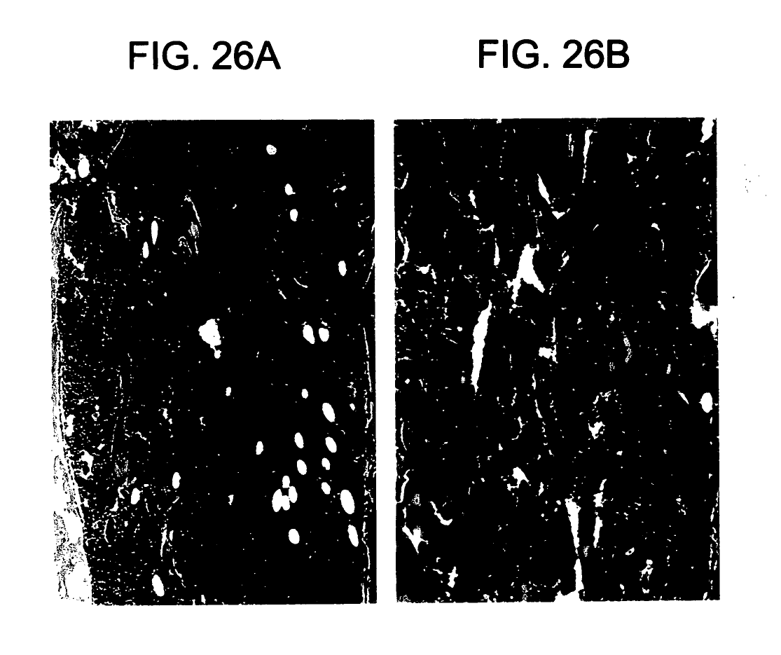

The distal femoral metaphysis showed increased bone density in a regions 1.1 to 2.65 mm in distance from the growth plate (FIGS. 25 and 26A-26B). This is a region where bone is rapidly removed by osteoclast-mediated bone resorption in mice. In these rapidly growing young mice, the increase in bone in this region observed with OPG treatment is consistent with an inhibition of bone resorption.

D. Effects of Osteoprotegerin on Bone Loss Induced by Ovariectomy in the Rat

Twelve week old female Fisher rats were ovariectomized (OVX) or sham operated and dual xray absorptiometry (DEXA) measurements made of the bone density in the distal femoral metaphysis. After 3 days recovery period, the animals received daily injections for 14 days as follows: Ten sham operated animals received vehicle (phosphate buffered saline); Ten OVX animals received vehicle (phosphate buffered saline); Six OVX animals received OPG-Fc 5mg/kg SC; Six OVX animals received pamidronate (PAM) 5 mg/kg SC; Six OVX animals received estrogen (ESTR) 40 ug/kg SC. After 7 and 14 days treatment the animals had bone density measured by DEXA. Two days after the last injection the animals were killed and the right tibia and femur removed for histological evaluation.

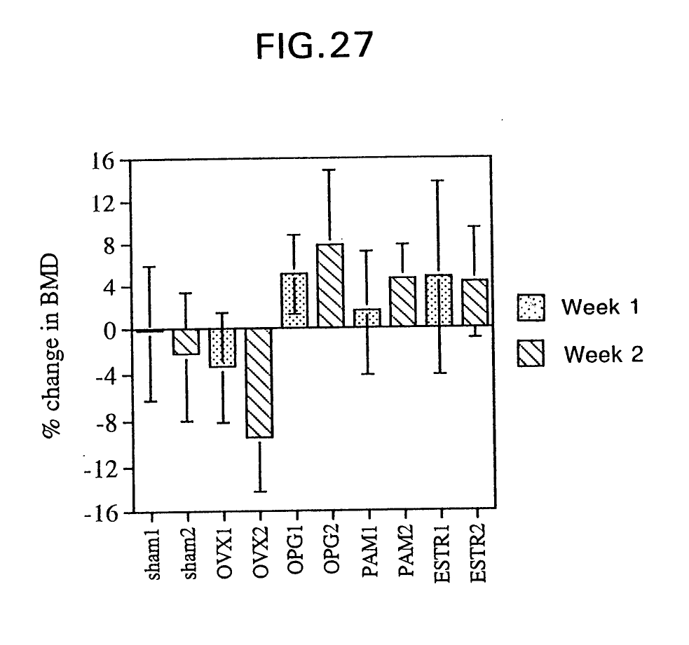

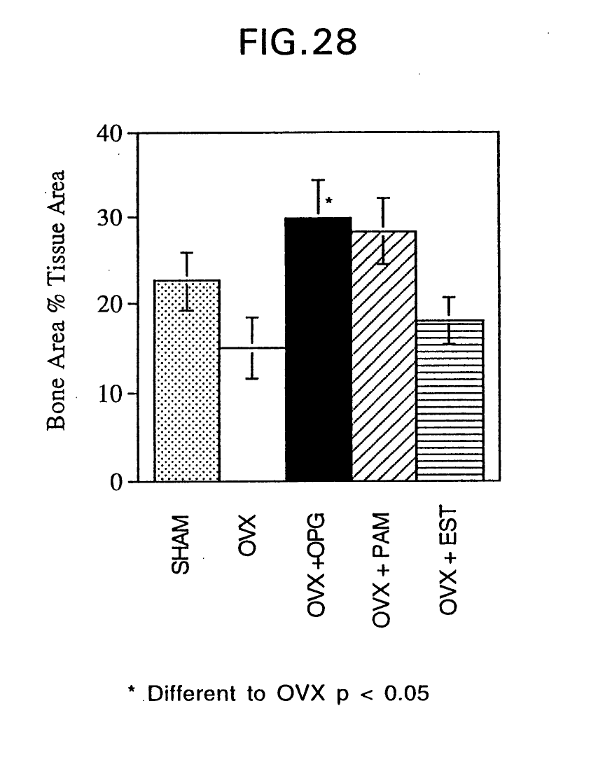

The DEXA measurements of bone density showed a trend to reduction in the bone density following ovariectomy that was blocked by OPG-Fc. Its effects were similar to the known antiresorptive agents estrogen and pamidronate. (FIG. 27). The histomorphometric analysis confirmed these observations with OPG-Fc treatment producing a bone density that was significantly higher in OVX rats than that seen in untreated OVX rats (FIG. 28). These results confirm the activity of OPG in the bone loss associated with withdrawal of endogenous estrogen following ovariectomy.

In vivo Summary

The in vivo actions of recombinant OPG parallel the changes seen in OPG transgenic mice. The reduction in osteoclast number seen in the OPG transgenic is reproduced by injecting recombinant OPG locally over the calvaria in both normal mice and in mice treated with IL-1 α or IL-1 β. The OPG transgenic mice develop an osteopetrotic phenotype with progressive filling of the marrow cavity with bone and unremodelled cartilage extending from the growth plates from day 1 onward after birth. In normal three week old (growing) mice, OPG treatments also led to retention of bone and unremodelled cartilage in regions of endochondral bone formation, an effect observed radiographically and confirmed histologically. Thus, recombinant OPG produces phenotypic changes in normal animals similar to those seen in the transgenic animals and the changes are consistent with OPG-induced inhibition of bone resorption. Based on in vitro assays of osteoclast formation, a significant portion of this inhibition is due to impaired osteoclast formation. Consistent with this hypothesis, OPG blocks ovariectomy-induced osteoporosis in rat. Bone loss in this model is known to be mediated by activated osteoclasts, suggesting a role for OPG in treatment of primary osteoporosis.

### EXAMPLE 12

### Pegylation Derivatives of OPG

Preparation of N-terminal PEG-OPG Conjugates by Reductive Alkylation

HuOPG met [22-194] P25A was buffer exchanged into 25-50 mM NaOAc, pH 4.5-4.8 and concentrated to 2-5 mg/ml. This solution was used to conduct OPG reductive alkylation with monofunctional PEG aldehydes at 5-7 C. PEG monofunctional aldehydes, linear or branched, MW=1 to 57 kDa (available from Shearwater Polymers) were added to the OPG solution as solids in amounts constituting 2-4 moles of PEG aldehyde per mole of OPG. After dissolution of polymer into the protein solution, sodium cyanoborohydride was added to give a final concentration of 15 to 20 mM in the reaction mixture from 1-1.6 M freshly prepared stock solution in cold DI water. The progress of the reaction and the extent of OPG PEGylation was monitored by size exclusion HPLC on a G3000SW_{XL} column (Toso Haas) eluting with 100 mM NaPO₄, 0.5 M NaCl, 10% ethanol, pH 6.9. Typically the reaction was allowed to proceed for 16-18 hours, after which the reaction mixture was diluted 6-8 times and the pH lowered to 3.5-4. The reaction mixture was fractionated by ion exchange chromatography (HP SP HiLoad 16/10, Pharmacia) eluting with 20 mM NaOAc pH 4 with a linear gradient to 0.75M NaCl over 25 column volumes at a flow rate of 30 cm/h. Fractions of mono-, di- or poly-PEGylated OPG were pooled and characterized by SEC HPLC and SDS-PAGE. By N-terminal sequencing, it was determined that the monoPEG-OPG conjugate, the major reaction product in most cases, was 98% N-terminally PEG-modified OPG.

This procedure was generally used to prepare the following N-terminal PEG-OPG conjugates (where OPG is HuOPG met [22-194] P25A: 5 kD monoPEG, 10 kD mono branched PEG, 12 kD monoPEG, 20 kD monoPEG, 20 kD mono branched PEG, 25 kD monoPEG, 31 kD monoPEG, 57 kD monoPEG, 12 kD diPEG, 25 kD diPEG, 31 kD diPEG, 57 kD diPEG, 25 kD triPEG.

Preparation of PEG-OPG Conjugates by Acylation

HuOPG met [22-194] P25A was buffer exchanged into 50 mM BICINE buffer, pH 8 and concentrated to 2-3 mg/ml. This solution was used to conduct OPG acylation with monofunctional PEG N-hydroxysuccinimidyl esters at room temperature. PEG N-hydroxysuccinimidyl esters, linear or branched, MW=1 to 57 kDa (available from Shearwater Polymers) were added to the OPG solution as solids in amounts constituting 4-8 moles of PEG N-hydroxysuccinimidyl ester per mole of OPG. The progress of the reaction and the extent of OPG PEGylation was monitored by size exclusion HPLC on a G3000SW_{XL} column (Toso Haas) eluting with 100 mM NaPO₄, 0.5 M NaCl, 10% ethanol, pH 6.9. Typically the reaction was allowed to proceed for 1 hour, after which the reaction mixture was diluted 6-8 times and the pH lowered to 3.5-4. The reaction mixture was fractionated by ion exchange chromatography (HP SP HiLoad 16/10, Pharmacia) eluting with 20 mM NaOAc pH 4 with a linear gradient to 0.75M NaCl over 25 column volumes at a flow rate of 30 cm/h. Fractions of mono-, di- or poly- PEGylated OPG were pooled and characterized by SEC HPLC and SDS-PAGE.

This procedure was generally used to prepare the following PEG-OPG conjugates: 5 kD polyPEG, 20 kD polyPEG, 40 kD poly branched PEG, 50 kD poly PEG.

Preparation of Dimeric PEG-OPG

HuOPG met [22-194] P25A is prepared for thiolation at 1-3 mg/ml in a phosphate buffer at near neutral pH. S-acetyl mecaptosuccinic anhydride (AMSA) is added in a 3-7 fold molar excess while maintaining pH at 7.0 and the rxn stirred at 4° C. for 2 hrs. The monothiolated-OPG is separated from unmodified and polythiolated OPG by ion exchange chromatography and the protected thiol deprotected by treatment with hydroxylamine. After deprotection, the hydroxylamine is removed by gel filtration and the resultant monothiolated-OPG is subjected to a variety of thiol specific crosslinking chemistries. To generate a disulfide bonded dimer, the thiolated OPG at >1 mg/ml is allowed to undergo air oxidation by dialysis in slightly basic phosphate buffer. The covalent thioether OPG dimer was prepared by reacting the bis-maleimide crosslinker, N,N-bis(3-maleimido propianyl)-2-hydroxy 1,3 propane with the thiolated OPG at >1 mg/ml at a 0.6× molar ratio of crosslinker:OPG in phosphate buffer at pH 6.5. Similarly, the PEG dumbbells are produced by reaction of substoichiometric amounts of bis-maleimide PEG crosslinkers with thiolated OPG at >1 mg/ml in phosphate buffer at pH 6.5. Any of the above dimeric conjugates may be further purified using either ion exchange or size exclusion chromatographies.

Dimeric PEG-OPG conjugates (where OPG is HuOPG met [22-194] P25A prepared using the above procedures include disulfide-bonded OPG dimer, covalent thioether OPG dimer with an aliphatic amine type crosslinker, 3.4 kD and 8kD PEG dumbbells and monobells.

PEG-OPG conjugates were tested for activity in vitro using the osteoclast maturation assay described in Example 11A and for activity in vivo by measuring increased bone density after injection into mice as described in Example 11C. The in vivo activity is shown below in Table 2.

TABLE 2
In vivo biological activity of Pegylated OPG

| | Increase in Tibial |
|---|---|
| OPG Construct | Bone Density |
| muOPG met [22-194] | − |
| muOPG met [22-194] 5k PEG | + |
| muOPG met [22-194] 20k PEG | + |
| huOPG met [22-194] P25A | − |
| huOPG met [22-194] P25A 5k PEG | + |
| huOPG met [22-194] P25A 20k PEG | + |
| huOPG met [22-194] P25A 31k PEG | + |
| huOPG met [22-194] P25A 57k PEG | + |
| huOPG met [22-194] P25A 12k PEG | + |
| huOPG met [22-194] P25A 20k Branched PEG | + |
| huOPG met [22-194] P25A 8k PEG dimer | + |
| huOPG met [22-194] P25A disulfide crosslink | + |

### EXAMPLE 13

Effects of OPG-Fc During the Course of Adjuvant Arthritis in Lewis Rats

The aim of these studies is to investigate whether CHO produced OPG-Fc protects against adjuvant arthritis-associated bone mineral density loss in male Lewis rats.

Animals

Male Lewis rats (Charles River, Wilmington Mass.) 8-9 weeks of age (n=6) at the time of mycobacteria in oil injection, were used. Two rats were housed per cage in an air conditioned environment (room temperature 23±2 C, relative humidity 50±20%) that illuminated from 6:30 am to 6:30 p.m. Animals were fed a commercial rodent chow (#8640, Tek Lab, Madison Wis.); calcium and phosphorus contents were 1.2% and 1.0%, respectively. All animals were sacrificed by carbon dioxide inhalation.

Induction and Measurement of Adjuvant Arthritis

Adjuvant arthritis (AdA) was induced by a single injection of a suspension of Mycobacterium tuberculosis (Difco Laboratories, Detroit Mis.) in paraffin oil (Crescent Chemical Co., Hauppauge, N.Y.). Mycobacteria were grounded in a mortar to fine powder and suspended in paraffin oil (10 mg/ml). The suspension was dispersed evenly just before injection of 0.05 ml at the base of tail. Severity of inflammation was monitored by measuring the volume of hindpaws using volume displacement technique. The extent of inflammation was calculated as increase in paw volume compared to Day 0. In addition, body weight was measured daily.

OPG Treatment and DEXA Bone Mass Measurement

Male Lewis (normal and adjuvant-induced) rats received varying doses of OPG-Fc (22-194) by subcutaneous daily injection (See graphs below for dosing) from day 9 to day 15. At the end of the experiment (day 16) bone mass measurement (DexaScans) of the tibiotarsal region was performed with a Hologic QDR 4500 dual-energy x-ray absorptiometer.

Statistical Analysis

All results were expressed as the mean±standard deviation of the mean. The p value of 0.05 was used in the calculation to determine whether there were any significant differences between any two groups. Statistical significance of difference was assessed by analysis of variance based on a Mann Whitney U test using Statsoft software (Statsoft, Tulsa, Okla.).

Results

OPG-Fc Inhibits Loss of Bone Mineral Density in Adjuvant Arthritis

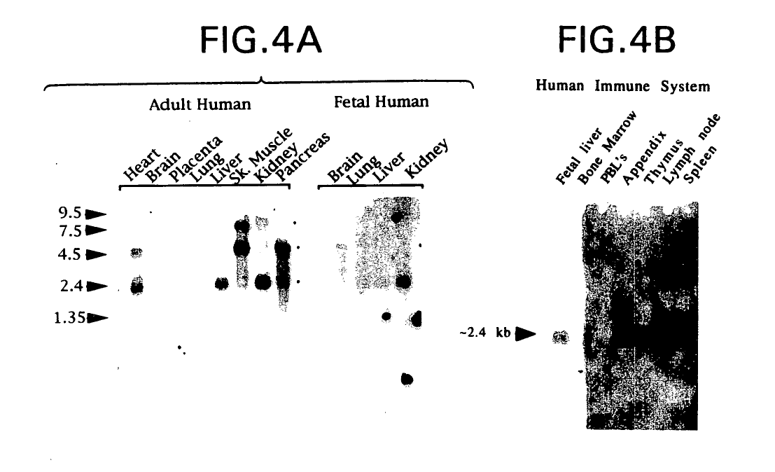

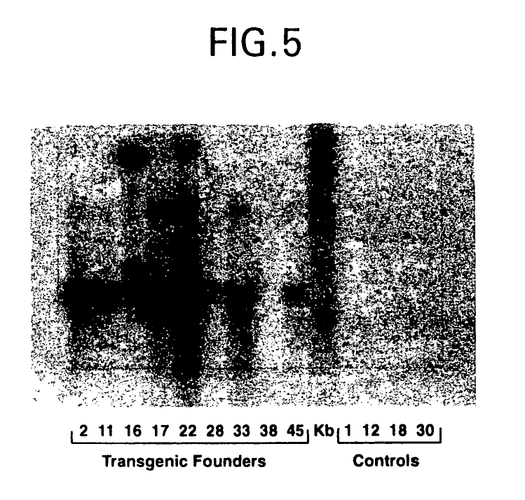

To study the effects of OPG-Fc on BMD in adjuvant arthritis, paws from two experiments were analyzed by DEXA. The results of BMD measurements on the tibiotarsal region are shown in FIGS. 2 and 4. Bone protective effects were observed in rats with adjuvant-arthritis treated with OPG-Fc via subcutaneous daily injection (from day 9 to day 15 after mycobacteria injection). Treatment with OPG-Fc at 4, 1, 0.25, 0.06, 0.016, and 0.004 mg/kg showed 100%, 100%, 100%, 86%, 22, and 22% inhibition of bone mineral density loss respectively. Treatment of the intermediate and high doses of OPG-Fc (4 - 0.06 mg/kg) showed a statistically significant difference in BMD when compared to the OPG placebo treated control group (P<0.05).

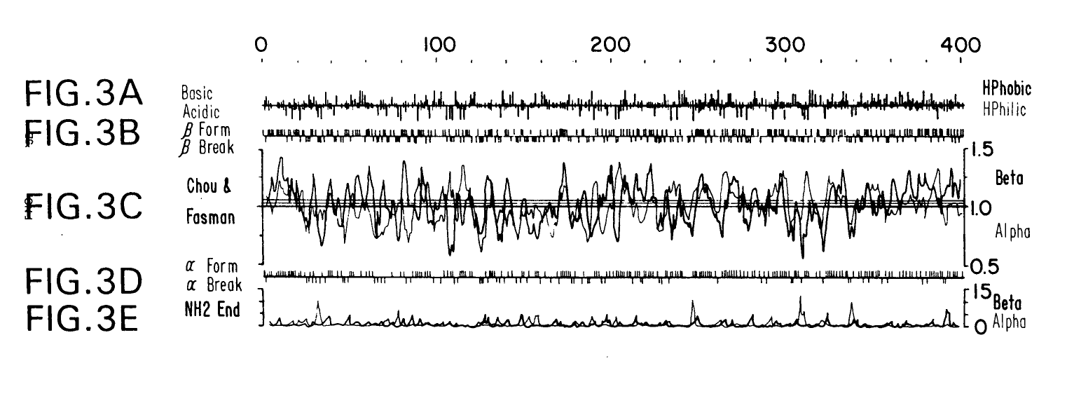

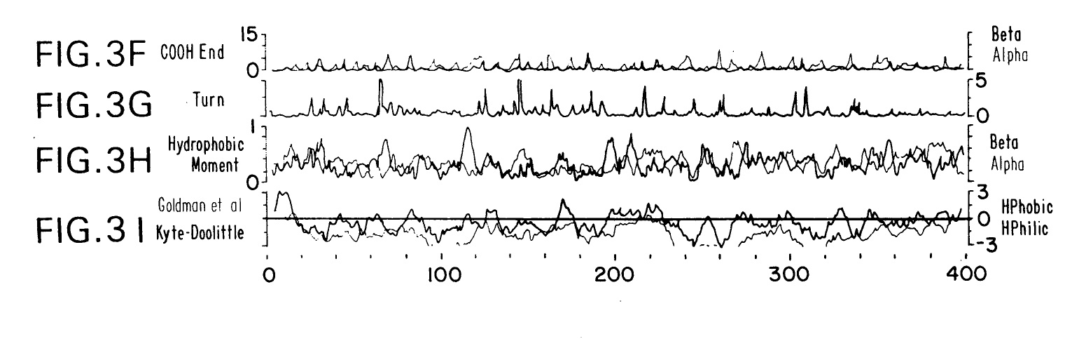

However, treatment with OPG-Fc (at all doses) had no statistically significant effect on the severity of inflammation (FIGS. 1 and 3, AUC) or loss of body weight (data on file).

Conclusion

In conclusion, the results demonstrate that OPG-Fc have great efficacy in preventing bone density loss in the tibiotarsal region in arthritic rats. The inhibitory effects of OPG-Fc against bone changes occurred without any anti-inflammatory actions.

### EXAMPLE 14

### Combination Treatment with OPG-Fc and sTNF-RI on Adjuvant Arthritis in Male Lewis Rats

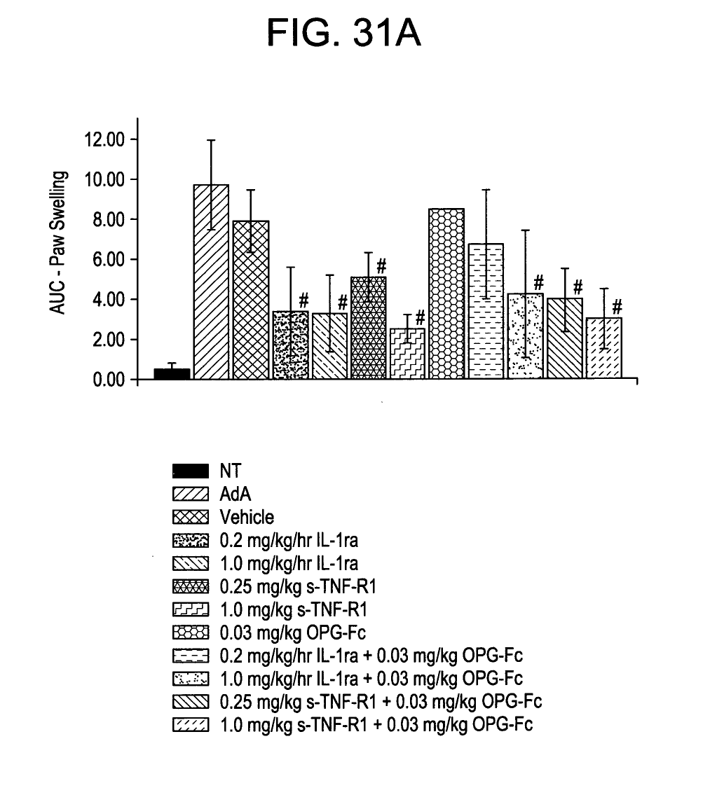

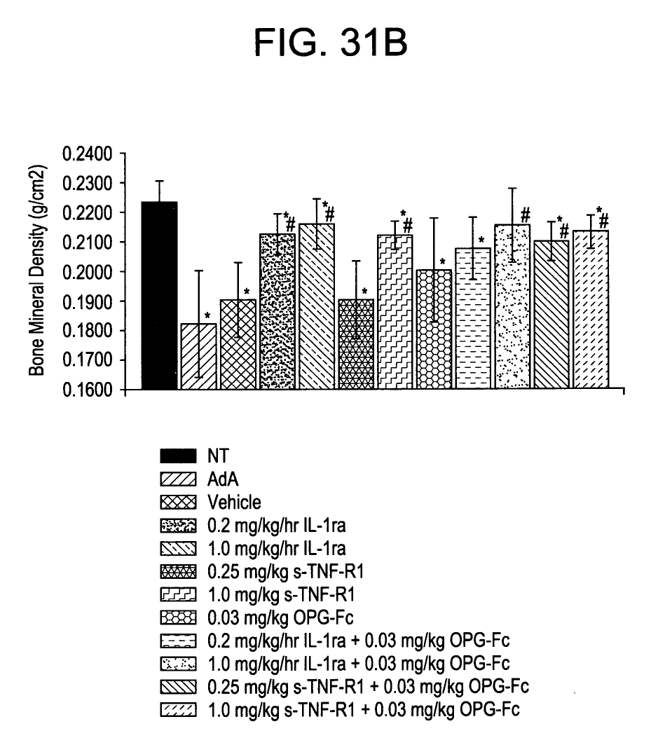

Male Lewis rats were injected with 0.5 mg heat-killed Mycobacterium tuberculosis H37Ra in mineral oil at the base of the tail. Rats were monitored for paw swelling and weight loss. Arthritis (paw swelling) developed after about 10 days. Paw swelling was calculated daily relative to paw volume on day 9 (beginning of treatment) and the area under the curve (AUC) from day 9 to 15 is given in the graph (FIG. 31A). On day 16 at the end of the experiment DexaScans of the rats were taken and the calcaneus was evaluated for loss of bone mineral density (BMD) as shown in FIG. 31B.

While the invention has been described in what is considered to be its preferred embodiments, it is not to be limited to the disclosed embodiments, but on the contrary, is intended to cover various modifications and equivalents included within the spirit and scope of the appended claims, which scope is to be accorded the broadest interpretation so as to encompass all such modifications and equivalents.

1-16. (canceled)

17. A method of treating bone loss, which comprises administering an IL-1 inhibitor, a TNF-α inhibitor, and an OPG protein wherein “OPG protein” refers to an antibody to OPG ligand or a polypeptide comprising conserved residues from residues 22 to 185 of SEQ ID NOS: 121,123, and 125.

18. The method of claim 17, wherein the TNF-α inhibitor comprises TNFR-I, sTNFR-II, sTNFR fragments, or sTNFR-Fc, wherein “sTNFR” refers to sTNFR-I or sTNFR-II.

19. The method of claim 17, wherein the TNF-α inhibitor comprises sTNFR-I, sTNFR-II, sTNFR fragments, or sTNFR-Fc.

20. The method of claim 17, wherein the TNF-α inhibitor comprises 30 kD PEG-sTNFR-I.

21. The method of claim 18, wherein the sTNFR fragment is a 2.6 kD sTNFR-I fragment.

22. The method of claim 21, wherein the sTNFR-I fragment comprises 30 kD PEG.

23. The method of claim 17, wherein the TNF-α inhibitor comprises sTNFR-II linked to an Fc region.

24. The method of claim 17, wherein the TNF-α inhibitor is etanercept.

25-38. (canceled)

39. The method of any of claims 17 to 24, wherein the bone loss results from arthritis.

40. The method of any of claims 17 to 24, wherein the bone loss results from multiple sclerosis.

41. The method of any of claims 17 to 24, wherein the bone loss results from osteoporosis.

42. The method of any of claims 17 to 24, wherein the bone loss results from osteomyelitis.

43-61. (canceled)


SEQUENCE LISTING (178 sequences)

SEQ ID NO: 1 - DNA, 36 bp, Artificial Sequence
  Not I restriction site
  misc_feature (28)..(35): N = any random nucleic acid
aaaggaaggaaaaaagcggccgctacannnnnnnnt

SEQ ID NO: 2 - DNA, 16 bp, Artificial Sequence
  Not I restriction site
tcgacccacgcgtccg

SEQ ID NO: 3 - DNA, 12 bp, Artificial Sequence
  Not I restriction site
gggtgcgcaggc

SEQ ID NO: 4 - DNA, 18 bp, Artificial Sequence
  Not I restriction site
tgtaaaacgacggccagt

SEQ ID NO: 5 - DNA, 18 bp, Artificial Sequence
  Not I restriction site
caggaaacagctatgacc

SEQ ID NO: 6 - DNA, 20 bp, Artificial Sequence
  Not I restriction site
caattaaccctcactaaagg

SEQ ID NO: 7 - DNA, 23 bp, Rattus rattus
gcattatgacccagaaaccggac

SEQ ID NO: 8 - DNA, 23 bp, Rattus rattus
aggtagcgcccttcctcacattc

SEQ ID NO: 9 - DNA, 30 bp, Artificial Sequence
  Oligonucleotide primer
gactagtcccacaatgaacaagtggctgtg

SEQ ID NO: 10 - DNA, 45 bp, Artificial Sequence
  Oligonucleotide primer
ataagaatgcggccgctaaactatgaaacagcccagtgaccattc

SEQ ID NO: 11 - DNA, 21 bp, Artificial Sequence
  Oligonucleotide primer
gcctctagaaagagctgggac

SEQ ID NO: 12 - DNA, 21 bp, Artificial Sequence
  Oligonucleotide primer
cgccgtgttccatttatgagc

SEQ ID NO: 13 - DNA, 24 bp, Rattus rattus
atcaaaggcagggcatacttcctg

SEQ ID NO: 14 - DNA, 24 bp, Rattus rattus
gttgcactcctgtttcacggtctg

SEQ ID NO: 15 - DNA, 24 bp, Rattus rattus
caagacaccttgaagggcctgatg

SEQ ID NO: 16 - DNA, 24 bp, Rattus rattus
taacttttacagaagagcatcagc

SEQ ID NO: 17 - DNA, 33 bp, Rattus rattus
agcgcggccgcatgaacaagtggctgtgctgcg

SEQ ID NO: 18 - DNA, 31 bp, Rattus rattus
agctctagagaaacagcccagtgaccattcc

SEQ ID NO: 19 - DNA, 24 bp, Rattus rattus
gtgaagctgtgcaagaacctgatg

SEQ ID NO: 20 - DNA, 24 bp, Rattus rattus
atcaaaggcagggcatacttcctg

SEQ ID NO: 21 - DNA, 24 bp, Homo sapiens
cagatcctgaagctgctcagtttg

SEQ ID NO: 22 - DNA, 33 bp, Homo sapiens
agcgcggccgcggggaccacaatgaacaagttg

SEQ ID NO: 23 - DNA, 33 bp, Homo sapiens
agctctagaattgtgaggaaacagctcaatggc

SEQ ID NO: 24 - DNA, 39 bp, Artificial Sequence
  Not I restriction site
atagcggccgctgagcccaaatcttgtgacaaaactcac

SEQ ID NO: 25 - DNA, 45 bp, Artificial Sequence
  Not I restriction site
tctagagtcgacttatcatttacccggagacagggagaggctctt

SEQ ID NO: 26 - DNA, 38 bp, Mus musculus
cctctgagctcaagcttccgaggaccacaatgaacaag

SEQ ID NO: 27 - DNA, 43 bp, Mus musculus
cctctgcggccgctaagcagcttattttcacggattgaacctg

SEQ ID NO: 28 - DNA, 38 bp, Mus musculus; identical to SEQ ID NO: 26

SEQ ID NO: 29 - DNA, 24 bp, Homo sapiens
tccgtaagaaacagcccagtgacc

SEQ ID NO: 30 - DNA, 31 bp, Mus musculus
cctctgcggccgctgttgcatttcctttctg

SEQ ID NO: 31 - protein, 19 aa, Mus musculus
ETLPPKYLHYDPETGHQLL

SEQ ID NO: 32 - DNA, 21 bp, Mus musculus
tcccttgccctgaccactctt

SEQ ID NO: 33 - DNA, 34 bp, Mus musculus
cctctgcggccgcacacacgttgtcatgtgttgc

SEQ ID NO: 34 - DNA, 21 bp, Mus musculus
tcccttgccctgaccactctt

SEQ ID NO: 35 - DNA, 34 bp, Mus musculus
cctctgcggccgccttttgcgtggcttctctgtt

SEQ ID NO: 36 - DNA, 37 bp, Homo sapiens
cctctgagctcaagcttggtttccggggaccacaatg

SEQ ID NO: 37 - DNA, 38 bp, Homo sapiens
cctctgcggccgctaagcagcttatttttactgaatgg

SEQ ID NO: 38 - DNA, 37 bp, Homo sapiens; identical to SEQ ID NO: 36

SEQ ID NO: 39 - DNA, 33 bp, Homo sapiens
cctctgcggccgccagggtaacatctattccac

SEQ ID NO: 40 - DNA, 35 bp, Mus musculus
ccgaagcttccaccatgaacaagtggctgtgctgc

SEQ ID NO: 41 - DNA, 40 bp, Mus musculus
cctctgtcgactattataagcagcttattttcacggattg

SEQ ID NO: 42 - DNA, 21 bp, Mus musculus
tcccttgccctgaccactctt

SEQ ID NO: 43 - DNA, 35 bp, Mus musculus
cctctgtcgacttaacacacgttgtcatgtgttgc

SEQ ID NO: 44 - DNA, 21 bp, Mus musculus
tcccttgccctgaccactctt

SEQ ID NO: 45 - DNA, 35 bp, Mus musculus
cctctgtcgacttacttttgcgtggcttctctgtt

SEQ ID NO: 46 - DNA, 1548 bp, Artificial Sequence
  pAMG21
tgcacgcattgcatacgtaccagaggggtacgctctcatcccttgacggtccgtagtttattttgctttccgagtcagctttctgacccggaaagcaaaatagacaacaaacagccacttgcgagaggactcatcctgtttaggcggccctcgcctaaacttgcaacgcttcgttgccgggcctcccaccgcccgtcctgcgggcggtatttgacggtccgtagtttaattcgtcttccggtaggactgcctaccggaaaaacgcaaagatgtttgagaaaacaaataaaaagatttatgtaagtttatacctgcagcatgaattgaaaatttcatacccgttagttaacgaggacaattttaacgaaatctttatgaaaccgtcgccaaacaacataactcaaagtaaacgcgtaaccaatttacctttcactggcacgcgaatgatgtcggattataaaaactttatagggttctcgaaaaaggaagcgtacgggtgcgatttgtaagaaaaagagaaaaccaatttagcaacaaactaaataataaacgatataaataaaaagctattaatagttgatctcttccttgttaattaccatacaagtatgtgcgtacatttttatttgatagatatatcaacagaaagagacttacacgttttgattcgtaaggcttcggtaataatcgtcatacttatccctttgatttgggtcactattctggactactaaagcgaagaaattaatgtaaacctctaaaaaataaatgtcgtaacaaaagtttatataaggttaattagccacttactaacctcaatcttattagatgatatcctagtataaaataatttaatcgcagtagtattataacggaggtaaaaaatcccattaataggtcttaactttatagtctaaattggtatcttactcctatttactagcgctcatttattataagtgttacatggtaaaatcagtatagtctattcgtaactaattatagtaataacgaagatgtccgaaattaaaataattaataagacattcacagcagccgtaaatacagaaagtatgggtagagaaataggaatggataacaaacagcgttcaaaacgcacaatatatagtaattttgccattatctaactgtaaactaagattatttaacctaaaaacagtgtgataatatagcgaactttatgttaacaaattgtattcatggacatcctagcatgtccaaatgcgttcttttaccaaacaatatcagctaattagctaaactaagatctaaacaaaattgattaatttcctccttattgtataccaattgcgcaaccttaagctcgagtgatcacagctggacgtcccatggtaccttcgaatgagctcctaggcgcctttcttcttcttcttcttctttcgggctttccttcgactcaaccgacgacggtggcgactcgttattgatcgtattggggaaccccggagatttgcccagaactccccaaaaaacgactttcctccttggcgagaagtgcgagaagtg

SEQ ID NO: 47 - DNA, 48 bp, Homo sapiens
ccggcggacatttatcacacagcagctgatgagaagtttcttcatcca

SEQ ID NO: 48 - DNA, 55 bp, Artificial Sequence
  pAMG21
cgatttgattctagaaggaggaataacatatggttaacgcgttggaattcggtac

SEQ ID NO: 49 - DNA, 49 bp, Artificial Sequence
  pAMG21
taaactaagatcttcctccttattgtataccaattgcgcaaccttaagc

SEQ ID NO: 50 - DNA, 1546 bp, Artificial Sequence
  pAMG21
  misc_feature (1, 2, 1545 )..(1546): Unique AatII and SacII sticky ends
gcgtaacgtatgcatggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatggacgtcgtacttaacttttaaagtatgggcaatcaattgctcctgttaaaattgctttagaaatactttggcagcggtttgttgtattgagtttcatttgcgcattggttaaatggaaagtgaccgtgcgcttactacagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttctctgaatgtgcaaaactaagcattccgaagccattattagcagtatgaatagggaaactaaacccagtgataagacctgatgatttcgcttctttaattacatttggagattttttatttacagcattgttttcaaatatattccaattaatcggtgaatgattggagttagaataatctactataggatcatattttattaaattagcgtcatcataatattgcctccattttttagggtaattatccagaattgaaatatcagatttaaccatagaatgaggataaatgatcgcgagtaaataatattcacaatgtaccattttagtcatatcagataagcattgattaatatcattattgcttctacaggctttaattttattaattattctgtaagtgtcgtcggcatttatgtctttcatacccatctctttatccttacctattgtttgtcgcaagttttgcgtgttatatatcattaaaacggtaatagattgacatttgattctaataaattggatttttgtcacactattatatcgcttgaaatacaattgtttaacataagtacctgtaggatcgtacaggtttacgcaagaaaatggtttgttatagtcgattaatcgatttgattctagatttgttttaactaattaaaggaggaataacatatggttaacgcgttggaattcgagctcactagtgtcgacctgcagggtaccatggaagcttactcgaggatccgcggaaagaagaagaagaagaagaaagcccgaaaggaagctgagttggctgctgccaccgctgagcaataactagcataaccccttggggcctctaaacgggtcttgaggggttttttgctgaaaggaggaaccgctcttcacgctcttcacgc

SEQ ID NO: 51 - DNA, 47 bp, Artificial Sequence
  pAMG22
tatgaaacatcatcaccatcaccatcatgctagcgttaacgcgttgg

SEQ ID NO: 52 - DNA, 49 bp, Artificial Sequence
  pAMG22
actttgtagtagtggtagtggtagtacgatcgcaattgcgcaaccttaa

SEQ ID NO: 53 - DNA, 141 bp, Artificial Sequence
  pAMG22
ctaattccgctctcacctaccaaacaatgcccccctgcaaaaaataaattcatataaaaaacatacagataaccatctgcggtgataaattatctctggcggtgttgacataaataccactggcggtgatactgagcacat

SEQ ID NO: 54 - DNA, 147 bp, Artificial Sequence
  pAMG22
tgcagattaaggcgagagtggatggtttgttacggggggacgttttttatttaagtatattttttgtatgtctattggtagacgccactatttaatagagaccgccacaactgtatttatggtgaccgccactatgactcgtgtagc

SEQ ID NO: 55 - DNA, 55 bp, Artificial Sequence; identical to SEQ ID NO: 48
  pAMG22

SEQ ID NO: 56 - DNA, 49 bp, Artificial Sequence; identical to SEQ ID NO: 49
  pAMG22

SEQ ID NO: 57 - DNA, 668 bp, Artificial Sequence
  pAMG22
tgcacgcattgcatacgtaccagaggggtacgctctcatcccttgacggtccgtagtttattttgctttccgagtcagctttctgacccggaaagcaaaatagacaacaaacagccacttgcgagaggactcatcctgtttaggcggccctcgcctaaacttgcaacgcttcgttgccgggcctcccaccgcccgtcctgcgggcggtatttgacggtccgtagtttaattcgtcttccggtaggactgcctaccggaaaaacgcaaagatgtttgagaaaacaaataaaaagatttatgtaagtttatacctgcagagtattaaaaattttttaagtaaactgtttacgattttaagaactaattataagagttaacactcgcgagtgttaaatagctaaactaagatctaaactcaattgattaatttcctccttattgtataccaattgcgcaaccttaagctcgagtgatcacagctggacgtcccatggtaccttcgaatgagctcctaggcgcctttcttcttcttcttcttctttcgggctttccttcgactcaaccgacgacggtggcgactcgttattgatcgtattggggaaccccggagatttgcccagaactccccaaaaaacgactttcctccttggcgagaagtgcgagaagtg

SEQ ID NO: 58 - DNA, 726 bp, Artificial Sequence
  pAMG22
gcgtaacgtatgcatggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggggcctcccaccgcccgtcctgcgggcggtatttgacggtccgtagtttaattcgtcttcgccatcctgacggatggcctttttgcgtttctacaaactcttttgtttatttttctaaatacattcaaatatggacgtctcataatttttaaaaaattcatttgacaaatgctaaaattcttgattaatattctcaattgtgagcgctcacaatttatcgatttgattctagatttgttttaactaattaaaggaggaataacatatggttaacgcgttggaattcgagctcactagtgtcgacctgcagggtaccatggaagcttactcgaggatccgcggaaagaagaagaagaagaagaaagcccgaaaggaagctgagttggctgctgccaccgctgagcaataactagcataaccccttggggcctctaaacgggtcttgaggggttttttgctgaaaggaggaaccgctcttcacgctcttcacgc

SEQ ID NO: 59 - DNA, 44 bp, Homo sapiens
tacgcactggatccttataagcagcttatttttactgattggac

SEQ ID NO: 60 - DNA, 27 bp, Homo sapiens
gtcctcctggtacctacctaaaacaac

SEQ ID NO: 61 - DNA, 54 bp, Homo sapiens
tatggatgaagaaacttctcatcagctgctgtgtgataaatgtccgccgggtac

SEQ ID NO: 62 - protein, 19 aa, Homo sapiens
MDEETSHQLLCDKCPPGTY

SEQ ID NO: 63 - DNA, 84 bp, Artificial Sequence
  pAMG21
tatggaaacttttcctccaaaatatcttcattatgatgaagaaacttctcatcagctgctgtgtgataaatgtccgccgggtac

SEQ ID NO: 64 - DNA, 78 bp, Artificial Sequence
  pAMG21
ccggcggacatttatcacacagcagctgatgagaagtttcttcatcataatgaagatattttggaggaaaagtttcca

SEQ ID NO: 65 - DNA, 44 bp, Artificial Sequence
  pAMG21-MuOPG
tacgcactggatccttataagcagcttattttcacggattgaac

SEQ ID NO: 66 - DNA, 38 bp, Artificial Sequence
  pAMG21-MuOPG
gtgctcctggtacctacctaaaacagcactgcacagtg

SEQ ID NO: 67 - DNA, 84 bp, Artificial Sequence
  pAMG21-MuOPG
tatggaaactctgcctccaaaatacctgcattacgatccggaaactggtcatcagctgctgtgtgataaatgtgctccgggtac

SEQ ID NO: 68 - DNA, 78 bp, Artificial Sequence
  pAMG21-MuOPG
ccggagcacatttatcacacagcagctgatgaccagtttccggatcgtaatgcaggtattttggaggcagagtttcca

SEQ ID NO: 69 - DNA, 54 bp, Mus musculus
tatggacccagaaactggtcatcagctgctgtgtgataaatgtgctccgggtac

SEQ ID NO: 70 - DNA, 48 bp, Mus musculus
ccggagcacatttatcacacagcagctgatgaccagtttctgggtcca

SEQ ID NO: 71 - DNA, 87 bp, Artificial Sequence
  pAMG21
tatgaaagaaactctgcctccaaaatacctgcattacgatccggaaactggtcatcagctgctgtgtgataaatgtgctccgggtac

SEQ ID NO: 72 - DNA, 81 bp, Artificial Sequence
  pAMG21
ccggagcacatttatcacacagcagctgatgaccagtttccggatcgtaatgcaggtattttggaggcagagtttctttca

SEQ ID NO: 73 - DNA, 71 bp, Artificial Sequence
  pAMG21
gttctcctcatatgaaacatcatcaccatcaccatcatgaaactctgcctccaaaatacctgcattacgat

SEQ ID NO: 74 - DNA, 43 bp, Mus musculus
gttctcctcatatgaaagaaactctgcctccaaaatacctgca

SEQ ID NO: 75 - DNA, 76 bp, Mus musculus
tacgcactggatccttaatgatggtgatggtgatgatgtaagcagcttattttcacggattgaacctgattcccta

SEQ ID NO: 76 - DNA, 47 bp, Mus musculus
gttctcctcatatgaaatacctgcattacgatccggaaactggtcat

SEQ ID NO: 77 - DNA, 43 bp, Homo sapiens
gttctcctattaatgaaatatcttcattatgatgaagaaactt

SEQ ID NO: 78 - DNA, 40 bp, Homo sapiens
tacgcactggatccttataagcagcttatttttactgatt

SEQ ID NO: 79 - DNA, 40 bp, Mus musculus
gttctcctcatatggaaactctgcctccaaaatacctgca

SEQ ID NO: 80 - DNA, 43 bp, Mus musculus
tacgcactggatccttatgttgcatttcctttctgaattagca

SEQ ID NO: 81 - DNA, 18 bp, Artificial Sequence
  pAMG21
ccggaaacagataatgag

SEQ ID NO: 82 - DNA, 18 bp, Artificial Sequence
  pAMG21
gatcctcattatctgttt

SEQ ID NO: 83 - DNA, 30 bp, Artificial Sequence
  pAMG21
ccggaaacagagaagccacgcaaaagtaag

SEQ ID NO: 84 - DNA, 30 bp, Artificial Sequence
  pAMG21
gatccttacttttgcgtggcttctctgttt

SEQ ID NO: 85 - DNA, 12 bp, Artificial Sequence
  pAMG21
tatgttaatgag

SEQ ID NO: 86 - DNA, 14 bp, Artificial Sequence
  pAMG21
gatcctcattaaca

SEQ ID NO: 87 - DNA, 21 bp, Artificial Sequence
  pAMG21
tatgttccggaaacagttaag

SEQ ID NO: 88 - DNA, 23 bp, Artificial Sequence
  pAMG21
gatccttaactgtttccggaaca

SEQ ID NO: 89 - DNA, 36 bp, Artificial Sequence
  pAMG21
tatgttccggaaacagtgaatcaactcaaaaataag

SEQ ID NO: 90 - DNA, 38 bp, Artificial Sequence
  pAMG21
gatccttatttttgagttgattcactgtttccggaaca

SEQ ID NO: 91 - DNA, 100 bp, Artificial Sequence
  pAMG21
ctagcgacgacgacgacaaagaaactctgcctccaaaatacctgcattacgatccggaaactggtcatcagctgctgtgtcataaatgtgctccgggtac

SEQ ID NO: 92 - DNA, 92 bp, Artificial Sequence
  pAMG21
ccggagcacatttatcacacagcagctgatgaccagtttccggatcgtaatgcaggtattttggaggcagagtttctttgtcgtcgtcgtcg

SEQ ID NO: 93 - DNA, 26 bp, Artificial Sequence
  pAMG21-huOPG
acaaacacaatcgatttgatactaga

SEQ ID NO: 94 - DNA, 50 bp, Artificial Sequence
  pAMG21-huOPG
tttgttttaactaattaaaggaggaataaaatatgagaggatcgcatcac

SEQ ID NO: 95 - DNA, 50 bp, Artificial Sequence
  pAMG21-huOPG
catcaccatcacgaaaccttcccgccgaaatacctgcactacgacgaaga

SEQ ID NO: 96 - DNA, 49 bp, Artificial Sequence
  pAMG21-huOPG
aacctcccaccagctgctgtgcgacaaatgcccgccgggtacccaaaca

SEQ ID NO: 97 - DNA, 26 bp, Artificial Sequence
  pAMG21-huOPG
tgtttgggtacccggcgggcatttgt

SEQ ID NO: 98 - DNA, 50 bp, Artificial Sequence
  pAMG21-huOPG
cgcacagcagctggtgggaggtttcttcgtcgtagtgcaggtatttcggc

SEQ ID NO: 99 - DNA, 49 bp, Artificial Sequence
  pAMG21-huOPG
gggaaggtttcgtgatggtgatggtgatgccatcctctcatattttatt

SEQ ID NO: 100 - DNA, 50 bp, Artificial Sequence
  pAMG21-huOPG
cctcctttaattagttaaaacaaatctagtatcaaatcgattgtgtttgt

SEQ ID NO: 101 - DNA, 59 bp, Homo sapiens
acaaacacaatcgatttgatactagatttgttttaactaattaaaggaggaataaaatg

SEQ ID NO: 102 - DNA, 48 bp, Homo sapiens
ctaattaaaggaggaataaaatgaaagaaacttttcctccaaaatatc

SEQ ID NO: 103 - DNA, 31 bp, Homo sapiens
tgtttgggtacccggcggacatttatcacac

SEQ ID NO: 104 - DNA, 59 bp, Homo sapiens; identical to SEQ ID NO: 101

SEQ ID NO: 105 - DNA, 54 bp, Homo sapiens
ctaattaaaggaggaataaaatgaaaaaaaaagaaacttttcctccaaaatatc

SEQ ID NO: 106 - DNA, 31 bp, Homo sapiens; identical to SEQ ID NO: 103

SEQ ID NO: 107 - DNA, 44 bp, Artificial Sequence
  PCR primer for Fc-hOPG fusion protein.
cagcccgggtaaaatggaaacgtttcctccaaaatatcttcatt

SEQ ID NO: 108 - DNA, 44 bp, Artificial Sequence
  PCR primer for FchOPG fusion protein.
cgtttccattttacccgggctgagcgagaggctcttctgcgtgt

SEQ ID NO: 109 - DNA, 45 bp, Artificial Sequence
  Fc/muOPG
cgctcagcccgggtaaaatggaaacgttgcctccaaaatacctgc

SEQ ID NO: 110 - DNA, 39 bp, Artificial Sequence
  Fc/muOPG
ccattttacccgggctgagcgagaggctcttctgcgtgt

SEQ ID NO: 111 - DNA, 36 bp, Artificial Sequence
  muOPG
gaaaataagatgcttagctgcagctgaaccaaaatc

SEQ ID NO: 112 - DNA, 34 bp, Artificial Sequence
  muOPG
cagctgcagctaagcagcttattttcacggattg

SEQ ID NO: 113 - DNA, 36 bp, Artificial Sequence
  huOPG
aaaaataagctgcttagctgcagctgaaccaaaatc

SEQ ID NO: 114 - DNA, 35 bp, Artificial Sequence
  huOPG
cagctgcagctaagcagcttatttttactgattgg

SEQ ID NO: 115 - DNA, 102 bp, Artificial Sequence
  pAMG21-huOPG
  misc_feature: Linker with XbaI and KpnI sites inserted into human sequence.
ctagaaggaggaataacatatggaaacttttgctccaaaatatcttcattatgatgaagaaactagtcatcagctgctgtgtgataaatgtccgccgggtac

SEQ ID NO: 116 - DNA, 94 bp, Artificial Sequence
  huOPG
ccggcggacatttatcacacagcagctgatgactagtttcttcatcataatgaagatattttggagcaaaagtttccatatgttattcctcctt

SEQ ID NO: 117 - DNA, 62 bp, Artificial Sequence
  huOPG
ctagaaggaggaataacatatggaaacttttcctgctaaatatcttcattatgatgaagaaa

SEQ ID NO: 118 - DNA, 62 bp, Artificial Sequence
  huOPG
ctagtttcttcatcataatgaagatatttagcaggaaaagtttccatatgttattcctcctt

SEQ ID NO: 119 - protein, 51 aa, Homo sapiens
YHYYDQNGRMCEECHMCQPGHFLVKHCKQPKRDTVCHKPCEPGVTYTDDWH

SEQ ID NO: 120 - DNA, 2432 bp, Rattus rattus
  CDS (124)..(1326)
atcaaaggcagggcatacttcctgttgcccagaccttatataaaacgtcatgttcgcctgggcagcagagaagcacctagcactggcccagcggctgccgcctgaggtttccagaggaccacaatgaacaagtggctgtgctgtgcactcctggtgttcttggacatcattgaatggacaacccaggaaacctttcctccaaaatacttgcattatgacccagaaaccggacgtcagctcttgtgtgacaaatgtgctcctggcacctacctaaaacagcactgcacagtcaggaggaagacactgtgtgtcccttgccctgactactcttatacagacagctggcacacgagtgatgaatgcgtgtactgcagccccgtgtgcaaggaactgcagaccgtgaaacaggagtgcaaccgcacccacaaccgagtgtgcgaatgtgaggaagggcgctacctggagctcgaattctgcttgaagcaccggagctgtcccccaggcttgggtgtgctgcaggctgggaccccagagcgaaacacggtttgcaaaagatgtccggatgggttcttctcaggtgagacgtcatcgaaagcaccctgtaggaaacacaccaactgcagctcacttggcctcctgctaattcagaaaggaaatgcaacacatgacaatgtatgttccggaaacagagaagcaactcaaaattgtgaaatagatgtcaccctgtgcgaagaggcattcttcaggtttgctgtgcctaccaagattataccgaattggctgagtgttctggtggacagtttgcctgggaccaaagtgaatgcagagagtgtagagaggataaaacggagacacagctcgcaagagcaaactttccagctacttaagctgtggaagcatcaaaacagagaccaggaaatggtgaagaagatcatccaagacattgacctctgtgaaagcagtgtgcaacggcatatcggccacgcgaacctcaccacagagcagctccgcatcttgatggagagcttgcctgggaagaagatcagcccagacgagattgagagaacgagaaagacctgcaaacccagcgagcagctcctgaagctactgagcttgtggaggatcaaaaatggagaccaagacaccttgaagggcctgatgtacgcactcaagcacttgaaagcataccactttcccaaaaccgtcacccacagtctgaggaagaccatcaggttcttgcacagcttcaccatgtaccgattgtatcagaaactctttctagaaatgatagggaatcaggttcaatcagtgaagataagctgcttatagttaggaatggtcactgggctgtttcttcaggatgggccaacactgatggagcagatggctgcttctccggctcttgaaatggcagttgattcctttctcatcagttggtgggaatgaagatcctccagcccaacacacacactggggagtctgagtcaggagagtgaggcaggctatttgataattgtgcaaagctgccaggtgtacacctagaaagtcaagcaccctgagaaagaggatatttttataacctcaaacataggccctttccttcctctccttatggatgagtactcagaaggcttctactatcttctgtgtcatccctagatgaaggcctcttttatttatttttttattctttttttcggagctggggaccgaacccagggccttgcgcttgcgaggcaagtgctctaccactgagctaaatctccaacccctgaaggcctctttctttctgcctctgatagtctatgacattcttttttctacaattcgtatcaggtgcacgagccttatcccatttgtaggtttctaggcaagttgaccgttagctatttttccctctgaagatttgattcgagttgcagacttggctagacaagcaggggtaggttatggtagtttatttaacagactgccaccaggagtccagtgtttcttgttcctctgtagttgtacctaagctgactccaagtacatttagtatgaaaaataatcaacaaattttattccttctatcaacattggctagctttgtttcagggcactaaaagaaactactatatggagaaagaattgatattgcccccaacgttcaacaacccaatagtttatccagctgtcatgcctggttcagtgtctactgactatgcgccctcttattactgcatgcagtaattcaactggaaatagtaataataataatagaaataaaatctagactccattggatctctctgaatatgggaatatctaacttaagaagctttgagatttcagttgtgttaaaggcttttattaaaaagctgatgctcttctgtaaaagttactaatatatctgtaagactattacagtattgctatttatatccatccag

SEQ ID NO: 121 - protein, 401 aa, Rattus rattus
MNKWLCCALLVFLDIIEWTTQETFPPKYLHYDPETGRQLLCDKCAPGTYLKQHCTVRRKTLCVPCPDYSYTDSWHTSDECVYCSPVCKELQTVKQECNRTHNRVCECEEGRYLELEFCLKHRSCPPGLGVLQAGTPERNTVCKRCPDGFFSGETSSKAPCRKHTNCSSLGLLLIQKGNATHDNVCSGNREATQNCEIDVTLCEEAFFRFAVPTKIIPNWLSVLVDSLPGTKVNAESVERIKRRHSSQEQTFQLLKLWKHQNRDQEMVKKIIQDIDLCESSVQRHIGHANLTTEQLRILMESLPGKKISPDEIERTRKTCKPSEQLLKLLSLWRIKNGDQDTLKGLMYALKHLKAYHFPKTVTHSLRKTIRFLHSFTMYRLYQKLFLEMIGNQVQSVKISCL

SEQ ID NO: 122 - DNA, 1325 bp, Mus musculus
  misc_feature (11)..(11): At position 11, R is a purine.
  CDS (91)..(1293)
ccttatataaracgtcatgattgcctgggctgcagagacgcacctagcactgacccagcggctgcctcctgaggtttcccgaggaccacaatgaacaagtggctgtgctgcgcactcctggtgctcctggacatcattgaatggacaacccaggaaacccttcttccaaagtacttgcattatgacccagaaactggtcatcagctcctgtgtgacaaatgtgctcctggcacctacctaaaacagcactgcacagtgaggaggaagacattgtgtgtcccttgccctgaccactcttatacggacagctggcacaccagtgatgagtgtgtgtattgcagcccagtgtgcaaggaactgcagtccgtgaagcaggagtgcaaccgcacccacaaccgagtgtgtgagtgtgaggaagggcgttacctggagatcgaattctgcttgaagcaccggagctgtcccccgggctccggcgtggtgcaagctggaaccccagagcgaaacacagtttgcaaaaaatgtccagatgggttcttctcaggtgagacttcatcgaaagcaccctgtataaaacacacgaactgcagcacatttggcctcctgctaattcagaaaggaaatgcaacacatgacaactgttgttccggaaacagagaagccacgcaaaagtgtggaatagatgtcaccctgtgtgaagaggccttcttcaggtttgctgttcctaccaagattataccaaattggctgagtgttttggtggacagtttgcctgggaccaaagtgaatgccgagagtgtagagaggataaaacggagacacagctcacaagagcaaaccttccagctgctgaagctgtggaaacatcaaaacagagaccaggaaatggtgaagaagatcatccaagacattgacctctgtgaaagcagcgtgcagcggcatctcggccactcgaacctcaccacagagcagcttcttgccttgatggagagcctgcctgggaagaagatcagcccagaagagattgagagaacgagaaagacctgcaaatcgagcgagcagctcctgaagctactcagtttatggaggatcaaaaatggtgaccaagacaccttgaagggcctgatgtatgccctcaagcacttgaaaacatcccactttcccaaaactgtcacccacagtctgaggaagaccatgaggttcctgcacagcttcacaatgtacagactgtatcagaagctctttttagaaatgatagggaatcaggttcaatccgtgaaaataagctgcttataactaggaatggtcactgggctgtttcttca

SEQ ID NO: 123 - protein, 401 aa, Mus musculus
MNKWLCCALLVLLDIIEWTTQETLLPKYLHYDPETGHQLLCDKCAPGTYLKQHCTVRRKTLCVPCPDHSYTDSWHTSDECVYCSPVCKELQSVKQECNRTHNRVCECEEGRYLEIEFCLKHRSCPPGSGVVQAGTPERNTVCKKCPDGFFSGETSSKAPCIKHTNCSTFGLLLIQKGNATHDNCCSGNREATQKCGIDVTLCEEAFFRFAVPTKIIPNWLSVLVDSLPGTKVNAESVERIKRRHSSQEQTFQLLKLWKHQNRDQEMVKKIIQDIDLCESSVQRHLGHSNLTTEQLLALMESLPGKKISPEEIERTRKTCKSSEQLLKLLSLWRIKNGDQDTLKGLMYALKHLKTSHFPKTVTHSLRKTMRFLHSFTMYRLYQKLFLEMIGNQVQSVKISCL

SEQ ID NO: 124 - DNA, 1356 bp, Homo sapiens
  misc_feature (63)..(63): At position 63, Y is a pyrimidine.
  CDS (95)..(1297)
gtatatataacgtgatgagcgtacgggtgcggagacgcaccggcgcgctcgcccagccgccgyctccaagcccctgaggtttccggggaccacaatgaacaagttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagtccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaagcccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagcttcacaatgtacaaattgtatcagaagttatttttagaaatgataggtaaccaggtccaatcagtaaaaataagctgcttataactggaaatggccattgagctgtttcctcacaattggcgagatcccatggatgataa

SEQ ID NO: 125 - protein, 401 aa, Homo sapiens
MNKLLCCALVFLDISIKWTTQETFPPKYLHYDEETSHQLLCDKCPPGTYLKQHCTAKWKSVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKAQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 126 - protein, 139 aa, Homo sapiens
CPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSC

SEQ ID NO: 127 - DNA, 48 bp, Artificial Sequence
  huOPG
acctacttctttgaagagtagtcgacgacacactatttacaggcggcc

SEQ ID NO: 128 - protein, 219 aa, Rattus rattus
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPET

SEQ ID NO: 129 - protein, 280 aa, Rattus rattus
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMTRTQRWKSKLYSIVCGKSTPEKEGELEGTTTKPLAPNPSFSPTPGFT

SEQ ID NO: 130 - protein, 207 aa, Rattus rattus
MLRLIALLVCVVYVYGDDVPYSSNQGKCGGHDYEKDGLCCASCHPGFYASRLCGPGSNTVCSPCEDGTFTASTNHAPACVSCRGPCTGHLSESQPCDRTHDRVCNCSTGNYCLLKGQNGCRICAPQTKCPAGYGVSGHTRAGDTLCEKCPPHTYSDSLSPTERCGTSFNYISVGFNLYPVNETSCTTTAGHNEVIKTKEFTVTLNYT

SEQ ID NO: 131 - protein, 227 aa, Rattus rattus
MAPVAVWAALAVGLELWAAAHALPAQVAFTPYAPEPGSTCRLRETTDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASRDAVCTSTSPTRSMAPGAVHLPQPVSTRSQHT

SEQ ID NO: 132 - protein, 197 aa, Rattus rattus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLV

SEQ ID NO: 133 - protein, 208 aa, Rattus rattus
MNKWLCCALLVFLDIIEWTTQETFPPKYLHYDPETGRQLLCDKCAPGTYLKQHCTVRRKTLCVPCPDYSYTDSWHTSDECVYCSPVCKELQTVKQECNRTHNRVCECEEGRYLELEFCLKHRSCPPGLGVLQAGTPERNTVCKRCPDGFFSGETSSKAPCRKHTNCSSLGLLLIQKGNATHDNVCSGNREATQNCGIDVTLCEEAFFR

SEQ ID NO: 134 - protein, 224 aa, Rattus rattus
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLI

SEQ ID NO: 135 - protein, 202 aa, Rattus rattus
MYVWVQQPTAFLLLGLSLGVTVKLNCVKDTYPSGHKCCRECQPGHGMVSRCDHTRDTVCHPCEPGFYNEAVNYDTCKQCTQCNHRSGSELKQNCTPTEDTVCQCRPGTQPRQDSSHKLGVDCVPCPPGHFSPGSNQACKPWTNCTLSGKQIRHPASNSVCEDRSLLATLLWETQRTTFRPTTVPSTTVWPRTSQLPSTPTLV

SEQ ID NO: 136 - protein, 191 aa, Rattus rattus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFL

SEQ ID NO: 137 - DNA, 54 bp, Artificial Sequence; identical to SEQ ID NO: 61
  huOPG

SEQ ID NO: 138 - protein, 120 aa, Homo sapiens
HALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPL

SEQ ID NO: 139 - protein, 380 aa, Homo sapiens
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKAQDIVKKIIQDIDLCENSVQRHIGHANLTFEQLRSLMESLPGKKVGAEDIEKTIKACKPSDQILKLLSLWRIKNGDQDTLKGLMHALKHSKTKHFPKTVTQSLKKTIRFLHSFTMYKLYQKLFLEMIGNQVQSVKISCL

SEQ ID NO: 140 - DNA, 30 bp, Artificial Sequence
  huOPG
tggaccacccagaagtaccttcattatgac

SEQ ID NO: 141 - DNA, 30 bp, Artificial Sequence
  huOPG
gtcataatgaaggtacttctgggtggtcca

SEQ ID NO: 142 - DNA, 31 bp, Artificial Sequence
  huOPG
ggaccacccagcttcattatgacgaagaaac

SEQ ID NO: 143 - DNA, 31 bp, Artificial Sequence
  huOPG
gtttcttcgtcataatgaagctgggtggtcc

SEQ ID NO: 144 - DNA, 29 bp, Artificial Sequence
  huOPG
gtggaccacccaggacgaagaaacctctc

SEQ ID NO: 145 - DNA, 29 bp, Artificial Sequence
  huOPG
gagaggtttcttcgtcctgggtggtccac

SEQ ID NO: 146 - DNA, 29 bp, Artificial Sequence
  huOPG
cgtttcctccaaagttccttcattatgac

SEQ ID NO: 147 - DNA, 29 bp, Artificial Sequence
  huOPG
gtcataatgaaggaactttggaggaaacg

SEQ ID NO: 148 - DNA, 32 bp, Artificial Sequence
  huOPG
ggaaacgtttcctgcaaagtaccttcattatg

SEQ ID NO: 149 - DNA, 32 bp, Artificial Sequence
  huOPG
cataatgaaggtactttgcaggaaacgtttcc

SEQ ID NO: 150 - DNA, 27 bp, Artificial Sequence
  muOPG
cacgcaaaagtcgggaatagatgtcac

SEQ ID NO: 151 - DNA, 27 bp, Artificial Sequence
  muOPG
gtgacatctattcccgacttttgcgtg

SEQ ID NO: 152 - DNA, 25 bp, Artificial Sequence
  muOPG
caccctgtcggaagaggccttcttc

SEQ ID NO: 153 - DNA, 25 bp, Artificial Sequence
  muOPG
gaagaaggcctcttccgacagggtg

SEQ ID NO: 154 - DNA, 24 bp, Artificial Sequence
  muOPG
tgacctctcggaaagcagcgtgca

SEQ ID NO: 155 - DNA, 24 bp, Artificial Sequence
  muOPG
tgcacgctgctttccgagaggtca

SEQ ID NO: 156 - DNA, 24 bp, Artificial Sequence
  muOPG
cctcgaaatcgagcgagcagctcc

SEQ ID NO: 157 - DNA, 25 bp, Artificial Sequence
  muOPG
cgatttcgaggtctttctcgttctc

SEQ ID NO: 158 - DNA, 33 bp, Artificial Sequence
  muOPG
ccgtgaaaataagctcgttataactaggaatgg

SEQ ID NO: 159 - DNA, 33 bp, Artificial Sequence
  muOPG
ccattcctagttataacgagcttattttcacgg

SEQ ID NO: 160 - DNA, 38 bp, Artificial Sequence; identical to SEQ ID NO: 26
  muOPG

SEQ ID NO: 161 - DNA, 44 bp, Artificial Sequence
  muOPG
cctctctcgagtcaggtgacatctattccacacttttgcgtggc

SEQ ID NO: 162 - DNA, 38 bp, Artificial Sequence; identical to SEQ ID NO: 26
  muOPG

SEQ ID NO: 163 - DNA, 38 bp, Artificial Sequence
  muOPG
cctctctcgagtcaaggaacagcaaacctgaagaaggc

SEQ ID NO: 164 - DNA, 38 bp, Artificial Sequence; identical to SEQ ID NO: 26
  muOPG

SEQ ID NO: 165 - DNA, 38 bp, Artificial Sequence
  muOPG
cctctctcgagtcactctgtggtgaggttcgagtggcc

SEQ ID NO: 166 - DNA, 38 bp, Artificial Sequence; identical to SEQ ID NO: 26
  muOPG

SEQ ID NO: 167 - DNA, 38 bp, Artificial Sequence
  muOPG
cctctctcgagtcaggatgttttcaagtgcttgagggc

SEQ ID NO: 168 - protein, 16 aa, Artificial Sequence
  pAMG22
MKHHHHHHHASVNALE

SEQ ID NO: 169 - protein, 70 aa, Homo sapiens
ALLVFLDIIEWTTQETFPPKYLHYDPETGRQLLCDKCAPGTYLKQHCTVRRKTLCVPCPDYSYTDSWHTS

SEQ ID NO: 170 - protein, 48 aa, Homo sapiens
YLHYDPETGRQLLCDKCAPGTYLKQHCTVRRKTLCVPCPDYSYTDSWH

SEQ ID NO: 171-173 - skipped (3 entries)

SEQ ID NO: 174 - protein, 139 aa, Homo sapiens
PPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKH

SEQ ID NO: 175 - protein, 380 aa, Mus musculus
ETLPPKYLHYDPETGHQLLCDKCAPGTYLKQHCTVRRKTLCVPCPDHSYTDSWHTSDECVYCSPVCKELQSVKQECNRTHNRVCECEEGRYLEIEFCLKHRSCPPGSGVVQAGTPERNTVCKKCPDGFFSGETSSKAPCIKHTNCSTFGLLLIQKGNATHDNVCSGNREATQKCGIDVTLCEEAFFRFAVPTKIIPNWLSVLVDSLPGTKVNAESVERIKRRHSSQEQTFQLLKLWKHQNRDQEMVKKIIQDIDLCESSVQRHLGHSNLTTEQLLALMESLPGKKISPEEIERTRKTCKSSEQLLKLLSLWRIKNGDQDTLKGLMYALKHLKTSHFPKTVTHSLRKTMRFLHSFTMYRLYQKLFLEMIGNQVQSVKISCL

SEQ ID NO: 176 - DNA, 6037 bp, Homo sapiens
  CDS (4760)..(6025)
catgggaaatgtcagagtggagaaccacaccgagtgccactgcagcacttgttattatcacaaatcctaatagtttgcagtgggccttgctgatgatggctgacttgctcaaaaggaaaattaatttgtccagtgtctatggctttgtgagataaaaccctccttttccttgccataccatttttaacctgctttgagaatatactgcagctttattgcttttctccttatcctacaatataatcagtagtcttgatcttttcatttggaatgaaatatggcatttagcatgaccataaaaagctgattccactggaaataaagtcttttaaatcatcactctatcactgaattctaattttttctgaaaagtttcaagccagttacttttgataggattaacggaagggagtgagccagtgggtgaggtgggttcccatgtagtcaatggcctaatactggagaatcttattctaaccaagccttccagagcaagctgtgagcccctcagacagtgggctactcatgagacagtccattggggtaaaggaagaaaatataacttctatttctattcatttgcacattgtctttagatgcccatttgggtgagttttatagaagtacagctacattaaaaaatagaactgataatagataaggctttaaaaaaacttcattcatcaccagtttgtcaagattccatttcaaagtgaaaaaccaatttctaacgggttggtaaacacagcagatggcagggtgaaaaattaaagtgagtgcatgtacctttaaagaaacactgaaatgcacacacattacttaacctgctcattcatttatttacatatagtcttgggtgtacaaaatttagaaataaatacatatgggggcggggccttagctgcacaaataggatgcgcggcgggccttggtaggggcggagccttagctgcacaaataggatgcgcggcgggccttggtgggggcggggcctaagctgcgcaagtggtacacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatctgaaacactgaaattgtctgcttctcatcttcagtgagattccaaaggatagtacagtgacagaacaagaataggcactctctacaaaaaaaagaaagaaaaaactaagtaatagcaagcataatagctactgttaagaactcagagataatgaattgagaatggatactgcttgaaatgaaaatttaataagttagaaactaaactttataaaaataaaaaaatgagcattaaaaaaaaaaaaaaaaaaaaaaaaaaacccccccccccccccctgcagccaagctagcttggaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggtcgaggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccgtcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgctgcaacaagcttctagaccaccatgaacaagttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagaccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaagtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataactcgac

SEQ ID NO: 177 - protein, 422 aa, Homo sapiens
MNKLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 51 aa, Homo sapiens; identical to SEQ ID NO: 119